(19) United States
(12) Patent Application Publication
(10) Pub. No.: US 2010/0047827 A1
(43) Pub. Date: Feb. 25, 2010

(54) NOVEL SPECIFIC CELL BINDERS

(75) Inventors: Jarmo Laine, Helsinki (FI); Tero Satomaa, Helsinki (FI); Jari Natunen, Vantaa (FI); Annamari Heiskanen, Helsinki (FI); Maria Blomqvist, Itasalmi (FI); Anne Olonen, Lahti (FI); Juhani Saarinen, Helsinki (FI); Sari Titinen, Vantaa (FI); Ulla Impola, Helsinki (FI); Olli Aitio, Helsinki (FI); Leena Valmu, Helsinki (FI); Ulla Impola, Helsinki (FI); Olli Aitio, Helsinki (FI); Leena Valmu, Helsinki (FI); Suvi Natunen, Vantaa (FI); Hanna Salo, Helsinki (FI)

Correspondence Address: BIRCH STEWART KOLASCH & BIRCH, PO BOX 747, FALLS CHURCH, VA, 22040-0747, US

(73) Assignees: SUOMEN PUNAINEN RISTI, VERIPALVELU, Helsinki (FI); GLYKOS FINLAND LTD., Helsinki (FI)

(21) Appl. No.: 12/522,858

(22) PCT Filed: Jan. 18, 2008
(86) PCT No.: PCT/FI2008/050019

§ 371 Date: Sep. 17, 2009

(30) Foreign Application Priority Data
Jan. 18, 2007 (FI) 20075033
May 10, 2007 (FI) 20070368
Aug. 28, 2007 (FI) 20070650

(51) Int. Cl. G01N 33/53; C12N 5/07

(52) U.S. Cl. 435/7.21; 435/325

(57) ABSTRACT

The invention describes reagents and methods for specific binders to glycan structures of stem cells. Furthermore the invention is directed to screening of additional binding reagents against specific glycan epitopes on the surfaces of the stem cells. The preferred binders of the glycans structures includes proteins such as enzymes, lectins and antibodies.

### FIELD OF THE INVENTION

The invention describes reagents and methods for specific binders to glycan structures of specific types of human cells. Furthermore the invention is directed to screening of additional binding reagents against specific glycan epitopes on the surfaces of the mesenchymal cells (mesenchymal stem cells and cells differentiated thereof). The preferred binders of the glycans structures includes proteins such as enzymes, lectins and antibodies.

### BACKGROUND OF THE INVENTION

Stem Cells

Stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

The first evidence for the existence of stem cells came from studies of embryonic carcinoma (EC) cells, the undifferentiated stem cells of teratocarcinomas, which are tumors derived from germ cells. These cells were found to be pluripotent and immortal, but possess limited developmental potential and abnormal karyotypes (Rossant and Papaioannou, Cell Differ 15,155-161, 1984). ES cells, on the other hand, are thought to retain greater developmental potential because they are derived from normal embryonic cells, without the selective pressures of the teratocarcinoma environment.

Pluripotent embryonic stem cells have traditionally been derived principally from two embryonic sources. One type can be isolated in culture from cells of the inner cell mass of a pre-implantation embryo and are termed embryonic stem (ES) cells (Evans and Kaufman, Nature 292,154-156, 1981; U.S. Pat. No. 6,200,806). A second type of pluripotent stem cell can be isolated from primordial germ cells (PGCS) in the mesenteric or genital ridges of embryos and has been termed embryonic germ cell (EG) (U.S. Pat. No. 5,453,357, U.S. Pat. No. 6,245,566). Both human ES and EG cells are pluripotent. This has been shown by differentiating cells in vitro and by injecting human cells into immunocompromised (SCUM) mice and analyzing resulting teratomas (U.S. Pat. No. 6,200,806). The term “stem cell” as used herein means stem cells including embryonic stem cells or embryonic type stem cells and stem cells diffentiated thereof to more tissue specific stem cells, adults stem cells including mesenchymal stem cells and blood stem cells such as stem cells obtained from bone marrow or cord blood.

The present invention provides novel markers and target structures and binders to these for mesenchymal cells including mesenchymal stem cells and cells differentiated thereof. From other types of cells such as hematopoietic CD34+ cells certain terminal structures such as terminal sialylated type two N-acetyllactosamines such as NeuNAcα3Galβ4GlcNAc (Magnani J. U.S. Pat. No. 6,362,010) low expression of Slex type structures NeuNAcα3Galβ4(Fucα3)GlcNAc (Xia L et al Blood (2004) 104 (10) 3091-6) has been indicated. Due to cell type specificity of glycosylation these are not relevant to mesenchymal stem cells The invention describes structures such as NeuNAcα3Galβ4GlcNAc from specific characteristic O-glycans and N-glycans.

Human ES, EG and EC cells, as well as primate ES cells, express alkaline phosphatase, the stage-specific embryonic antigens SSEA-3 and SSEA-4, and surface proteoglycans that are recognized by the TRA-1-60; and TRA-1-81 antibodies. All these markers typically stain these cells, but are not entirely specific to stem cells, and thus cannot be used to isolate stem cells from organs or peripheral blood.

The SSEA-3 and SSEA-4 structures are known as galactosylgloboside and sialylgalactosylgloboside, which are among the few suggested structures on embryonal stem cells, though the nature of the structures in not ambigious. Some low specificity plant lectin reagents have been reported in binding of embryonal stem cell like materials. Venable et al 2005, (Dev. Biol. 5:15) measured binding of the lectins from SSEA-4 antibody positive subpopulation of embryonal stem cells and Wearne K A et al Glycobiology (2006) 16 (10) 981-990 studied lectin binding to ES cells. An antibody called K21 has been suggested to bind a sulfated polysaccharide on embryonal carcinoma cells (Badcock G et alCancer Res (1999) 4715-19. Due to cell type, species, tissue and other specificity aspects of glycosylation (Furukawa, K., and Kobata, A. (1992) Curr. Opin. Struct. Biol. 3, 554-559, Gagneux, and Varki, A. (1999) Glycobiology 9, 747-755;Gawlitzek, M. et al. (1995), J. Biotechnol. 42, 117-131; Goelz, S., Kumar, R., Potvin, B., Sundaram, S., Brickelmaier, M., and Stanley, P. (1994) J. Biol. Chem. 269, 1033-1040; Kobata, A (1992) Eur. J. Biochem. 209 (2) 483-501.) This result does not indicate the presence of the structure on native embryonal stem cells. The present invention is directed to human mesenchymal cells.

The present invention revealed specifc structures by mass spectrometric profiling, NMR spectrometry and binding reagents including glycan modifying enzymes. The lectins are in general low specificity molecules. The present invention revealed binding epitopes larger than the previously described monosaccharide epitopes. The larger epitopes allowed us to design more specific binding substances with typical binding specificities of at least disaccharides. The invention also revealed lectin reagents with useful specificities for analysis of stem cells.

General methods for separation and use of stem cells are known in the art.

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. In addition, the ability to isolate stem cells may serve in the treatment of lymphomas and leukemias, as well as other neoplastic conditions where the stem cells are purified from tumor cells in the bone marrow or peripheral blood, and reinfused into a patient after myelosuppressive or myeloablative chemotherapy.

Multiple adult stem cell populations have been discovered from various adult tissues. In addition to hematopoietic stem cells, neural stem cells were identified in adult mammalian central nervous system (Ourednik et al. Clin. Genet. 56, 267, 1999). Adult stem cells have also been identified from epithelial and adipose tissues (Zuk et al. Tissue Engineering 7, 211, 2001). Mesenchymal stem cells (MSCs) have been cultured from many sources, including liver and pancreas (Hu et al. J. Lab Clin Med. 141, 342-349, 2003). Recent studies have demonstrated that certain somatic stem cells appear to have the ability to differentiate into cells of a completely different lineage (Pfendler K C and Kawase E, Obstet Gynecol Surv 58, 197-208, 2003). Monocyte derived (Zhao et al. Proc. Natl. Acad. Sci. USA 100, 2426-2431, 2003) and mesodermal derived (Schwartz et al. J. Clin. Invest 109, 1291-1301, 2002) cells that possess some multipotent characteristics were identified. The presence of multipotent “embryonic-like” progenitor cells in blood was suggested also by in-vivo experiments following bone marrow transplantations (Zhao et al. Brain Res Protoc 11, 38-45, 2003). However, such multipotent “embryonic-like” stem cells cannot be identified and isolated using the known markers.

The possibility of recovering fetal cells from the maternal circulation has generated interest as a possible means, non-invasive to the fetus, of diagnosing fetal anomalies (Simpson and Elias, J. Am. Med. Assoc. 270, 2357-2361, 1993). Prenatal diagnosis is carried out widely in hospitals throughout the world. Existing procedures such as fetal, hepatic or chorionic biopsy for diagnosis of chromosomal disorders including Down's syndrome, as well as single gene defects including cystic fibrosis are very invasive and carry a considerable risk to the fetus. Amniocentesis, for example, involves a needle being inserted into the womb to collect cells from the embryonic tissue or amniotic fluid. The test, which can detect Down's syndrome and other chromosomal abnormalities, carries a miscarriage risk estimated at 1%. Fetal therapy is in its very early stages and the possibility of early tests for a wide range of disorders would undoubtedly greatly increase the pace of research in this area. Thus, relatively non-invasive methods of prenatal diagnosis are an attractive alternative to the very invasive existing procedures. A method based on maternal blood should make earlier and easier diagnosis more widely available in the first trimester, increasing options to parents and obstetricians and allowing for the eventual development of specific fetal therapy.

The present invention provides methods of identifying, characterizing and separating stem cells having characteristics of mesenchymal stem (MSC) cells and differentiated derivatives thereof for diagnostic, therapy and tissue engineering. In particular, the present invention provides methods of identifying, selecting and separating mesenchymal cells or to reagents for use in diagnosis and tissue engineering methods. The present invention provides for the first time a specific marker/binder/binding agent that can be used for identification, separation and characterization of valuable stem cells from tissues and organs, overcoming the ethical and logistical difficulties in the currently available methods for obtaining embryonic and other stem cells.

The present invention overcomes the limitations of known binders/markers for identification and separation of mesenchymal cells by disclosing a very specific type of marker/binder structures, with high specificity. In other aspect of the invention, a specific binder/marker/binding agent is provided which does not react, i.e. is not expressed on the mesenchymal cells but on potential contaminating cell type, thus enabling positive selection of contaminating and negative selection of stem cells.

By way of exemplification, the binder to Formula (I) are now disclosed as useful for identifying, selecting and isolating mesenchymal cells including blood derived mesenchymal cells, which have the capability of differentiating into varied cell lineages.

According to one aspect of the present invention a novel method for identifying mesenchymal cells in peripheral blood, cord blood, bone marrow and other organs is disclosed. According to this aspect an mesenchymal cell binder/marker is selected based on its selective expression in mesenchymal cells its absence in other differentiated cells and/or stem cells. Thus, glycan structures expressed in stem cells are used according to the present invention as selective binders/markers for isolation of pluripotent or multipotent stem cells from blood, tissue and organs. Preferably the blood cells and tissue samples are of mammalian origin, more preferably human origin.

According to a specific embodiment the present invention provides a method for identifying a selective mesenchymal cell binder/marker comprising the steps of:

A method for identifying a selective stem cell binder to a glycan structure of Formula (I) which comprises:

i. selecting a glycan structure exhibiting specific expression in/on stem cells and absence of expression in/on differentiated cells and/or other contaminating cells; ii. and confirming the binding of binder to the glycan structure in/on stem cells.

By way of a non-limiting example, adult, mesenchymal, embryonal type, or hematopoietic stem cells selected using the binder may be used in regenerating the hematopoietic or ther tissue system of a host deficient in any class of stem cells. A host that is diseased can be treated by removal of bone marrow, isolation of stem cells and treatment with drugs or irradiation prior to re-engraftment of stem cells. The novel markers of the present invention may be used for identifying and isolating various stem cells; detecting and evaluating growth factors relevant to stem cell self-regeneration; the development of stem cell lineages; and assaying for factors associated with stem cell development.

### BRIEF DESCRIPTION OF THE DRAWINGS

FIG. 1. The N-glycome of human bone marrow MSC:s.

a) MALDI-TOF mass spectrum of the neutral N-glycan fraction from MSC:s.

b) Schematic representation of the relative signal intensities (% of total signals) of 50 most abundant glycan signals (positive mode) from MSC:s and osteoblasts differentiated from them.

c) MALDI-TOF mass spectrum of the acidic N-glycan fraction from MSC:s.

d) Schematic representation of the relative signal intensities (% of total signals) of 50 most abundant glycan signals (negative mode) from MSC:s and osteoblasts differentiated from them.

The structures shown are based on known biosynthetic routes, NMR-analysis and exoglycosidase experiments. The columns indicate the mean abundance of each glycan signal (% of the total glycan signals). Proposed N-glycan monosaccharide compositions are indicated on the x-axis: S: NeuAc, H: Hex, N: HexNAc, F: dHex, Ac: acetyl. The mass spectrometric glycan profile was rearranged and the glycan signals grouped in the main N-glycan structure classes. The isolated N-glycan fractions of the mesenchymal stem cells were structurally analyzed by proton NMR spectroscopy to characterize the major N-glycan core and backbone structures, and specific exoglycosidase digestions with α-mannosidase (Jack beans), α1,2-and α1,3/4-fucosidases (X. manihotis/recombinant), β1,4-galactosidase (S. pneumoniae), and neuraminidase (A. ureafaciens) to characterize the non-reducing terminal epitopes. Structures proposed for the major N-glycan signals are indicated by schematic drawings in the bar diagram. The major sialylated N-glycan structures are based on the trimannosyl core with or without core fucosylation as demonstrated in the NMR analysis. Galactose linkages or branch specificity of the antennae are not specified in the present data. The Lewis x structure can be detected in the same cells by staining with specific binding reagent.

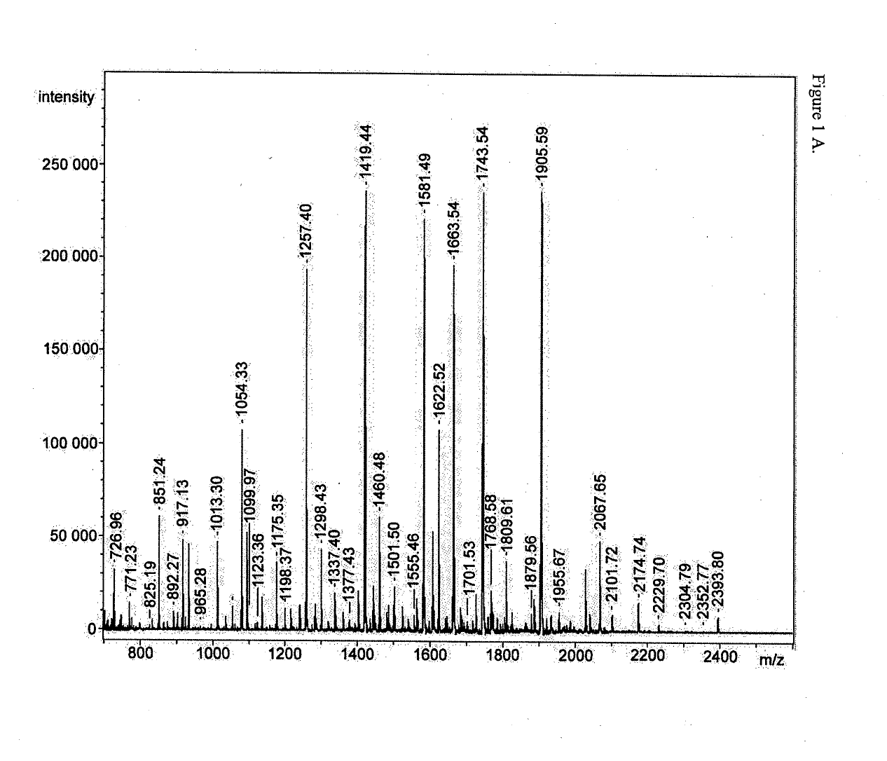

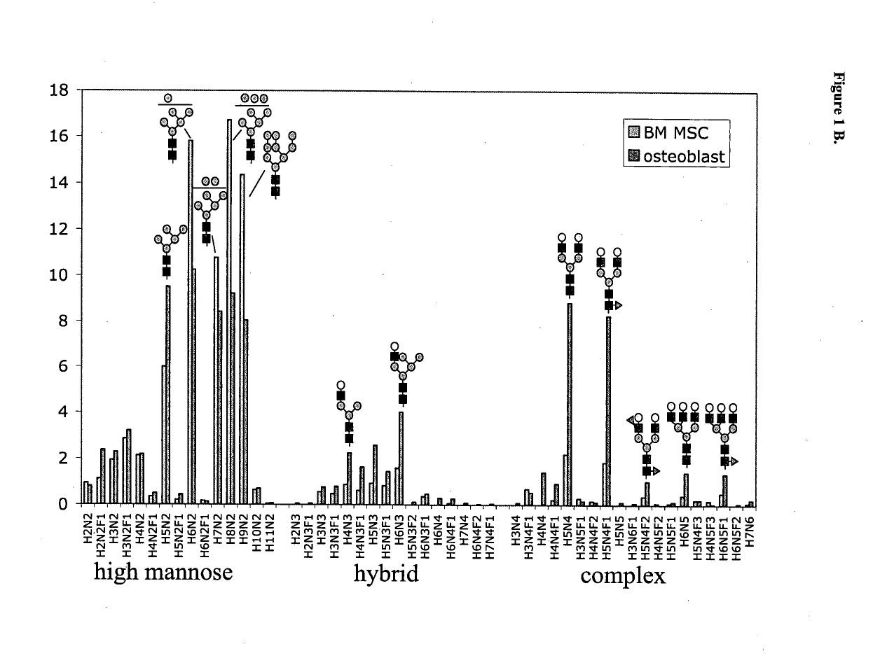

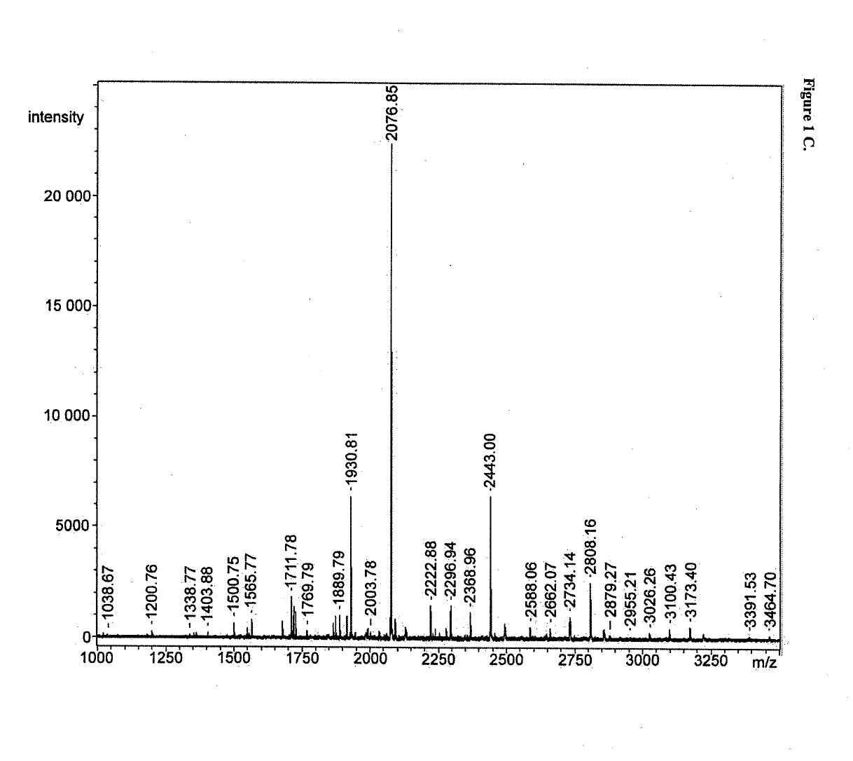

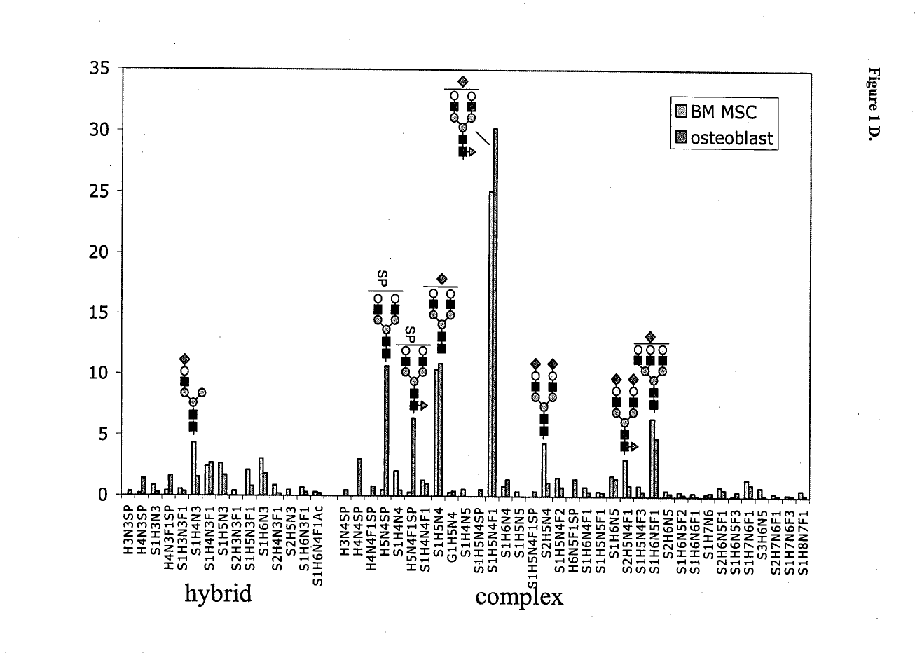

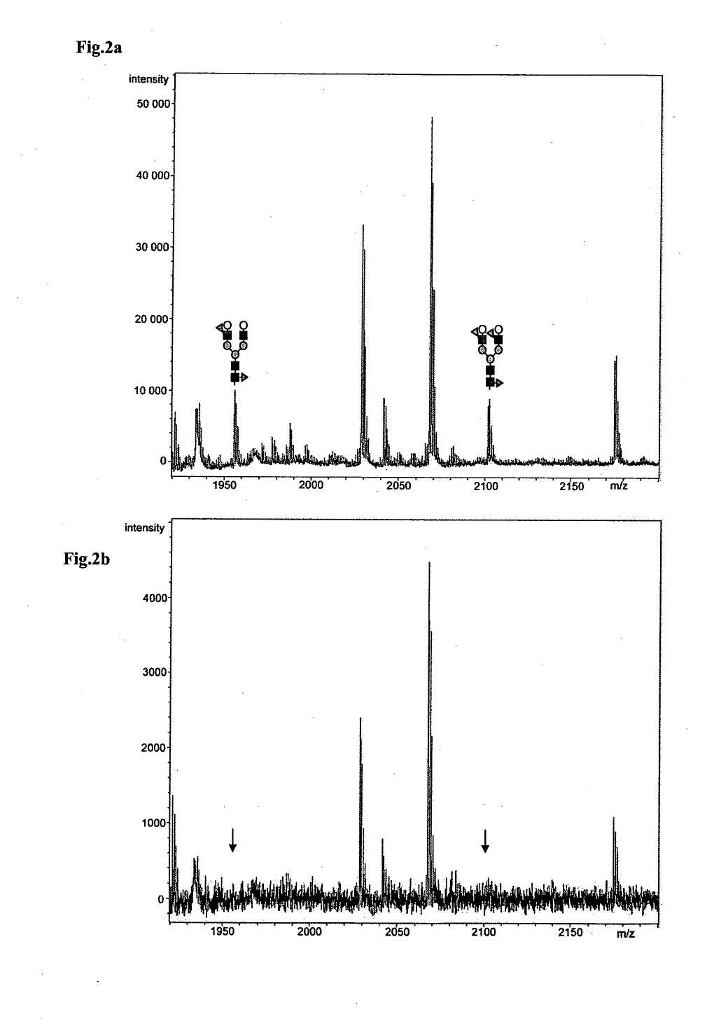

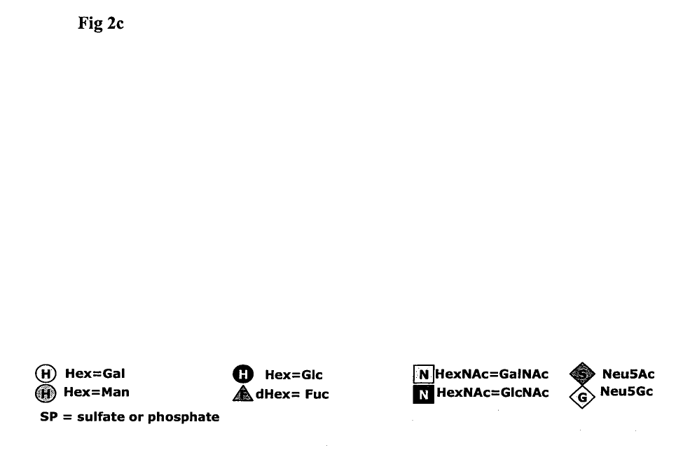

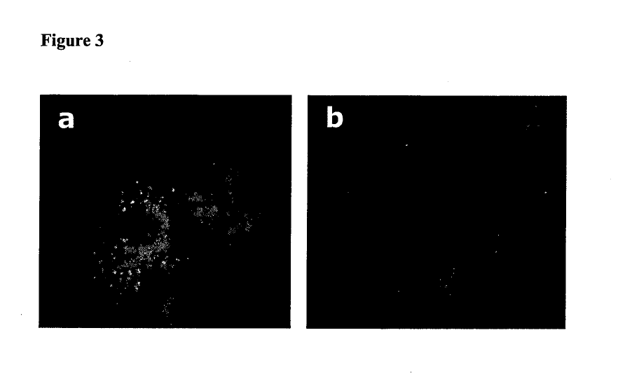

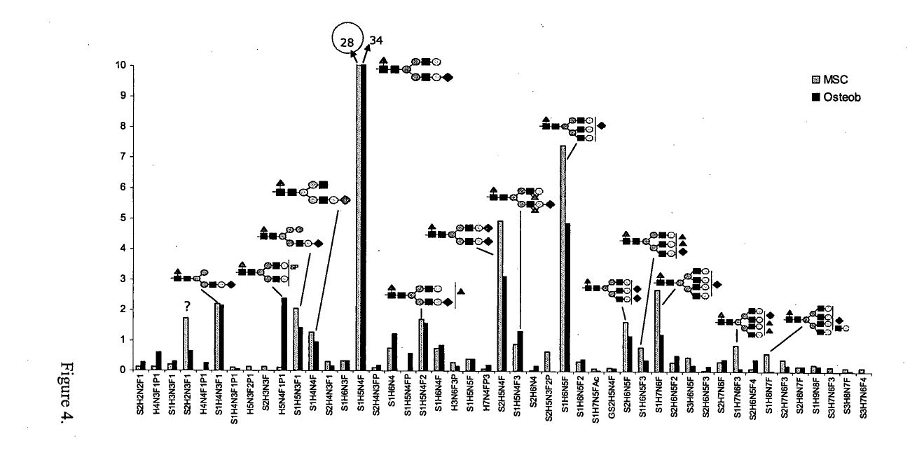

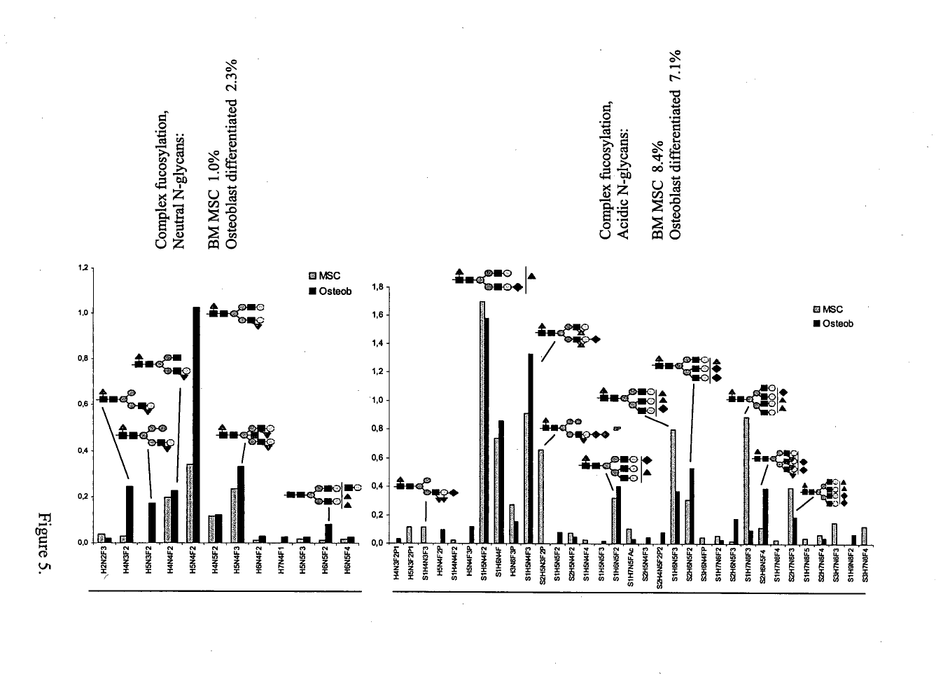

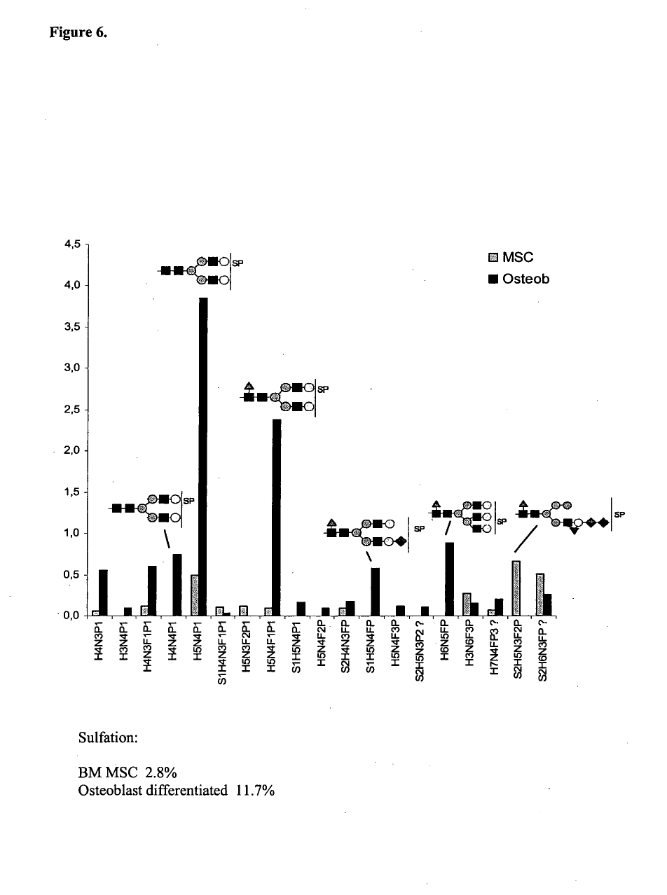

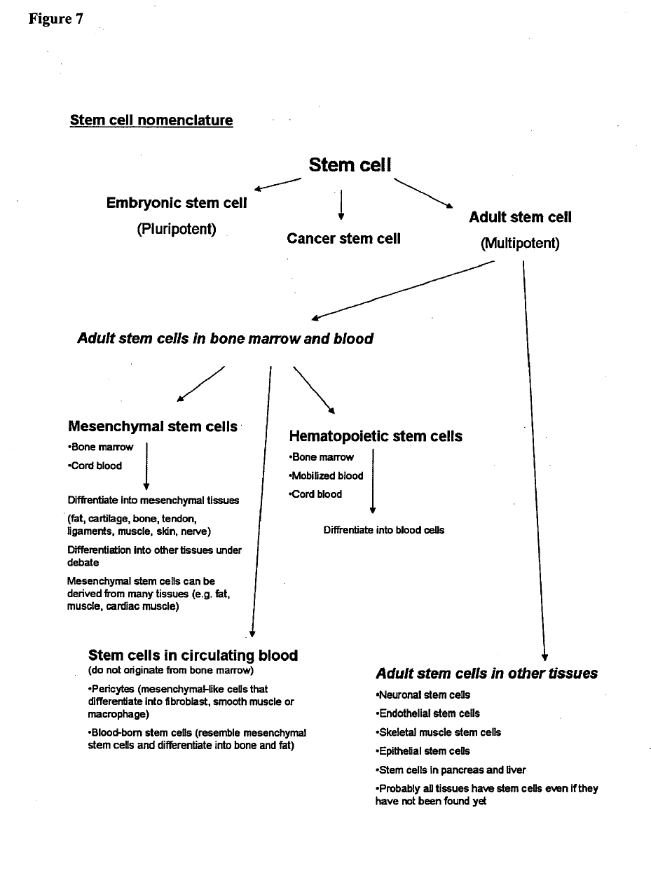

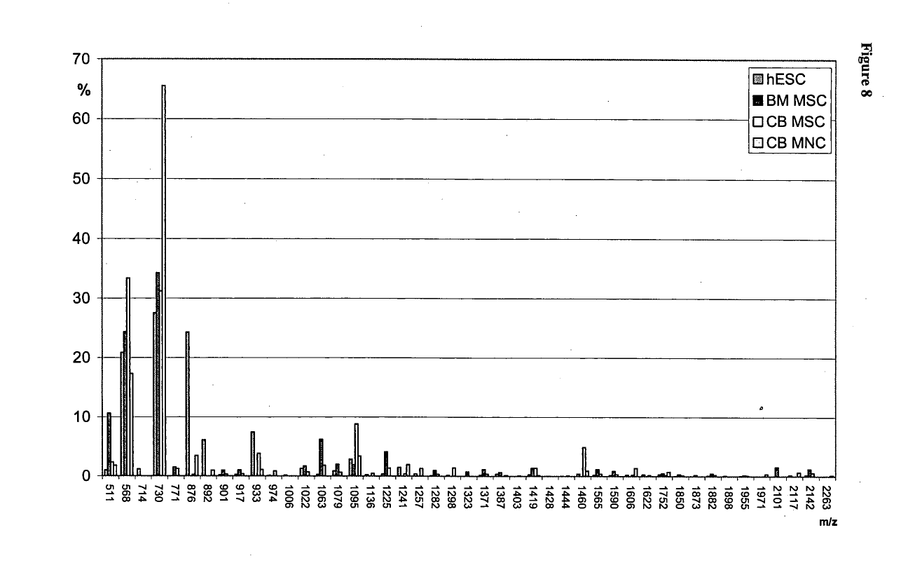

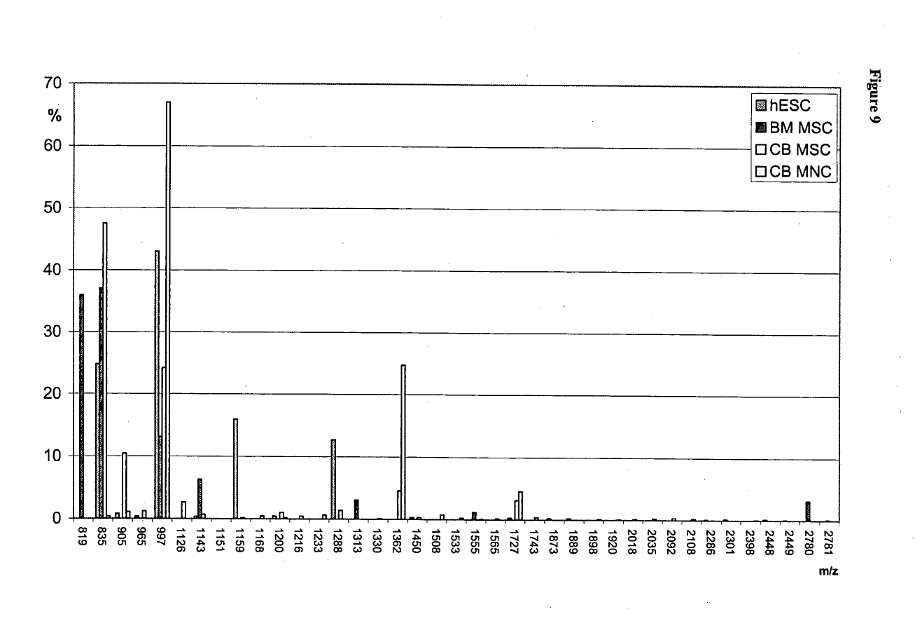

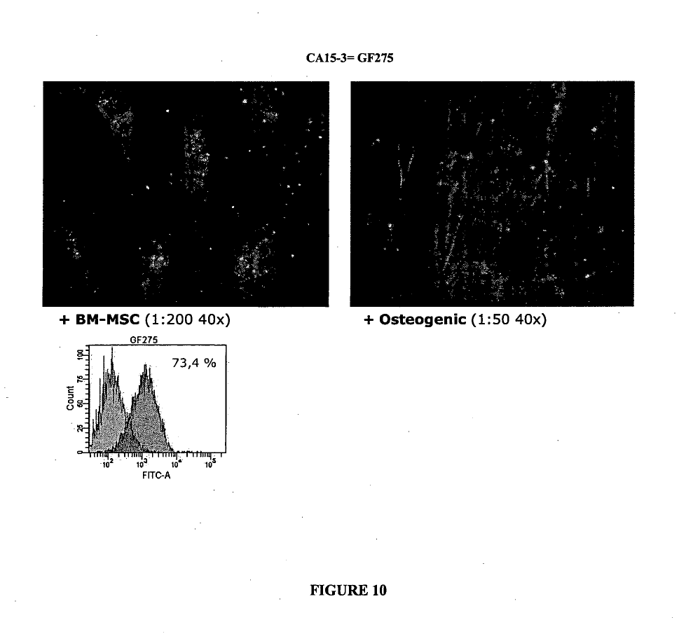

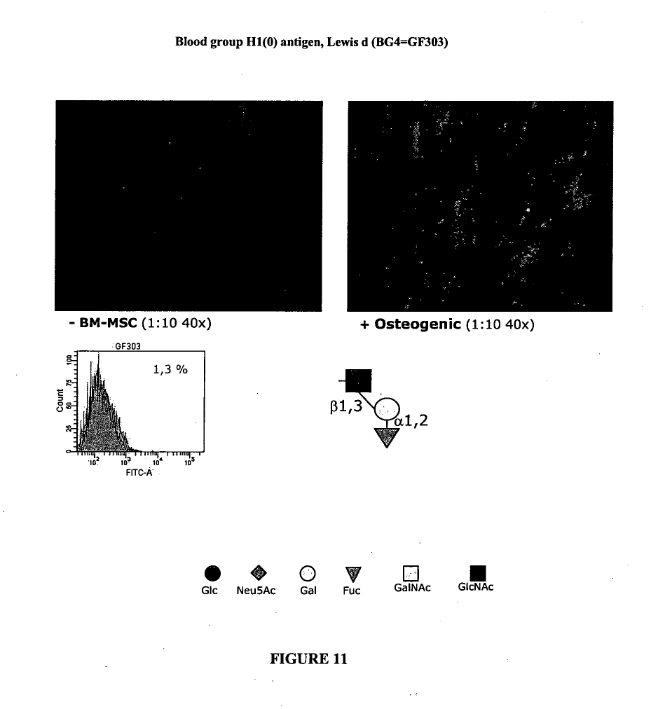

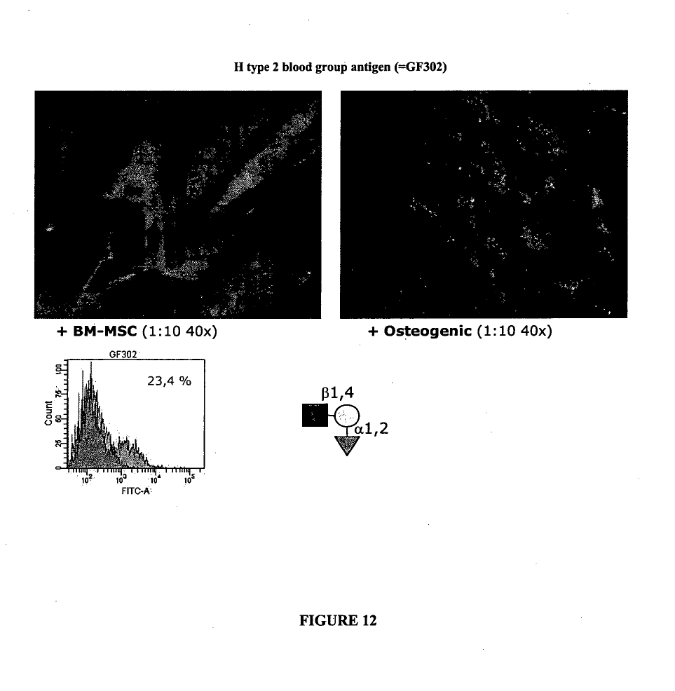

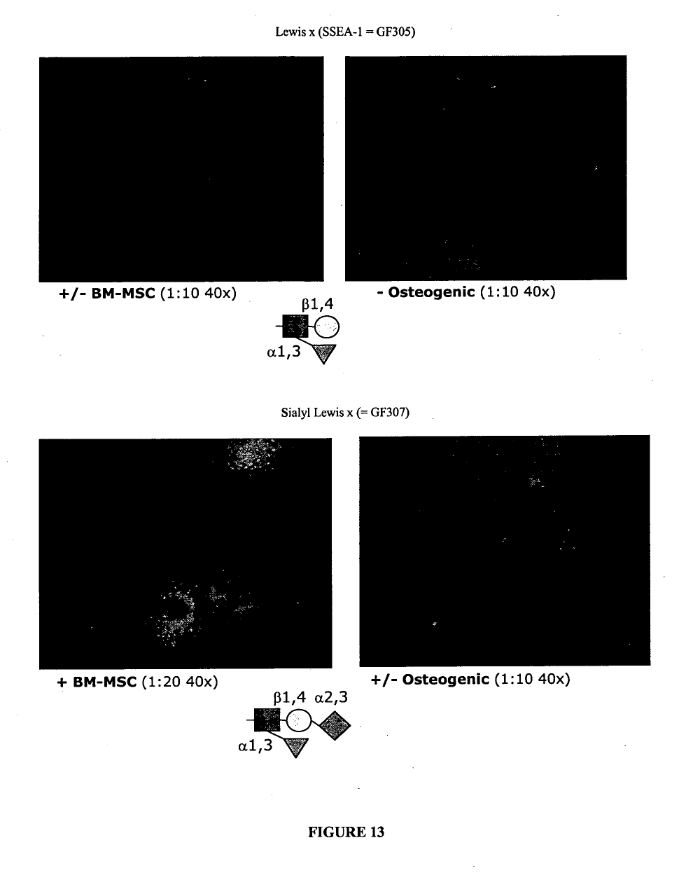

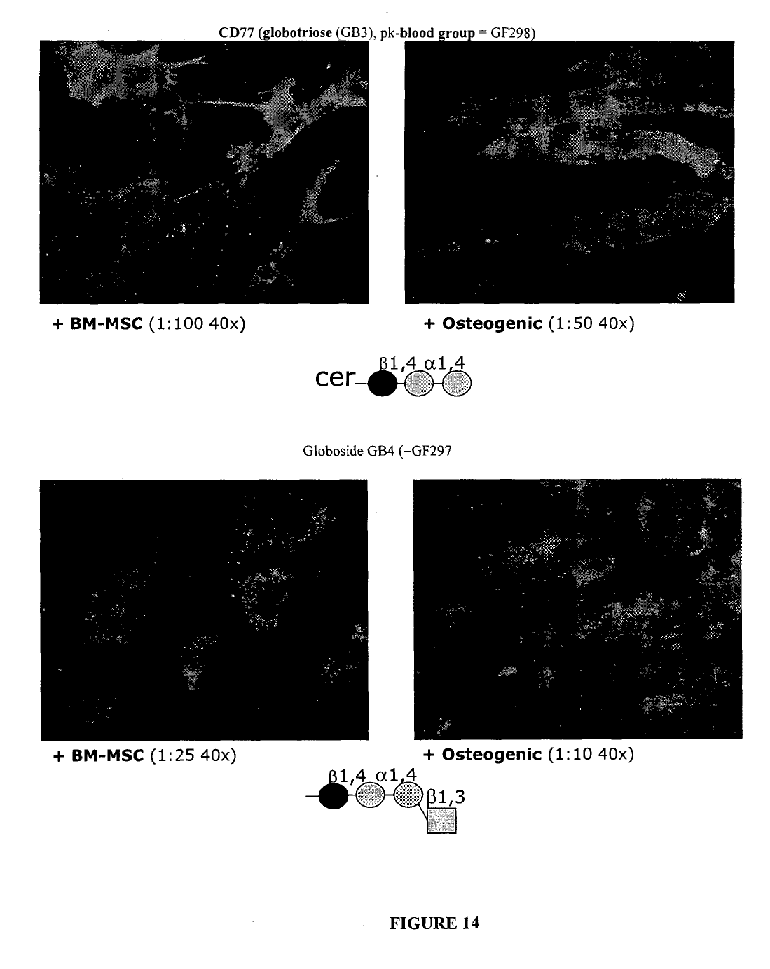

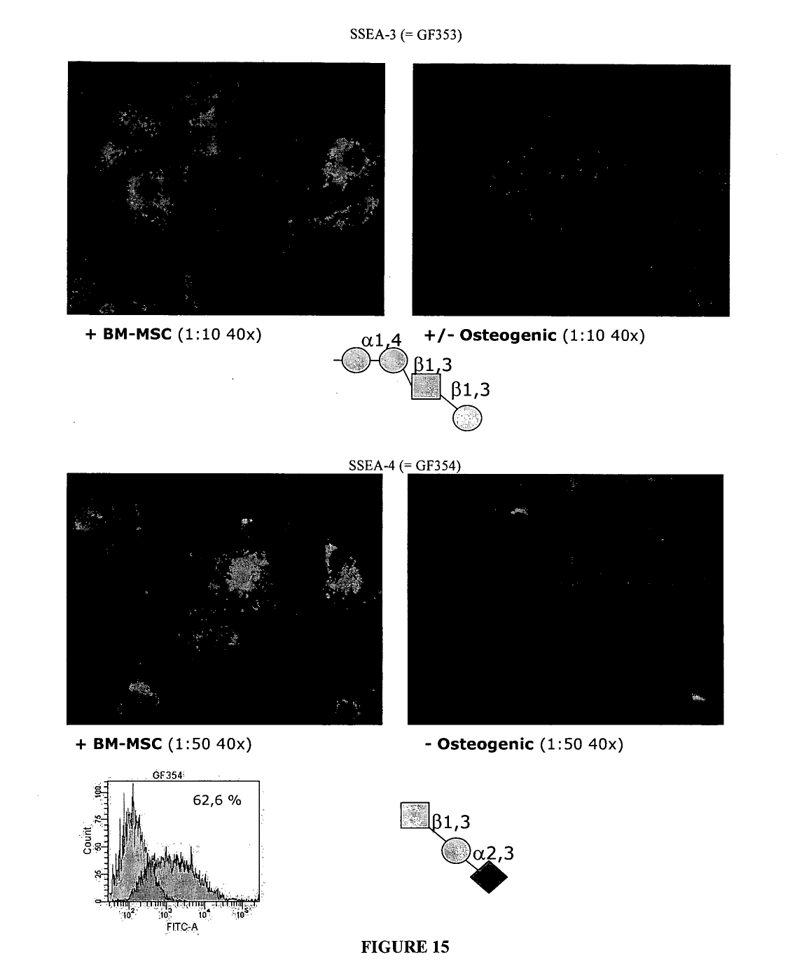

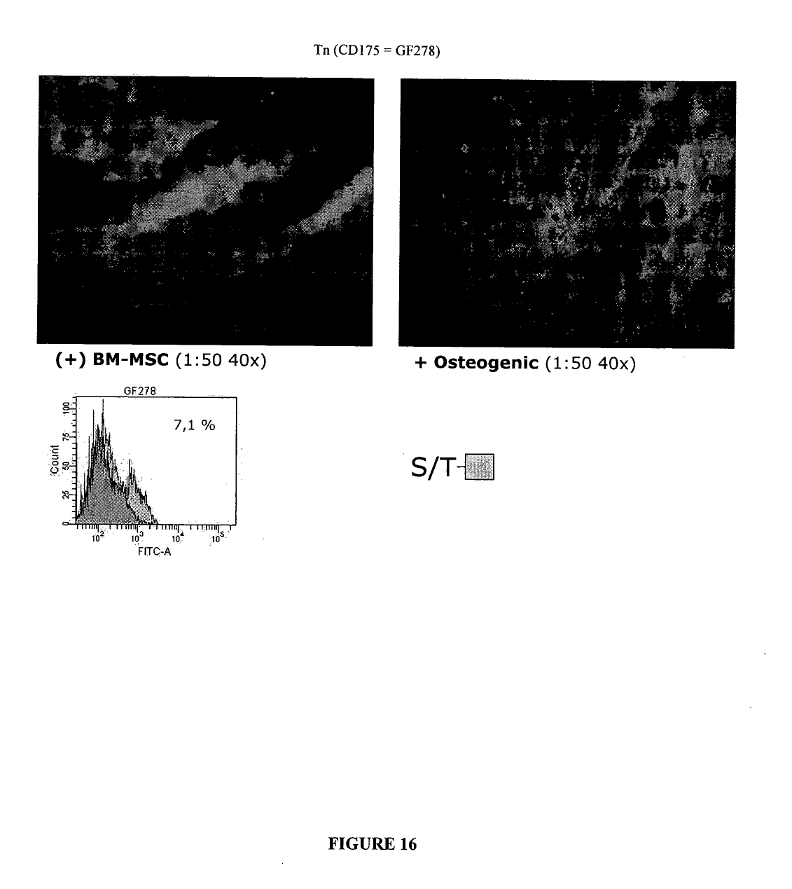

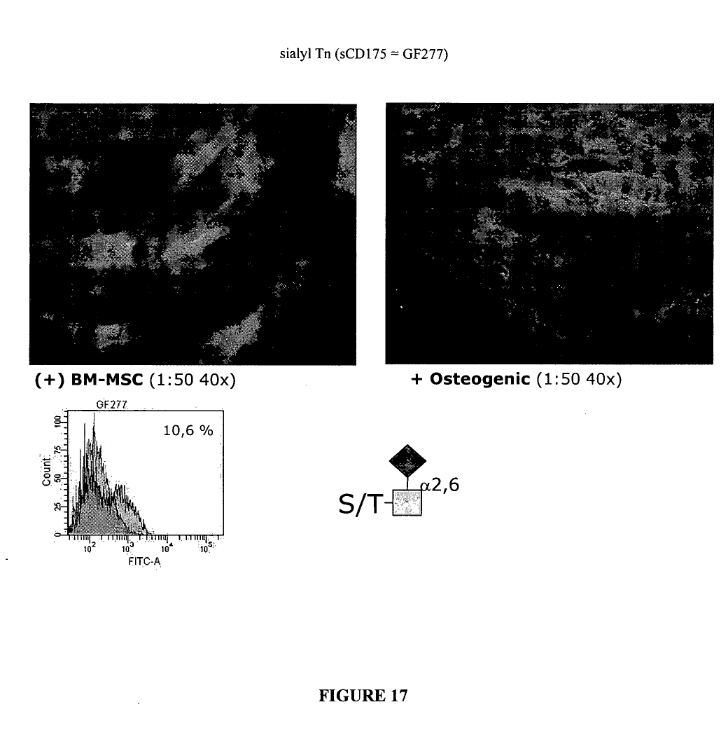

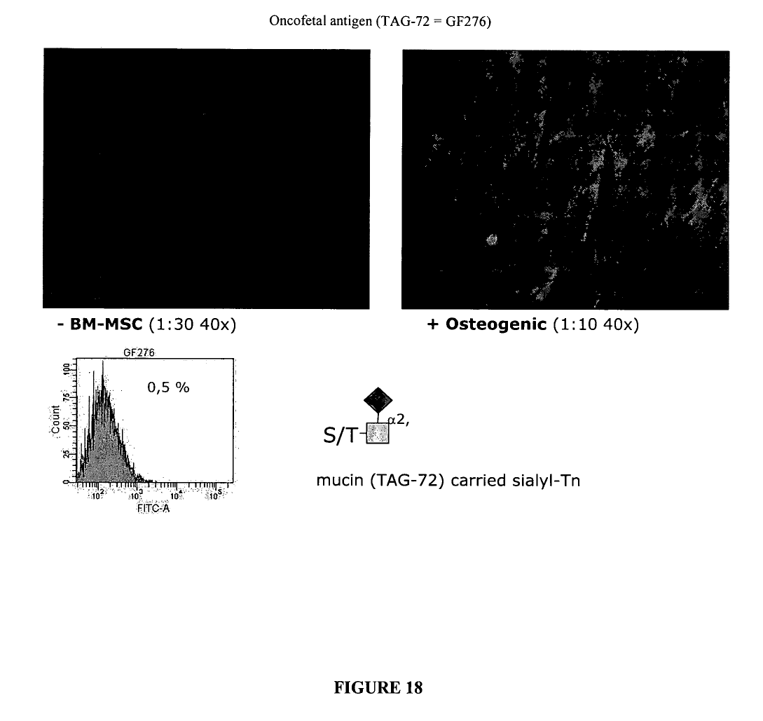

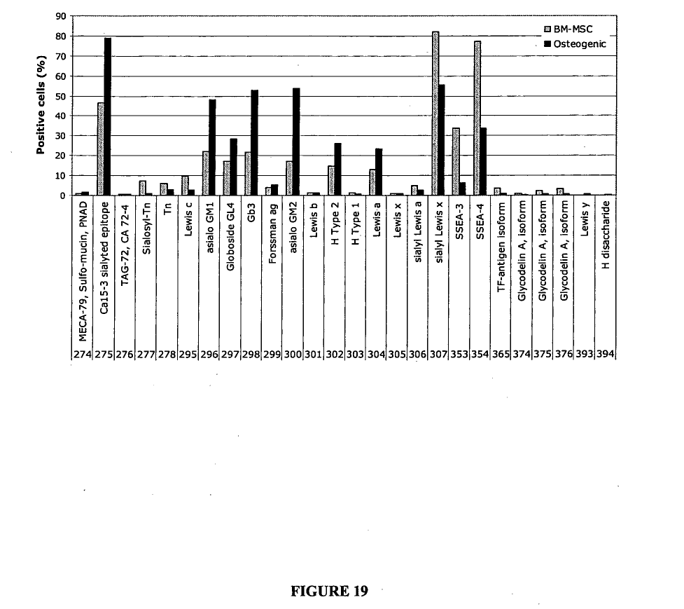

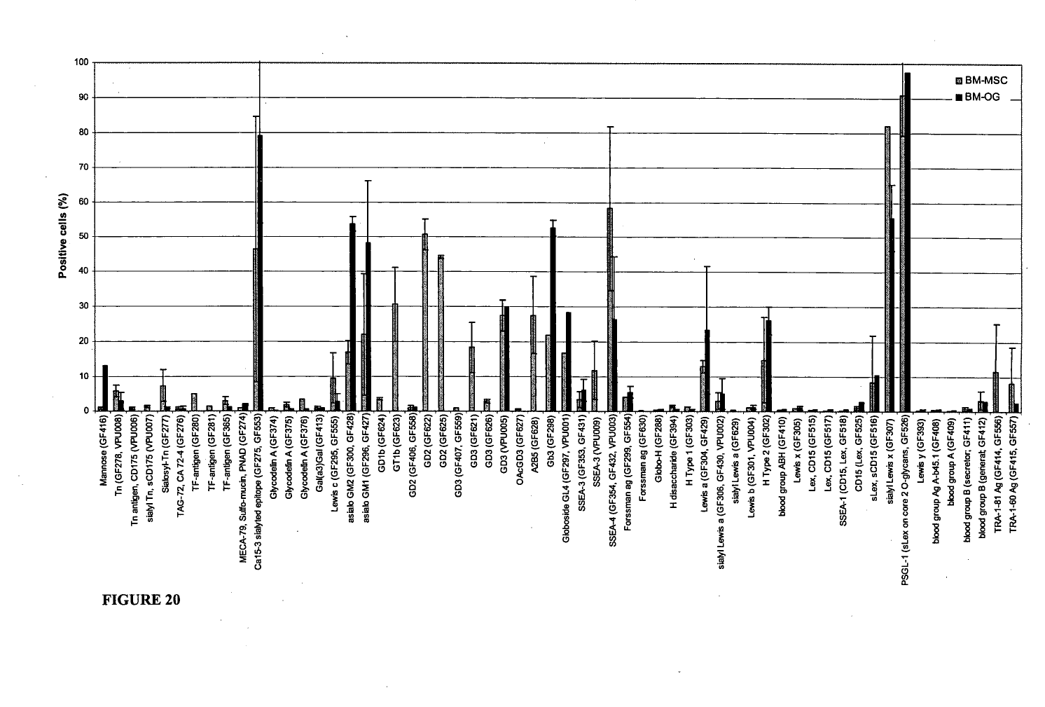

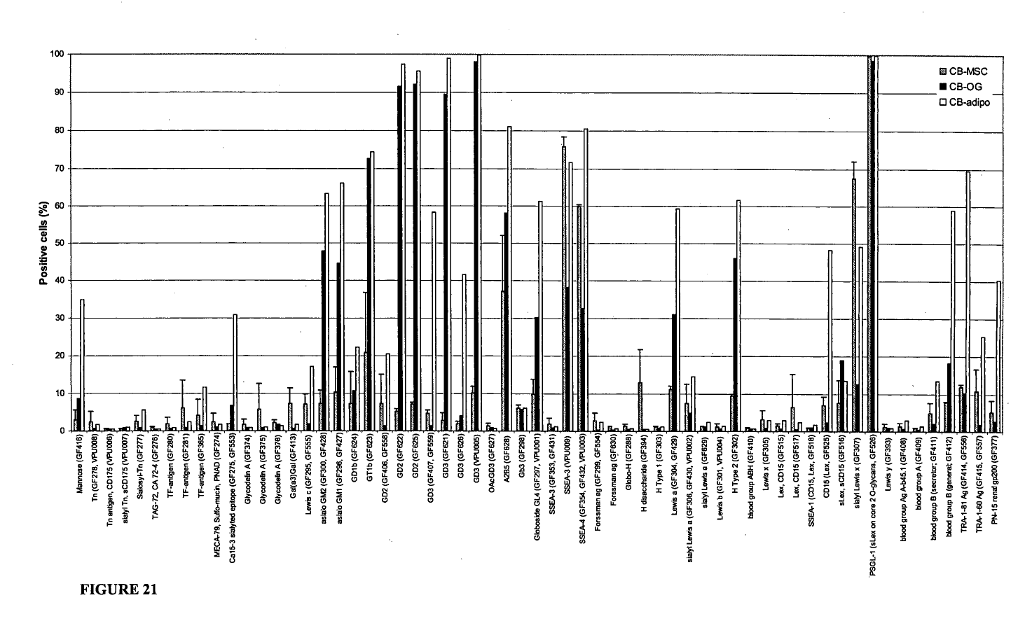

FIG. 2. α3/4-fucosidase treatment of the neutral N-glycan fraction from mesenchymal stem cells. The reaction indicates the presence of structures with Formula Galβ4/3(Fucα3/4)GlcNAc. Lewis x, Galβ4(Fucα3)GlcNAc, structures were revealed by other experiments to be major structures of this type Part of the MALDI-TOF mass spectrum a) before treatment; b) after treatment. Panel c shows the colour code of monosaccharide residues and single letter symbols of monosaccharide residues used in FIG. 1 and FIG. 2.

FIG. 3. Immunofluorescent staining with anti-sialyl Lewis x antibody reveals that the structure Neu5Acα3Galβ4(Fucα3)GlcNAc is a major mesenchymal cell marker associated with stem cell state.

a) bone marrow MSC:s

b) osteoblasts differentiated from bone marrow MSC:s

FIG. 4. Fucosylated acidic N-glycans of bone marrow mesenchymal stem cells (BM MSC) analyzed by MALDI-TOF mass spectrometric profiling. A preferred terminal structure type is sialyl-Lewis x, Neu5Acα3Galβ4(Fucα3)GlcNAc.

FIG. 5. Complex fucosylated neutral (upper panel) and acidic (lower panel) N-glycans of BM MSC analyzed by MALDI-TOF mass spectrometric profiling. The Complex fucosylated (Fuc≧2) N-glycans of human mesenchymal stem cells and changes in their relative abundance during differentiation. The group includes preferred structures Lewis x, Galβ4(Fucα3)GlcNAc, and sialyl-Lewis x, Neu5Acα3Galβ4(Fucα3)GlcNAc.

FIG. 6. Sulfated N-glycans and phosphorylated N-glycans of BM MSC analyzed by MALDI-TOF mass spectrometric profiling. Sulfated N-glycans of human mesenchymal stem cells change in their relative abundance during differentiation.

FIG. 7. Stem cell nomenclature used to describe the present invention.

FIG. 8. MALDI-TOF mass spectrometric profile of isolated human stem cell neutral glycosphingolipid glycans. x-axis: approximate m/z values of [M+Na]⁺ ions as described in Table. y-axis: relative molar abundance of each glycan component in the profile. hESC, BM MSC, CB MSC, CB MNC: stem cell samples as described in the text.

FIG. 9. MALDI-TOF mass spectrometric profile of isolated human stem cell acidic glycosphingolipid glycans. x-axis: approximate m/z values of [M−H]⁻ ions as described in Table. y-axis: relative molar abundance of each glycan component in the profile. hESC, BM MSC, CB MSC, CB MNC: stem cell samples as described in the text.

FIG. 10. Immunostaining of CA15-3 in MSC and osteogenically differentiated cells (sialylated carbohydrate epitope in MUC-1, =GF275). Punctate staining is seen in MSC and more cell membrane localized staining pattern in osteogenically differentiated cells (6 weeks of differentiation, confluent culture). The FACS analysis shows the percentace of MSCs expressing GF275 immunostaining. Majority (more than 80-90%) of osteogenically differentiated cells express GF275

FIG. 11. Immunostaining of MSC and osteogenically differentiated cells. Blood group H1(0) antigen, Lewis d (BG4=GF303). No clear staining is seen in MSC whereas osteogenically differentiated cells show clear immunostaining in more than 70-90% of cells.

FIG. 12. H type 2 blood group antigen (=GF302) immunostaining of MSC and osteogenically differentiated MSCs. The immunostaining in MSCs is seen in approx. 20-75% of both cell types.

FIG. 13. Lewis x (SSEA-1=GF305) immunostaining of MSC and osteogenically differentiated MSCs. Very few cells, less than 10% stain with GF305 in MSCs. Osteogenically differentiated cells do not show or show very little of immunostaining. Sialyl Lewis x (=GF307) immunostaining of MSC and osteogenically differentiated MSCs. Sialyl Lewis x immunostaining decreases when MSC differentiate into osteogenic direction.

FIG. 14. CD77 (globotriose (GB3), pk-blood group=GF298) immunostaining of MSC and osteogenically differentiated MSCs. (Subpopulations of) MSCs and osteogenic direction differentiated MSCs express CD77. Globoside GB4 (=GF297) immunostaining of MSC and osteogenically differentiated MSCs. More punctuate staining of GB4 can be seen in MSCs than in osteogenically differentiated cells.

FIG. 15. SSEA-3 (=GF353) and SSEA-4 (=GF354) immunostaining of MSC and osteogenically differentiated MSCs. SSEA-3 immunostaining decreases when MSC differentiate into osteogenic direction. SSEA-4 (=GF354) immunostaining decreases when MSC differentiate into osteogenic direction.

FIG. 16. Tn (CD175=GF278) immunostaining of MSC and osteogenically differentiated MSCs. Few (5-45%) MSCs express CD175 compared to MSCs differentiated into osteogenic direction.

FIG. 17. sialyl Tn (sCD175=GF277) immunostaining of MSC and osteogenically differentiated MSCs. Few MSCs express sialyl Tn, 5-45%. Osteogenically differentiated cells express more or mainly the epitope.

FIG. 18. Oncofetal antigen (TAG-72=GF276) immunostaining of MSC and osteogenically differentiated MSCs. TAG-72 immunostaining increases or is upregulated when MSC differentiate into osteogenic direction.

FIG. 19: Results of FACS analysis of bone BM-MSCs and osteogenic cells derived thereof. FACS results are shown as an average percentage of positive cells in a cell population (n=1-3 individual experiment(s)).

FIG. 20. FACS analysis of BM-MSC and cells differentiated into osteogenic direction.

FIG. 21. FACS analysis of CB-MSC and cells differentiated into osteogenic and adipogenic direction.

### SUMMARY OF THE INVENTION

The present invention is directed to analysis of broad glycan mixtures from stem cell samples by specific binder (binding) molecules.

The present invention is specifically directed to glycomes of mesenchymal cells (mesenchymal stem cells and cells diffrentiated thereof) according to the invention comprising glycan material with monosaccharide composition for each of glycan mass components according to the Formula I:

R₁Hexβz{R₃}ₙ₁HexNAcXyR₂ (I),

wherein X is nothing or a glycosidically linked disaccharide epitope β4(Fucα6)ₙGN,

wherein n is 0 or 1;

Hex is Gal or Man or GlcA;

HexNAc is GlcNAc or GalNAc;

y is anomeric linkage structure α and/or β or a linkage from a derivatized anomeric carbon,

z is linkage position 3 or 4, with the provision that when z is 4, then HexNAc is GlcNAc and Hex is Man or Hex is Gal or Hex is GlcA, and

when z is 3, then Hex is GlcA or Gal and HexNAc is GlcNAc or GalNAc;

R₁ indicates 1-4 natural type carbohydrate substituents linked to the core structures,

R₂ is reducing end hydroxyl, a chemical reducing end derivative or a natural asparagine linked N-glycoside derivative including asparagines, N-glycoside aminoacids and/or peptides derived from proteins, or a natural serine or threonine linked O-glycoside derivative including asparagines, N-glycoside aminoacids and/or peptides derived from proteins;

R3 is nothing or a branching structure representing GlcNAcβ6 or an oligosaccharide with GlcNAcβ6 at its reducing end linked to GalNAc, when HexNAc is GalNAc, or

R3 is nothing or Fucα4, when Hex is Gal, HexNAc is GlcNAc, and z is 3, or R3 is nothing or Fucα3, when z is 4.

Typical glycomes comprise of subgroups of glycans, including N-glycans, O-glycans, glycolipid glycans, and neutral and acidic subglycomes.

The invention is directed to diagnosis of clinical state of stem cell samples, based on analysis of glycans present in the samples. The invention is especially directed to separating stem cells and malignant cells, preferentially to differentiation between stem cells and cancerous cells and detection of cancerous changes in stem cell lines and preparations.

The invention is further directed to structural analysis of glycan mixtures present in mesenchymal cell samples.

### DESCRIPTION OF THE INVENTION

Glycomes—Novel Glycan Mixtures from Stem Cells

The present invention revealed novel glycans of different sizes from stem cells. The stem cells contain glycans ranging from small oligosaccharides to large complex structures. The analysis reveals compositions with substantial amounts of numerous components and structural types. Previously the total glycomes from these rare materials has not been available and nature of the releasable glycan mixtures, the glycomes, of stem cells has been unknown.

The invention revealed that the glycan structures on cell surfaces vary between the various populations of the early human cells, the preferred target cell populations according to the invention. It was revealed that the cell populations contained specifically increased “reporter structures”.

The glycan structures on cell surfaces in general have been known to have numerous biological roles. Thus the knowledge about exact glycan mixtures from cell surfaces is important for knowledge about the status of cells. The invention revealed that multiple conditions affect the cells and cause changes in their glycomes. The present invention revealed novel glycome components and structures from human mesenchymal cells. The invention revealed especially specific terminal Glycan epitopes, which can be analyzed by specific binder molecules.

Related data and specification was presented in PCT FI 2006/050336, FCT/FI2006/050483, and FCT/FI2006/050485 included fully as reference.

The present invention revealed novel mesenchymal stem cell specific glycans, with specific monosaccharide compositions and associated with differentiation status of stem cells and/or several types of stem cells and/or the differentiation levels of one stem cell type and/or lineage specific differences between stem cell lines.

N-Glycan Structures and Compositions Associated with Differentiation of Stem Cells

The invention revealed specific glycan monosaccharide compositions and corresponding structures, which associated with
i) non-differentiated human mesenchymal stem cells, hMSCs or
ii) differentiated cells derived from the hMSCs, preferably osteoblast or adipocyte type cells.

It is realized that the structures revealed are useful for the characterization of the cells at different stages of development. The invention is directed to the use of the structures as markers for differentiation of mesenchymal stem cells. The invention is further directed to the use of the specific glycans as markers enriched or increased at specific level of differentiation for the analysis of the cells at specific differentiation level.

The invention is further directed to analysis of the general status of the cells as it is realized that the glycosylation is likely to change, when any condition affecting the cells is changed. The invention is further directed to the analysis of the differentiation status of the cells, when the differentiation is expected to be associated with any of the following conditions: change of cell culture conditions including nutritional conditions, growth factor types or amounts, amount of gases available, pH of the cell culture medium; protein, lipid, or carbohydrate content of a medium; physical factors affecting the cells including pressure, shaking, temperature, storage in lowered temperature, freezing and/or thawing and conditions associated with it; contact with different cell culture container surfaces, cell culture matrixes including polymers and gels, and contact with other cell types or materials secreted by these.

N-Glycan Structures and Compositions are Associated with Individual Specific Differences Between Stem Cell Lines or Batches.

The invention further revelead that specific glycan types are presented in the mesenchymal stem cell preparations in varying manner. Most of the altering glycan types are associated on a specific differentiation stage. It is realized that such individually varying glycans are useful for characterization of individual stem cell lines and batches. The specific structures of an individual cell preparation are useful for comparison and standardization of stem cell lines and cells prepared thereof. The specific structures of an individual cell preparation are used for characterization of usefulness of specific stem cell line or batch or preparation for stem cell therapy in a patient, who may have antibodies or cell mediated immune defence recognizing the individually varying glycans.

The invention is especially directed to analysis of glycans with large and moderate individual variations in glycomes.

Analysis Methods by Mass Spectrometry or Specific Binding Reagents

The invention is specifically directed to the recognition of the terminal structures by either specific binder reagents and/or by mass spectrometric profiling of the glycan structures. The preferred methods includes recognition of N-glycans, preferably a biantennary, or triantennary N-glycan is recognized by mass spectrometry and/or binder reagent. Preferably the N-glycan is recognized by mass spectrometry and the binder reagent is preferably a glycosidase enzyme.

In a preferred embodiment the invention is directed to the recognition of the structures and/or compositions based on mass spectrometric signals corresponding to the structures.

The preferred binder reagents are directed to characteristic epitopes of the structures such as terminal epitopes and/or characteristic branching epitopes, such as fucosylated structures including sialyl-Lewis x and Lewis x structures and sulfated structures. The invention is directed to specific antibodies recognizing the preferred terminal epitopes, the invention is further directed to other binders with the same or similar specificity, preferably with the same specificity as the preferred antibodies.

The preferred binder is a protein or peptide binding to carbohydrate, preferably a lectin, enzyme or antibody or a carbohydrate binding fragment thereof. In a preferred embodiment the binder is an antibody, more preferably a monoclonal antibody.

In a preferred embodiment the invention is directed to a monoclonal antibody specifically recognizing at least one of the terminal epitope structures according to the invention.

The mass spectrometric profiling of released N-glycans revealed characteristic changes in the glycan profiles. The mass spectrometric method allows detection of multiple glycans and glycan type simultaneously. The mass profiles reveal individual glycan structures specific for specific cell types. The invention is especially directed to the recongnition of the glycan structures from very low amounts of material such as from 1000 to 5 000 000 cells, preferably between 10 000 and million cells and most preferably between 100 000 and million cells.

Use of the Binding Reagents for the Analysis of Cellular Interactions

It is realized that the carbohydrate structures on cell surfaces are associated with contacts with other cells and surrounding cellular matrix. Therefore the identified cell surface glycan structures and especially binding reagents specifically recognizing these are useful for the analysis of the cells. The preferred analysis method includes the step of contacting the cell with a binding reagent and evaluating the effect of the binding reagent to the cell. In a preferred embodiment the cells are contacted with the binder under cell culture condition. In a preferred embodiment the binder is represented in multivalent or more preferably polyvalent form or in another preferred embodiment in surface attached form. The effect may be change in the growth characteristics or cellular signalling in the cells.

Preferred Terminal Structural Epitopes

The invention is directed to the use of type II N-acetyllactosamine type structures including closely homologous structures, such as LacdiNAc (GalNAcβ4GlcNAc) and lactosyl (Galβ4Glc) structures for the evaluation of mesenchymal stem cells and derivatives thereof.

The invention is preferably directed to evaluating the status of a human mesenchymal stem cell preparation comprising the step of detecting the presence of a glycan structure or a group of glycan structures in said preparation, wherein said glycan structure or a group of glycan structures is according to Formula LN1

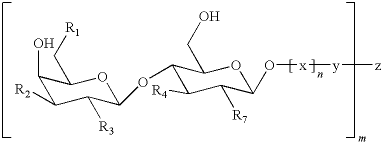

wherein

X is linkage position

R₁, and R₂, are OH or glycosidically linked monosaccharide residue Sialic acid,

preferably Neu5Acα or Neu5Gcα, most preferably Neu5Acα or sulfate ester groups or

R₃, is OH or glycosidically linked monosaccharide residue Fucα(L-fucose) or N-acetyl (N-acetamido, NCOCH₃);

R₄, is OH or glycosidically linked monosaccharide residue Fucα(L-fucose),

R7 is N-acetyl or OH

X is natural oligosaccharide backbone structure from the cells, preferably N-glycan,

O-glycan or glycolipid structure; or X is nothing, when n is 0,

Y is linker group preferably oxygen for O-glycans and O-linked terminal oligosaccharides and glycolipids and N for N-glycans or nothing when n is 0;

Z is the carrier structure, preferably natural carrier produced by the cells, such as protein or lipid, which is preferably a ceramide or branched glycan core structure on the carrier or H;

n is an integer 0 or 1, and m is an integer from 1 to 1000, preferably 1 to 100, and most preferably 1 to 10 (the number of the glycans on the carrier) and with the provision that when R7 is N-acetyl then 6 position hydroxyl of the GlcNAc residue may be substituted by sulfate ester.

The invention is further directed to the structures according to the Formula LN2

[Mα]ₘGalβ1-4[Nα]ₙGlcNAcβxMan

wherein

wherein m, n and p are integers 0, or 1, independently,

x is linkage position selected from the group 2, 4 or 6

M and N are substituents or monosaccharide residues being
I. independently nothing (free hydroxyl groups at the positions) and/or
II. SA which is Sialic acid linked to 3-position or 6-position of Gal and/or
III. Fuc (L-fucose) residue linked to 2-position of Gal and/or 3 position of GlcNAc, and/or
IV. Sulfate ester on position 3 or 6-of Gal and/or position 6 of GlcNAc,

with the provision that when sialic acid is linked to position 6, then preferably n is 0,

The invention is further directed to the structures according to the Formula LN3

[Mα]ₘGalβ1-4[Nα]ₙGlcNAcβ2Man,

wherein the variables are as described for Formula LN2 and the structure is preferably linked to N-glycan core.

The specifically preferred structures are fucosylated structures according to the Formula LN4

[Mα]ₘGalβ1-4(Fucα3)ₙGlcNAcβ2Man,

wherein M is α3-linked sialic acid (SAα3) preferably Neu5Acα3 or Fucα2.

The preferred LN4 structure is a N-glycan linked structure being:

Lewis x structure, Galβ1-4(Fucα3)GlcNAcβ2Man, or

sialyl-Lewis x structure Neu5Acα3Galβ1-4(Fucα3)GlcNAcβ2Man.

Another preferred structure group includes a structure according to the Formula LN4a

[SAα3]ₘGaloβ1-4GlcNAcβ2Man,

wherein SA is sialic acid preferably Neu5Ac and

and the structure is a N-glycan linked

type II LacNAc structure, Galβ1-4GlcNAcβ2Man, or

sialyl-type II LacNAc structure Neu5Acα3Galβ1-4GlcNAcβ2Man

The invention is further directed to structures according to the Formula LN5

[SE3/6]ₘGalβ1-4[SE6]ₙGlcNAcβ2Man,

wherein SE is sulfate ester and 3/6 indicates either 3 or 6 and

the structure comprises at least one sulfate residue.

The invention is further directed to structures according LN2 are selected from the group consisting of Galβ4GlcNAcβ2Man, Galβ4(Fucα3)GlcNAcβ2Man, Fucα2Galβ4GlcNAcβ2Man, SAα6Galβ4GlcNAcβ2Man, and SAα3Galβ4GlcNAcβ2Man.

The isomeric fucosylated and sialylated structures, H type II Fucα2Galβ4GlcNAcβ2Man, and SAα6Galβ4GlcNAcβ2Man are preferred as controls for the other structures. The structures are also associated with certain differentiated cell populations.

In a preferred embodiment the structure is H type II structure associated with differentiated cells.

The invention is directed to the method further involving the recognition of a triantennary terminal structure according to the Formula LN4b

[SAα3]ₘGalβ1-4GlcNAcβ4Man,

wherein SA is sialic acid preferably Neu5Ac and

and the structure is a N-glycan linked

type II LacNAc structure, Galβ1-4GlcNAcβ4Man, or

sialyl-type II LacNAc structure Neu5Acα3Galβ1-4GlcNAcβ4Man.

Analysis of N-Glycans of Mesenchymal Stem Cells and Differentiated Variants Thereof

MALDI-TOF mass spectrometric analysis of mesenchymal cell N-glycans is shown in FIG. 1. In panel a) MALDI-TOF mass spectrum of the neutral N-glycan fraction from MSC:s and in panel b) Schematic representation of the relative signal intensities (% of total signals) of 50 most abundant glycan signals (positive mode) from MSC:s and osteoblasts differentiated from them.

The panel c) of FIG. 1 shows MALDI-TOF mass spectrum of the acidic N-glycan fraction from MSC:s. and panel d) Schematic representation of the relative signal intensities (% of total signals) of 50 most abundant glycan signals (negative mode) from MSC:s and osteoblasts differentiated from them. The comparision of the relative intensities in panel b) and d) allowed the determination of structures specific for non-differentiated cells and for differentiated cells.

FIG. 1 further indicates colour symbol coded structures of the N-glycans. The symbols are used essentially similarily to those used by the Consortium for Functional Glycomics.

Briefly, in Tables 5 and 6 the reducing end of the N-glycans is on the left, β1-4 linkages (Manβ4,GlcNAcβ4,Galβ4) and GlcNAcβ2 are indicated by horizontal line -, 1-6 linkages (Manα6, NeuAc/sialic acidα6, GlcNAcβ6) are indicated by line upwards /, except Fucα6 above above reducing end GlcNAc, 1-3 linkages (Manα3,Fucα3,Neu5Ac/Neu5Gc/sialic acidα3), are indicated by \, Fucα2 is indicated by vertical line below Galβ, or in the cases where H— structures and GlcNAc fucosylation are alternative structures in the same epitope, line is drawn to both residues. SP represent a sulfate or phosphoryl ester linked to a LacNAc unit, part of the SP symbols are represented as mirror images. The Tables 5 and 6 include representative structures and it is realized that isomeric structures exist, for example when N-glycans carry different terminal epitopes the actual branch location of sialyl, fucosyl or sulfate moieties with regard to two or more N-acetyllactosamines is not definitely indicated, but includes isomeric variants(s). Formulas written for preferred monosaccharide compositions can be used for verification of the structures written with symbols. The same structures have been turned 90 degrees counterclockwise in FIGS. 1 and 2, the reducing end points downwards, the linkages of similar or same oligosaccharides are represented in Tables 7 and 8.

The glycan structures comprising multiple isomeric structures are indicated by line and separated monosaccharide or disaccharide (LacNAc) elements, the sialic acid residues (Neu5Ac and Neu5Gc) are linked preferably to terminal Gal residues, fucose to Gal or GlcNAc and LacNAc to Gal (another LacNAc unit) as described in the invention.

The structures shown are based on known biosynthetic routes, NMR-analysis and exoglycosidase experiments. The columns indicate the mean abundance of each glycan signal (% of the total glycan signals). Proposed N-glycan monosaccharide compositions are indicated on the x-axis: S: NeuAc, H: Hex, N: HexNAc, F: dHex, Ac: acetyl, SP sulfate of phosphate. The mass spectrometric glycan profile was rearranged and the glycan signals grouped in the main N-glycan structure classes. Glycan signals in the group ‘Other’ are marked with m/z ratio of their [M+Na]+ (left panel) or [M−H]− ions (right panel) and monosaccharide compositions. The isolated N-glycan fractions of the mesenchymal stem cells were structurally analyzed by proton NMR spectroscopy to characterize the major N-glycan core and backbone structures, and specific exoglycosidase digestions with α-mannosidase (Jack beans), α1,2-and α1,3/4-fucosidases (X. manihotis/recombinant), β1,4-galactosidase (S. pneumoniae), and neuraminidase (A. ureafaciens) to characterize the non-reducing terminal epitopes. Structures proposed for the major N-glycan signals are indicated by schematic drawings in the bar diagram. The major sialylated N-glycan structures are based on the trimannosyl core with or without core fucosylation as demonstrated in the NMR analysis. The Lewis x structure can be detected in the same cells by staining with a specific binding reagent.

Preferred Terminal Non-Fucosylated Structures

Type 2 N-Acetyllactosamine Structures

The preferred complex type epitopes on N-glycans includes type 2 N-acetyllactosamine structure epitopes of biantennary N-glycans Galβ4GlcNAcβ2, Galβ4GlcNAcβ2Man, Galβ4GlcNAcβ2Manα, Galβ4GlcNAcβ2Manα3, Galβ4GlcNAcβ2Manα6 and Galβ4GlcNAcβ2Manα3/6. Galactosidase analysis revealed that the structures are present on both arms of biantennary N-glycans.

Sialyl-Type 2 N-Acetyllactosamine Structures

The preferred complex type epitopes on N-glycans include sialyl-type 2 N-acetyllactosamine structural epitopes of biantennary N-glycans Neu5Acα3Galβ4GlcNAcβ2, Neu5Acα3 Galβ4GlcNAcβ2Man, Neu5Acα3Galβ4GlcNAcβ2Manα, Neu5Acα3 Galβ4GlcNAcβ2Manα3, Neu5Acα3Galβ4GlcNAcβ2Manα6 and Neu5Acα3Galβ4GlcNAcβ2Manα3/6.

Preferred Fucosylated Structure Types

The invention revealed fucosylated glycan structures in N-glycomes of the mesenchymal cells. The preferred structure types includes terminal structures comprising α3/4 linked fucoses revealed by specific fucosidase digestion. These includes especially type II structures Lewis x and sialyl Lewis x and also Lewis a and sialyl Lewis a. The major linkage type of galactose as β4 and terminal α3-sialylation were revealed by specific glycosidase digestions. The terminal structure types were analyzed from various glycan types from the mesenchymal cells of the invention. The invention is directed to specific antibodies known to recognize Lewis x (e.g. Dubet et al abstract Glycobiology Society Meeting 2006, Los Angeles) and sialyl-Lewis x on specific preferred N-glycan structures according to the invention.

The invention is further directed to the use and testing/selection of antibodies specific for the structures on O-glycans or glycolipids for the analysis of mesenchymal type stem cells. The invention is further directed to lower specificity antibodies and/or other binding reagents recognizing the terminal epitopes on all or at least two glycan classes selected from the group N-glycans, O-glycans and glycolipids. The invention is further directed to the use of the antibodies and/or other corresponding binder reagents for methods including the step of binding of the reagent to the cells including cell sorting, cell manipulation or cell culture.

Fucosylated Structures on Complex Type N-Glycans

The invention is especially directed to the fucosylated structures carried on complex type N-glycans (referred also as Complex fucosylated structures). The terminal epitopes in the complex fucosylated structures are mainly linked to Manα-residues of N-glycan core structures, the linkage is β2-linkage in biantennary structures, and preferably in triantennary structures also β4-linkage, and in tetra-antennary and more branched structures further include β6-linkage. The invention further revealed unusually large N-glycans, which carry polylactosamine structures where lactosamines are linked to each other with β3 and/or β6 linkages forming epitopes like Galβ4GlcNAcβ3/6Galβ4GlcNAcβ2, which can be further sialylated and/or fucosylated.

The invention revealed especially biantennary but also triantennary and larger N-glycans and the invention is in a preferred embodiment especially directed to these N-glycans carrying fucose residues.

The preferred complex type epitopes on N-glycans includes Lewis x structure epitopes of biantennary N-glycans Galβ4(Fucα3)GlcNAcβ2, Galβ4(Fucα3)GlcNAcβ2Man, Galβ4(Fucα3)GlcNAcβ2Manα, Galβ4(Fucα3)GlcNAcβ2Manα3, Galβ4(Fucα3)GlcNAcβ2Manα6 and Galβ4(Fucα3)GlcNAcβ2Manα3/6. Fucosidase analysis revealed that Lewis x structures are present on both arms of biantennary N-glycans.

The preferred complex type epitopes on N-glycans include sialyl-Lewis x structure epitopes of biantennary N-glycans Neu5Acα3Galβ4(Fucα3)GlcNAcβ2, Neu5Acα3Galβ4(Fucα3)GlcNAcβ2Man, Neu5Acα3Galβ4(Fucα3)GlcNAcβ2Manα, Neu5Acα3Galβ4(Fucα3)GlcNAcβ2Manα3, Neu5Acα3Galβ4(Fucα3)GlcNAcβ2Manα6 and Neu5Acα3Galβ4(Fucα3)GlcNAcβ2Manα3/6.

FIG. 2 shows α3/4-fucosidase treatment of the neutral N-glycan fraction from mesenchymal stem cells. The reaction indicates the presence of structures with Formula Galβ4/3(Fucα3/4)GlcNAc. Lewis x, Galβ4(Fucα3)GlcNAc, or Lewis a structures were revealed by other experiments to be major structures of this type. Part of the MALDI-TOF mass spectrum a) before treatment; b) after treatment. Panel c shows the colour code of monosaccharide residues and single letter symbols of monosaccharide residues used in FIG. 1 and FIG. 2.

FIG. 3 reveals immunofluorescent staining with anti-sialyl Lewis x antibody (GF 307) reveals that the structure Neu5Acα3Galβ4(Fucα3)GlcNAc is a major mesenchymal cell marker associated with stem cell state. In panel a) bone marrow MSC:s are stained effectively and panel b) shows no or very little binding on the osteoblasts differentiated from bone marrow MSC:s by the specific anti-sialyl-Lewis x antibody.

FIG. 4 shows fucosylated acidic N-glycans of bone marrow mesenchymal stem cells (BM MSC) analyzed by MALDI-TOF mass spectrometric profiling. A preferred terminal structure type is sialyl-Lewis x, Neu5Acα3Galβ4(Fucα3)GlcNAc.

FIG. 5. shows selected complex fucosylated neutral (upper panel) and acidic (lower panel) N-glycans of BM MSC analyzed by MALDI-TOF mass spectrometric profiling. The Complex fucosylated (Fuc≧2) N-glycans of human mesenchymal stem cells and changes in their relative abundance during differentiation. The group includes preferred structures Lewis x, Galβ4(Fucα3)GlcNAc, and sialyl-Lewis x, Neu5Acα3Galβ4(Fucα3)GlcNAc. The level of fucosylation on complex type N-glycan increases during differentiation and the invention is in a preferred embodiment directed to use of the amount of fucosylated structures on N-glycans for characterization of the mesenchymal cells

Sulfated N-Acetyllactosamine Structures

The invention further revealed that sulfation on complex type N-glcyans is very characteristic to differentiated osteoblast type cells as shown in FIG. 6. Sulfated N-glycans and phosphorylated N-glycans of BM MSC analyzed by MALDI-TOF mass spectrometric profiling. Sulfated N-glycans of human mesenchymal stem cells change in their relative abundance during differentiation.

The invention is especially directed to terminal sulfated N-acetyllactosamine (LacNAc) structures comprising sulfate on 3- and/or 6-position Gal and/or 6 position of GlcNAc. The LacNAc is preferably type 2 LacNAc Galβ4GlcNAc, and even more preferably a N-glycan linked type II N-acetyllactosamine.

Combination of Terminal N-Glycan Structures and Complete N-Glycans

It is realized that the terminal type 2 N-acetyllactosamines are linked to N-glycan core structures and can be recognized by high specificity reagents or by mass spectrometry or combinations thereof as part of larger N-glycan structures. The mass spectrometric analysis is also directed to recognition of specific terminal structures based on mass spectrometric signals and/or corresponding monosaccharide compositions when the connection of the structures and the signals or compositions is established as in present invention for the mesenchymal cells.

Methods and reagents and combination thereof recognizing terminal epitopes of N-glycans are also in a preferred embodiment used for recognizing specific N-glycan structures. It is realized that methods directed to the complete N-glycan structures effectively characterize the stem cells.

Structures Associated with Nondifferentiated Human Mesenchymal Stem Cells

The Tables 1 and 3 show specific structure groups with specific monosaccharide compositions associated with the differentiation status of human mesenchymal stem cells.

For the preferred assignment of the structures corresponding to preferred monosaccharide composition of preferred altering or variable glycans see Tables 5 and 6. The structures correspond to the mass numbers and monosaccharide compositions of Tables 1-4, and glycosidase Table number 9 and monosaccharide; and compositions and structures described for glycans in Figures.

Analysis of Individual Specific Variation in Glycan Signal

Variation between glycan signals in the 5 measured MSC lines was measured as proportion of standard deviation to the average glycan signal. Most variation was detected (Tables 2 and 4):
a) in the neutral fraction in multifucosylated glycans, in glycans with terminal N-acetylhexosamine, and in glycans with terminal hexose;
b) in the acidic fraction in multifucosylated glycans, in multisialylated glycans, in glycans with terminal N-acetylhexosamine, and in glycans with sulfate esters.

In conclusion, there is most inter-cell line variation in N-glycan fucosylation, sialylation, sulphation, and glycan backbone formation with terminal N-acetylhexosamine.

The Structures Present in Higher Amount in hMSCs than in Corresponding Differentiated Cells

The invention revealed novel structures present in higher amounts in hMSCs than in corresponding differentiated cells.

The preferred hMSC enriched glycan groups are represented by groups hMSC 1 to hMSC 8, corresponding to several types of N-glycans. The glycans are preferred in the order from hMSC 1 to hMSC 8, based on the relative specificity for the non-differentiated hMSCs, the differences in expression are shown in Tables 1 and 3. The glycans are grouped based on similar composition and similar structures present to group comprising Complex type N-glycans, or High-Mannose type N-glycans and other preferred glycan groups.

Complex Type Glycans

hMSC 1, Disialylated Biantennary-Size Complex-Type N-Glycans

Specific expression in hMSCs was revealed for a specific group of biantennary complex type N-glycan structures. This group includes disialylated glycans including S2H5N4, S2H5N4F1, and S2H5N4F2.

Preferred Structural Subgroups of the Biantennary Complex Type Glycans Include NeuAc Comprising Glycans, and Fucosylated Glycans.

NeuAc Comprising Glycans

The sialylated glycans include NeuAc comprising glycans that shares the composition:

S₂H₅N₄F_{q}

Wherein H is preferably Gal or Man and N is GlcNAc, S is Neu5Ac and F is Fuc, q is an integer from 0 to 3.

The group comprises disialylated glycans with all levels of fucosylation. The preferred subgroups of this category include low fucosylation level glycans comprising no or one fucose residue (low fucosylation) and glycans with two or three fucose residues.

Preferred Biantennary Structures with Low Fucosylation

The preferred biantennary structures according to the invention include structures according to the Formula:

[NeuAcα]₀₋₁GalβGNβ2Manα3 ([NeuAcα]₀₋₁GalβGNβ2Manα6)Manβ4GNβ4(Fucα6)₀₋₁GN,

The GalβGlcNAc structures are preferably Galβ4GlcNAc-structures (type II N-acetyllactosamine antennae). The presence of type 2 structures was revealed by specific β4-linkage cleaving galactosidase (D. pneumoniae).

In a preferred embodiment the sialic acid is NeuAcα3- and the glycan comprises the NeuAc linked to Manα3-arm or Manα6-arm of the molecule. The assignment is based on the presence of α3-linked sialic acid revealed by specific sialidase digestion and by binders eg. MAA.

NeuAcα3GalβGNβ2Manα3/6([NeuAcα]₀₋₁GalβGNβ2Manα6/3)Manβ4GNβ4(Fucα6)₀₋₁GN, more preferably type II structures:

NeuAcα3Galβ4GNβ2Manα3/6([NeuAcα]₀₋₁Galβ4GNβ2Manα6/3)Manβ4GNβ4(Fucα6)₀₋₁GN.

The invention thus revealed preferred terminal epitopes, NeuAcα3GalβGN, NeuAcα3GalβGNβ2Man, NeuAcα3GalβGNβ2Manα3/6, to be recognized by specific binder molecules. It is realized that higher specificity preferred for application in context of similar structures can be obtained by using a binder that recognizes larger epitopes and thus differentiating e.g. between N-glycans and other glycan types in the context of the terminal epitopes.

Preferred Difucosylated and Sialylated Structures

Preferred difucosylated sialylated structures include structures, wherein the one fucose is in the core of the N-glycan and

a) one fucose on one arm of the molecule, and sialic acid is on the other arm (antenna of the molecule and the fucose is in Lewis x or H-structure:

Galβ4(Fucα3)GNβ2Manα3/6(NeuNAcαGalβGNβ2Manα6/3)Manβ4GNβ4(Fucα6)GN, and/or

Fucα2GalβGNβ2Manα3/6(NeuNAcαGalβGNβ2Manα6/3)Manβ4GNβ4(Fucα6)GN,

and when the sialic acid is α3-linked preferred antennary structures contain preferably the sialyl-lactosamine on α3-linked or α6-linked arm of the molecule according to formula:

Galβ4(Fucα3)GNβ2Manα6(NeuNAcα3Galβ4GNβ2Manα3)Manβ4GNβ4(Fucα6)GN, and/or

Fucα2GalβGNβ2Manα6(NeuNAcα3Galβ4GNβ2Manα3)Manβ4GNβ4(Fucα6)GN, and/or

Galβ4(Fucα3)GNβ2Manα3(NeuNAcα3Galβ4GNβ2Manα6)Manβ4GNβ4(Fucα6)GN, and/or

Fucα2GalβGNβ2Manα3(NeuNAcα3Galβ4GNβ2Manα6)Manβ4GNβ4(Fucα6)GN.

It is realized that the structures, wherein the sialic acid and fucose are on different arms of the molecules can be recognized as characteristic specific epitopes.

b) Fucose and NeuAc are on the same arm in the structure:

NeuNAcα3 Galβ3/4(Fucα4/3)GNβ2Manα3/6(GalβGNβ2Manα6/3)Manβ4GNβ4(Fucα6)GN, more preferably the structure is a N-glycan sialyl-Lewis x structure:

NeuNAcα3 Galβ4(Fucα3)GNβ2Manα3/6(GalβGNβ2Manα6/3)Manβ4GNβ4(Fucα6)GN

Preferred Sialylated Trifucosylated Structures

Preferred sialylated trifucosylated structures include glycans comprising core fucose and the terminal sialyl-Lewis x or sialyl-Lewis a, preferably sialyl-Lewis x due to the relatively high abundance presence of type 2 lactosamines, or Lewis y on either arm of the biantennary N-glycan according to the formulae:

NeuNAcα3 Galβ4(Fucα3)GNβ2Manα3/6([Fucα]GalβGNβ2Manα6/3)Manβ4GNβ4(Fucα6)GN, and/or

Fucα2Galβ4(Fucα3)GNβ2Manα3/6(NeuNAcα3/6GalβGNβ2Manα6/3)Manβ4GNβ4(Fucα6)GN. NeuNAc is preferably α3-linked on the same arm as fucose due to known biosynthetic preference and sialidase analysis. Preferably the structure comprises NeuNAcα3.

hMSC 5, Disialylated Hybrid-Type, Monoantennary, and Other Glycans

including S2H5N3F2P1, S2H5N3F1, S2H5N3, S2H6N3F1P1, S2H3N3F1, S2H3N3, S2H4N3, and S2H4N3F1, which correspond to unusual amount of sialic acid on regular core structures described for other glycan groups.

further including very unusual glycan compositions also corresponding to characteristic mass spectrometric signals S2H4N2F1, S2H3N2F1, S2H2N2, and S2H1N3F1

The preferred glycans include complex fucosylated glycans that shares the composition:

S₂HₚN₃F_{q}Pₛ

Wherein H is preferably Gal or Man and N is GlcNAc, S is Neu5Ac, F is Fuc, P is sulfate residue, p is an integer from 1 to 6, r is an integer from 2 to 3, q is an integer from 0 to 2; and s is an integer 0 or 1.

The unusual sialic acid structures include numerous possible variants known in the nature.

hMSC 6, Large Monosialylated Complex-Type N-Glycans

including S1H6N5, S1H6N5F1, S1H6N5F2, S1H6N5F3, S1H6N5F4, S1H6N6F1, S1H7N6F1, S1H7N6F2, S1H7N6F3, S1H7N6F4, S1H7N6F5, S1H8N7, S1H8N7F1, S1H8N7F3, and S1H11N10

The sialylated glycans include NeuAc comprising glycans that shares the composition:

S₁HₚNᵣF_{q}

Wherein H is preferably Gal or Man and N is GlcNAc, S is Neu5Ac, F is Fuc, P is sulfate residue, p is an integer from 6 to 11, preferably 6-8 or 11, r is an integer from 5 to 10, preferably 5-7 or 10 and q is an integer from 0 to 4.

An unusual feature in this group of glycans is presence of only single sialic acid residue (NeuNAc/Neu5Ac) in glycans comprising multiple N-acetyllactosamine units. The monosialylation indicates branch specific sialylation of multiantennary structures and presence of repetetive N-acetyllactosamines (LacNAcs providing only limited amount of sialylation sites). Terminal sialic acid structures are observable by specific lectins.

This group includes N-glycans comprising three LacNAc units with core composition H6N5, four LacNAc units with core composition H7N6, five LacNAc units with core composition H8N7, and eight LacNAc units with core composition H11N10. The glycans of this group includes multiantennary N-glycans and poly-N-cetyllactosamine comprising glycans. The presence of eight N-acetyllactosamine units indicates poly-N-acetyllactosamine structures.

The preferred structures in this group comprising S1H6N5F1-4 include tri-LacNac molecules triantennary N-glycans and elongated biantennary N-glycans. In a preferred embodiment the group includes

a) triantennary N-glycan comprising β1,4-linked N-acetyllactosamine branch, preferably linked to Manα6-arm of the N-glycan (mgat4 product N-glycan)

Gβ4GNβ2Mα3(Gβ4GNβ2{G≈4GNβ4}Mα6)Mβ4GNβ4(Fα6)GN,

wherein G is Gal, Gn is GlcNAc, M is Man, and F is Fuc and ( ) and { } indicated branches in the structure, and one of the LacNAc units comprises terminal Neu5Acα3-unit linked to Gal and each may LacNAc unit may comprise Fucα3 residue linked to GlcNAc or Fucα2 residue linked to Gal, which is not sialylated, so that the structure may comprise 1-3 fucose residues. and/or

b) poly-N-acetyllactosamine elongated biantennary complex-type N-glycans, wherein a LacNAc unit is linked to terminal Gal of a regular binatennary structure.

[Gβ4GNβ3]ₙ₁Gβ4GNβ2Mα3([Gβ4GNβ3]ₙ₂Gβ4GNβ2Mα6)Mβ4GNβ4(Fα6)GN,

wherein G is Gal, Gn is GlcNAc, M is Man, and F is Fuc and ( ) indicates a branch in the structure and [ ] indicates elongating LacNAc unit either present or absent, n1 and n2 are integers being either 0 or 1 independently and

either of the non-reducing end terminal LacNAc units comprises terminal Neu5Acα3-unit linked to Gal and each LacNAc unit may comprise Fucα3 residue linked to GlcNAc units or Fucα2 residue linked to Gal, which is not sialylated, so that the structure may comprise 1-3 fucose residues.

hMSC 7, Monosialylated Hybrid-Type and Monoantennary N-Glycans

including monoantennary glycans S1H3N3, S1H4N3, G1H4N3, S1H4N3F1, S1H4N3F3, and S1H4N3F1P1;

and hybrid-type glycans S1H5N3, G1H5N3, S1H5N3F1, S1H6N3, and S1H7N3

The preferred glycans include hybrid type and monoantennary glycans that shares the composition:

S₁HₚN3F_{q}Pₛ

Wherein H is preferably Gal or Man and N is GlcNAc, S is Neu5Ac or Neu5Gc, preferably Neu5Ac, F is Fuc, P is sulfate residue (SP in Tables 5 and 6), p is an integer from 3 to 7, q is an integer from 0, 1 or 3; and s is an integer 0 or 1.

The invention revealed characteristic monosialylated structures comprising only one LacNAc, preferably type II LacNAc unit. Based on biosynthetic consideration the sialyl-lacNAc unit is preferably linked to Manα3-structure in the N-glycan core. Thus this data reveals novel preferred type II sialyl N-acetyllactosamine structure epitopes SAα3/6Galβ4GlcNAcβ2Manα3, more preferably SAα3Galβ4GlcNAcβ2Manα3, wherein SA is Neu5Ac or Neu5Gc, more preferably Neu5Ac.

The preferred core structure for H3-7N3(F) glycans is:

Galβ4GlcNAcβ2Manα3({Manα}ₚManα6)Manβ4GlcNAcβ4(Fucα6)_{q}GlcNAc,

Wherein p is anteger from 0 to 3 indicating presence of α3, and/or a6 and/or a2-linked Man residues as present in monoantennary (p is 0)/hybrid type (p is 1-3) N-glycans, q is an integer 0 or 1, preferably additional fucose is Fucα2 linked to Gal, and/or Fucα3 linked to GlcNAc; and sulfate is linked to Gal or GlcNAc and sialic acid to Gal on the LacNAc units as described by the invention

more preferentially with type II N-acetyllactosamine antennae

hMSC 8, Complex-Fucosylated Sialylated Glycans

Including S1H7N6F3, S2H7N6F3, S3H7N6F3, S1H7N6F4, S2H7N6F4, S3H7N6F4, S1H7N6F5, S1H6N5F2, S1H6N5F3, S1H6N5F4, S1H5N4F2, S2H5N4F2, S1H4N3F3, S2H3N5F2, S1H5N4F4, S2H3N4F2, S1H4N4F2, S1H7N7F3, S1H7N6F2, S2H5N3F2P1, H5N3F2PI, and H3N6F3P1

A preferred group of N-glycans includes structures comprising more than one fucose residue. The structures comprise at least one fucose residue linked to LacNAc unit as described by the invention. The core structures are described for other groups and fucose residues are linked to LacNAc units as described by the invention.

The preferred glycans include complex fucosylated glycans that shares the composition:

SₙHₚNᵣF_{q}Pₛ

Wherein H is preferably Gal or Man and N is GlcNAc, S is Neu5Ac, F is Fuc, P is sulfate residue (SP in Tables 5 and 6),

n is an integer from 0 to 2; p is an integer from 3 to 8, r is an integer from 3 to 7, q is an integer from 2 to 4; and s is an integer 0 or 1.

High Mannose Type Glycans

hMSC 2, Large High-Mannose Type N-Glycans

The invention is directed to the group of Large high-mannose type N-glycans including non-fucosylated structures H6N2, H7N2, H8N2, and H9N2; and a fucosylated structure including H6N2F1.

The preferred high Mannose type glycans are according to the formula LHM:

[Mα2]ₙ₁Mα3{[Mα2]ₙ₃Mα6}Mα6{[Mα2]ₙ₆[Mα2]ₙ₇Mα3}Mβ4GNβ4[Fucα6]ₙ₈GNyR₂

wherein n1, n3, n6, and n7 and n8 are either independently 0 or 1;

with the provision that when n8 is 1 then the glycan comprises 6 Mannose residues, preferably n6 and n3 are 0 and either of n1 or n7 is 0.

y is anomeric linkage structure α and/or β or linkage from derivatized anomeric carbon, and

R₂ is reducing end hydroxyl, chemical reducing end derivative or natural asparagine N-glycoside derivative such as asparagine N-glycosides including aminoacid and/or peptides derived from protein;

[ ] indicates determinant either being present or absent depending on the value of n1, n3, n6, n7; and

{ } indicates a branch in the structure;

M is D-Man, GN is N-acetyl-D-glucosamine., y is anomeric structure or linkage type, preferably beta to Asn.

The preferred non-fucosylated structures in this group include:

Manα2Manα6(Manα2Manα3)Manα6(Manα2Manα2Manα3)Manβ4GNβ4GN,

Manα2Manα6([Manα2]ₙ₃Manα3)Manα6([Manα2]ₙ₆Manα2Manα3)Manβ4GNβ4GN,

Manα2Manα6(Manα3)Manα6(Manα2Manα2Manα3)Manβ4GNβ4GN

Manα2Manα6(Manα2Manα3)Manα6(Manα2Manα3)Manβ4GNβ4GN

Manα2Manα6(Manα3)Manα6(Manα2Manα3)Manβ4GNβ4GN

The preferred fucosylated structures includes

[Manα2]ₙ₁Manα6(Manα3)Manα6([Manα2]ₙ₇Manα3)Manβ4GNβ4(Fucα6)GN,

Manα2Manα6(Manα3)Manα6(Manα3)Manβ4GNβ4(Fucα6)GN,

Manα6(Manα3)Manα6(Manα2Manα3)Manβ4GNβ4(Fucα6)GN,

hMSC 4, Glucosylated High-Mannose Type N-Glycans

The preferred group of glucosylated high-mannose type N-glycans includes H10N2, H11N2, and H12N2

The group of glucosylated high-mannose type glycans is continuous to high-mannose glycans. The group of glycans is involved in quality control in ER of cells. The presence of glucosylated high-mannose glycans is considered to correspond to protein synthesis activity and folding efficiency in the cells. The terminal glucose residue is characteristic structure for glycans of this group and in a preferred embodiment the invention is directed to the recognition of the terminal Glc residues by specific binding agents. It is further realized that reagents recognizing high mannos glycan also recognize this structure especially when the recognition is directed to terminal Manα2-structures on non-glucosylated arms of the molecule

The invention revealed substantially more of this type of glycans in mesenchymal stem cells than in differentiated cells, especially osteogenically differentiated bone marrow derived stem cells.

The preferred structures are according to the Formula:

Mα2Mα6(Mα2Mα3)Mα6([Gα2]ₙ₁[Gα3]ₙ₂[Gα3]ₙ₃Mα2Mα2Mα3)Mβ4GNβ4GN,

wherein n1, n2 and n3 are either 0 or 1, idenpendently

wherein M is mannose, G is glucose, and GN is N-acetylglucosamine residue

hMSC 3, Soluble Oligomannose Glycans

including H2N1, H3N1, H4N1, H5N1, H6N1, H7N1, H8N1, and H9N1

Structures and Compositions Associated with Differentiated Mesenchymal Cells

The invention revealed novel structures present in higher amount in differentiated mesenchymal stem cells than in corresponding non-differentiated hMSCs.

The preferred glycan groups are represented in groups Diff 1 to Diff 7, corresponding to several types of N-glycans. The glycans are preferred in the order from Diff 1 to Diff 7, based on the relative specificity for the non-differentiated hMSCs, the differences in the expression are shown in Table 1.

Diff 1, Sulfated Glycans

Including biantennary-size complex-type glycans H5N4P1, H5N4F1P1, S2H5N4F1P1, H5N4F2P1, H5N4F3P1, S1H5N4P1, S1H5N4F1P1;

Large complex-type glycans H6N5F1P1, S2H6N5F1P1, H7N6F1P1, H6N5F3P1, and S1H6N5F1P1;

Terminal Hex containing glycans H6N4F3P1, G1H6N4P1, and H7N4P1;

Terminal HexNAc containing glycans S2H4N5F2P2, H4N4F1P1, H3N6F1P1, H4N5F2P1, H3N5F1P1, H3N4P1, H3N4F1P1, and and H4N4P1;

And hybrid-type or monoantennary glycans S2H4N3F1P1, H4N3F1P1, H4N3P1, H5N3F1P1, H4N3F2P1, S1H3N3F1P2, H3N3F1P1, H3N3P1, and S2H5N3P2;

And high-mannose type glycans including H10N2F1P2, which are preferentially phosphorylated.

The preferred sulfated glycans comprise N-glycan core and preferred type N-acetyllactosamine unit or units which are sulfated, in case or theminal HexNAc units such as GlcNAc, or GalNAc,4GlcNAc these may be further sulfated. The presence of sulfate residue on the lactosamine/GlcNAc comprising N-glycans was analyzed by high resolution mass spectrometry and/or specific phosphatase enzyme digestion. The glycans may further comprise Neu5Ac and fucose residues.

The sulfated glycans include complex type and related glycans that shares the composition:

SₙHₚNᵣF_{q}Pₛ

Wherein H is preferably Gal or Man and N is GlcNAc, S is Neu5Ac, F is Fuc, P is sulfate residue (SP in Tables 5 and 6),

n is an integer from 0 to 2; p is an integer from 3 to 7, r is an integer from 3 to 6, q is an integer from 0, 1 or 3; and s is an integer 1 or 2.

The sulfated glycans Large complex-type glycans H6N5F1P1, S2H6N5F1P1, H7N6F1P1, H6N5F3P1, and S1H6N5F1P1 include complex type and related glycans that shares the composition:

SₙHₚNᵣF_{q}Pᵢ

Wherein H is preferably Gal or Man and N is GlcNAc, S is Neu5Ac, F is Fuc, P is sulfate residue (SP in Tables 5 and 6),

n is an integer from 0 to 2; p is an integer from 6 to 7, r is an integer from 5 to 6, and q is an integer 1 or 3. The preferred core structures with core composition H6N5-comprising glycans was described for hMSC 6, glycans with composition of H7N6 comprise four LacNAc units as tetraantennary and/or poly-lacNAc comprising structure. The diasialylate structure comprises two Neu5Ac units at terminal LacNAc units and one fucose residue is in a preferred embodiment linked to the core of the N-glycan.

The preferred sulfated biantennary N-glycans include glycans that shares the composition:

SₙH₅N₄F_{q}Pᵢ

Wherein H is preferably Gal or Man and N is GlcNAc, S is Neu5Ac and F is Fuc, n is an integer from 0 or 2; q is an integer from 0 to 3.

The preferred structures are as described for biantennary N-glycans in hMSC groups, but the glycans further comprise a sulfate group linked to N-acetyllactosamine unit as described for preferred sulfates terminal N-glycan structure comprising terminal type 2 LacNAc units. The presence of a disialylated structure indicates that the glycans comprise at least part of the sulphate residues linked to 6-position of GlcNAc and/or Gal residue.

The preferred core structures of the glycans has been represented in Tables and in other preferred groups, the invention is further directed to following preferred core structure groups comprising sulphated LacNAc or GlcNAc:

The preferred core H4H5 structures, H4N5 and H4N5F2, include following preferred structures comprising LacdiNAc:

[Fucα]ₙ₃{Gal[NAc]ₙ₁βGNβ2Manα3(Gal[NAc]ₙ₂βGNβ2Manα6)Manβ4GNβ4(Fucα6)ₙ₂GN,

wherein n1 and n2 are either 0 or 1, so that either n1 or n2 is 0 and the other is 1 and n3 is either 0 or 1. The fucose residue forms preferably Lewis x or fucosylated LacdiNAc structure GalNAcβ4(Fucα3)GlcNAc.

Preferred core structures of hybrid-type N-glycans, including H5N3, according to the Formula:

[Galβ]ₙ₁GlcNAcβ2Manα3(Manα3/6[Manα6/3]ₙ₃Manα6)Manβ4GlcNAcβ4(Fucα6)ₙ₂GlcNAc

Wherein n1 and n2 and n3 are either 0 or 1, so that there is 5 hexose (Gal/Man) units.

The preferred H5N3 comprising structures comprise core structure according to the Formula

GlcNAcβ2Manα3(Manα3[Manα6]Manα6)Manβ4GlcNAcβ4(Fucα6)ₙ₂GlcNAc

Wherein n2 is either 0 or 1.

Terminal HexNAc monoantennary N-glycans, with core structure compositions H3N3F1;

preferentially includes core structures (Galβ4)₀₋₁GlcNAcβ2Manα3([Manα6]₀₋₁)Manβ4GlcNAcβ4(Fucα6)GlcNAc, more preferentially with type II N-acetyllactosamine antennae (without Manα6 branch), wherein galactose residue is β1,4-linked.

Diff 2, Low-Mannose Type N-Glycans

Including non-fucosylated glycans H1N2, H3N2, and H4N2;

And fucosylated glycans H2N2F1, H3N2F1, and H4N2F1

Diff 3, Small High-Mannose Type (Man5) N-Glycans

comprising non-fucosylated H5N2 and fucosylated H5N2F1

Diff 4, Neutral Hybrid-Type and Monoantennary N-Glycans

Including monoantennary glycans H2N3, H2N3F1, H3N3, H3N3F1, H3N3F2;

Hybrid-type and/or monoantennary glycans H4N3 and H4N3F1;

And hybrid-type glycans H4N3F2, H5N3, H5N3F1, H5N3F2, H6N3, H6N3F1, and H7N3

Diff 5, Neutral Complex-Type N-Glycans

Including biantennary-size complex-type glycans H5N4, H5N4F1, H5N4F2, and H5N4F3;

Large complex-type glycans H6N5, H6N5F1, H6N5F2, H6N5F3, H6N5F4, H7N6, H7N6F1, and H8N7;

Terminal HexNAc containing glycans H5N5, H5N5F1, H5N5F2, H5N5F3, H6N6, H3N4, H4N4, H4N4F1, H4N4F2, H4N5, H4N5F2, and H3N6F1;

Terminal Hex containing glycans H6N4, H6N4F1, H7N4, H6N4F2, H7N4F1, and H8N4.

Preferred core structures of the glycans has been described in context of other glycan groups and for H4N5 (Diff 1) and H5N5 structures below.

Diff6 is found in Table 1.

The glycans comprising core composition H=N=5 type are preferably terminal HexNAc comprising N-glycans, including H5N5F1, H5N5, H5N5F3

Comprising the binatennary N-glycan core structure and terminal HexNAc, especially terminal GlcNAc glycans linked to the core of the N-glycan

Diff 7, Monosialylated Biantennary-Size Complex-Type N-Glycans Including G1H5N4, S1H5N4P1, S1H5N4F1, G1H5N4F1, S1H5N4F1P1, and S1H5N4F3

S₁H₅N₄F_{q}Pₛ

Wherein H is preferably Gal or Man and N is GlcNAc, S is Neu5Ac or Neu5Gc, preferably Neu5Ac and F is Fuc and P is sulfate residue,

q is an integer from 0 to 3, preferably 0, 1 or 3, s is an integer 0 or 1.

The preferred core structures of the biantennary N-glycans are described in other groups according ot the invention. The glycans comprise one preferred sialyl-LacNAc unit and one LacNAc unit, which may be further sulphated and/or fucosylated.

Preferred N-Glycan Structure Types

The invention revealed N-glycans with common core structure of N-glycans, which change according to differentiation and/or between individual cell lines. For assignment of the structures see also TABLE 5 and 6. The structures correspond also to the mass numbers and monosaccharide compositions of Tables 1-4, glycosidase Table number 9 and monosaccharide compositions and structures described of glycans changing in context of differentiation and in Figures. Monosaccharide composition corresponding to a glycan structure is obtained by indicating Gal and Man as Hex (or H in shorter presentation), the number of Hex units is sum of amount of Man and Gal residue; and GlcNAc (or GalNAc) residue as HexNAc or N and indicating the number of fucose residues (F), sialic acid residues (S/Neu5Ac or G/Neu5Gc), Ac indicates O-acetyl residues and possible sulfate or phosphoryl residues are indicated with number after SP or P sharing similar molecular weight. The N-glycans of mesenchymal stem cells comprise the core structure comprising Man B4GlcNAc structure in the core structure of N-linked glycan according to the Formula CGN:

[Manα3]ₙ₁(Manα6)ₙ₂Manβ4GlcNAcβ4(Fucα6)ₙ₃GlcNAcxR,
wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures (for the analysis of the status of stem cells and/or manipulation of the stem cells), wherein xR indicates reducing end structure of N-glycan linked to protein or peptide such as βAsn or βAsn-peptide or βAsn-protein, or free reducing end of N-glycan or chemical derivative of the reducing end produced for analysis.

The preferred Mannose type glycans are according to the formula:

[Mα2]ₙ₁[Mα3]ₙ₂{[Mα2]ₙ₃[Mα6)]ₙ₄}[Mα6]ₙ₅{[Mα2]ₙ₆[Mα2]ₙ₇[Mα3]ₙ₈}Mβ4GNβ4[{Fucα6}]ₘGNyR₂ Formula M2:

wherein n1, n2, n3, n4, n5, n6, n7, n8, and m are either independently 0 or 1; with the provision that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; y is anomeric linkage structure (x and/or 0 or linkage from derivatized anomeric carbon, and

R₂ is reducing end hydroxyl, chemical reducing end derivative or natural asparagine N-glycoside derivative such as asparagine N-glycosides including asparagines N-glycoside amino acid and/or peptides derived from protein;

[ ] indicates determinant either being present or absent depending on the value of n1, n2, n3, n4, n5, n6, n7, n8, and m; and

{ } indicates a branch in the structure;

M is D-Man, GN is N-acetyl-D-glucosamine and Fuc is L-Fucose,

and the structure is optionally a high mannose structure, which is further substituted by glucose residue or residues linked to mannose residue indicated by n6.

Several preferred low Man glycans described above can be presented in a single Formula:

[Mα3]ₙ₂{[Mα6)]ₙ₄}[Mα6]ₙ₅{[Mα3]ₙ₈}Mβ4GNβ4[{Fucα6}]ₘGNyR₂

wherein n2, n4, n5, n8, and m are either independently 0 or 1; with the provision that when n5 is 0, also n2, and n4 are O;the sum of n2, n4, n5, and n8 is less than or equal to (m+3); [ ] indicates determinant either being present or absent depending on the value of n2, n4, n5, n8, and m; and

{ } indicates a branch in the structure;

y and R2 are as indicated above.

Preferred non-fucosylated low-mannose glycans are according to the formula:

[Mα3]ₙ₂([Mα6)]ₙ₄)[Mα6]ₙ₅{[Mα3]ₙ₈}Mβ4GNβ4GNyR₂

wherein n2, n4, n5, n8, and m are either independently 0 or 1,

with the provision that when n5 is 0, also n2 and n4 are 0, and preferably either n2 or n4 is 0,

[ ] indicates determinant either being present or absent depending on the value of, n2, n4, n5, n8,

{ } and ( ) indicates a branch in the structure,

y and R2 are as indicated above.

Preferred Individual Structures of Non-Fucosylated Low-Mannose Glycans

Special Small Structures

Small non-fucosylated low-mannose structures are especially unusual among known N-linked glycans and characteristic glycan group useful for separation of cells according to the present invention. These include:

Mβ4GNβ4GNyR₂

Mα6Mβ4GNβ4GNyR₂

Mα3Mβ4GNβ4GNyR₂ and

Mα6{Mα3}Mβ4GNβ4GNyR₂.

Mβ4GNβ4GNyR₂ trisaccharide epitope is a preferred common structure alone and together with its mono-mannose derivatives Mα6Mβ4GNβ4GNyR₂ and/or Mα3Mβ4GNβ4GNyR₂, because these are characteristic structures commonly present in glycomes according to the invention. The invention is specifically directed to the glycomes comprising one or several of the small non-fucosylated low-mannose structures. The tetrasaccharides are in a specific embodiment preferred for specific recognition directed to α-linked, preferably α3/6-linked Mannoses as preferred terminal recognition element.

Special Large Structures

The invention further revealed large non-fucosylated low-mannose structures that are unusual among known N-linked glycans and have special characteristic expression features among the preferred cells according to the invention. The preferred large structures include

[Mα3]ₙ₂([Mα6]ₙ₄)Mα6{Mα3}Mβ4GNβ4GNyR₂

more specifically

Mα6Mα6{Mα3}Mβ4GNβ4GNyR₂

Mα3Mα6{Mα3}Mβ4GNβ4GNyR₂ and

Mα3(Mα6)Mα6{Mα3}Mβ4GNβ4GNyR₂.

The hexasaccharide epitopes are preferred in a specific embodiment as rare and characteristic structures in preferred cell types and as structures with preferred terminal epitopes. The heptasaccharide is also preferred as a structure comprising a preferred unusual terminal epitope Mα3(Mα6)Mα useful for analysis of cells according to the invention.

Preferred fucosylated low-mannose glycans are derived according to the formula:

[Mα3]ₙ₂{[Mα6]ₙ₄}[Mα6]ₙ₅{[Mα3]ₙ₈}Mβ4GNβ4(Fucα6)GNyR₂

wherein n2, n4, n5, n8, and m are either independently 0 or 1,with the provision that when n5 is 0, also n2 and n4 are 0,

[ ] indicates determinant either being present or absent depending on the value of n2, n4, n5, n8, and m;

{ } and ( ) indicate a branch in the structure.

Preferred Individual Structures of Fucosylated Low-Mannose Glycans

Small fucosylated low-mannose structures are especially unusual among known N-linked glycans and form a characteristic glycan group useful for separation of cells according to the present invention. These include:

Mβ4GNβ4(Fucα6)GNyR₂

Mα6Mβ4GNβ4(Fucα6)GNyR₂

Mα3Mβ4GNβ4(Fucα6)GNyR₂ and

Mα6{Mα3}Mβ4GNβ4(Fucα6)GNyR₂.

Mβ4GNβ4(Fucα6)GNyR₂ tetrasaccharide epitope is a preferred common structure alone and together with its monomannose derivatives Mα6Mβ4GNβ4(Fucα6)GNyR₂ and/or Mα3Mβ4GNβ4(Fucα6)GNyR₂, because these are commonly present characteristic structures in glycomes according to the invention. The invention is specifically directed to the glycomes comprising one or several of the small fucosylated low-mannose structures. The tetrasaccharides are in a specific embodiment preferred for specific recognition directed to α-linked, preferably α3/6-linked Mannoses as preferred terminal recognition element.

Special Large Structures

The invention further revealed large fucosylated low-mannose structures that are unusual among known N-linked glycans and have special characteristic expression features among the preferred cells according to the invention. The preferred large structures include

[Mα3]ₙ₂([Mα6]ₙ₄)Mα6{Mα3}Mβ4GNβ4(Fucα6)GNyR₂ more specifically

Mα6Mα6{Mα3}Mβ4GNβ4(Fucα6)GNyR₂

Mα3Mα6{Mα3}Mβ4GNβ4(Fucα6)GNyR₂ and

Mα3(Mα6)Mα6{Mα3}Mβ4GNβ4(Fucα6)GNyR₂.

The heptasaccharide epitopes are preferred in a specific embodiment as rare and characteristic structures in preferred cell types and as structures with preferred terminal epitopes. The octasaccharide is also preferred as structure comprising a preferred unusual terminal epitope Mα3(Mα6)Mα useful for analysis of cells according to the invention.

Preferred Non-Reducing End Terminal Mannose-Epitopes

The inventors revealed that mannose-structures can be labeled and/or otherwise specifically recognized on cell surfaces or cell derived fractions/materials of specific cell types. The present invention is directed to the recognition of specific mannose epitopes on cell surfaces by reagents binding to specific mannose structures on cell surfaces.

The preferred reagents for recognition of any structures according to the invention include specific antibodies and other carbohydrate recognizing binding molecules. It is known that antibodies can be produced for the specific structures by various immunization and/or library technologies such as phage display methods representing variable domains of antibodies. Similarly with antibody library technologies, including aptamer technologies and including phage display for peptides, exist for synthesis of library molecules such as polyamide molecules including peptides, especially cyclic peptides, or nucleotide type molecules such as aptamer molecules.

The invention is specifically directed to specific recognition of high-mannose and low-mannose structures according to the invention. The invention is specifically directed to recognition of non-reducing end terminal Manox-epitopes, preferably at least disaccharide epitopes, according to the formula:

[Mα2]ₘ₁[Mαx]ₘ₂[Mα6]ₘ₃{{[Mα2]ₘ₉[Mα2]ₘ₈[Mα3]ₘ₇}ₘ₁₀(Mβ4[GN]ₘ₄)ₘ₅}ₘ₆yR₂

wherein m1, m2, m3, m4, m5, m6, m7, m8, m9 and m10 are independently either 0 or 1; with the provision that when m3 is 0, then m1 is 0, and when m7 is 0 then either m1-5 are 0 and m8 and m9 are 1 forming a Mα2Mα2-disaccharide, or both m8 and m9 are 0;

y is anomeric linkage structure α and/or β or linkage from derivatized anomeric carbon, and

R₂ is reducing end hydroxyl or chemical reducing end derivative and x is linkage position 3 or 6 or both 3 and 6 forming branched structure,

{ } indicates a branch in the structure.

The invention is further directed to terminal Mα2-containing glycans containg at least one Mα2-group and preferably Mα2-group on each branch so that m1 and at least one of m8 or m9 is 1. The invention is further directed to terminal Mα3 and/or Mα6-epitopes without terminal Mα2-groups, when all m1, m8 and m9 are 1.

The invention is further directed in a preferred embodiment to the terminal epitopes linked to a Mβ-residue and for application directed to larger epitopes. The invention is especially directed to Mβ4GN-comprising reducing end terminal epitopes.

The preferred terminal epitopes comprise typically 2-5 monosaccharide residues in a linear chain. According to the invention short epitopes comprising at least 2 monosaccharide residues can be recognized under suitable background conditions and the invention is specifically directed to epitopes comprising 2 to 4 monosaccharide units and more preferably 2-3 monosaccharide units, even more preferred epitopes include linear disaccharide units and/or branched trisaccharide non-reducing residue with natural anomeric linkage structures at reducing end. The shorter epitopes may be preferred for specific applications due to practical reasons including effective production of control molecules for potential binding reagents aimed for recognition of the structures.

The shorter epitopes such as Mα2M is often more abundant on target cell surface as it is present on multiple arms of several common structures according to the invention.

Preferred Disaccharide Epitopes Include

Manα2Man, Manα3Man, Manα6Man, and more preferred anomeric forms Manα2Manα, Manα3Manβ, Manα6Manβ, Manα3Manα and Manα6Manα.

Preferred branched trisaccharides include Manα3(Manα6)Man, Manα3(Manα6)Manβ, and Manα3(Manα6)Manα.

The invention is specifically directed to the specific recognition of non-reducing terminal Manα2-structures especially in context of high-mannose structures.

The invention is specifically directed to following linear terminal mannose epitopes:

a) preferred terminal Manα2-epitopes including following oligosaccharide sequences:

Manα2Man,

Manα2Manα,

Manα2Manα2Man, Manα2Manα3Man, Manα2Manα6Man,

Manα2Manα2Manα, Manα2Manα3Manβ, Manα2Manα6Manα,

Manα2Manα2Manα3Man, Manα2Manα3Manα6Man, Manα2Manα6Manα6Man

Manα2Manα2Manα3Manβ, Manα2Manα3Manα6Manβ,

Manα2Manα6Manα6Manβ;

The invention is further directed to recognition of and methods directed to non-reducing end terminal Manα3- and/or Manα6-comprising target structures, which are characteristic features of specifically important low-mannose glycans according to the invention. The preferred structural groups include linear epitopes according to b) and branched epitopes according to the c3) especially depending on the status of the target material.

b) preferred terminal Manα3- and/or Manα6-epitopes including following oligosaccharide sequences:

Manα3Man, Manα6Man, Manα3Manβ, Manα6Manβ, Manα3Manα, Manα6Manα,

Manα3Manα6Man, Manα6Manα6Man, Manα3Manα6Manβ, Manα6Manα6Manβ and to following:

c) branched terminal mannose epitopes are preferred as characteristic structures of especially high-mannose structures (c1 and c2) and low-mannose structures (c3), the preferred branched epitopes including:

c1) branched terminal Manα2-epitopes

Manα2Manα3(Manα2Manα6)Man, Manα2Manα3(Manα2Manα6)Manα,

Manα2Manα3(Manα2Manα6)Manα6Man,

Manα2Manα3(Manα2Manα6)Manα6Manβ,

Manα2Manα3(Manα2Manα6)Manα6(Manα2Manα3)Man,

Manα2Manα3(Manα2Manα6)Manα6(Manα2Manα2Manα3)Man,

Manα2Manα3(Manα2Manα6)Manα6(Manα2Manα3)Manβ

Manα2Manα3(Manα2Manα6)Manα6(ManαManα2Manα3)Manβ

c2) branched terminal Manα2- and Manα3 or Manα6-epitopes according to formula when m1 and/or m8 and/m9 is 1 and the molecule comprise at least one nonreducing end terminal Manα3 or Manα6-epitope

c3) branched terminal Manα3 or Manα6-epitopes

Manα3(Manα6)Man, Manα3(Manα6)Manβ, Manα3(Manα6)Manα,

Manα3(Manα6)Manα6Man, Manα3(Manα6)Manα6Manβ,

Manα3(Manα6)Manα6(Manα3)Man, Manα3(Manα6)Manα6(Manα3)Manβ

The present invention is further directed to increase the selectivity and sensitivity in recognition of target glycans by combining recognition methods for terminal Manα2 and Manα3 and/or Manα6-comprising structures. Such methods would be especially useful in the context of cell material according to the invention comprising both high-mannose and low-mannose glycans.

Complex Type N-Glycans

According to the present invention, complex-type structures are preferentially identified by mass spectrometry, preferentially based on characteristic monosaccharide compositions, wherein HexNAc≧4 and Hex≧3. In a more preferred embodiment of the present invention, 4≦HexNAc≦20 and 3≦Hex≦21, and in an even more preferred embodiment of the present invention, 4≦HexNAc≦10 and 3≦Hex≦11. The complex-type structures are further preferentially identified by sensitivity to endoglycosidase digestion, preferentially N-glycosidase F detachment from glycoproteins. The complex-type structures are further preferentially identified in NMR spectroscopy based on characteristic resonances of the Manα3(Manα6)Manβ4GlcNAcβ4GlcNAc N-glycan core structure and GlcNAc residues attached to the Manα3 and/or Manα6 residues.

Beside Mannose-type glycans the preferred N-linked glycomes include GlcNAcβ2-type glycans including Complex type glycans comprising only GlcNAcβ2-branches and Hydrid type glycan comprising both Mannose-type branch and GlcNAcβ2-branch.

GlcNAcβ2-Type Glycans

The invention revealed GlcNAcβ2Man structures in the glycomes according to the invention. Preferably GlcNAcβ2Man-structures comprise one or several of GlcNAcβ2Manα-structures, more preferably GlcNAcβ2Manα3- or GlcNAcβ2Manα6-structure.

The Complex type glycans of the invention comprise preferably two GlcNAcβ2Manα structures, which are preferably GlcNAcβ2Manα3 and GlcNAcβ2Manα6. The Hybrid type glycans comprise preferably GlcNAcβ2Manα3-structure.

The present invention is directed to at least one of natural oligosaccharide sequence structures and structures truncated from the reducing end of the N-glycan according to the Formul CO1 (also referred as GNβ2):

[R₁GNβ2]ₙ₁[Mα3]ₙ₂{[R₃]ₙ₃[GNβ2]ₙ₄Mα6}ₙ₅Mβ4GNXyR₂,

with optionally one or two or three additional branches according to formula [RₓGNβz]ₙₓ linked to Mα6-, Mα3-, or Mβ4, and Rₓ may be different in each branch

wherein n1, n2, n3, n4, n5 and nx, are either 0 or 1, independently,

with the provision that when n2 is 0 then n1 is 0 and when n3 is 1 and/or n4 is 1 then n5 is also 1, and at least n1 or n4 is 1, or n3 is 1;

when n4 is 0 and n3 is 1 then R₃ is a mannose type substituent or nothing and

wherein X is a glycosidically linked disaccharide epitope β4(Fucα6)ₙGN, wherein n is 0 or 1, or X is nothing and

y is anomeric linkage structure α and/or β or linkage from derivatized anomeric carbon, and

R₁, Rₓ and R₃ indicate independently one, two or three natural substituents linked to the core structure,

R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

Elongation of GlcNAcβ2-Type Structures Forming Complex/Hydrid Type Structures

The substituents R₁, Rₓ and R₃ may form elongated structures. In the elongated structures R₁, and Rₓ represent substituents of GlcNAc (GN) and R₃ is either substituent of GlcNAc or when n4 is 0 and n3 is 1 then R3 is a mannose type substituent linked to Manα6-branch forming a Hybrid type structure. The substituents of GN are monosaccharide Gal, GalNAc, or Fuc and/or acidic residue such as sialic acid or sulfate or phosphate ester.

GlcNAc or GN may be elongated to N-acetyllactosaminyl also marked as GalβGN or di-N-acetyllactosdiaminyl GalNAcβGlcNAc, preferably GalNAcβ4GlcNAc. LNβ2M can be further elongated and/or branched with one or several other monosaccharide residues such as galactose, fucose, SA or LN-unit(s) which may be further substituted by SAα-strutures,

and/or Mα6 residue and/or Mα3 residue can be further substituted by one or two β6-,

and/or β4-linked additional branches according to the formula;

and/or either of Mα6 residue or Mα3 residue may be absent;

and/or Mα6-residue can be additionally substituted by other Manα units to form a hybrid type structures;

and/or Manβ4 can be further substituted by GNβ4,

and/or SA may include natural substituents of sialic acid and/or it may be substituted by other SA-residues preferably by α8- or α9-linkages.

The SAα-groups are linked to either 3- or 6-position of neighboring Gal residue or on 6-position of GlcNAc, preferably 3- or 6-position of neighboring Gal residue. In separately preferred embodiments the invention is directed to structures comprising solely 3-linked SA or 6-linked SA, or mixtures thereof.

Preferred Complex Type Structures

Incomplete Monoantennary N-Glycans

The present invention revealed incomplete Complex monoantennary N-glycans, which are unusual and useful for characterization of glycomes according to the invention. The most of the incomplete monoantennary structures indicate potential degradation of biantennary N-glycan structures and are thus preferred as indicators of cellular status. The incomplete Complex type monoantennary glycans comprise only one GNβ2-structure.

The invention is specifically directed to structures according to the Formula CO1 or Formula GNb2 above when only n1 is 1 or n4 is 1 and mixtures of such structures.

The preferred mixtures comprise at least one monoantennary complex type glycans

A) with a single branch likely from a degradative biosynthetic process:

R₁GNβ2Mα3β4GNXyR₂

R₃GNβ2Mα6Mβ4GNXyR₂ and

B) with two branches comprising mannose branches

B 1) R₁GNβ2Mα3{Mα6}ₙ₅Mβ4GNXyR₂

B2) Mα3{R₃GNβ2Mα6}ₙ₅Mβ4GNXyR₂

The structure B2 is preferred over A structures as product of degradative biosynthesis, it is especially preferred in context of lower degradation of Manα3-structures. The structure B1 is useful for indication of either degradative biosynthesis or delay of biosynthetic process.

Biantennary and Multiantennary Structures

The inventors revealed a major group of biantennary and multiantennary N-glycans from cells according to the invention. The preferred biantennary and multiantennary structures comprise two GNβ2 structures. These are preferred as an additional characteristic group of glycomes according to the invention and are represented according to the Formula CO2:

R₁GNβ2Mα3{R₃GNβ2Mα6}Mβ4GNXyR₂

with optionally one or two or three additional branches according to formula [RₓGNβz]ₙₓ linked to Mα6-, Mα3-, or Mβ4 and Rₓ may be different in each branch

wherein nx is either 0 or 1,

and other variables are according to the Formula CO1.

Preferred Biantennary Structure

A biantennary structure comprising two terminal GNβ-epitopes is preferred as a potential indicator of degradative biosynthesis and/or delay of biosynthetic process.

The more preferred structures are according to the Formula CO2 when R₁ and R₃ are nothing.

Elongated Structures

The invention revealed specific elongated complex type glycans comprising Gal and/or GalNAc-structures and elongated variants thereof. Such structures are especially preferred as informative structures because the terminal epitopes include multiple informative modifications of lactosamine type, which characterize cell types according to the invention.

The present invention is directed to at least one of natural oligosaccharide sequence structure or group of structures and corresponding structure(s) truncated from the reducing end of the N-glycan according to the Formula CO3:

[R₁Gal[NAc]ₒ₂βz2]ₒ₁GNβ2Mα3{[R₁Gal[NAc]ₒ₄βz2]ₒ₃GNβ2Mα6}Mβ4GNXyR₂,

with optionally one or two or three additional branches according to formula [RₓGNβz1]ₙₓ linked to Mα6-, Mα3-, or Mβ4 and Rₓ may be different in each branch

wherein nx, o1, o2, o3, and o4 are either 0 or 1, independently,

with the provision that at least o1 or o3 is 1, in a preferred embodiment both are 1;

z2 is linkage position to GN being 3 or 4, in a preferred embodiment 4;

z1 is linkage position of the additional branches;

R₁, Rx and R₃ indicate one or two a N-acetyllactosamine type elongation groups or nothing,

{ } and ( ) indicates branching which may be also present or absent,

other variables are as described in Formula GNb2.

Galactosylated Structures

The inventors characterized useful structures especially directed to digalactosylated structure

GalβzGNβ2Mα3{GalβzGNβ2Mα6}Mβ4GNXyR₂,

and monogalactosylated structures:

GalβzGNβ2Mα3{GNβ2Mα6}Mβ4GNXyR₂,

GNβ2Mα3{GalβzGNβ2Mα6}Mβ4GNXyR₂,

and/or elongated variants thereof preferred for carrying additional characteristic terminal structures useful for characterization of glycan materials

R₁GalβzGNβ2Mα3{R₃GalβzGNβ2Mα6}Mβ4GNXyR₂

R₁GalβzGNβ2Mα3{GNβ2Mα6}Mβ4GNXyR₂, and

GNβ2Mα3{R₃GalβzGNβ2Mα6}Mβ4GNXyR₂.

Preferred elongated materials include structures wherein R₁ is a sialic acid, more preferably NeuNAc or NeuGc.

LacdiNAc-Structure Comprising N-Glycans

The present invention revealed for the first time LacdiNAc, GalNAcβGlcNAc structures from the cell according to the invention. Preferred N-glycan lacdiNAc structures are included in structures according to the Formula CO1, when at least one the variable o2 and o4 is 1.

The Major Acidic Glycan Types

The acidic glycomes mean glycomes comprising at least one acidic monosaccharide residue such as sialic acids (especially NeuNAc and NeuGc) forming sialylated glycome, HexA (especially GlcA, glucuronic acid) and/or acid modification groups such as phosphate and/or sulfate esters.

According to the present invention, presence of sulfate and/or phosphate ester (SP) groups in acidic glycan structures is preferentially indicated by characteristic monosaccharide compositions containing one or more SP groups. The preferred compositions containing SP groups include those formed by adding one or more SP groups into non-SP group containing glycan compositions, while the most preferential compositions containing SP groups according to the present invention are selected from the compositions described in the acidic N-glycan fraction glycan group Tables of the present invention. The presence of phosphate and/or sulfate ester groups in acidic glycan structures is preferentially further indicated by the characteristic fragments observed in fragmentation mass spectrometry corresponding to loss of one or more SP groups, the insensitivity of the glycans carrying SP groups to sialidase digestion. The presence of phosphate and/or sulfate ester groups in acidic glycan structures is preferentially also indicated in positive ion mode mass spectrometry by the tendency of such glycans to form salts such as sodium salts as described in the Examples of the present invention. Sulfate and phosphate ester groups are further preferentially identified based on their sensitivity to specific sulphatase and phosphatase enzyme treatments, respectively, and/or specific complexes they form with cationic probes in analytical techniques such as mass spectrometry.

Sialylated Complex N-Glycan Glycomes

The present invention is directed to at least one of natural oligosaccharide sequence structures and structures truncated from the reducing end of the N-glycan according to the Formula

[{SAα3/6}ₛ₁LNβ2]ᵣ₁Mα3{({SAα3/6}ₛ₂LNβ2)ᵣ₂Mα6}_{r8 {M[β}4GN[β4{Fucα6}ᵣ₃GN]ᵣ₄]ᵣ₅}ᵣ₆ (I)

with optionally one or two or three additional branches according to formula

{SAα3/6}ₛ₃LNβ, (Ilb)

wherein r1, r2, r3, r4, r5, r6, r7 and r8 are either 0 or 1, independently,

wherein s1, s2 and s3 are either 0 or 1, independently,

with the provision that at least r1 is 1 or r2 is 1, and at least one of s1, s2 or s3 is 1.

LN is N-acetyllactosaminyl also marked as GalβGN or di-N-acetyllactosdiaminyl

GalNAcβGlcNAc preferably GalNAcβ4GlcNAc, GN is GlcNAc, M is mannosyl-,

with the provision that LNβ2M or GNβ2M can be further elongated and/or branched with one or several other monosaccharide residues such as galactose, fucose, SA or LN-unit(s) which may be further substituted by SAα-strutures,

and/or one LN, can be truncated to GNβ

and/or Mα6 residue and/or Mα3 residue can be further substituted by one or two β6-,

and/or β4-linked additional branches according to the formula,

and/or either of Mα6 residue or Mα3 residue may be absent;

and/or Mα6- residue can be additionally substituted by other Manα units to form a hybrid type structures

and/or Manβ4 can be further substituted by GNβ4,

and/or SA may include natural substituents of sialic acid and/or it may be substituted by other SA-residues preferably by α8- or α9-linkages.

( ), { }, [ ] and [ ] indicate groups either present or absent in a linear sequence. { } indicates branching which may be also present or absent.

The SAα-groups are linked to either 3- or 6-position of neighboring Gal residue or on 6-position of GlcNAc, preferably 3- or 6-position of neighboring Gal residue. In separately preferred embodiments the invention is directed structures comprising solely 3-linked SA or 6-linked SA, or mixtures thereof In a preferred embodiment the invention is directed to glycans wherein r6 is 1 and r5 is 0, corresponding to N-glycans lacking the reducing end GlcNAc structure.

The LN unit with its various substituents can be represented in a preferred general embodiment by the formula:

[Gal(NAc)ₙ₁α3]ₙ₂{Fucα2}ₙ₃Gal(NAc)ₙ₄β3/4{Fucα4/3}ₙ₅GlcNAcβ

wherein n1, n2, n3, n4, and n5 are independently either 1 or 0,

with the provision that the substituents defined by n2 and n3 are alternative to the presence of SA at the non-reducing end terminal structure;

the reducing end GlcNAc-unit can be further β3- and/or β6-linked to another similar LN-structure forming a poly-N-acetyllactosamine structure with the provision that for this LN-unit n2, n3 and n4 are 0,

the Gal(NAc)β and GlcNAcβ units can be ester linked a sulfate ester group;

( ) and [ ] indicate groups either present or absent in a linear sequence; { }indicates branching which may be also present or absent.

LN unit is preferably Galβ4GN and/or Galβ3GN. The inventors revealed that hMSCs can express both types of N-acetyllactosamine, and therefore the invention is especially directed to mixtures of both structures. Furthermore, the invention is directed to special relatively rare type 1 N-acetyllactosamines, Galβ3GN, without any non-reducing end/site modification, also called lewis c-structures, and substituted derivatives thereof, as novel markers of hMSCs.

Hybrid Type Structures

According to the present invention, hybrid-type or monoantennary structures are preferentially identified by mass spectrometry, preferentially based on characteristic monosaccharide compositions, wherein HexNAc=3 and Hex≧2. In a more preferred embodiment of the present invention 2≦Hex≦11, and in an even more preferred embodiment of the present invention 2≦Hex≦9. The hybrid-type structures are further preferentially identified by sensitivity to exoglycosidase digestion, preferentially α-mannosidase digestion when the structures contain non-reducing terminal α-mannose residues and Hex≧3, or even more preferably when Hex≧4, and to endoglycosidase digestion, preferentially N-glycosidase F detachment from glycoproteins. The hybrid-type structures are further preferentially identified in NMR spectroscopy based on characteristic resonances of the Manα3(Manα6)Manβ4GlcNAcβ4GlcNAc N-glycan core structure, a GlcNAcβ residue attached to a Manα residue in the N-glycan core, and the presence of characteristic resonances of non-reducing terminal α-mannose residue or residues.

The monoantennary structures are further preferentially identified by insensitivity to α-mannosidase digestion and by sensitivity to endoglycosidase digestion, preferentially N-glycosidase F detachment from glycoproteins. The monoantennary structures are further preferentially identified in NMR spectroscopy based on characteristic resonances of the Manα3Manβ4GlcNAcβ4GlcNAc N-glycan core structure, a GlcNAcβ residue attached to a Mana. residue in the N-glycan core, and the absence of characteristic resonances of further non-reducing terminal α-mannose residues apart from those arising from a terminal α-mannose residue present in a ManαManβ sequence of the N-glycan core.

The invention is further directed to the N-glycans when these comprise hybrid type structures according to the Formula HY1:

R₁GNβ2Mα3{[R_{3]}ₙ₃Mα6}Mβ4GNXyR₂,

wherein n3, is either 0 or 1, independently,

and wherein X is glycosidically linked disaccharide epitope β4(Fucα6)ₙGN, wherein

n is 0 or 1, or X is nothing and

y is anomeric linkage structure α and/or β or linkage from derivatized anomeric carbon, and

R₁ indicate nothing or substituent or substituents linked to GlcNAc,

R₃ indicates nothing or Mannose-substituent(s) linked to mannose residue, so that each of R₁, and R₃ may correspond to one, two or three, more preferably one or two, and most preferably at least one natural substituents linked to the core structure,

R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

Preferred Hybrid Type Structures

The preferred hydrid type structures include one or two additional mannose residues on the preferred core stucture.

R₁GNβ2Mα3{[Mα3]ₘ₁([Mα6])ₘ₂Mα6}Mβ4GNXyR₂, Formula HY2

wherein and m1 and m2 are either 0 or 1, independently,

{ } and ( ) indicates branching which may be also present or absent, other variables are as described in Formula HY1.

Furthermore the invention is directed to structures comprising additional lactosamine type structures on GNβ2-branch. The preferred lactosamine type elongation structures includes N-acetyllactosamines and derivatives, galactose, GalNAc, GlcNAc, sialic acid and fucose.

Preferred structures according to the formula HY2 include:

Structures containing non-reducing end terminal GlcNAc as a specific preferred group of glycans

GNβ2Mα3{Mα3Mα6}Mβ4GNXyR₂,

GNβ2Mα3{Mα6Mα6}Mβ4GNXyR₂,

GNβ2Mα3{Mα3(Mα6)Mα6}Mβ4GNXyR₂,

and/or elongated variants thereof

R₁GNβ2Mα3{Mα3Mα6}Mβ4GNXyR₂,

R₁GNβ2Mα3{Mα6Mα6}Mβ4GNXyR₂,

R₁GNβ2Mα3{Mα3(Mα6)Mα6}Mβ4GNXyR₂,

[R₁Gal[NAc]ₒ₂βz]ₒ₁GNβ2Mα3{[Mα3]ₘ₁[(Mα6)]ₘ₂Mα6}ₙ₅Mα4GNXyR₂, Formula HY3

wherein n5, m1, m2, o1 and o2 are either 0 or 1, independently,

z is linkage position to GN being 3 or 4, in a preferred embodiment 4,

R₁ indicates one or two a N-acetyllactosamine type elongation groups or nothing,

{ } and ( ) indicates branching which may be also present or absent,

other variables are as described in Formula HY1.

Preferred structures according to the formula HY3 include especially structures containing non-reducing end terminal Galβ, preferably Galβ3/4 forming a terminal N-acetyllactosamine structure. These are preferred as a special group of Hybrid type structures, preferred as a group of specific value in characterization of balance of Complex N-glycan glycome and High mannose glycome:

GalβzGNβ2Mα3{Mα3Mα6}Mβ4GNXyR₂,

GalβzGNβ2Mα3{Mα6Mα6}Mβ4GNXyR₂,

GalβzGNβ2Mα3{Mα3(Mα6)Mα6}Mβ4GNXyR₂,

and/or elongated variants thereof preferred for carrying additional characteristic terminal structures useful for characterization of glycan materials

R₁GalβzGNβ2Mα3{Mα3Mα6}Mβ4GNXyR₂,

R₁GalβzGNβ2Mα3{Mα6Mα6}Mβ4GNXyR₂,

R₁GalβzGNβ2Mα3{Mα3(Mα6)Mα6}Mβ4GNXyR₂. Preferred elongated materials include structures wherein R₁ is a sialic acid, more preferably NcuNAc or NcuGc.

Recognition of Structures from Glycome Materials and On Cell Surfaces by Binding Methods

The present invention revealed that beside the physicochemical analysis by NMR and/or mass spectrometry several methods are useful for the analysis of the structures.

The invention is especially directed to a method:
i) Recognition by molecules binding glycans referred as the binders These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived thereof, when the proteins are selected from the group of monoclonal antibody, glycosidase, glycosyl transferring enzyme, plant lectin, animal lectin or a peptide mimetic thereof, and wherein the binder may include a detectable label structure.

The genus of enzymes in carbohydrate recognition is continuous to the genus of lectins (carbohydrate binding proteins without enzymatic activity).

a) Native glycosyltransferases (Rauvala et al.(1983) PNAS (USA) 3991-3995) and glycosidases (Rauvala and Hakomori (1981) J. Cell Biol. 88, 149-159) have lectin activities.

b) The carbohydrate binding enzymes can be modified to lectins by mutating the catalytic amino acid residues (see WO9842864; Aalto J. et al. Glycoconjugate J. (2001, 18(10); 751-8; Mega and Hase (1994) BBA 1200 (3) 331-3).

c) Natural lectins, which are structurally homologous to glycosidases are also known indicating the continuity of the genus enzymes and lectins (Sun, Y-J. et al. J. Biol. Chem. (2001) 276 (20) 17507-14).

The genus of the antibodies as carbohydrate binding proteins without enzymatic acitivity is also very close to the concept of lectins, but antibodies are usually not classified as lectins.

Obviousness of the Peptide Concept and Continuity with the Carbohydrate Binding Protein Concept

It is further realized that proteins consist of peptide chains and thus the recognition of carbohydrates by peptides is obvious. E.g. it is known in the art that peptides derived from active sites of carbohydrate binding proteins can recognize carbohydrates (e.g. Geng J-G. et al (1992) J. Biol. Chem. 19846-53).

As described above antibody fragment are included in description and genetically engineed variants of the binding proteins. The obvious genetically engineered variants would include truncated or fragment peptides of the enzymes, antibodies and lectins.

Revealing Cell or Differentiation and Individual Specific Terminal Variants of Structures

The invention is directed to use the glycomics profiling methods for the revealing structural features with on-off changes as markers of specific differentiation stage or quantitative difference based on quantitative comparison of glycomes. The individual specific variants are based on genetic variations of glycosyltransferases and/or other components of the glycosylation machinery preventing or causing synthesis of individual specific structure.

Terminal Structural Epitopes

We have previously revealed glycome compositions of human glycomes, here we provide structural terminal epitopes useful for the characterization of mesenchymal stem cell glycomes, especially by specific binders.

The examples of characteristic altering terminal structures includes expression of competing terminal epitopes created as modification of key homologous core Galβ-epitopes, with either the same monosaccharides with difference in linkage position Galβ3GlcNAc, and analogue with either the same monosaccharides with difference in linkage position Galβ4GlcNAc; or the with the same linkage but 4-position epimeric backbone Galβ3GalNAc. These can be presented by specific core structures modifying the biological recognition and function of the structures. Another common feature is that the similar Galβ-structures are expressed both as protein linked (O— and N-glycan) and lipid linked (glycolipid structures). As an alternative for α2-fucosylation the terminal Gal may comprise NAc group on the same 2 position as the fucose. This leads to homologous epitopes GalNAcβ4GlcNAc and yet related GalNAcβ3Gal-structure on characteristic special glycolipid according to the invention.

The invention is directed to novel terminal disaccharide and derivative epitopes from human stem cells, preferably mesenchymal stem cells. It should be realized that glycosylations are species, cell and tissue specific and results from cancer cells usually differ dramatically from normal cells, thus the vast and varying glycosylation data obtained from human embryonal carcinomas are not actually relevant or obvious to human embryonal stem cells, or any mesenchymal cells (unless accidentally appeared similar). Additionally the exact differentiation level of teratocarcinomas cannot be known, so comparison of terminal epitope under specific modification machinery cannot be known. The terminal structures by specific binding molecules including glycosidases and antibodies and chemical analysis of the structures.

The present invention reveals group of terminal Gal(NAc)β1-3/4Hex(NAc) structures, which carry similar modifications by specific fucosylation/NAc-modification, and sialylation on corresponding positions of the terminal disaccharide epitopes. It is realized that the terminal structures are regulated by genetically controlled homologous family of fucosyltransferases and sialyltransferases. The regulation creates a characteristic structural patterns for communication between cells and recognition by other specific binder to be used for analysis of the cells. The key epitopes are presented in the TABLE 19. The data reveals characteristic patterns of the terminal epitopes for each types of cells, such as for example expression of type I and Type II lactosamine and derivatives differentiation specifically and similar modifications of multiple backbone structures such as Fucα2-structures on type 1 lactosamine (Galβ3GlcNAc), similarily β3-linked core I Galβ3GlcNAcα, and type 4 structure which is present on specific type of glycolipids and expression of α3-fucosylated structures. E.g. terminal type lactosamine and poly-lactosamines differentiate mesenchymal stem cells from other types. The terminal Galβ-structure information is preferably combined with information about the sialylated and/or fucosylated Galβ-structures and/or information about GalNAc comprising O-glycan core structures comprising GalNAc and/or glycolipid structures.

The invention is directed especially to high specificity binding molecules such as monoclonal antibodies for the recognition of the structures.

The structures can be presented by Formula T1. The formula describes first monosaccharide residue on left, which is a β-D-galactopyranosyl structure linked to either 3 or 4-position of the α- or β-D-(2-deoxy-2-acetamido)galactopyranosyl structure, when R₅ is OH, or β-D-(2-deoxy-2-acetamido)glucopyranosyl, when R₄ comprises O—. The unspecified stereochemistry of the reducing end in formulas T1 and T2 is indicated additionally (in claims) with curved line. The sialic acid residues can be linked to 3 or 6-position of Gal or 6-position of GlcNAc and fucose residues to position 2 of Gal or 3- or 4-position of GlcNAc or position 3 of Glc.

Formula T1:

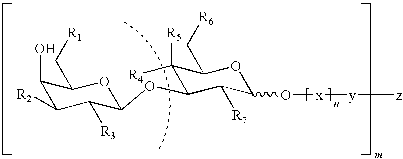

wherein

X is linkage position

R₁, R₂, and R₆ are OH or glycosidically linked monosaccharide residue Sialic acid, preferably Neu5Acα2 or Neu5Gc α2, most preferably Neu5Acα2 or

R₃, is OH or glycosidically linked monosaccharide residue Fucα1 (L-fucose) or N-acetyl (N-acetamido, NCOCH₃);

R₄, is H, OH or glycosidically linked monosaccharide residue Fucα1 (L-fucose),

R₅ is OH, when R₄ is H, and R₅ is H, when R₄ is not H;

R7 is N-acetyl or OH

X is natural oligosaccharide backbone structure from the cells, preferably N-glycan,

O-glycan or glycolipid structure; or X is nothing, when n is 0,

Y is linker group preferably oxygen for O-glycans and O-linked terminal oligosaccharides and glycolipids and N for N-glycans or nothing when n is 0;

Z is the carrier structure, preferably natural carrier produced by the cells, such as protein or lipid, which is preferably a ceramide or branched glycan core structure on the carrier or H;

The arch indicates that the linkage from the galactopyranosyl is either to position 3 or to position 4 of the residue on the left and that the R4 structure is in the other position 4 or 3;

n is an integer 0 or 1, and m is an integer from 1 to 1000, preferably 1 to 100, and most preferably 1 to 10 (the number of the glycans on the carrier),

With the provisions that one of R2 and R3 is OH or R3 is N-acetyl,

R6 is OH, when the first residue on left is linked to position 4 of the residue on right:

X is not Galα4Galβ4Glc, (the core structure of SSEA-3 or 4) or R3 is Fucosyl

R7 is preferably N-acetyl, when the first residue on left is linked to position 3 of the residue on right.

Preferred terminal β3-linked subgroup is represented by Formula T2 indicating the situation, when the first residue on the left is linked to the 3 position with backbone structures Gal(NAc)β3Gal/GlcNAc.

Formula T2

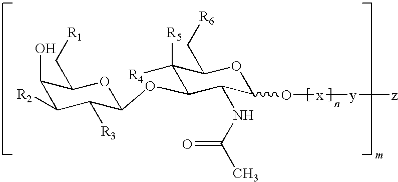

Wherein the variables including R₁ to R₇ are as described for T1

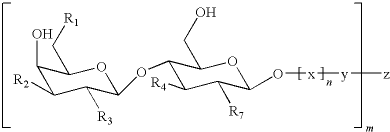

Preferred terminal β4-linked subgroup is represented by the Formula T3:

Wherein the variables including R₁ to R₄ and R₇ are as described for T1 with the provision that R₄, is OH or glycosidically linked monosaccharide residue Fucocl (L-fucose),

Alternatively the epitope of the terminal structure can be represented by Formulas T4 and T5

Core Galβ-epitopes formula T4:

Galβ1-xHex(NAc)ₚ,

x is linkage position 3 or 4,

and Hex is Gal or Glc

with provision

p is 0 or 1

when x is linkage position 3, p is 1 and HexNAc is GlcNAc or GalNAc,

and when x is linkage position 4, Hex is Glc.

The core Galβ1-3/4 epitope is optionally substituted to hydroxyl by one or two structures SAα or Fucα, preferably selected from the group

Gal linked SAα3 or SAα6 or Fucα2, and

Glc linked Fucα3 or GlcNAc linked Fucα3/4.

[Mα]ₘGalβ1-x[Nα]ₙHex(NAc)ₚ, Formula T5

wherein m, n and p are integers 0, or 1, independently

Hex is Gal or Glc,

X is linkage position

M and N are monosaccharide residues being independently nothing (free hydroxyl groups at the positions) and/or

SA which is Sialic acid linked to 3-position of Gal or/and 6-position of HexNAc and/or

Fuc (L-fucose) residue linked to 2-position of Gal and/or 3 or 4 position of HexNAc, when Gal is linked to the other position (4 or 3), and HexNAc is GlcNAc, or 3-position of Glc when Gal is linked to the other position (3),

with the provision that sum of m and n is 2

preferably m and n are 0 or 1, independently.

The exact structural details are essential for optimal recognition by specific binding molecules designed for the analysis and/or manipulation of the cells.

The terminal key Galβ-epitopes are modified by the same modification monosaccharides NeuX (X is 5 position modification Ac or Gc of sialic acid) or Fuc, with the same linkage type alfa( modifying the same hydroxyl-positions in both structures.

NeuXα3, Fucα2 on the terminal Galβ of all the epitopes and

NeuXα6 modifying the terminal Galβ of Galβ4GlcNAc, or HexNAc, when linkage is 6 competing

or Fucα modifying the free axial primary hydroxyl left in GlcNAc (there is no free axial hydroxyl in GalNAc-residue).

The preferred structures can be divided to preferred Galβ1-3 structures analogously to T2,

[Mα]ₘGalβ1-3[Nα]ₙHexNAc, Formula T6:

Wherein the variables are as described for T5.

The preferred structures can be divided to preferred Galβ1-4 structures analogously to T4,

[Mα]ₘGalβ1-4[Nα]ₙGlc(NAc)ₚ, Formula T7:

Wherein the variables are as described for T5.

These are preferred type II N-acetyllactosamine structures and related lactosylderivatives, in a preferred embodiment p is 1 and the structures includes only type 2 N-acetyllactosamines. The invention revealed that the these are very useful for recognition of specific subtypes of mesenchymal cells, preferably mesenchymal stem cells, differentiated variants thereof (tissue type specifically differentiated mesenchymal stem cells). It is notable that various fucosyl- and or sialic acid modification created characteristic pattern for the stem cell type.

Preferred Type I and Type II N-Acetyllactosamine Structures

The preferred structures can be divided to preferred type one (I) and type two (II) N-acetyllactosamine structures comprising oligosaccharide core sequence Galβ1-3/4 GlcNAc structures analogously to T4,

[Mα]ₘGalβ1-3/4[Nα]ₙGlcNAc, Formula T8:

Wherein the variables are as described for T5.

The preferred structures can be divided to preferred Galβ1-3 structures analogously to T8,

[Mα]ₘGalβ1-3[Nα]ₙGlcNAc Formula T9:

Wherein the variables are as described for T5.

These are preferred type I N-acetyllactosamine structures. The invention revealed that the these are very useful for recognition of specific subtypes of mesenchymal cells, preferably mesenchymal stem cells, or differentiated variants thereof (tissue type specifically differentiated mesenchymal stem cells). It is notable that various fucosyl- and or sialic acid modification created characteristic pattern for the cell or stem cell type.

The preferred structures can be divided to preferred Galβ1-4GlcNAc core sequence comprising structures analogously to T8,

[Mα]ₘGalβ1-4[Nα]ₙGlcNAc Formula T10:

Wherein the variables are as described for T5.

These are preferred type II N-acetyllactosamine structures. The invention revealed that the these are very useful for recognition of specific subtypes of stem cells, preferably mesenchymal stem cells, or differentiated variants thereof (tissue type specifically differentiated mesenchymal stem cells).

It is notable that various fucosyl- and or sialic acid modificationally N-acetyllactosamine structures create especially characteristic pattern for the stem cell/cell type. The invention is further directed to use of combinations of binder reagents recognizing at least two different type I and type II acetyllactosamines including at least one fucosylated or sialylated varient and more preferably at least two fucosylated variants or two sialylated variants

Preferred structures comprising terminal Fucα2/3/4-structures

The invention is further directed to use of combinations binder reagents recognizing:
a) type I and type II acetyllactosamines and their fucosylated variants, and in a preferred embodiment
b) non-sialylated fucosylated and even more preferably
c) fucosylated type I and type II N-acetyllactosamine structures preferably comprising Fucα2-terminal and/or Fucα3/4-branch structure and even more preferably
d) fucosylated type I and type II N-acetyllactosamine structures preferably comprising Fucα2-terminal

for the methods according to the invention of various stem cells and differentiated variants thereof, especially mesenchymal stem cells and differentiated variants thereof.

Preferred subgroups of Fucα2-structures includes monofucosylated H type and H type II structures, and difucosylated Lewis b and Lewis y structures.

Preferred subgroups of Fucα3/4-structures includes monofucosylated Lewis a and Lewis x structures, sialylated sialyl-Lewis a and sialyl-Lewis x-structures and difucosylated Lewis b and Lewis y structures.

Preferred type II N-acetyllactosamine subgroups of Fucα3-structures includes monofucosylated Lewis x structures, and sialyl-Lewis x-structures and Lewis y structures.

Preferred type I N-acetyllactosamine subgroups of Fucα4-structures includes monofucosylated Lewis a, sialyl-Lewis a and difucosylated Lewis b structures.

The invention is further directed to use of at least two differently fucosylated type one and or and two N-acetyllactosamine structures preferably selected from the group monofucosylated or at least two difucosylated, or at least one monofucosylated and one difucosylated structures.

The invention is further directed to use of combinations of binder reagents recognizing fucosylated type I and type II N-acetyllactosamine structures together with binders recognizing other terminal structures comprising Fucα2/3/4-comprising structures, preferably Fucα2-terminal structures, preferably comprising Fucα2Galβ3GalNAc-terminal, more preferably Fucα2Galβ3GalNAcα/β and in especially preferred embodiment antibodies recognizing Fucα2Galβ3GalNAcβ-preferably in terminal structure of Globo structures.

Preferred Globo- and Ganglio Core Type-Structures

The invention is further directed to general formula comprising globo and gangliotype Glycan core structures according to formula

[M]ₘGalβ1-x[Nα]ₙHex(NAc)ₚ, Formula T11

wherein m, n and p are integers 0, or 1, independently

Hex is Gal or Glc, X is linkage position;

M and N are monosaccharide residues being independently nothing (free hydroxyl groups at the positions) and/or

SAα which is Sialic acid linked to 3-position of Gal or/and 6-position of HexNAc

Galα linked to 3 or 4-position of Gal, or

GalNAcβ linked to 4-position of Gal and/or

Fuc (L-fucose) residue linked to 2-position of Gal

and/or 3 or 4 position of HexNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently, and

with the provision that when M is Galα then there is no sialic acid linked to Galβ1, and

n is 0 and preferably x is 4.

with the provision that when M is GalNAcβ, then there is no sialic acid α6-linked to Galβ1, and n is 0 and x is 4.

The invention is further directed to general formula comprising globo and gangliotype Glycan core structures according to formula

[M][SAα3]ₙGalβ1-4Glc(NAc)ₚ, Formula T12

wherein n and p are integers 0, or 1, independently

M is Galα linked to 3 or 4-position of Gal, or GalNAβ linked to 4-position of Gal

and/or SAα is Sialic acid branch linked to 3-position of Gal

with the provision that when M is Galα then there is no sialic acid linked to Galβ1 (n is 0).

The invention is further directed to general formula comprising globo and gangliotype

Glycan core structures according to formula

[M][SAα]ₙGalβ1-4Glc, Formula T13

wherein n and p are integer 0, or 1, independently

M is Galα linked to 3 or 4-position of Gal, or

GalNAcβ linked to 4-position of Gal and/or

SAα which is Sialic acid linked to 3-position of Gal

with the provision that when M is Galα then there is no sialic acid linked to Galβ1 (n is 0).

The invention is further directed to general formula comprising globo type Glycan core structures according to formula

Galα3/4Galβ1-4Glc. Formula T14

The preferred Globo-type structures includes Galα3/4Galβ1-4Glc,

GalNAcβ3Galα3/4Galβ4Glc, Galα4Galβ4Glc (globotriose, Gb3), Galα3Galβ4Glc (isoglobotriose), GalNAcβ3Galα4Galβ4Glc (globotetraose, Gb4 (or G14)), and

Fucα2Galβ3GalNAcβ3Galα3/4Galβ4Glc. or

when the binder is not used in context of mesenchymal stem cells or the binder is used together with another preferred binder according to the invention, preferably an other globo-type binder the preferred binder targets further includes

Galβ3GalNAcβ3Galα4Galβ4Glc (SSEA-3 antigen) and/or

NeuAcα3Galβ3GalNAcβ3Galα4Galβ4Glc (SSEA-4 antigen) or terminal non-reducing end di or trisaccharide epitopes thereof.

The preferred globotetraosylceramide antibodies does not recognize non-reducing end elongated variants of GalNAcβ3Galα4Galβ4Glc. The antibody in the examples has such specificity as . . . ?

The invention is further directed to binders for specific epitopes of the longer oligosaccharide sequences including preferably NeuAcα3Galβ3GalNAc, NeuAcα3Galβ3GalNAcβ, NeuAcα3Galβ3GalNAcβ3Galα4Gal when these are not linked to glycolipids and novel fucosylated target structures:

Fucα2Galβ3GalNAcβ3Galα3/4Gal,Fucα2Galβ3GalNAcβ3Galoα, Fucα2Galβ3GalN Acβ3Gal, Fucα2Galβ3GalNAcβ3, and Fucα2Galβ3GalNAc.

The invention is further directed to general formula comprising globo and gangliotype Glycan core structures according to formula

[GalNAcβ4][SAα]ₙGalβ1-4Glc, Formula T15

wherein n and p are integer 0, or 1, independently GalNAcβ linked to 4-position of Gal and/or SAα which is Sialic acid branch linked to 3-position of Gal.

The preferred Ganglio-type structures includes GalNAcβ4Galβ1-4Glc, GalNAcβ4[SAα3]Galβ1-4Glc, and Galβ3GalNAcβ4[SAα3]Galβ1-4Glc.

The preferred binder target structures further include glycolipid and possible glycoprotein conjugates of of the preferred oligosaccharide sequences. The preferred binders preferably specifically recognizes at least di- or trisaccharide epitope.

GalNAcα-Structures

The invention is further directed to recognition of peptide/protein linked GalNAcα-structures according to the Formula T16:

[SAα6]ₘGalNAcα[Ser/Thr]ₙ-[Peptide]ₚ,

wherein m, n and p are integers 0 or 1, independently,

wherein SA is sialic acid preferably NeuAc,Ser/Thr indicates linking serine or threonine residues. Peptide indicates part of peptide sequence close to linking residue, with the provision that either m or n is 1.

Ser/Thr and/or Peptide are optionally at least partiallt necessary for recognition for the binding by the binder. It is realized that when Peptide is included in the specificity, the antibody have high specificity involving part of a protein structure. The preferred antigen sequences of sialyl-Tn: SAα6GalNAcα, SAα6GalNAcαSer/Thr, and SAα6GalNAcαSer/Thr-Peptide and Tn-antigen: GalNAcαSer/Thr, and GalNAcαSer/Thr-Peptide. The invention is further directed to the use of combinations of the GalNAcα-structures and combination of at least one GalNAcα-structure with other preferred structures.

Combinations of Preferred Binder Groups

The present invention is especially directed to combined use of at least a)fucosylated, preferably α2/3/4-fucosylated structures and/or b) globo-type structures and/or c) GalNAcα-type structures. It is realized that using a combination of binders recognizing strctures involving different biosynthesis and thus having characteristic binding profile with a stem cell population. More preferably at least one binder for a fucosylated structure and and globostructures, or fucosylated structure and GalNAcα-type structure is used, most preferably fucosylated structure and globostructure are used.

Fucosylated and Non-Modified Structures

The invention is further directed to the core disaccharide epitope structures when the structures are not modified by sialic acid (none of the R-groups according to the Formulas T1-T3 or M or N in formulas T4-T7 is not a sialic acid. The invention is in a preferred embodiment directed to structures, which comprise at least one fucose residue according to the invention. These structures are novel specific fucosylated terminal epitopes, useful for the analysis of stem cells according to the invention. Preferably native stem cells are analyzed.

The preferred fucosylated structures include novel α3/4fucosylated markers of human stem cells such as (SAα3)₀ₒᵣ₁Galβ3/4(Fucα4/3)GlcNAc including Lewis x and and sialylated variants thereof.

Among the structures comprising terminal Fucα1-2 the invention revealed especially useful novel marker structures comprising Fucα2Galβ3GalNAcα/β and Fucα2Galβ3(Fucα4)₀ₒᵣ₁GlcNAcβ, these were found to be present in mesenchymal cells (Table 19). A especially preferred antibody/binder group among this group is antibodies specific for Fucα2Galβ3GlcNAcβ, preferred for high stem cell specificity. Another preferred structural group includes Fucα2Gal comprising glycolipids revealed to form specific structural group.

Among the antibodies recognizing Fucα2Galβ4GlcNAcβ substantial variation in binding was revealed likely based on the carrier structures, the invention is especially directed to antibodies recognizing this type of structures, when the specificity of the antibody is similar to the ones binding to the mesenchymal cell structures with fucose. The invention is preferably directed to antibodies recognizing Fucα2Galβ4GlcNAcβ on N-glycans, revealed as common structural type in terminal epitope Table 19. In a separate embodiment the antibody of the non-binding clone is directed to the recognition of other cell types.

The preferred non-modified structures includes Galβ4Glc, Galβ3GlcNAc, Galβ3GalNAc, Galβ4GlcNAc, Galβ3GlcNAcβ, Galβ3GalNAcβ/α, and Galβ4GlcNAcβ. These are preferred novel core markers characteristics for the various stem cells, especially mesencymal cells. Preferably the structure is carried by a glycolipid core structure according to the invention or it is present on an O-glycan. The non-modified markers are preferred for the use in combination with at least one fucosylated or/and sialylated structure for analysis of cell status.

Additional preferred non-modified structures includes GalNAcβ-structures includes terminal LacdiNAc, GalNAcβ4GlcNAc, preferred on N-glycans and GalNAcβ3Gal GalNAcβ3Gal present in globoseries glycolipids as terminal of globotetraose structures.

Among these characteristic subgroup of Gal(NAc)β3-comprising Galβ3GlcNAc, Galβ3GalNAc, Galβ3GlcNAcβ, Galβ3GalNAcβ/α, and GalNAcβ3Gal GalNAcβ3Gal and

the characteristic subgroup of Gal(NAc)β4-comprising Galβ4Glc, Galβ4GlcNAc, and Galβ4GlcNAc are separately preferred.

Preferred Sialylated Structures

The preferred sialylated structures includes characteristic SAα3Galβ-structures SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, and SAα3Galβ4GlcNAcβ; and biosynthetically partially competing SAα6Galβ-structures SAα6Galβ4Glc, SAα6Galβ4Glcβ; SAα6Galβ4GlcNAc and SAα6Galβ4GlcNAcβ; and disialo structures SAα3Galβ3(SAα6)GalNAcβ/α, and SAα3GalP3(SAα6)GlcNAcβ.

The invention is preferably directed to specific subgroup of Gal(NAc)β3-comprising

SAα3Galβ3GlcNAc, SAα3Galβ3GalNAc, SAα3Galβ4GlcNAc,

SAα3Galβ3GlcNAcβ, SAα3Galβ3GalNAcβ/α and

SAα3Galβ3(SAα6)GalNAcβ/α, and

Gal(NAc)β4-comprising sialylated structures. SAα3Galβ4Glc, and

SAα3Galβ4GlcNAcβ; and SAα6Galβ4Glc, SAα6Galβ4Glcβ; SAα6Galβ4GlcNAc and SAα6Galβ4GlcNAcβ

These are preferred novel regulated markers characteristics for the various mesencymal stem cells or differentiated derivatives thereof.

Use Together with a Terminal ManαMan-Structure

The terminal non-modified or modified epitopes are in preferred embodiment used together with at least one ManαMan-structure. This is preferred because the structure is in different N-glycan or glycan subgroup than the other epitopes.

Core Structures of the Terminal Epitopes

It is realized that the target epitope structures are most effectively recognized on specific N-glycans, O-glycan, or on glycolipid core structures.

Elongated Epitopes—Next Monosaccharide/Structure on the Reducing End of the Epitope

The invention is especially directed to optimized binders and production thereof, when the binding epitope of the binder includes the next linkage structure and even more preferably at least part of the next structure (monosaccharide or aminoacid for O-glycans or ceramide for glycolipid) on the reducing side of the target epitope. The invention has revealed the core structures for the terminal epitopes as shown in the Examples and ones summarized in Table 19.

It is realized that antibodies with longer binding epitopes have higher specificity and thus will recognize that desired cells or cell derived components more effectively. In a preferred embodiment the antibodies for elongated epitopes are selected for effective analysis of mesenchymal type stem cells.

The invention is especially directed to the methods of antibody selection and optionally further purification of novel antibodies or other binders using the elongated epitopes according to the invention. The preferred selection is performed by contacting the glycan structure (synthetic or isolated natural glycan with the specific sequence) with a serum or an antibody or an antibody library, such as a phage display library. Data about these methods are well known in the art and available from internet for example by searching pubmed-medical literature database (www.ncbi.nlm.nih.gov/entrez) or patents e.g. in espacenet (fi.espacenet.com). The specific antibodies are especially preferred for the use of the optimized recognition of the glycan type specific terminal structures as shown in the Examples and ones summarized in the Table 19.

It is further realized that part of the antibodies according to the invention and shown in the examples have specificity for the elongated epitopes. The inventors found out that for example Lewis x epitope can be recognized on N-glycan by certain terminal Lewis x specific antibodies, but not so effectively or at all by antibodies recognizing Lewis xβ1-3Gal present on poly-N-acetyllactosamines or neolactoseries glycolipids.

N-Glycans

The invention is especially directed to recognition of terminal N-glycan epitopes on biantennary N-glycans. The preferred non-reducing end monosaccharide epitope for N-glycans comprise β2Man and its reducing end further elongated variants β2Man, β2Manα, β2Manα3, and β2Manα6

The invention is especially directed to recognition of Lewis x on N-glycan by N-glycan Lewis x specific antibody described by Ajit Varki and colleagues Glycobiology (2006) Abstracts of Glycobiology society meeting 2006 Los Angeles, with possible implication for neuronal cells, which are not directed (but disclaimed) with this type of antibody by the present invention.

Invention is further directed to antibodies with speficity of type 2 N-acetyllactosamineβ2Man recognizing biantennary N-glycan directed antibody as described in Ozawa H et al (1997) Arch Biochem Biophys 342, 48-57.

O-Glycans, Reducing end Elongated Epitopes

The invention is especially directed to recognition of terminal O-glycan epitopes as terminal core I epitopes and as elongated variants of core I and core II O-glycans. The preferred non-reducing end monosaccharide epitope for O-glycans comprise:

a) Core I epitopes linked to αSer/Thr-[Peptide]₀₋₁,

wherein Peptide indicates peptide which is either present or absent. The invention is preferabl

b) Preferred core II-type epitopes

R1β6[R2β3Galβ3]ₙGalNAcαSer/Thr, wherein n is=or 1 indicating possible branch in the structure and R1 and R2 are preferred positions of the terminal epitopes, R1 is more preferred

c) Elongated Core I epitope

β3Gal and its reducing end further elongated variants β3Galβ3GalNAcα, β3 Galβ3GalNAcαSer/Thr

O-glycan core I specific and ganglio/globotype core reducing end epitopes have been described in (Saito S et al. J Biol Chem (1994) 269, 5644-52), the invention is preferably directed to similar specific recognition of the epitopes according to the invention.

O-glycan core II sialyl-Lewis x specific antibody has been described in Walcheck B et al. Blood (2002) 99, 4063-69.

Peptide specificity including antibodies for recognition of O-glycans includes mucin specific antibodies further recognizing GalNAcalfa (Tn) or Galb3GalNAcalfa (T/TF) structures (Hanisch F-G et al (1995) cancer Res. 55, 4036-40; Karsten U et al. Glycobiology (2004) 14, 681-92).

Glycolipid Core Structures

The invention is furthermore directed to the recognition of the structures on lipid structures. The preferred lipid core structures include:
a) βCer (ceramide) for Galβ4Glc and its fucosyl or sialyl derivatives
b) β3/6Gal for type I and type II N-acetyllactosamines on lactosyl Cer-glycolipids, preferred elongated variants includes β3/6[Rβ6/3]ₙGalβ, β3/6[Rβ6/3]ₙGalβ4 and β3/6[Rβ6/3]ₙGalβ4Glc, which may be further branched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4Glc
c) α3/4Gal, for globoseries epitopes, and elongated variants α3/4Galβ, α3/4Galβ4Glc preferred globoepitopes have elongated epitopes α4Gal, α4Galβ, α4Galβ4Glc, and
   preferred isogloboepitopes have elongated epitopes α3Gal, α3Galβ, α3Galβ4Glc
d) β4Gal for ganglio-series epitopes comprising, and preferred elongated variants include β4Galβ, and β4Galβ4Glc

O-glycan core specific and ganglio/globotype core reducing end epitopes have been described in (Saito S et al. J Biol Chem (1994) 269, 5644-52), the invention is preferably directed to similar specific recognition of the epitopes according to the invention.

Poly-N-Acetyllactosamines

Poly-N-acetyllactosamine backbone structures on O-glycans, N-glycans, or glycolipids comprise characteristic structures similar to lactosyl(cer) core structures on type I (lactoseries) and type II (neolacto) glycolipids, but terminal epitopes are linked to another type I or type II N-acetyllactosamine, which may from a branched structure. Preferred elongated epitopes include:

β3/6Gal for type I and type II N-acetyllactosamines epitope, preferred elongated variants includes R1B3/6[R2β6/3]ₙGalβ, R1β3/6[R2β6/3]ₙGalβ3/4 and R1β3/6[R2β6/3]ₙGalβ3/4GlcNAc, which may be further branched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4GlcNAc.

Numerous antibodies are known for linear (i-antigen) and branched poly-N-acetyllactosamines (I-antigen), the invention is further directed to the use of the lectin PWA for recognition of I-antigens and to the use of lectin STA for recognition of i-antigen. The inventors revealed that poly-N-acetyllactosamines are characteristic structures for specific types of human mesenchymal cells. Another preferred binding regent, enzyme endo-beta-galactosidase was used for characterization poly-N-acetyllactosamines on glycolipids and on glycoprotein of the stem cells. The enzyme revealed characteristic expression of both linear and branched poly-N-acetyllactosamine, which further comprised specific terminal modifications such as fucosylation and/or sialylation according to the invention on specific types of stem cells.

Combinations of Elongated core Epitopes

It is realized that stronger labeling may be obtained if the same terminal epitope is recognized by antibody binding to target structure present on two or three of the major carrier types O-glycans, N-glycans and glycolipids. It is further realized that in context of such use the terminal epitope must be specific enough in comparison to the epitopes present on possible contaminating cells or cell matrials. It is further realized that there is highly terminally specific antibodies, which allow binding to on several elongation structures.

The invention revealed each elongated binder type useful in context of stem cells. Thus the invention is directed to the binders recognizing the terminal structure on one or several of the elongating structures according to the invention.

Preferred Group of Monosaccharide Elongation Structures

The invention is directed to use of binders with elongated specificity, when the binders recognize or is able to bind at least one reducing end elongation monosaccharide epitope according to the formula

AxHex(NAc)ₙ,

wherein A is anomeric structure alfa or beta, X is linkage position 2, 3, 4, or 6

And Hex is hexopyranosyl residue Gal, or Man, and n is integer being 0 or 1, with the provisions that when n is 1 then AxHexNAc is β4GalNAc or β6GalNAc, when Hex is Man, then AxHex is β2Man, and when Hex is Gal, then AxHex is β3Gal or β6Gal. Beside the monosaccharide elongation structures αSer/Thr are preferred reducing end elongation structures for reducing end GalNAc-comprising O-glycans and βCer is preferred for lactosyl comprising glycolipid epitopes.

The preferred subgroups of the elongation structures includes i) similar structural epitopes present on O-glycans, polylactosamine and glycolipid cores: β3/6Gal or β6GalNAc; with preferred further subgroups ia) β6GalNAc/β6Gal and ib) β3Gal; ii) N-glycan type epitope β2Man; and iii) globoseries epitopes α3Gal or α4Gal. The groups are preferred for structural similarity on possible cross reactivity within the groups, which can be used for increasing labeling intensity when background materials are controlled to be devoid of the elongated structure types.

Useful binder specificities including lectin and elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritsumei.ac.ip/epitope/, which list monoclonal antibody glycan specificities).

Preferred Binder Molecules

The present invention revealed various types of binder molecules useful for characterization of cells according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and/or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

Modulation of Cells by the Binders

The invention revealed that the specific binders directed to a cell type can be used to modulate cells. In a preferred embodiment the (stem) cells are modulated with regard to carbohydrate mediated interactions. The invention revealed specific binders, which change the glycan structures and thus the receptor structure and function for the glycan, these are especially glycosidases and glycosyltransferring enzymes such as glycosyltransferases and/or transglycosylating enzymes. It is further realized that the binding of a non-enzymatic binder as such select and/or manipulate the cells. The manipulation typically depends on clustering of glycan receptors or affects of the interactions of the glycan receptors with counter receptors such as lectins present in a biological system or model in context of the cells. The invention further reveled that the modulation by the binder in context of cell culture has effect about the growth velocity of the cells.

Preferred Combinations of the Binders

The invention revealed useful combination of specific terminal structures for the analysis of status of a cells. In a preferred embodiment the invention is directed to measuring the level of two different terminal structures according to the invention, preferably by specific binding molecules, preferably at least by two different binders. In a preferred embodiment the binder molecules are directed to structures indicating modification of a terminal receptor glycan structures, preferably the structures represent sequential (substrate structure and modification thereof, such as terminal Gal-structure and corresponding sialylated structure) or competing biosynthetic steps (such as fucosylation and sialylation of terminal Galβ or terminal Galβ3GlcNAc and Galβ4GlcNAc). In another embodiment the binders are directed to three different structures representing sequential and competing steps such as such as terminal Gal-structure and corresponding sialylated structure.

The invention is further directed to recognition of at least two different structures according to the invention selected from the groups of non-modified (non-sialylated or non-fucosylated) Gal(NAc)β3/4-core structures according to the invention, preferred fucosylated structures and preferred sialylated structures according to the invention. It is realized that it is useful to recognize even 3, and more preferably 4 and even more preferably five different structures, preferably within a preferred structure group.

Target Structures for Specific Binders and Examples of the Binding Molecules

Combination of Terminal Structures with Specific Glycan Core Structures

It is realized that part of the structural elements are specifically associated with specific glycan core structure. The recognition of terminal structures linked to specific core structures are especially preferred, such high specificity reagents have capacity of recognition almost complete individual glycans to the level of physicochemical characterization according to the invention. For example many specific mannose structures according to the invention are in general quite characteristic for N-glycan glycomes according to the invention. The present invention is especially directed to recognition of terminal epitopes.

Common Terminal Structures on Several Glycan Core Structures

The present invention revealed that there are certain common structural features on several glycan types and that it is possible to recognize certain common epitopes on different glycan structures by specific reagents when specificity of the reagent is limited to the terminal structure without specificity for the core structure. The invention especially revealed characteristic terminal features for specific cell types according to the invention. The invention realized that the common epitopes increase the effect of the recognition. The common terminal structures are especially useful for recognition in the context with possible other cell types or material, which do not contain the common terminal structure in substantial amount.

The invention revealed the presence of the terminal structures on specific core structures such as N-glycan, O-glycan and/or glycolipids. The invention is preferably directed to the selection of specific binders for the structures including recognition of specific glycan core types.

The invention is further directed to glycome compositions of protein linked glycomes such as N-glycans and O-glycans and glycolipids each composition comprising specific amounts of glycan subgroups. The invention is further directed to the compositions when these comprise specific amount of Defined terminal structures.

Specific Preferred Structural Groups

The present invention is directed to recognition of oligosaccharide sequences comprising specific terminal monosaccharide types, optionally further including a specific core structure. The preferred oligosaccharide sequences are in a preferred embodiment classified based on the terminal monosaccharide structures. The invention further revealed a family of terminal (non-reducing end terminal) disaccharide epitopes based on β-linked galactopyranosylstructures, which may be further modified by fucose and/or sialic acid residues or by N-acetylgroup, changing the terminal Gal residue to GalNAc. Such structures are present in N-glycan, O-glycan and glycolipid subglycomes. Furhtermore the invention is directed to terminal disaccharide epitopes of N-glycans comprising terminal ManαMan.

The structures were derived by mass spectrometric and optionally NMR analysis and by high specificity binders according to the invention, for the analysis of glycolipid structures permethylation and fragmentation mass spectrometry was used. Biosynthetic analysis including known biosynthetic routes to N-glycans, O-glycans and glycolipids was additionally used for the analysis of the glycan compositions.

Structures with Terminal Mannose Monosaccharide

Preferred mannose-type target structures have been specifically classified by the invention. These include various types of high and low-mannose structures and hybrid type structures according to the invention.

The Preferred Terminal Manα-Target Structure Enitones

The invention revealed the presence of Manα on low mannose N-glycans and high mannose N-glycans. Based on the biosynthetic knowledge and supporting this view by analysis of mRNAs of biosynthetic enzymes and by NMR-analysis the structures and terminal epitopes could be revealed:

Manα2Man, Manα3Man, Manα6Man and Manα3(Manα6)Man, wherein the reducing end Man is preferably either α- or β-linked glycoside and α-linked glycoside in case of Manα2Man:

The general struture of terminal Manα-structures is Manαx(Manαy)_{z}Manα/β

Wherein x is linkage position 2, 3 or 6, and y is linkage position 3 or 6,

z is integer 0 or 1, indicating the presence or the absence of the branch,

with the provision that x and y are not the same position and

when x is 2, the z is 0 and reducing end Man is preferably α-linked;

The low-mannose structures includes preferably non-reducing end terminal epitopes with structures with α3- and/or α6-mannose linked to another mannose residue Manαx(Manαy)_{z}Manα/β

wherein x and y are linkage positions being either 3 or 6,

z is integer 0 or 1, indicating the presence or the absence of the branch,

The high mannose structure includes terminal α2-linked Mannose:

Manα2Man(α) and optionally on or several of the terminal α3- and/or α6- mannose-structures as above.

The presence of terminal Manα-structures is regulated in stem cells and the proportion of the high-Man-structures with terminal Manα2-structures in relation to the low Man structures with Manα3/6- and/or to complex type N-glycans with Gal-backbone epitopes varies cell type specifically.

The data indicated that binder revealing specific terminal Manα2Man and/or Manα3/6Man is very useful in characterization of mesenchymal cells. The prior science has not characterized the epitopes as specific signals of cell types or status. The invention is especially directed to the measuring the levels of both low-Man and high-Man structures, preferably by quantifying two structure type the Manα2Man-structures and the Manα3/6Man-structures from the same sample.

The invention is especially directed to high specificity binders such as enzymes or monoclonal antibodies for the recognition of the terminal Manα-structures from the preferred stem cells according to the invention. The invention is especially preferably directed to detection of the structures from adult stem cells more preferably mesenchymal stem cells, especially from the surface of mesenchymal stem cells and in separate embodiment from blood derived mesenchymal cells, with separately preferred groups of cord blood and bone marrow stem and mesenchymal cells. In a preferred embodiment the cord blood and/or peripheral blood stem cell is not hematopoietic stem cell.

Low or Uncharacterised Specificity Binders

Preferred for recognition of terminal mannose structures includes mannose-monosaccharide binding plant lectins. The invention is in preferred embodiment directed to the recognition of stem cells such as mesenchymal stem cells or mesenchymal cells by a Manα-recognizing lectin such as lectin PSA (with also specificity for core fucose structures. In a preferred embodiment the recognition is directed to the intracellular glycans in permebilized cells. In another embodiment the Manα-binding lectin is used for intact non-permeabilized cells to recognize terminal Manα-from contaminating cell population such as fibroblast type cells or feeder cells as shown in corresponding Examples.

Preferred High Specificity Binders

Include

i) Specific mannose residue releasing enzymes such as linkage specific mannosidases, more preferably an α-mannosidase or β-mannosidase.

Preferred α-mannosidases includes linkage specific α-mannosidases such as α-Mannosidases cleaving preferably non-reducing end terminal, an example of preferred mannosidases is jack bean α-mannosidase (Canavalia ensiformis; Sigma, USA) and homologous α-mannosidases

α2-linked mannose residues specifically or more effectively than other linkages, more preferably cleaving specifically Manα2-structures; or

α3-linked mannose residues specifically or more effectively than other linkages, more preferably cleaving specifically Manα3-structures; or

α6-linked mannose residues specifically or more effectively than other linkages, more preferably cleaving specifically Manα6-structures;

Preferred β-mannosidases includes β-mannosidases capable of cleaving β4-linked mannose from non-reducing end terminal of N-glycan core Manβ4GlcNAc-structure without cleaving other β-linked monosaccharides in the glycomes.

ii) Specific binding proteins recognizing preferred mannose structures according to the invention. The preferred reagents include antibodies and binding domains of antibodies (Fab-fragments and like), and other engineered carbohydrate binding proteins. The invention is directed to antibodies recognizing MS2B1 and more preferably MS3B2-structures.

Mannosidase analyses of neutral N-glycans. Examples of detection of mannosylated glycans by α-mannosidase binder and mass spectrometric profiling of the glycans of cord blood and peripheral blood mesenchymal cells and differentiated cells in Example 1; indicate presence of all types of Manβ4, Manα3/6 terminal structures of Man₁₋₄GlcNAcβ4(Fucα6)₀₋₁GlcNAc- comprising low Mannose glycans as described by the invention.

Lectin Binding

α-linked mannose was demonstrated in Example 2 for human mesenchymal cells by lectins Hippeastrum hybrid (HHA) and Pisum sativum (PSA, also especially core fucose recognizing). Lectin results suggests that hMSCs express mannose, more specifically α-linked mannose residues on their surface glycoconjugates such as N-glycans. Possible α-mannose linkages include α1→2, α1→3, and α1→6. The lower binding of Galanthus nivalis (GNA) lectin suggests that some α-mannose linkages on the cell surface are more prevalent than others. The combination of the terminal Manα-recognizing low affinity reagents appears to be useful and correspond to results optained by mannosidase screening; NMR and mass spectrometric results.

Mannose-binding lectin labelling. Labelling of the mesenchymal cells in Example 2 was also detected with human serum mannose-binding lectin (MBL) coupled to fluorescein label. This indicate that ligands for this innate immunity system component may be expressed on in vitro cultured BM MSC cell surface. The present invention is especially directed to analysis of terminal Manα-on cell surfaces as the structure is ligand for MBL and other lectins of innate immunity. It is further realized that terminal Manα-structures would direct cells in blood circulation to mannose receptor comprising tissues such as Kupfer cells of liver. The invention is especially directed to control of the amount of the structure by binding with a binder recognizing terminal Manα-structure.

In a preferred embodiment the present invention is directed to the testing of presence of ligands of lectins present in human, such as lectins of innate immunity and/or lectins of tissues or leukocytes, on stem cells by testing of the binding of the lectin (purified or preferably a recombinant form of the lectin, preferably in labeled form) to the stem cells. It is realized that such lectins includes especially lectins binding Manα and Galβ/GalNAcβ-structures (terminal non-reducing end or even α6-sialylated forms) according to the invention.

Mannose Binding Antibodies

A high-mannose binding antibody has been described for example in Wang LX et al (2004) 11 (1) 127-34. Specific antibodies for short mannosylated structures such as the trimannosyl core structure have also been published.

Structures with Terminal Gal-Monosaccharide

Preferred galactose-type target structures have been specifically classified by the invention. These include various types of N-acetyllactosamine structures according to the invention.

Low or Uncharacterised Specificity Binders for Terminal Gal

Preferred for recognition of terminal galactose structures includes plant lectins such as ricin lectin (ricinus communis agglutinin RCA), and peanut lectin(/agglutinin PNA). The low resolution binders have different and broad specificities.

Preferred High Specificity Binders Include

i) Specific galactose residue releasing enzymes such as linkage specific galactosidases, more preferably α-galactosidase or β-galactosidase.

Preferred α-galactosidases include linkage galactosidases capable of cleaving Galα3Gal-structures revealed from specific cell preparations

Preferred β-galactosidases includes β-galactosidases capable of cleaving

β4-linked galactose from non-reducing end terminal Galβ4GlcNAc-structure without cleaving other β-linked monosaccharides in the glycomes and

β3-linked galactose from non-reducing end terminal Galβ3GlcNAc-structure without cleaving other β-linked monosaccharides in the glycomes

ii) Specific binding proteins recognizing preferred galactose structures according to the invention. The preferred reagents include antibodies and binding domains of antibodies (Fab-fragments and like), and other engineered carbohydrate binding proteins and animal lectins such as galectins.

Specific Binder Experiments and Examples for Galβ-Structures

Specific exoglycosidase analysis for the structures are included in Examples for mesenchymal cells and for glycolipids in Example 7. Sialylation level analysis related to terminal Galβ and Sialic acid expression is in Example 4.

Preferred enzyme binders for the binding of the Galβ-epitopes according to the invention includes β1,4-galactosidase e.g from S. pneumoniae (rec. in E. coli, Calbiochem, USA), β1,3-galactosidase (e.g rec. in E. coli, Calbiochem); glycosyltransferases: α2,3-(N)-sialyltransferase (rat, recombinant in S. frugiperda, Calbiochem), α1,3-fucosyltransferase VI (human, recombinant in S. frugiperda, Calbiochem), which are known to recognize specific N-acetyllactosamine epitopes, Fuc-TVI especially Galβ4GlcNAc.

Plant low specificity lectins, such as RCA, PNA, ECA, STA, and PWA, data is in Example 2 for MSCs, Example 3 for cord blood, effects of the lectin binders for the cell proliferation is in Example 6, cord blood cell selection is in Examples.

In example 8 there is antibody labeling of especially fucosylated and galactosylated structures.

Poly-N-acetyllactosamine sequences. Labelling of the cells by pokeweed (PWA) and labelling by Solanum tuberosum (STA) lectins would reveal that the cells express poly-N-acetyllactosamine sequences on their surface glycoconjugates such as N- and/or O-glycans and/or glycolipids. The results further suggest that cell surface poly-N-acetyllactosamine chains contain both linear and branched sequences.

Structures with Terminal GalNAc-Monosaccharide

Preferred GalNAc-type target structures have been specifically revealed by the invention. These include especially LacdiNAc, GalNAcβGlcNAc-type structures according to the invention.

Low or Uncharacterised Specificity Binders for Terminal GalNAc

Several plant lectins has been reported for recognition of terminal GalNAc. It is realized that some GalNAc-recognizing lectins may be selected for low specificity reconition of the preferred LacdiNAc-structures.

The low specificity binder plant lectins such as Wisteria floribunda agglutinin and Lotus tetragonolobus agglutinin bind to oligosaccharide sequences Srivatsan J. et al. Glycobiology (1992) 2 (5) 445-52: Do, K Y et al. Glycobiology (1997) 7 (2) 183-94; Yan, L., et al (1997) Glycoconjugate J. 14 (1) 45-55. The article also shows that the lectins are useful for recognition of the structures, when the cells are verified not to contain other structures recognized by the lectins.

In a preferred embodiment a low specificity leactin reagent is used in combination with another reagent verifying the binding.

Preferred High Specificity Binders Include

i) The invention revealed that β-linked GalNAc can be recognized by specific β-N-acetylhexosaminidase enzyme in combination with β-N-acetylhexosaminidase enzyme.

This combination indicates the terminal monosaccharide and at least part of the linkage structure.

Preferred β-N-acetylehexosaminidase, includes enzyme capable of cleaving β-linked GalNAc from non-reducing end terminal GalNAcβ4/3-structures without cleaving α-linked HexNAc in the glycomes; preferred N-acetylglucosaminidases include enzyme capable of cleaving β-linked GlcNAc but not GalNAc.

Specific binding proteins recognizing preferred GalNAcβ4, more preferably GalNAcβ4GlcNAc, structures according to the invention. The preferred reagents include antibodies and binding domains of antibodies (Fab-fragments and like), and other engineered carbohydrate binding proteins.

Examples antibodies recognizing LacdiNAc-structures includes publications of Nyame A. K. et al. (1999) Glycobiology 9 (10) 1029-35; van Remoortere A. et al (2000) Glycobiology 10 (6) 601-609; and van Remoortere A. et al (2001) Infect. Immun. 69 (4) 2396-2401. The antibodies were characterized in context of parasite (Schistosoma) infection of mice and humans, but according to the present invention these antibodies can also be used in screening of mesenchymal stem cells. The present invention is especially directed to selection of specific clones of LacdiNac recognizing antibodies specific for the subglycomes and glycan structures present in N-glycomes of the invention.

The articles disclose antibody binding specificities similar to the invention and methods for producing such antibodies, therefore the antibody binders are obvious for person skilled in the art. The immunogenicity of certain LacdiNAc-structures are demonstrated in human and mice.

The use of glycosidase in recognition of the structures in known in the prior art similarity as in the present invention for example in Srivatsan J. et al. (1992) 2 (5) 445-52.

Structures with Terminal GlcNAc-Monosaccharide

Preferred GlcNAc-type target structures have been specifically revealed by the invention. These include especially GlcNAcβ-type structures according to the invention.

Low or Uncharacterised Specificity Binders for Terminal GlcNAc

Several plant lectins has been reported for recognition of terminal GlcNAc. It is realized that some GlcNAc-recognizing lectins may be selected for low specificity recognition of the preferred GlcNAc-structures.

Preferred High Specific High Specificity Binders Include
i) The invention revealed that β-linked GlcNAc can be recognized by specific β-N-acetylglucosaminidase enzyme.

Preferred β-N-acetylglucosaminidase includes enzyme capable of cleaving β-linked GlcNAc from non-reducing end terminal GlcNAcβ2/3/6-structures without cleaving β-linked GalNAc or α-linked HexNAc in the glycomes;

ii) Specific binding proteins recognizing preferred GlcNAcβ2/3/6, more preferably GlcNAcβ2Manα, structures according to the invention. The preferred reagents include antibodies and binding domains of antibodies (Fab-fragments and like), and other engineered carbohydrate binding proteins.

Specific Binder Experiments and Examples for Terminal HexNAc(GalNAc/GlcNAc and GlcNAc Structures

Specific exoglycosidase analysis for the structures are included in Example 1 for mesenchymal cells and for glycolipids in Example 7.

Plant low specificity lectin, such as WFA and GNAII, and data is in Example 2 for MSCs, effects of the lectin binders for the cell proliferation is in Example 6.

Preferred enzymes for the recognition of the structures includes general hexosaminidase β-hexosaminidase from Jack beans (C. ensiformis, Sigma, USA) and and specific N-acetylglucosaminidases or N-acetylgalactosaminidases such as β-glucosaminidase from S. pneumoniae (rec. in E. coli, Calbiochem, USA). Combination of these allows determination of LacdiNAc.

The invention is further directed to analysis of the structures by specific monoclonal antibodies recognizing terminal GlcNAcβ-structures such as described in Holmes and Greene (1991) 288 (1) 87-96, with specificity for several terminal GlcNAc structures. The invention is specifically directed to the use of the terminal structures according to the invention for selection and production of antibodies for the structures.

Verification of the target structures includes mass spectrometry and permethylation/fragmentation analysis for glycolipid structures

Structures with Terminal Fucose-Monosaccharide

Preferred fucose-type target structures have been specifically classified by the invention. These include various types of N-acetyllactosamine structures according to the invention. The invention is further more directed to recognition and other methods according to the invention for lactosamine similar α6-fucosylated epitope of N-glycan core, GlcNAcβ4(Fucα6)GlcNAc. The invention revealed such structures recognizeable by the lectin PSA (Kornfeld (1981) J Biol Chem 256, 6633-6640; Cummings and Kornfeld (1982) J Biol Chem 257, 11235-40) are present e.g. in embryonal stem cells and mesenchymal stem cells.

Low or Uncharacterised Specificity Binders for Terminal Fuc

Preferred for recognition of terminal fucose structures includes fucose monosaccharide binding plant lectins. Lectins of Ulex europeaus and Lotus tetragonolobus has been reported to recognize for example terminal Fucoses with some specificity binding for α2-linked structures, and branching α3-fucose, respectively. Data is in Example 2 for MSCs, and effects of the lectin binders for the cell proliferation is in Example 6.

Preferred High Specificity Binders Include

i) Specific fucose residue releasing enzymes such as linkage fucosidases, more preferably α-fucosidase.

Preferred α-fucosidases include linkage fucosidases capable of cleaving Fucα2Gal-, and Galβ4/3(Fucα3/4)GlcNAc-structures revealed from specific cell preparations.

Specific exoglycosidase and for the structures are included in Example 1 for mesenchymal cells, and for glycolipids in Example 7. Preferred fucosidases includes α1,3/4-fucosidase e.g. α1,3/4-fucosidase from Xanthomonas sp. (Calbiochem, USA), and α1,2-fucosidase e.g α1,2-fucosidase from X. manihotis (Glyko),

ii) Specific binding proteins recognizing preferred fucose structures according to the invention. The preferred reagents include antibodies and binding domains of antibodies (Fab-fragments and like), and other engineered carbohydrate binding proteins and animal lectins such as selectins recognizing especially Lewis type structures such as Lewis x, Galβ4(Fucα3)GlcNAc, and sialyl-Lewis x, SAα3Galβ4(Fucα3)GlcNAc.

The preferred antibodies includes antibodies recognizing specifically Lewis type structures such as Lewis x, and sialyl-Lewis x. More preferably the Lewis x-antibody is not classic SSEA-1 antibody, but the antibody recognizes specific protein linked Lewis x structures such as Galβ4(Fucα3)GlcNAcβ2Manα-linked to N-glycan core.

iii) the invention is further directed to reconition of α6-fucosylated epitope of N-glycan core, GlcNAcβ4(Fucα6)GlcNAc. The invention directed to recognition of such structures by structures by the lectin PSA or lentil lectin (Kornfeld (1981) J Biol Chem 256, 6633-6640) or by specific monoclonal antibodies (e.g. Srikrishna G. et al (1997) J Biol Chem 272, 25743-52). The invention is further directed to methods of isolation of cellular glycan components comprinsing the glycan epitope and isolation stem cell N-glycans, which are not bound to the lectin as control fraction for further characterization.

Structures with Terminal Sialic Acid-Monosaccharide

Preferred sialic acid-type target structures have been specifically classified by the invention.

Low or Uncharacterised Specificity Binders for Terminal Sialic Acid

Preferred for recognition of terminal sialic acid structures includes sialic acid monosaccharide binding plant lectins.

Preferred High Specific High Specificity Binders Include

i) Specific sialic acid residue releasing enzymes such as linkage sialidases, more preferably α-sialidases.

Preferred α-sialidases include linkage sialidases capable of cleaving SAα3Gal- and SAα6Gal -structures revealed from specific cell preparations by the invention. Preferred low specificity lectins, with linkage specificity include the lectins, that are specific for SAα3Gal-structures, preferably being Maackia amurensis lectin and/or lectins specific for SAα6Gal-structures, preferably being Sambucus nigra agglutinin.

ii) Specific binding proteins recognizing preferred sialic acid oligosaccharide sequence structures according to the invention. The preferred reagents include antibodies and binding domains of antibodies (Fab-fragments and like), and other engineered carbohydrate binding proteins and animal lectins such as selectins recognizing especially Lewis type structures such as sialyl-Lewis x, SAα3Galβ4(Fucα3)GlcNAc or sialic acid recognizing Siglec-proteins. The preferred antibodies includes antibodies recognizing specifically sialyl-N-acetyllactosamines, and sialyl-Lewis x.

Preferred antibodies for NeuGc-structures includes antibodies recognizes a structure NeuGcα3Galβ4Glc(NAc)_{0 or 1} and/or GalNAcβ4[NeuGcα3]Galβ4Glc(NAc)_{0 or 1}, wherein [ ] indicates branch in the structure and ( )_{0 or 1} a structure being either present or absent. In a preferred embodiment the invention is directed recognition of the N-glycolyl-Neuraminic acid structures by antibody, preferably by a monoclonal antibody or human/humanized monoclonal antibody. A preferred antibody contains the variable domains of P3-antibody.

Specific Binder Experiments and Examples for α3/6 Sialylated Structures

Specific exoglycosidase analysis for the structures are included in Example 1 for mesenchymal cells, and for glycolipids in Example 7. Sialylation level analysis related to terminal Galβ and Sialic acid expression is in Example 4.

Preferred enzyme binders for the binding of the Sialic acid epitopes according to the invention includes: sialidases such as general sialidase α2,3/6/8/9-sialidase from A. ureafaciens (Glyko), and α2,3-Sialidases such as: α2,3-sialidase from S. pneumoniae (Calbiochem, USA). Other useful sialidases are known from E. coli, and Vibrio cholerae.

α1,3-fucosyltransferase VI (human, recombinant in S. frugiperda, Calbiochem), which are known to recognize specific N-acetyllactosamine epitopes, Fuc-TVI especially including SAα3Galβ4GlcNAc.

Plant low specificity lectin, such as MAA and SNA, and data is in Example 2 for MSCs, Example 3 for cord blood, effects of the lectin binders for the cell proliferation is in Example 6, cord blood cell selection is in Examples.

In example 8 there is antibody labeling of sialylstructures.

Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described. In a further preferred embodiment, the present invention is directed to using galectin ligand structures, derivatives thereof, or ligand-mimicking reagents to uses described in the present invention in stem cell type specific fashion.

The invention is in a preferred embodiment directed to the recognition of terminal N-acetyllactosamines from cells by galectins as described above for recognition of Galβ4GlcNAc and Galβ3GlcNAc structures: The results further correlate with the glycan analysis results showing abundant galectin ligand expression in stem cells and mesenchymal cells, especially non-reducing terminal β-Gal and type II LacNAc, poly-LacNAc, β1,6-branched poly-LacNAc, and complex-type N-glycan expression.

Specific Technical Aspects of Stem Cell Glycome Analysis

Isolation of Glycans and Glycan Fractions

Glycans of the present invention can be isolated by the methods known in the art. A preferred glycan preparation process consists of the following steps:

1° isolating a glycan-containing fraction from the sample,

2° . . . Optionally purification the fraction to useful purity for glycome analysis

The preferred isolation method is chosen according to the desired glycan fraction to be analyzed. The isolation method may be either one or a combination of the following methods, or other fractionation methods that yield fractions of the original sample:

1° extraction with water or other hydrophilic solvent, yielding water-soluble glycans or glycoconjugates such as free oligosaccharides or glycopeptides,

2° extraction with hydrophobic solvent, yielding hydrophilic glycoconjugates such as glycolipids,

3° N-glycosidase treatment, especially Flavobacterium meningosepticum N-glycosidase F treatment, yielding N-glycans,

4° alkaline treatment, such as mild (e.g. 0.1 M) sodium hydroxide or concentrated ammonia treatment, either with or without a reductive agent such as borohydride, in the former case in the presence of a protecting agent such as carbonate, yielding β-elimination products such as O-glycans and/or other elimination products such as N-glycans,

5° endoglycosidase treatment, such as endo-β-galactosidase treatment, especially Escherichia freundii endo-β-galactosidase treatment, yielding fragments from poly-N-acetyllactosamine glycan chains, or similar products according to the enzyme specificity, and/or

6° protease treatment, such as broad-range or specific protease treatment, especially trypsin treatment, yielding proteolytic fragments such as glycopeptides.

The released glycans are optionally divided into sialylated and non-sialylated subfractions and analyzed separately. According to the present invention, this is preferred for improved detection of neutral glycan components, especially when they are rare in the sample to be analyzed, and/or the amount or quality of the sample is low. Preferably, this glycan fractionation is accomplished by graphite chromatography.

According to the present invention, sialylated glycans are optionally modified in such manner that they are isolated together with the non-sialylated glycan fraction in the non-sialylated glycan specific isolation procedure described above, resulting in improved detection simultaneously to both non-sialylated and sialylated glycan components. Preferably, the modification is done before the non-sialylated glycan specific isolation procedure. Preferred modification processes include neuraminidase treatment and derivatization of the sialic acid carboxyl group, while preferred derivatization processes include amidation and esterification of the carboxyl group.

Glycan Release Methods

The preferred glycan release methods include, but are not limited tβ, the following methods:

Free glycans—extraction of free glycans with for example water or suitable water-solvent mixtures.

Protein-linked glycans including O- and N-linked glycans—alkaline elimination of protein-linked glycans, optionally with subsequent reduction of the liberated glycans. Muc in-type and other Ser/Thr O-linked glycans—alkaline β-elimination of glycans, optionally with subsequent reduction of the liberated glycans. N-glycans—enzymatic liberation, optionally with N-glycosidase enzymes including for example N-glycosidase F from C. meningosepticum, Endoglycosidase H from Streptomyces, or N-glycosidase A from almonds.

Lipid-linked glycans including glycosphingolipids—enzymatic liberation with endoglycoceramidase enzyme; chemical liberation; ozonolytic liberation.

Glycosaminoglycans—treatment with endo-glycosidase cleaving glycosaminoglycans such as chondroinases, chondroitin lyases, hyalurondases, heparanases, heparatinases, or keratanases/endo-beta-galactosidases; or use of O-glycan release methods for O-glycosidic Glycosaminoglycans; or N-glycan release methods for N-glycosidic glycosaminoglycans or use of enzymes cleaving specific glycosaminoglycan core structures; or specific chemical nitrous acid cleavage methods especially for amine/N-sulphate comprising glycosaminoglycans

Glycan fragments—specific exo- or endoglycosidase enzymes including for example keratanase, endo-β-galactosidase, hyaluronidase, sialidase, or other exo- and endoglycosidase enzyme; chemical cleavage methods; physical methods

Preferred Target Cell Populations and Types for Analysis According to the Invention

Early Human Cell Populations

Human Stem Cells and Multipotent Cells

Under broadest embodiment the present invention is directed to all types of human mesenchymal cells and mesenchymal stem cells, meaning fresh and cultured human mesenchymal cells. The cells according to the invention do not include traditional cancer cell lines, which may differentiate to resemble natural cells, but represent non-natural development, which is typically due to chromosomal alteration or viral transfection. Mesenchymal cells include all types of non-malignant multipotent cells capable of differentiating to other cell types. The stem cells have special capacity stay as stem cells after cell division, the self-reneval capacity. Preferred types of mesenchymal cells are blood tissue derived mesenchymal cells such as cord blood cells and/or bone marrow derived cells.

Under the broadest embodiment for the human mesenchymal cells, the present invention describes novel special glycan profiles and novel analytics, reagents and other methods directed to the glycan profiles. The invention shows special differences in cell populations with regard to the novel glycan profiles of human stem cells.

The present invention is further directed to the novel structures and related inventions with regard to the preferred cell populations according to the invention. The present invention is further directed to specific glycan structures, especially terminal epitopes, with regard to specific preferred cell population for which the structures are new.

Preferred Types of Mesenchymal Early Human Cells

The invention is directed to specific types of mesenchymal early human cells based on the tissue origin of the cells and/or their differentiation status.

The present invention is specifically directed to the early human cell populations meaning multipotent mesenchymal cells and cell populations derived thereof based on origins of the cells including the age of donor individual and tissue type from which the cells are derived, including preferred cord blood as well as bone marrow from older individuals or adults.

Preferred differentiation status based classification includes preferably “solid tissue progenitor” cells, more preferably “mesenchymal-stem cells”, or cells differentiating to solid tissues or capable of differentiating to cells of either ectodermal, mesodermal, or endodermal, more preferentially especially to mesenchymal stem cells.

The invention is further directed to classification of the early human cells based on the status with regard to cell culture and to two major types of cell material. The present invention is preferably directed to two major cell material types of early human cells including fresh, frozen and cultured cells.

Cord Blood Cells, Embryonal-Type Cells and Bone Marrow Cells

The present invention is specifically directed to mesenchymal early human cell populations meaning multipotent cells and cell populations derived thereof based on the origin of the cells including the age of donor individual and tissue type from which the cells are derived.
a) from early age-cells such 1) as neonatal human, directed preferably to cord blood and related material, and 2) embryonal cell-type material
b) from stem and progenitor cells from older individuals (non-neonatal, preferably adult), preferably derived from human “blood related tissues” comprising, preferably bone marrow cells.

Cells Differentiating to Solid Tissues, Preferably to Mesenchymal Stem Cells

The invention is specifically under a preferred embodiment directed to cells, which are capable of differentiating to non-hematopoietic tissues, referred as “solid tissue progenitors”, meaning to cells differentiating to cells other than blood cells. More preferably the cell population produced for differentiation to solid tissue are “mesenchymal-type cells”, which are multipotent cells capable of effectively differentiating to cells of mesodermal origin, more preferably mesenchymal stem cells.

Most of the glycosylation prior art is directed to hematopoietic cells with characteristics quite different from the mesenchymal-type cells and mesenchymal stem cells according to the invention.

Preferred solid tissue progenitors according to the invention includes selected mesenchymal multipotent cell populations of cord blood, mesenchymal stem cells cultured from cord blood, mesenchymal stem cells cultured/obtained from bone marrow and mesenchymal cells derived from embryonal-type cells. In a more specific embodiment the preferred solid tissue progenitor cells are mesenchymal stem cells, more preferably “blood related mesenchymal cells”, even more preferably mesenchymal stem cells derived from bone marrow or cord blood.

Under a specific embodiment CD34+ comprising stem cells as a more hematopoietic stem cell type of cord blood or CD34+ cells in general are excluded from the solid tissue progenitor cells.

Early Blood Cell Populations and Corresponding Mesenchymal Stem Cells

Cord Blood

The early blood cell populations include blood cell materials enriched with multipotent cells. The preferred early blood cell populations include peripheral blood cells enriched with regard to multipotent cells, bone marrow blood cells, and cord blood cells. In a preferred embodiment the present invention is directed to mesenchymal stem cells derived from early blood or early blood derived cell populations, preferably to the analysis of the cell populations.

Bone Marrow

Another separately preferred group of early blood cells is bone marrow blood cells. These cells do also comprise multipotent cells. In a preferred embodiment the present invention is directed to directed to mesenchymal stem cells derived from bone marrow cell populations, preferably to the analysis of the cell populations.

Preferred Subpopulations of Mesenchymal Early Human Blood Derived Cells

The present invention is specifically directed to subpopulations of early human cells. In a preferred embodiment the subpopulations are produced by selection by an antibody and in another embodiment by cell culture favouring a specific cell type. In a preferred embodiment the cells are produced by an antibody selection method preferably from early blood cells. Preferably the early human blood cells are derived from cord blood cells.

Preferably the homogenous cell populations are selected by binding a specific binder to a cell surface marker of the cell population. In a preferred embodiment the homogenous cells are selected by a cell surface marker having lower correlation with CD34-marker and higher correlation with mesenchymal cell markers on cell surfaces.

The present invention is in a preferred embodiment directed to native cells, meaning non-genetically modified cells. Genetic modifications are known to alter cells and background from modified cells. The present invention further directed in a preferred embodiment to fresh non-cultivated cells.

The invention is directed to use of the markers for analysis of cells of special differentiation capacity, the cells being preferably derived from human blood cells or more preferably human cord blood bone marrow or peripheral blood cells.

Preferred Purity of Reproducibly Highly Purified Mononuclear Complete Cell Populations from Human Cord Blood

The present invention is specifically directed to production of purified mesenchymal cell populations from human cord blood. As described above, production of highly purified complete cell preparations from human cord blood has been a problem in the field. In the broadest embodiment the invention is directed to biological equivalents of human cord blood according to the invention, when these would comprise similar markers and which would yield similar cell populations when separated similarly as the CD 133+ cell population and equivalents according to the invention or when cells equivalent to the cord blood is contained in a sample further comprising other cell types. It is realized that characteristics similar to the cord blood can be at least partially present before the birth of a human. The inventors found out that it is possible to produce highly purified cell populations from early human cells with purity useful for exact analysis of sialylated glycans and related markers.

Preferred Bone Marrow Derived Mesenchymal Cells

The present invention is directed to mesenchymal multipotent cell populations or early human blood cells from human bone marrow. Most preferred are bone marrow derived mesenchymal stem cells. In a preferred embodiment the invention is directed to mesenchymal stem cells differentiating to cells of structural support function such as bone and/or cartilage.

A variety of factors previously mentioned influence ability of stem cells to survive, replicate, and differentiate. For example, in terms of nutrients the amino acid taurine under certain conditions preferentially inhibits murine bone marrow cells from forming osteoclasts (Koide, et al., 1999, Arch Oral Biol 44:711-719), the amino acid L-arginine stimulates erythrocyte differentiation and proliferation of erythroid progenitors (Shima, et al., 2006, Blood 107:1352-1356), extracellular ATP acting through P2Y receptors mediates a wide variety of changes to both hematopoietic and non-hematopoietic stem cells (Lee, et al., 2003, Genes Dev 17:1592-1604), arginine-glycine-aspartic acid attached to porous polymer scaffolds increase differentiation and survival of osteoblast progenitors (Hu, et al., 2003, J Biomed Mater Res A 64:583-590), each of which is incorporated by reference herein in its entirety. Accordingly, one skilled in the art would know to use various types of nutrients for inducing differentiation, or maintaining viability, of certain types of stem cells and/or progeny thereof.

Mesenchymal Cell Populations Derived from Embryonal-Type Cells

The present invention is specifically directed to methods directed to mesenchymal cells derived from embryonal-type cell populations, preferably the mesenchymal cells are similar or equivalent of blood tissue/cells derived mesenchymal cells, In a preferred embodiment the use does not involve commercial or industrial use of human embryos nor involve destruction of human embryos. The invention is under a specific embodiment directed to use of embryonal cells and embryo derived materials such as embryonal stem cells, whenever or wherever it is legally acceptable. It is realized that the legislation varies between countries and regions.

The present invention is further directed to use of embryonal-related, discarded or spontaneously damaged material, which would not be viable as human embryo and cannot be considered as a human embryo. In yet another embodiment the present invention is directed to use of accidentally damaged embryonal material, which would not be viable as human embryo and cannot be considered as human embryo. The invention is further directed to cell derived from reprogrammed embryonal like cell derived cells such as human fibroblasts derived cells of Yamanaka Science 2007.

It is further realized that early human blood derived from human cord or placenta after birth and removal of the cord during normal delivery process is ethically uncontroversial discarded material, forming no part of human being.

The invention is further directed to cell materials equivalent to the cell materials according to the invention. It is further realized that functionally and even biologically similar cells may be obtained by artificial methods including cloning technologies.

Mesenchymal Cells and Mesenchymal Multipotent/Stem Cells

The invention is directed to “mesenchymal cells” meaning mesenchymal stem cells and cell differentiated thereof The present invention is further directed to mesenchymal stem cells or multipotent cells as preferred cell population according to the invention. The preferred mesencymal stem cells include cells derived from early human cells, preferably human cord blood or from human bone marrow. In a preferred embodiment the invention is directed to mesenchymal stem cells differentiating to cells of structural support function such as bone and/or cartilage, or to cells forming soft tissues such as adipose tissue.

The differentiated mesenchymal cells includes differentiated cell types derived from the mesenchymal stem cells such cells of structural support function such as bone and/or cartilage, or to cells forming soft tissues such as adipose tissue. The differentiated cells are in a preferred embodiment cells which can be transferred to tissues and which have capacity to incorporated to the tissue. The diferentiated cells may have further capacity for differentiation to the target tissue cells types. In a preferred embodiemnt the differentiated cell are produced in vitro from the mesenchymal stem cells, preferably by in vitro cell culture method. The cell culture method causes the differentiation of mesenchymal stem cells totally or partially to a more specific tissue type cells, in a preferred embodiment the differentiation occurs in rane simila as known in the art for differnetiation of stem cells and/or in the range of differentiation of differentiated cells in the examples such as from a few weeks to months e.g two weeks to 6 month, preferably 1-3 months and it is relized that the differentiation may be optimized to occur in shorter time frame.

Control of Cell Status and Potential Contaminations by Glycosylation Analysis

Control of Cell Status

Control of Raw Material Cell Population

The present invention is directed to control of glycosylation of cell populations to be used in therapy.

The present invention is specifically directed to control of glycosylation of cell materials, preferably when
1) there is difference between the origin of the cell material and the potential recipient of transplanted material. In a preferred embodiment there are potential inter-individual specific differences between the donor of cell material and the recipient of the cell material. In a preferred embodiment the invention is directed to animal or human, more preferably human specific, individual person specific glycosylation differences. The individual specific differences are preferably present in mononuclear cell populations of early human cells, early human blood cells and embryonal type cells. The invention is preferably not directed to observation of known individual specific differences such as blood group antigens changes on erythrocytes.
2) There is possibility in variation due to disease specific variation in the materials. The present invention is specifically directed to search of glycosylation differences in the early cell populations according to the present invention associated with infectious disease, inflammatory disease, or malignant disease. Part of the inventors have analysed numerous cancers and tumors and observed similar types glycosylations as certain glycosylation types in the early cells. It is however realized that there is clear difference of the therapeutically useful non-malignat mesenchymal cells according to the invention and harmful cancer cells with variations betrween cell types and individual samples. Cancer cause currently non-predictable alterations of cell glycosylation, which may in part accidentially be similar an in most parts different from the other natural glycosylation on level of glycome and even on level of epitopes of single glycan, and therefore thorough analysis to differente these is useful.
3) There is for a possibility of specific inter-individual biological differences in the animals, preferably humans, from which the cell are derived for example in relation to species, strain, population, isolated population, or race specific differences in the cell materials.
4) When it has been established that a certain cell population can be used for a cell therapy application, glycan analysis can be used to control that the cell population has the same characteristics as a cell population known to be useful in a clinical setting.

Time Dependent Changes During Cultivation of Cells

Furthermore during long term cultivation of cells spontaneous mutations may be caused in cultivated cell materials. It is noted that mutations in cultivated cell lines often cause harmful defects on glycosylation level.

It is further noticed that cultivation of cells may cause changes in glycosylation. It is realized that minor changes in any parameter of cell cultivation including quality and concentrations of various biological, organic and inorganic molecules, any physical condition such as temperature, cell density, or level of mixing may cause difference in cell materials and glycosylation. The present invention is directed to monitoring glycosylation changes according to the present invention in order to observe change of cell status caused by any cell culture parameter affecting the cells.

The present invention is in a preferred embodiment directed to analysis of glycosylation changes when the density of cells is altered. The inventors noticed that this has a major impact of the glycosylation during cell culture.

It is further realized that if there is limitations in genetic or differentiation stability of cells, these would increase probability for changes in glycan structures. Cell populations in early stage of differentiation have potential to produce different cell populations. The present inventors were able to discover glycosylation changes in early human cell populations.

Differentiation of Cell Lines

The present invention is specifically directed to observe glycosylation changes according to the present invention when differentiation of a cell line is observed. In a preferred embodiment the invention is directed to methods for observation of differentiation from early human cell or another preferred cell type according to the present invention to mesodermal types of stem cell

In case there is heterogeneity in cell material this may cause observable changes or harmful effects in glycosylation.

Furthermore, the changes in carbohydrate structures, even non-harmful or functionally unknown, can be used to obtain information about the exact genetic status of the cells.

The present invention is specifically directed to the analysis of changes of glycosylation, preferably changes in glycan profiles, individual glycan signals, and/or relative abundancies of individual glycans or glycan groups according to the present invention in order to observe changes of cell status during cell cultivation. Analysis of supporting/feeder cell lines

The present invention is specifically directed to observe glycosylation differences according to the present invention, on supporting/feeder cells used in cultivation of stem cells and early human cells or other preferred cell type. It is known in the art that some cells have superior activities to act as a support/feeder cells than other cells. In a preferred embodiment the invention is directed to methods for observation of differences on glycosylation on these supporting/feeder cells. This information can be used in design of novel reagents to support the growth of the stem cells and early human cells or other preferred cell type.

Contaminations or Alterations in Cells Due to Process Conditions

Conditions and Reagents Inducing Harmful Glycosylation or Harmful Glycosylation Related Effects to Cells During Cell Handling

The inventors further revealed conditions and reagents inducing harmful glycans to be expressed by cells with same associated problems as the contaminating glycans. The inventors found out that several reagents used in a regular cell purification processes caused changes in early human cell materials.

It is realized, that the materials during cell handling may affect the glycosylation of cell materials. This may be based on the adhesion, adsorption, or metabolic accumulation of the structure in cells under processing.

In a preferred embodiment the cell handling reagents are tested with regard to the presence glycan component being antigenic or harmfull structure such as cell surface NeuGc, Neu-O-Ac or mannose structure. The testing is especially preferred for human early cell populations and preferred subpopulations thereof.

The inventors note effects of various effector molecules in cell culture on the glycans expressed by the cells if absortion or metabolic transfer of the carbohydrate structures have not been performed. The effectors typically mediate a signal to cell for example through binding a cell surface receptor.

The effector molecules include various cytokines, growth factors, and their signalling molecules and co-receptors. The effector molecules may be also carbohydrates or carbohydrate binding proteins such as lectins.

Controlled Cell Isolation/Purification and Culture Conditions to Avoid Contaminations with Harmful Glycans or Other Alteration in Glycome Level

Stress Caused by Cell Handling

It is realized that cell handling including isolation/purification, and handling in context of cell storage and cell culture processes are not natural conditions for cells and cause physical and chemical stress for cells. The present invention allows control of potential changes caused by the stress. The control may be combined by regular methods may be combined with regular checking of cell viability or the intactness of cell structures by other means.

Examples of Physical and/or Chemical Stress in Cell Handling Step

Washing and centrifuging cells cause physical stress which may break or harm cell membrane structures. Cell purifications and separations or analysis under non-physiological flow conditions also expose cells to certain non-physiological stress. Cell storage processes and cell preservation and handling at lower temperatures affects the membrane structure. All handling steps involving change of composition of media or other solution, especially washing solutions around the cells affect the cells for example by altered water and salt balance or by altering concentrations of other molecules effecting biochemical and physiological control of cells.

Observation and Control of Glycome Changes by Stress in Cell Handling Processes

The inventors revealed that the method according to the invention is useful for observing changes in cell membranes which usually effectively alter at least part of the glycome observed according to the invention. It is realized that this related to exact organization and intact structures cell membranes and specific glycan structures being part of the organization.

The present invention is specifically directed to observation of total glycome and/or cell surface glycomes, these methods are further aimed for the use in the analysis of intactness of cells especially in context of stressfull condition for the cells, especially when the cells are exposed to physical and/or chemical stress. It is realized that each new cell handling step and/or new condition for a cell handling step is useful to be controlled by the methods according to the invention. It is further realized that the analysis of glycome is useful for search of most effectively altering glycan structures for analysis by other methods such as binding by specific carbohydrate binding agents including especially carbohydrate binding proteins (lectins, antibodies, enzymes and engineered proteins with carbohydrate binding activity).

Controlled Cell Preparation (Isolation or Purification) with Regard to Reagents

The inventors analysed process steps of common cell preparation methods. Multiple sources of potential contamination by animal materials were discovered.

The present invention is specifically directed to carbohydrate analysis methods to control of cell preparation processes. The present invention is specifically directed to the process of controlling the potential contaminations with animal type glycans, preferably N-glycolylneuraminic acid at various steps of the process.

The invention is further directed to specific glycan controlled reagents to be used in cell isolation

The glycan-controlled reagents may be controlled on three levels:
1. Reagents controlled not to contain observable levels of harmful glycan structure, preferably N-glycolylneuraminic acid or structures related to it
2. Reagents controlled not to contain observable levels of glycan structures similar to the ones in the cell preparation
3. Reagent controlled not to contain observable levels of any glycan structures.

The control levels 2 and 3 are useful especially when cell status is controlled by glycan analysis and/or profiling methods. In case reagents in cell preparation would contain the indicated glycan structures this would make the control more difficult or prevent it. It is further noticed that glycan structures may represent biological activity modifying the cell status.

Cell Preparation Methods Including Glycan-Controlled Reagents

The present invention is further directed to specific cell purification methods including glycan-controlled reagents.

Preferred Controlled Cell Purification Process

When the binders are used for cell purification or other process after which cells are used in method where the glycans of the binder may have biological effect the binders are preferably glycan controlled or glycan neutralized proteins.

The present invention is especially directed to controlled production of human early cells containing one or several following steps. It was realized that on each step using regular reagents in following process there is risk of contamination by extragenous glycan material. The process is directed to the use of controlled reagents and materials according to the invention in the steps of the process.

Preferred purification of cells includes at least one of the steps including the use of controlled reagent, more preferably at least two steps are included, more preferably at least 3 steps and most preferably at least steps 1, 2, 3, 4, and 6.
1. Washing cell material with controlled reagent.
2. When antibody based process is used cell material is in a preferred embodiment blocked with controlled Fc-receptor blocking reagent. It is further realized that part of glycosylation may be needed in a antibody preparation, in a preferred embodiment a terminally depleted glycan is used.
3. Contacting cells with immobilized cell binder material including controlled blocking material and controlled cell binder material. In a more preferred the cell binder material comprises magnetic beads and controlled gelatin material according the invention. In a preferred embodiment the cell binder material is controlled, preferably a cell binder antibody material is controlled. Otherwise the cell binder antibodies may contain even N-glycolylneuraminic acid, especially when the antibody is produced by a cell line producing N-glycolylneuraminic acid and contaminate the product.
4. Washing immobilized cells with controlled protein preparation or non-protein preparation.
In a preferred process magnetic beads are washed with controlled protein preparation, more preferably with controlled albumin preparation.
5. Optional release of cells from immobilization.
6. Washing purified cells with controlled protein preparation or non-protein preparation.

In a preferred embodiment the preferred process is a method using immunomagnetic beads for purification of early human cells, preferably purification of cord blood cells.

The present invention is further directed to cell purification kit, preferably an immunomagnetic cell purification kit comprising at least one controlled reagent, more preferably at least two controlled reagents, even more preferably three controlled reagents, even preferably four reagents and most preferably the preferred controlled reagents are selected from the group: albumin, gelatin, antibody for cell purification and Fc-receptor blocking reagent, which may be an antibody.

Contaminations with Harmful Glycans Such as Antigenic Animal Type Glycans

Several glycans structures contaminating cell products may weaken the biological activity of the product.

The harmful glycans can affect the viability during handling of cells, or viability and/or desired bioactivity and/or safety in therapeutic use of cells. The harmful glycan structures may reduce the in vitro or in vivo viability of the cells by causing or increasing binding of destructive lectins or antibodies to the cells. Such protein material may be included e.g. in protein preparations used in cell handling materials. Carbohydrate targeting lectins are also present on human tissues and cells, especially in blood and endothelial surfaces. Carbohydrate binding antibodies in human blood can activate complement and cause other immune responses in vivo. Furthermore immune defence lectins in blood or leukocytes may direct immune defence against unusual glycan structures.

Additionally harmful glycans may cause harmful aggregation of cells in vivo or in vitro. The glycans may cause unwanted changes in developmental status of cells by aggregation and/or changes in cell surface lectin mediated biological regulation.

Additional problems include allergenic nature of harmful glycans and misdirected targeting of cells by endothelial/cellular carbohydrate receptors in vivo.

Common Structural Features of All Glycomes and Preferred Common Subfeatures

The present invention reveals useful glycan markers for stem cells and combinations thereof and glycome compositions comprising specific amounts of key glycan structures. The invention is furthermore directed to specific terminal and core structures and to the combinations thereof.

The preferred glycome glycan structure(s) and/or glycomes from cells according to the invention comprise structure(s) according to the formula C0:

R₁Hexβz{R₃}ₙ₁Hex(NAc)ₙ₂XyR₂,

Wherein X is glycosidically linked disaccharide epitope β4(Fucα6)ₙGN, wherein n is 0 or 1, or X is nothing and

Hex is Gal or Man or GlcA,

HexNAc is GlcNAc or GalNAc,

y is anomeric linkage structure α and/or β or linkage from derivatized anomeric carbon,

z is linkage position 3 or 4, with the provision that when z is 4 then HexNAc is GlcNAc and then Hex is Man or Hex is Gal or Hex is GlcA, and

when z is 3 then Hex is GlcA or Gal and HexNAc is GlcNAc or GalNAc;

n1 is 0 or 1 indicating presence or absence of R3;

n2 is 0 or 1, indicating the presence or absence of NAc, with the proviso that n2 can be 0 only when Hexβz is Galβ4, and n2 is preferably 0, n2 structures are preferably derived from glycolipids;

R₁ indicates 1-4, preferably 1-3, natural type carbohydrate substituents linked to the core structures or nothing;

R₂ is reducing end hydroxyl, chemical reducing end derivative or natural asparagine N-glycoside derivative such as asparagine N-glycosides including asparagine N-glycoside aminoacids and/or peptides derived from protein, or natural serine or threonine linked O-glycoside derivative such as serine or threonine linked O-glycosides including asparagine N-glycoside aminoacids and/or peptides derived from protein, or when n2 is 1 R2 is nothing or a ceramide structure or a derivetive of a ceramide structure, such as lysolipid and amide derivatives thereof,

R3 is nothing or a branching structure respesenting a GlcNAcβ6 or an oligosaccharide with GlcNAcβ6 at its reducing end linked to GalNAc (when HexNAc is GalNAc); or

when Hex is Gal and HexNAc is GlcNAc, and when z is 3 then R3 is Fucα4 or nothing, and when z is 4 R3 is Fucα3 or nothing.

The preferred disaccharide epitopes in the glycan structures and glycomes according to the invention include structures Galβ4GlcNAc, Manβ4GlcNAc, GlcAβ4GlcNAc, Galβ3GlcNAc, Galβ3GalNAc, GlcAβ3GlcNAc, GlcAβ3GalNAc, and Galβ4Glc, which may be further derivatized from reducing end carbon atom and non-reducing monosaccharide residues and is in a separate embodiment branched from the reducing end residue. Preferred branched epitopes include Galβ4(Fucα3)GlcNAc, Galβ3(Fucα4)GlcNAc, and Galβ3(GlcNAcβ6)GalNAc, which may be further derivatized from reducing end carbon atom and non-reducing monosaccharide residues.

Preferred Epitopes for Methods According to the Invention

N-Acetyllactosamine Galβ83/4GlcNAc Terminal Epitopes

The two N-acetyllactosamine epitopes Galβ4GlcNAc and/or Galβ3GlcNAc represent preferred terminal epitopes present on stem cells or backbone structures of the preferred terminal epitopes for example further comprising sialic acid or fucose derivatisations according to the invention. In a preferred embodiment the invention is direted to fucosylated and/or non-substituted glycan non-reducing end forms of the terminal epitopes, more preferably to fucosylated and non-substutituted forms. The invention is especially directed to non-reducing end terminal (non-susbtituted) natural Galβ4GlcNAc and/or Galβ3GlcNAc-structures from human stem cell glycomes. The invention is in a specific embodiment directed to non-reducing end terminal fucosylated natural Galβ4GlcNAc and/or Galβ3GlcNAc-structures from human stem cell glycomes.

Preferred Fucosylated N-Acetyllactosamines

The preferred fucosylated epitopes are according to the Formula TF:

(Fucα2)ₙ₁Galβ3/4(Fucα4/3)ₙ₂GlcNAcβ-R

Wherein

n1 is 0 or 1 indicating presence or absence of Fucα2;

n2 is 0 or 1, indicating the presence or absence of Fucα4/3 (branch), and

R is the reducing end core structure of N-glycan, O-glycan and/or glycolipid.

The preferred structures thus include type 1 lactosamines (Galβ3GlcNAc based):

Galβ3(Fucα4)GlcNAc (Lewis a), Fucα2Galβ3GlcNAc H-type 1, structure and,

Fucα2Galβ3(Fucα4)GlcNAc (Lewis b) and

type 2 lactosamines (Galβ4GlcNAc based):

Galβ4(Fucα3)GlcNAc (Lewis x), Fucα2Galβ4GlcNAc H-type 2, structure and,

Fucα2Galβ4(Fucα3)GlcNAc (Lewis y).

The type 2 lactosamines (fucosylated and/or terminal non-substituted) form an especially preferred group in context of adult stem cells.and differentiated cells derived directly from these. Type 1 lactosamines (Galβ3GlcNAc—structures) are especially preferred in context of embryonal-type stem cells.

Lactosamines Galβ83/4GlcNAc and Glycolipid Structures Comprising Lactose Structures (Galβ84Glc)

The lactosamines form a preferred structure group with lactose-based glycolipids. The structures share similar features as products of β3/4Gal-transferases. The β3/4 galactose based structures were observed to produce characteristic features of protein linked and glycolipid glycomes.

The invention revealed that furthermore Galβ3/4GlcNAc-structures are a key feature of differentiation releated structures on glycolipids of various stem cell types. Such glycolipids comprise two preferred structural epitopes according to the invention. The most preferred glycolipid types include thus lactosylceramide based glycosphingolipids and especially lacto-(Galβ3GlcNAc), such as lactotetraosylceramide Galβ3 GlcNAcβ3Galβ4GlcβCer, prefered structures further including its non-reducing terminal structures selected from the group:

Galβ3(Fucα4)GlcNAc (Lewis a), Fucα2Galβ3GlcNAc (H-type 1), structure and,

Fucα2Galβ3(Fucα4)GlcNAc (Lewis b) or sialylated structure SAoα3Galβ3GlcNAc or

SAα3Galβ3(Fucα4)GlcNAc, wherein SA is a sialic acid, preferably Neu5Ac preferably replacing Galβ3GlcNAc of lactotetraosylceramide and its fucosylated and/or elogated variants such as preferably according to the Formula:

(Sacα3 )ₙ₅(Fucα2)ₙ₁Galβ3(Fucα4)ₙ₃GlcNAcβ3[Galβ3/4(Fucα4/3)ₙ₂GlcNAcβ3]ₙ₄Galβ4GlcβCer

wherein

n1 is 0 or 1, indicating presence or absence of Fucα2;

n2 is 0 or 1, indicating the presence or absence of Fucα4/3 (branch),

n3 is 0 or 1, indicating the presence or absence of Fucα4 (branch)

n4 is 0 or 1, indicating the presence or absence of (fucosylated) N-acetyllactosamine elongation;

n5 is 0 or 1, indicating the presence or absence of Sacox3 elongation;

Sac is terminal structure, preferably sialic acid, with α3-linkage, with the proviso that when Sac is present, n5 is 1, then n1 is 0

and

neolacto (Galβ4GlcNAc)-comprising glycolipids such as neolactotetraosylceramide Galβ4GlcNAcβ3Galβ4GlcβCer, preferred structures further including its non-reducing terminal Galβ4(Fucα3)GlcNAc (Lewis x),

Fucα2Galβ4GlcNAc H-type 2, structure and, Fucα2Galβ4(Fucα3)GlcNAc (Lewis y)

and

its fucosylated and/or elogated variants such as preferably

(Sacα3/6)ₙ₅(Fuc═2)ₙ₁Galβ4(Fucα3)ₙ₃GlcNAcβ3[Galβ4(Fucα3)ₙ₂GlcNAcβ3]ₙ₄Galβ4GlcβCer

n1 is 0 or 1 indicating presence or absence of Fucα2;

n2 is 0 or 1, indicating the presence or absence of Fucα3 (branch),

n3 is 0 or 1, indicating the presence or absence of Fucα3 (branch)

n4 is 0 or 1, indicating the presence or absence of (fucosylated) N-acetyllactosamine elongation,

n5 is 0 or 1, indicating the presence or absence of Sacα3/6 elongation;

Sac is terminal structure, preferably sialic acid (SA) with α3-linkage, or sialic acid with α6-linkage, with the proviso that when Sac is present, n5 is 1, then n1 is 0, and when sialic acid is bound by α6-linkage preferably also n3 is 0.

Preferred Stem Cell Glycosphingolipid Glycan Profiles, Compositions, and Marker Structures

The inventors were able to describe stem cell glycolipid glycomes by mass spectrometric profiling of liberated free glycans, revealing about 80 glycan signals from different stem cell types. The proposed monosaccharide compositions of the neutral glycans were composed of 2-7 Hex, 0-5 HexNAc, and 0-4 dHex. The proposed monosaccharide compositions of the acidic glycan signals were composed of 0-2 NeuAc, 2-9 Hex, 0-6 HexNAc, 0-3 dHex, and/or 0-1 sulphate or phosphate esters. The present invention is especially directed to analysis and targeting of such stem cell glycan profiles and/or structures for the uses described in the present invention with respect to stem cells.

The present invention is further specifically directed to glycosphingolipid glycan signals specific tostem cell types as described in the Examples. In a preferred embodiment, glycan signals typical to MSC, especially CB MSC, preferentially including 1460 and 1298, as well as large neutral glycolipids, especially Hex₂₋₃HexNAc₃Lac, more preferentially poly-N-acetyllactosamine chains, even more preferentially P 1,6-branched, and preferentially terminated with type II LacNAc epitopes as described above, are used in context of MSC according to the uses described in the present invention.

Terminal glycan epitopes that were demonstrated in the present experiments in stem cell glycosphingolipid glycans are useful in recognizing stem cells or specifically binding to the stem cells via glycans, and other uses according to the present invention, including terminal epitopes: Gal, Galβ4Glc (Lac), Galβ4GlcNAc (LacNAc type 2), Galβ3, Non-reducing terminal HexNAc, Fuc, α1,2-Fuc, α1,3-Fuc, Fucα2Gal, Fucα2Galβ4GlcNAc (H type 2), Fucα2Galβ4Glc (2′-fucosyllactose), Fucα3GlcNAc, Galβ4(Fucα3)GlcNAc (Lex), Fucα3Glc,

Galβ4(Fucα3)Glc (3-fucosyllactose), Neu5Ac, Neu5Acα2,3, and Neu5Acα2,6. The present invention is further directed to the total terminal epitope profiles within the total stem cell glycosphingolipid glycomes and/or glycomes.

The inventors were further able to characterize in hESC the corresponding glycan signals to SSEA-3 and SSEA-4 developmental related antigens, as well as their molar proportions within the stem cell glycome. The invention is further directed to quantitative analysis of such stem cell epitopes within the total glycomes or subglycomes, which is useful as a more efficient alternative with respect to antibodies that recognize only surface antigens. In a further embodiment, the present invention is directed to finding and characterizing the expression of cryptic developmental and/or stem cell antigens within the total glycome profiles by studying total glycan profiles, as demonstrated in the Examples for α1,2-fucosylated antigen expression in hESC in contrast to SSEA-1 expression in mouse ES cells.

The present invention revealed characteristic variations (increased or decreased expression in comparison to similar control cell or a contaminatiog cell or like) of both structure types in various cell materials according to the invention. The structures were revealed with characteristic and varying expression in three different glycome types: N-glycans, O-glycans, and glycolipids. The invention revealed that the glycan structures are a charateristic feature of stem cells and are useful for various analysis methods according to the invention. Amounts of these and relative amounts of the epitopes and/or derivatives varies between cell lines or between cells exposed to different conditions during growing, storage, or induction with effector molecules such as cytokines and/or hormones.

Preferred Epitopes and Antibody Binders Especially for Analysis of Mesenchymal Cells

The invention revelaed glycan structures and epitopes thereof which can be used to detect, isolate and evaluate the differentiation stage, and/or plucipotency of mesenchymal cells, preferably mesenchymal cells and especially mesenchymal stem cells. The detection can be performed in vitro, for FACS purposes and/or for cell lineage specific purposes. The binding reagents such as antibodies can be used to positively isolate and/or separate and/or enrich mesenchymal cells, preferably human stem cells from a mixture of cells comprising feeder or other contaminating cell types and mesenchymal cells or mesenchymal stem cells.

The staining intensity and cell number of stained stem cells, i.e. glycan structures of the present invention on stem cells indicates suitability and usefulness of the binder for isolation and differentiation marker. For example, low relative number of a glycan structure expressing cells may indicate lineage specificity and usefulness for selection of a subset and when selected/isolated from the colonies and cultured. Low number of expression is less than 5%, less than 10%, less than 15%, less than 20%, less than 30% or less than 40%. Further, low number of expression is contemplated when the expression levels are between 1-10%, 10%-20%, 15-25%, 20-40%, 25-35% or 35-50%. Typically, FACS analysis can be performed to enrich, isolate and/or select subsets of cells expressing a glycan structure(s).

High number of glycan expressing cells may indicate usefulness in pluripotency/multipotency marker and that the binder is useful in identifying, characterizing, selecting or isolating pluripotent or multipotent stem cells in a population of mammalian cells. High number of expression is more than 50%, more preferably more than 60%, even more preferably more than 70%, and most preferably more than 80%, 90 or 95%. Further, high number of expression is contemplated when the expression levels are between 50-60, 55%-65%, 60-70%, 70-80, 80-90%, 90-100 or 95-100%. Typically, FACS analysis can be performed to enrich, isolate and/or select subsets of cells expressing a glycan structure(s).

The percentage as used herein means ratio of how many cells express a glycan structure to all the cells subjected to an analysis or an experiment. For example, 20% stem cells expressing a glycan structure in a stem cell colony means that a binder, eg an antibody staining can be observed in about 20% of cells when assessed visually.

Mesenchymal Stem Cells and Differentiated Tissue Type Stem Cells Derived Thereof

Antibodies useful for evalution of differentiation status of mesenchymal stem cells.

Example 8 and Table 15 (lower part) shows labelling of mesenchymal stem cells and differentiated mesenchymal stem cells. In Example 20 and Table 26.

Invention revelead that structures recognized by antibody GF303, preferably Fucα2Galβ3GlcNAc, and GF276 appear during the differentiation of mesenchymal stem cells to osteogenically differentiated stem cells. It was further revelad, that the GalNAcα-group structures GF278, corresponding to Tn-antigen, and GF277, sialyl-Tn increase simultaneously.

The invention is further directed to the preferred uses according to the invention for binders to several target structures, which are characteristic to both mesenchymal stem cells (especially bone marrow derived) and the osteogenically differentiated mesenchymal stem cells. The preferred target structures include one GalNAcα-group structure recognizable by the antibody GF275, the antigen of the antibody is preferably sialylated O-glycan glycopeptide epitope as known for the antibody. The epitopes expressed in both mesenchymal and the osteonically differentiated stem cells further includes two characteristic globo-type antigen structures: the antigen of GF298, which binding correspond to globotriose(Gb3)-type antigens, and the antigen of GF297, which correspond to globotetraose(Gb4) type antigens. The invention has further revealed that terminal type two lactosamine epitopes are especially expressed in both types of mesenchymal stem cells and this was exemplified by staining both cell by antibody recognizing H type II antigen in Example 8 Table 15.

The invention is further directed to the preferred uses according to the invention for binders to several target structures which are substantially reduced or practically diminished/reduced to non-observable level when mesenchymal stem cells (especially bone marrow derived) differentiates to more differentiated, preferably osteogenically differentiated mesenchymal stem cells. These target structures include two globoseries structures, which are preferably Galactosyl-globoside type structure, recognized as antigen SSEA-3, and sialyl-galactosylgloboside type structure, recognized as antigen SSEA-4. The preferred reducing target structures further include two type two N-acetyllactosamine target structures Lewis x and sialyl-Lewis x. Globoside-type glycosphingolipid structures were detected by the inventors in MSC in minor but significant amounts compared to hESC in direct structural analysis, more specifically glycan signals corresponding to SSEA-3 and SSEA-4 glycan antigen monosaccharide compositions. These antigens were also detected by monoclonal antibodies in MSC. The present invention is therefore specifically directed to these globoside structures in context of MSC and cells derived from them in uses described in the invention.

In a preferred embodiment of the present invention, the antibodies or binders which bind to the same epitope than GF275, GF277, GF278, GF297, GF298, GF302, GF305, GF307, GF353, or GF354 are useful to detect/recognize, preferably bone marrow derived, mesenchymal stem cells (corresponding epitopes recognized by the antibodies are listed in Example 8). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells.

Other binders binding to stem cells, preferably human stem cells, comprise of binders which bind to the same epitope than GF275 (sialylated carbohydrate epitope of the MUC-1 glycoprotein). A more preferred antibody comprises of the antibody of clone BM3359 by Acris. This epitope is suitable and can be used to detect, isolate and evaluate of (mesenchymal) stem cells, preferably bome marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow, or differentiated in osteogenic direction from mixture of cells comprising other, bone marrow derived, cells.

Other binders binding to stem cells, preferably human stem cells, comprise of binders which bind to the same epitope than GF305 (Lewis x). A more preferred antibody comprises of the antibody of clone CBL144 by Chemicon. This epitope is suitable and can be used to detect, isolate and evaluate of (mesenchymal) stem cells, preferably bome marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells.

Other binders binding to stem cells, preferably human stem cells, comprise of binders which bind to the same epitope than GF307 (sialyl lewis x). A more preferred antibody comprises of the antibody of clone MAB2096 by Chemicon. This epitope is suitable and can be used to detect, isolate and evaluate of (mesenchymal) stem cells, preferably bome marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells.

In a preferred embodiment, the antibodies or binders which bind to the same epitope than GF305, GF307, GF353 or GF354 are useful for positive selection and/or enrichment of mesenchymal stem cells (corresponding epitopes recognized by the antibodies are listed in Example 8).

In another preferred embodiment of the present invention, antibodies or binders which bind to the same epitope than GF275, GF276, GF277, GF278, GF297, GF298, GF302, GF303, GF307 or GF353 are useful to detect/recognize differentiated, preferably bone marrow derived, mesenchymal stem cells and/or differentiated in osteogenic direction (corresponding epitopes recognized by the antibodies are listed in Example 8). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells.

Other binders binding to stem cells, preferably human stem cells, comprise of binders which bind to the same epitope than GF297 (globoside GL4). A more preferred antibody comprises of the antibody of clone ab23949 by Abcam. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF298 (human CD77; GB3). A more preferred antibody comprises of the antibody of clone SM1160 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF302 (H type 2 blood antigen). In a preferred embodiment, an antibody binds to Fucα2Galβ4GlcNAc epitope. A more preferred antibody comprises of the antibody of clone DM3015 by Acris. This epitope is suitable and can be used to detect, isolate and evaluate of undifferentiated (mesenchymal) stem cells, preferably bome marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

In a preferred embodiment of the present invention, antibodies or binders which bind to the same epitope than GF276, GF277, GF278, GF303, GF305, GF307, GF353, or GF354 are useful to detect/recognize, preferably bone marrow derived, mesenchymal stem cells and differentiated in osteogenic direction (corresponding epitopes recognized by the antibodies are listed in Example 8). These epitopes are suitable and can be used to detect, isolate and evaluate of (mesenchymal) stem cells, preferably bome marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow, or differentiated in osteogenic direction from mixture of cells comprising other, bone marrow derived, cells. Further, the binders which bind to the same epitope than GF276 or GF303, or antibodies GF276 and/or GF303 are particularly useful to detect, isolate and evaluate of osteogenically differentiated stem cells, in culture or in vivo (corresponding epitopes recognized by the antibodies are listed in Example 8).

Other binders binding to stem cells, preferably human stem cells, comprise of binders which bind to the same epitope than GF276 (oncofetal antigen). A more preferred antibody comprises of the antibody of clone DM288 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF277 (human sialosyl-Tn antigen; STn, sCD175). A more preferred antibody comprises of the antibody of clone DM3197 by Acris. This epitope is suitable and can be used to detect, isolate and evaluate of differentiated (mesenchymal) stem cells, preferably bome marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF278 (human sialosyl-Tn antigen; STn, sCD175 B1.1). A more preferred antibody comprises of the antibody of clone DM3218 by Acris. This epitope is suitable and can be used to detect, isolate and evaluate of differentiated (mesenchymal) stem cells, preferably bome marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF303 (blood group H1 antigen, BG4). In a preferred embodiment, an antibody binds to Fucα2Galβ3GlcNAc epitope. A more preferred antibody comprises of the antibody of clone ab3355 by Abcam. This epitope is suitable and can be used to detect, isolate and evaluate of differentiated (mesenchymal) stem cells, preferably bome marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Further, the antibodies or binders are useful to isolate and enrich stem cells for osteogenic lineage. This can be performed with positive selection, for example, with antibodies GF276, GF277, GF278, and GF303 (corresponding epitopes recognized by the antibodies are listed in Example 8). For negative depletion, a preferred epitope is the same as recognized with the antibodies GF296, GF300, GF304, GF305, GF307, GF353, or GF354. For negative depletion, a preferred epitope is the same as recognized with the antibody GF354 (SSEA-4) or GF307 (Sialyl Lewis x).

Miten adipojen diskutointi?

Comparison Between Different Stem Cell Types

The present data revealed that comparision of a group of type 1 and type two N-acetyllactosamines is useful method for characterization of stem cells such as mesenchymal stem cells and embryonal stem cells and or separating the cells from contaminating cell populations such as fibroblasts like feeder cells. The non-differentiated mesenchymal cell were devoid of type I N-acetyllactosamine antigens revealed from the hESC cells, while both cell types and and potential contaminating fibroblast have variable labelling with type II N-acetyllactosamine recognizing antibodies.

The term “mainly” indicates preferably at least 60%, more preferably at least 75% and most preferably at least 90%. In the context of stem cells, the term “mainly” indicates preferably at least 60%, more preferably at least 75% and most preferably at least 90% of cells expressing a glycan structure and useful for identifying, characterizing, selecting or isolating pluripotent or multipotent stem cells in a population of mammalian cells.

Uses of the Binders for Isolation of Cellular Components and Mixtures Thereof

The invention revealed novel binding reagents are in a preferred embodiment used for isolation of cellular components from stem cells comprising the novel target/marker structures. The isolated cellular are preferably free glycans or glycans conjugated to proteins or lipids or fragment thereof.

The invention is especially directed to isolation of the cellular components comprising the structures when the structures comprises one or several types glycan materials sele
a) Free glycans released from the stem cell materials and/or
b) Glycan conjugate material such as
   b1) glycoamino acid materials including
      b1a) glycoproteins
      b1b) glycopeptides including glyco-oligopeptides and glycopolypeptides and/or
   b2) lipid linked materials comprising the preferred carbohydrate structures revealed by the invention.

General Method for Isolation Cellular Components Comprising the Target Structures

The isolation of cellular components according to the invention means production of a molecular fraction comprising increased (or enriched) amount of the glycans comprising the target structures according to the invention in method comprising the step of binding of the binder molecule according to the invention to the corresponding target structures, which are glycan structures bound by the specific binder.

The process of isolation the fraction involving the contacting the binder molecule according to the invention with the corresponding target structures derived from stem cells and isolating the enriched target structure composition.

The preferred method to isolate cellular component includes following steps

1) Providing a stem cell sample.

2) Contacting the binder molecule according to the invention with the corresponding target structures.

3) Isolating the complex of the binder and target structure at least from part of cellular materials.

It is realized that the components are in general enriched in specific fractions of cellular structures such as cellular membrane fractions including plasma membrane and organelle fractions and soluble glycan comprising fractions such as soluble protein, lipid or free glycans fractions. It is realized that the binder can be used to total cellular fractions.

In a preferred embodiment the target structures are enriched within a fraction of cellular proteins such as cell surface proteins releasable by protease or detergent soluble membrane proteins.

The preferred target structure composition comprise glycoproteins or glycopeptides comprising glycan structure corresponding to the binder structure and peptide or protein epitopes specifically expressed in stem cells or in proportions characteristic to stem cells.

More preferably the invention is directed to purification of the target structure fraction in the isolation step. The purification is in a preferred mode of invention is at least partial purification. Preferably the target glycan containing material is purified at least two fold, preferably among the components of cell fraction wherein it is expressed. More preferred purification levels includes 5-fold and 10 fold purification, more preferably 100, and even more preferably 1000- fold purification. Preferably the purified fraction comprises at least 10% of the target glycan comprising molecules, even more preferably at least 30%, even more preferably at least 50%, even more preferably at least 70% pure and most preferably at least 90% pure. Preferably the % value is mole per cent in comparison to other non-target glycan comprising glycaconjugate molecules, more preferably the material is essentially devoid of other major organic contaminating molecules.

Preferred Purified Target Glycan Compositions and Target Glycan-Binder Complexes

The invention is also directed to isolated or purified target glycan-binder complexes and isolated target glycan molecule compositions, wherein the target glycans are enriched with a specific target structures according to the invention.

Preferably the purified target glycan-binder complex compositions comprises at least 10% of the target glycan comprising molecules in complex with binder, even more preferably at least 30%, even more preferably at least 50%, even more preferably at least 70% pure and most preferably at least 90% pure target glycan comprising molecules in complex with binder.

Preferably the purified target glycan composition comprises at least 10% of the target glycan comprising molecules, even more preferably at least 30%, even more preferably at least 50%, even more preferably at least 70% pure and most preferably at least 90% pure target glycan comprising molecules.

The invention is further directed to the enriched target glycan composition produced by the process of isolation the fraction involving the steps of the contacting the binder molecule according to the invention with the corresponding target structures derived from stem cell and isolating the enriched target structure.

Binder Technology for Purification of Target Glycans

The methods for affinity purification of cellular glycoproteins, glycopeptides, free oligosaccharides and other glycan conjugates are well-known in the art. The preferred methods include solid phase involving binder technologies such as affinity chromatography, precipitation such as immunoprecipitation, binder-magnetic methods such as immunomegnetic bead methods. Affinity chromatographies has been described for purification of glycopeptides by using lectins (Wang Y et al (2006) Glycobiology 16 (6) 514-23) or by antibodies or purification of glycoproteins/peptides by using antibodies (e.g. Prat M et al cancer Res (1989) 49, 1415-21; Kim Y D et al et al Cancer Res (1989) 49, 2379) and/or lectins (e.g. Cumming and Komfeld (1982) J Biol Chem 257, 11235-40; Yae E et al. (1991) 1078 (3) 369-76; Shibuya N et al (1988) 267 (2) 676-80; Gonchoroff D G et al. 1989, 35, 29-32; Hentges and Bause (1997) Biol Chem 378 (9) 1031-8). Specific methods have been developed for weakly binding antibodies even for recognition of free oligosaccharides as described e.g. in (Ohlson S et al. J Chromatogr A (1997) 758 (2) 199-208), Ohlson S et al. Anal Biochem (1988) 169 (1) 204-8). The methods may invove multiple steps by binders of different specificities as shown e.g. in (Cummings and Kornfeld (1982) J Biol Chem 257, 11235-40). Antibody or protein (lectin) binder affinity chromatography for oligosaccharide mixtures has been also described e.g. in (Kitagawa H et al. (1991) J Biochem 110 (49 598-604; Kitagawa H et al. (1989) Biochemistry 28 (22) 8891-7; Dakour J et al Arch Biochem Biophys (1988) 264, 203-13) and for glycolipids e.g. in (Bouhours D et al (1990) Arch Biochem Biophys 282 (1) 141-6). Further information of glycan directed affinity chromatography and/or useful lectin and antibody specificites is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands).

The methods includes normal pressure or in HPLC chromatographies and may include additional steps using traditional chromatographic methods or other protein and peptide purification methods, a preferred additional isolation methods is gel filtration (size exclusion) chromatography for isolation of especially lower Mw glycans and conjugates, preferably glycopeptides.

It is further known that isolated proteins and peptides can be recognized by mass spectrometric methods e.g. (Wang Y et al (2006) Glycobiology 16 (6) 514-23). The invention is specifically directed to use of the binders according to the invention for purification of glycans and/or their conjugates and recognition of the isolated component by methods such as mass spectrometry, peptide sequencing, chemical analysis, array analysis or other methods known in the art.

Revealing Presence Trypsin Sensitive Forms of Glycan Targets

The invention reveals in example 10 that part of the target structures of present glycan binders, especially monoclonal antibodies are trypsin sensitive. The antigen structures are essentially not observed or these are observed in reduced amount in FACS analysis of cell surface antigens when cells are treated (released from cultivation) by trypsin but observable after Versene treatment (0.02% EDTA in PBS). This was observed for example for labelling of mesenchymal stem cells by the antibody GF354, which has been indicated to bind SSEA-4 antigen. This target antigen structure has been traditionally considered to be sialyl-galactosylgloboside glycolipid, but obviously the antibody recognizes only an epitope at the non-reducing end of glycan sequence. The present invention is now especially directed to methods of isolation and characterization of mesenchymal stem cell glycopeptide bound glycan structure(s), which can be bound and enriched by the SSEA-4 antibodies, and to characterization of corresponding glycopeptides and glycoproteins. The invention is further directed to analysis of trypsin insensitive glycan materials from stem cell especially mesenchymal stem cells and embryonal stem cells.

The invention revealed also that major part of the sialyl-mucin type target of ab GF 275 is trypsin sensitive and minor part is not trypsin sensitive. The invention is directed to isolation of both trypsin sensitive and trypsin insensitive glycan fractions, preferably glycoprotein(s) and glycopeptides, by methods according to the invention. The invention is further directed to isolation and characterization of protein degrading enzyme (protease) sensitive likely glycopeptides and glycoproteins bound by antibody GF 302, preferably when the materials are isolated from mesenchymal stem cells.

As used herein, “binder”, “binding agent” and “marker” are used interchangeably.

Antibodies

Information about useful lectin and antibody specificites useful according to the invention and for reducing end elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritsumei.ac.jp/epitope/, which list monoclonal antibody specificities).

Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corγnebacterium parvum.

A monoclonal antibody to a peptide motif(s) may be prepared by using any technique which provides for the production of antibody molecules by continuous cell lines in culture. These include but are not limited to the hybridoma technique originally described by Kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-11, MPC11-X45-GTG 1.7 and S194/5XX0 BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81: 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgGl, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, New York (1989)]. The amplified CDR sequences are ligated into an appropriate plasmid. The plasmid comprising one, two, three, four, five and/or six cloned CDRs optionally contains additional polypeptide encoding regions linked to the CDR. Preferably, the antibody is any antibody specific for a glycan structure of Formula (I) or a fragment thereof. The antibody used in the present invention encompasses any antibody or fragment thereof, either native or recombinant, synthetic or naturally-derived, monoclonal or polyclonal which retains sufficient specificity to bind specifically to the glycan structure according to Formula (I) which is indicative of stem cells. As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′).sub.2 fragments, and Fv fragments.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and .beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, .sup. 125 I and amino acids comprising any radionuclides, including, but not limited to .sup. 14 C, .sup.3 H and .sup.35 S.

Antibodies to glycan structure(s) of Formula (I) may be obtained from any source. They may be commercially available. Effectively, any means which detects the presence of glycan structure(s) on the stem cells is with the scope of the present invention. An example of such an antibody is a H type 1 (clone 17-206; GF 287) antibody from Abcam.

The detection for the presence of glycan structure(s) according to Formula (I) on stem cell(s) may be conducted in any way to identify glycan structure according to Formula (I) on stem cell(s). Preferably the detection is by use of a marker or binding protein for glycan structure according to Formula (I) on stem cell(s). The binder/marker for glycan structure according to Formula (I) on stem cell(s) may be any of the markers discussed above. However, antibodies or binding proteins to glycan structure according to Formula (I) on stem cell(s) are particularly useful as a marker for glycan structure according to Formula (I) on stem cell(s).

Various techniques can be employed to separate or enrich the cells by initially removing cells of dedicated lineage. Monoclonal antibodies, binding proteins and lectins are particularly useful for identifying cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. The particular technique employed will depend upon efficiency of separation, associated cytotoxicity, ease and speed of performance, and necessity for sophisticated equipment and/or technical skill.

Procedures for separation or enrichment can include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation or enrichment techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye, Hoescht 33342).

Techniques providing accurate separation include, but are not limited to, FACS, which can have varying degrees of sophistication, e.g., a plurality of color channels, low angle and obtuse light scattering detecting channels, impedence channels, etc. Any method which can isolate and distinguish these cells according to levels of expression of glycan structure according to Formula (I) on stem cell(s) may be used.

In a first separation, typically starting with about 1.times.10.sup.10, preferably at about 5.times.10.sup.8-9 cells, antibodies or binding proteins or lectins to glycan structure according to Formula (I) on stem cell(s) can be labeled with at least one fluorochrome, while the antibodies or binding proteins for the various dedicated lineages, can be conjugated to at least one different fluorochrome. While each of the lineages can be separated in a separate step, desirably the lineages are separated at the same time as one is positively selecting for glycan structure according to Formula (I) on stem cell markers. The cells can be selected against dead cells, by employing dyes associated with dead cells (including but not limited to, propidium iodide (PI)).

To further enrich for any cell population, specific markers for those cell populations may be used. For instance, specific markers for specific cell lineages such as lymphoid, myeloid or erythroid lineages may be used to enrich for or against these cells. These markers may be used to enrich for HSCs or progeny thereof by removing or selecting out mesenchymal or keratinocyte stem cells.

The methods described above can include further enrichment steps for cells by positive selection for other stem cell specific markers. Suitable other positive stem cell markers include, but are not limited to, SSEA-3, SSEA-4, Tra 1-60, CD34.sup.+, Thy-1.sup.+, and c-kit.sup.+, these includes in part also markers for non-mesenchymal stem cell types which may be used for negative selection in context of a specific mesenchymal stem cell type devoid of the marker. By appropriate selection with particular factors and the development of bioassays which allow for self-regeneration of MSCs or progeny thereof and screening of the MSCs or progeny thereof as to their markers, a composition enriched for viable MSCs or progeny thereof can be produced for a variety of purposes.

Once the stem cells or MSC or progeny thereof population is isolated, further isolation techniques may be employed to isolate sub-populations within the MSCs or progeny thereof. Specific markers including cell selection systems such as FACS for cell lineages may be used to identify and isolate the various cell lineages.

In yet another aspect of the present invention there is provided a method of measuring the content of mesenchymal cells or MSC or their progeny said method comprising

obtaining a cell population comprising stem cells or progeny (differentiated cells) thereof,

combining the cell population with a binding protein or binder for glycan structure according to Formula (I) on stem cell(s) thereof;

selecting for those cells which are identified by the binding protein for glycan structure according to Formula (I) on stem cell(s) thereof; and

quantifying the amount of selected cells relative to the quantity of cells in the cell population prior to selection with the binding protein.

Binder-Label Conjugates

The present invention is specifically directed to the binding of the structures according to the present invention, when the binder is conjugated with “a label structure”. The label structure means a molecule observable in a assay such as for example a fluorescent molecule, a radioactive molecule, a detectable enzyme such as horse radish peroxidase or biotin/streptavidin/avidin. When the labelled binding molecule is contacted with the cells according to the invention, the cells can be monitored, observed and/or sorted based on the presence of the label on the cell surface. Monitoring and observation may occur by regular methods for observing labels such as fluorescence measuring devices, microscopes, scintillation counters and other devices for measuring radioactivity.

Use of Binder and Labelled Binder-Conjugates for Cell Sorting

The invention is specifically directed to use of the binders and their labelled cojugates for sorting or selecting human stem cells from biological materials or samples including cell materials comprising other cell types. The preferred cell types includes mesenchymal cells such as mesenchymal cells derived from cord blood, bone marrow, peripheral blood and embryonal stem cells and corresponding associated cells not being mesenchymal cells. The labels can be used for sorting cell types according to invention from other similar cells. In another embodiment the cells are sorted from different cell types such as blood cells or in context of cultured cells preferably feeder cells, for example in context of mesenchymal stem cells corresponding associated/feeder (supporting) non-mesenchymal cells or cells in tissues such as human bone marrow stromal cells associated with bone marrow mesenchymal stem cells. A preferred cell sorting method is FACS sorting. Another sorting methods utilized immobilized binder structures and removal of unbound cells for separation of bound and unbound cells.

Use of Immobilized Binder Structures

In a preferred embodiment the binder structure is conjugated to a solid phase. The cells are contacted with the solid phase, and part of the material is bound to surface. This method may be used to separation of cells and analysis of cell surface structures, or study cell biological changes of cells due to immobilization. In the analytics involving method the cells are preferably tagged with or labelled with a reagent for the detection of the cells bound to the solid phase through a binder structure on the solid phase. The methods preferably further include one or more steps of washing to remove unbound cells.

Preferred solid phases include cell suitable plastic materials used in contacting cells such as cell cultivation bottles, petri dishes and microtiter wells; fermentor surface materials, etc.

Specific Recognition Between Preferred Stem Cells and Contaminating Cells

The invention is further directed to methods of recognizing stem cells from differentiated cells such as feeder cells, preferably animal feeder cells and more preferably mouse feeder cells. It is further realized, that the present reagents can be used for purification of stem cells by any fractionation method using the specific binding reagents.

Preferred fractionation methods includes fluorecense activated cell sorting (FACS), affinity chromatography methods, and bead methods such as magnetic bead methods.

The invention is further directed to positive selection methods including specific binding to the mesenchymal cell population but not to contaminating cell population. The invention is further directed to negative selection methods including specific binding to the contaminating cell population but not to the mesenchymal cell population. In yet another embodiment of recognition of mesenchymal cells the mesenchymal cell population is recognized together with a homogenous cell population such as a feeder cell population, preferably when separation of other materials is needed. It is realized that a reagent for positive selection can be selected so that it binds mesenchymal cells as in the present invention and not to the contaminating cell population and a reagent for negative selection by selecting opposite specificity. In case of one population of cells according to the invention is to be selected from a novel cell population not studied in the present invention, the binding molecules according to the invention maybe used when verified to have suitable specificity with regard to the novel cell population (binding or not binding). The invention is specifically directed to analysis of such binding specificity for development of a new binding or selection method according to the invention.

The preferred specificities according to the invention include recognition of:
i) mannose type structures, especially alpha-Man structures like lectin PSA, preferably on the surface of contaminating cells

Manipulation of Cells by Binders

The invention is specifically directed to manipulation of cells by the specific binding proteins. It is realized that the glycans described have important roles in the interactions between cells and thus binders or binding molecules can be used for specific biological manipulation of cells. The manipulation may be performed by free or immobilized binders. In a preferred embodiment cells are used for manipulation of cell under cell culture conditions to affect the growth rate of the cells.

Stem Cell Nomenclature

The present invention is directed to analysis of all stem cell types, preferably human stem cells. A general nomenclature of the stem cells is described in FIG. 7. The alternative nomenclatura of the present invention describe early human cells which are in a preferred embodiment equivalent of adult stem cells (including cord blood type materials) as shown in FIG. 7. Adult stem cells in bone marrow and blood is equivalent for stem cells from “blood related tissues”.

Lectins for Manipulation of Stem Cells, Especially Under Cell Culture Conditions

The present invention is especially directed to use of lectins as specific binding proteins for analysis of status of stem cells and/or for the manipulation of stems cells.

The invention is specifically directed to manipulation of stem cells under cell culture conditions growing the stem cells in presence of lectins. The manipulation is preferably performed by immobilized lectins on surface of cell culture vessels. The invention is especially directed to the manipulation of the growth rate of stem cells by growing the cells in the presence of lectins, as show in Table 18.

The invention is in a preferred embodiment directed to manipulation of stem cells by specific lectins recognizing specific glycan marker structures according to invention from the cell surfaces. The invention is in a preferred embodiment directed to use of Gal recognizing lectins such as ECA-lectin or similar human lectins such as galectins for recognition of galectin ligand glycans identified from the cell surfaces. It was further realized that there is specific variations of galectin expression in genomic levels in stem cells, especially for galectins-1, -3, and -8.

Sorting of Stem Cells by Specific Binders Including Lectins

The invention revealed use of specific binders including lectin types recognizing cell surface glycan epitopes according to the invention for sorting of stem cells, especially by FACS methods, most preferred cell types to be sorted includes mesenchymal cells such as adult stem cells in blood and bone marrow, especially cord blood cells such as cord blood derived mesenchymal cells.

Preferred Structures of O-glycan Glycomes of Stem Cells

The present invention is especially directed to following O-glycan marker structures of stem cells:

Core 1 type O-glycan structures following the marker composition

NeuAc₂Hex₁HexNAc₁, preferably including structures SAα3Galβ3GalNAc and/or SAα3Galβ3(Saα6)GalNAc;

and Core 2 type O-glycan structures following the marker composition NeuAc₀₋₂Hex₂HexNAc₂dHex₀₋₁, more preferentially further including the glycan series NeuAc₀₋₂Hex₂₊ₙHexNAc₂₊ₙdHex₀₋₁, wherein n is either 1, 2, or 3 and more preferentially n is 1 or 2, and even more preferentially n is 1;

more specifically preferably including R₁Galβ4(R₃)GlcNAcβ6(R₂Galβ3)GalNAc, wherein R₁ and R₂ are independently either nothing or sialic acid residue, preferably α2,3-linked sialic acid residue, or an elongation with HexₙHexNAcₙ, wherein n is independently an integer at least 1, preferably between 1-3, most preferably between 1-2, and most preferably 1, and the elongation may terminate in sialic acid residue, preferably α2,3-linked sialic acid residue; and

R₃ is independently either nothing or fucose residue, preferably α1,3-linked fucose residue.

It is realized that these structures correlate with expression of β6GlcNAc-transferases synthesizing core 2 structures.

Preferred Branched N-Acetyllactosamine Type Glycosphingolipids

The invention furhter revealed branched, I-type, poly-N-acetyllactosamines with two terminal Galβ4-residues from glycolipids of human stem cells. The structures correlate with expression of β6GlcNAc-transferases capable of branching poly-N-acetyllactosamines and further to binding of lectins specific for branched poly-N-acetylalctosamines. It was further noticed that PWA-lectin had an activity in manipulation of stem cells, especially the growth rate thereof.

Preferred Qualitative and Quantitative Complete N-Glycomes of Stem Cells

Preferred Binders for Stem Cell Sorting and Isolation

The present invention is specifically directed to stem cell binding reagents, preferentially proteins, preferentially mannose-binding or α1,3/6-linked mannose-binding, poly-LacNAc binding, LacNAc-binding, and/or fucose- or preferentially α1,2-linked fucose-binding; in a preferred embodiment stem cell binding or nonbinding lectins, more preferentially GNA, STA, and/or UEA; and in a further preferred embodiment combinations thereof, to uses described in the present invention taking advantage of glycan-binding reagents that selectively either bind to or do not bind to stem cells.

Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described.

Analysis and Utilization of Poly-N-Acetyllactosamine Sequences and Non-Reducing Terminal Epitopes Associated with Different Glycan Types

The present invention is directed to poly-N-acetyllactosamine sequences (poly-LacNAc) associated with cell types accoriding to the present invention. The inventors found that different types of poly-LacNAc are characteristic to different cell types, as described in the Examples of the present invention. In particular, CB MNC are characterized by linear type 2 poly-LacNAc; MSC, especially mainly associated cell type CB MSC, are characterized by branched type 2 poly-LacNAc. The present invention is especially directed to the analysis and utilization of these glycan characteristics according to the present invention. The present invention is further directed to the analysis and utilization of the specific cell-type accociated glycan sequences revealed in the present Examples according to the present invention.

The present invention is directed to non-reducing terminal epitopes in different glycan classes including N- and O-glycans, glycosphingolipid glycans, and poly-LacNAc. The inventors found that especially the relative amounts of β1,4-linked Gal, β1,3-linked Gal, α1,2-linked Fuc, α1,3/4-linked Fuc, α-linked sialic acid, and α2,3-linked sialic acid are characteristically different between the studied cell types; and the invention is especially directed to the analysis and utilization of these glycan characteristics according to the present invention.

The present invention is further directed to analyzing fucosylation degree in O-glycans by comparing indicative glycan signals such as neutral O-glycan signals at m/z 771 and 917 as described in the Examples. The inventors found that low relative abundance of neutral O-glycan signal at m/z 917 compared to 771, indicates low fucosylation degree of the O-glycan sequences corresponding to the signal at m/z 771 and containing terminal β1,4-linked Gal. Signal at m/z 552, corresponds to Hex₁HexNAc₁dHex₁, including α1,2-fucosylated Core 1 O-glycan sequence. In CB MNC the glycan signal at m/z 917 is relatively abundant, indicating high fucosylation degree of the O-glycan sequences corresponding to the signal at m/z 771 and containing terminal β1,4-linked Gal. The preferred cell types analyzed in the present invention also had characteristic fucosylation degree of the stuctures.

Especially, the present invention is directed to analyzing terminal epitopes associated with poly-LacNAc in mesenchymal cells, more preferably when these epitopes are presented in the context of a poly-LacNAc chain, most preferably in O-glycans or glycosphingolipids. The present invention is further directed to analyzing such characteristic poly-LacNAc, terminal epitope, and fucosylation profiles according to the methods of the present invention, in glycan structural characterization and specific glycosylation type identification, and other uses of the present invention; especially when this analysis is done based on endo-β-galactosidase digestion, by studying the non-reducing terminal fragments and their profile, and/or by studying the reducing terminal fragments and their profile, as described in the Examples of the present invention. The inventors found that cell-type specific glycosylation features are efficiently reflected in the endo-β-galactosidase reaction products and their profiles. The present invention is further directed to such reaction product profiles and their analysis according to the present invention.

The inventors found that characteristic non-reducing poly-LacNAc associated sequences include in a preferred embodiment Fucα2Gal, Galβ3GlcNAc, Fucα2Galβ3GlcNAc, and α3′-sialylated Galβ3GlcNAc. The present invention is especially directed to analysis of such glycan structures according to the present methods, in context of mesenchymal stem cells and differentiation of stem cells, preferably in context of human embryonic stem cells and their differentiation.

The inventors further found that all three most thoroughly analyzed cellular glycan classes, N-glycans, O-glycans, and glycosphingolipid glycans, were differently regulated compared to each other, especially with regard to non-reducing terminal glycan epitopes and poly-LacNAc sequences as described in the Examples and Tables of the present invention. Therefore, combining quantitative glycan profile analysis data from more than one glycan class will yield significantly more information. The present invention is especially directed to combining glycan data obtained by the methods of the present invention, from more than one glycan class selected from the group of N-glycans, O-glycans, and glycosphingolipid glycans; more preferably, all three classes are analyzed; and use of this information according to the present invention. In a preferred embodiment, N-glycan data is combined with O-glycan data; and in a further preferred embodiment, N-glycan data is combined with glycosphingolipid glycan data.

Mesenchymal Stem Cell Markers

The present invention revaled in a specific embodiment glycan structures, which are markers for mesenchymal stem cells or differentiated cells, preferably osteogenically differentiated cells derived from the mesenchymal, preferably bone marrow mesenchymal stem cells.

The invention also revealed optimal conditions for the analysis, some antibodies (or binder types) preferring flow cytometry (FACS) conditions and some preferring conditions for immunohistochemistry. The invention also revealed that specific cell population can be fractionated by using the antibodies.

The invention is further directed to isolation and analysis of released cellular components (glycoproteins, glycopeptides, glycolipids or oligosaccharides) by using the specific antibody binding reagents. The invention is especially directed to trypsin sensitive and trypsin resistant components.

Preferred Markers Especially for Bone Marrow Mesenchymal Stem Cells

Marker Structures Mesenchvmal Stem Cells in Comparision to Differentiated Cells

The invention revealed 3 preferred high prevalence markers sLex, SSEA-3 and SSEA-4 and a second markers with lower but characteristic expression (STn and TN, pLn and sLea) for the mesenchymal stem cells in comparison to osteogenically differentiated cells.

The sLex, sLea and pLN belong to group of N-acetyllactosamine markers, the type 1 and type II N-acetyllactosamines for a characteristic panel of differentiation antigens of stem cells.

GalNAc type structures includes SSEA-3 and SSEA-4-type structures and mucin structures sTn and Tn. It is realized that the mucin type and globoseries type epitopes can be cross-reactive and include novel target structures.

The preferred mesenchymal stem cells markers especially for bone marrow mesenchymal stem cells thus are:
i) A preferred type II N-acetyllactosamine structure sialyl-Lewis x [SAα3Galβ4(Fucα3)GlcNAc, SA is sialic acid preferably Neu5Ac, sLex]
ii) stage specific embryonic antigen like structures SSEA-3 and SSEA-4, referred as SSEA-3 type and SSEA-4 type structures.
iii) Two mucin type epitopes sTn SAα6GalNAcα(Ser/Thr), and Tn GalNAcα(Ser/Thr), the specific antibodies are especially preferred in context of FACS analysis as mesenchymal cell markers

iv) Two type I N-acetyllactosamine structures Galβ3GlcNAc (pLN) and NeuNAcα3 Galβ3 (Fucα4)GlcNAc (sLea).

Preferred SSEA-3 and SSEA-4-Type Target Structures and Use Thereof

It is realized that the specific antibody clones used are especially useful for characterizing bone marrow mesenchymal stem cells and their differentiation to osteogenic structures. Futhermore the invention reveled that at least part of the SSEA-4 structures are different from the traditional cell surface glycolipid marker SSEA-4 (Neu5Acα3Galβ3GalNAcβ3Galα4Galβ4GlcβCer) as it is at least partially protease sensitive on cell surface. The protease sensitivity was about one third of mesenchymal cells with about 23% reduction of labelled cells in FACS analysis and even more dramatic on differentiated cells from which the marker was released practically totally with reduction of about 20% units, see FIG. 19, EXAMPLE 16. The invention is specifically directed to methods of cahracterization of the protease sensitive and insentive target molecules as described in Example 16.

Marker Structures for Differentiating/Differentiated Mesenchvmal Stem Cells

The invention revealed several structures, which are characteristic for differentiated mesenchymal stem cells, more preferably osteogenically differentiated mesenchymal stem cells.

The structures includes GalNAc comprising structures with epitopes known especially from glycolipids such as asialo GM1 and asialo GM2, and globotriose and globotetraose and on CA15.3 clone, which was indicated to recognise a sialylated epitope from mucin, preferably Muc 1 and specific fucosylated lactosamines including type I (Lewis a) and type II lactosamine H type 2.

i) asialoganglioside epitopes asialo-GM2 (GalNAcβ4Galβ4GlcβCer) and asialo GM1 (Galβ3GalNAcβ4Galβ4GlcβCer). It is realized that the antibodies do not necessarily recognize the whole oligosaccharide sequence but a terminal epitope. The invention is further directed to the recognition of similar shorter epitopes comprising terminal GalNAcβ4-, GalNAcβ4Gal-, GalNAcβ4Galβ4, and GalNAcβ4Galβ4Glc; and Galβ3 GalNAc, Galβ3GalNAcβ, Galβ3GalNAcβ4 and Galβ3GalNAcβ4Galβ4Glc. The invention is further preferably directed to the recognition of the following non-reducing end terminal epitopes on proteins: GalNAcβ4-, GalNAcβ4Gal-, GalNAcβ4Galβ4- and/or GalNAcβ4Galβ4GlcNAc; and terminal epitopes of asialo GM1: Galβ3GalNAc (in a specifc embodiment cross reactive with O-glycan core I) and/or Galβ3GalNAc3. It was shown that epitopes are protease sensitive and invention is in a specific embodiment directed to covalently protein linked epitopes. It is realized that Glc is likely not a protein linked structure, but e.g. GalNAcβ4Galβ4GlcNAc is corresponding protein epitope known from N-glycans and O-glycans. The asialoganglioside targets and antibodies are especially preferred for analysis of differentiated mesenchymal stem cells under the FACS and similar conditions.

ii) globoseries epitopes globotirasylceramide (Galα4Galβ4GlcβCer) and globotetrasoyl ceramide Gb4/G14 (GalNAcβ3Galα4Galβ4GlcβCer). The invention is further directed to the recognition of similar shorter epitopes comprising terminal oligosaccharide sequences: Galα4Gal, Galα4Galβ, Galα4Galβ4, and Galα4Galβ4Glc; and GalNAcβ3Gal, GalNAcβ3Galα, GalNAcβ3 Galα4Gal, GalNAcβ3Galα4Galβ, GalNAcβ3Galα4Galβ4, and GalNAcβ3Galα4Galβ4Glc. The two globoseries core structures were revealed by fax analysis to be essentially trypsin insensitive in mesenchymal cells. Therefore the invention is preferably directed to recognition of the structures/epitopes especially as lipid conjugates.

Interestingly Gb3 is trypsin sensitive in the osteogenically differentiated cells (54.3% versene, 4.9% trypsin). The invention is therefore directed to studies of trypsin sensitive Gb3-epitopes from osteogenically differentiated cells, in a preferred embodiment the epitopes includes the terminal epitopes without Glc-residue: Galα4Gal, Galα4Galβ, Galα4Galβ4 and a known similar protein linked epitope Galα4Galβ4GlcNAc.

iii) Mucin related epitope CA15-3. It is realized that the sialylated mucin epitope of CA15.3 would have partial similarity with oc-linked monosaccharide comprising globoseries structures and GalNAc/Galβ3GalNAc comprising asialo ganglioside structures.

An additional likely mucin type structure directed antibody GF276 (oncofetal antigen) is especially preferred for analysis of differentiated mesenchymal stem cells under immunohistochemistry and similar conditions.

Furhtermore the mucin antigens sTn SAα6GalNAcα(Ser/Thr), and Tn GalNAcα(Ser/Thr), and corresponding the specific antibodies are especially preferred for analysis of differentiated mesenchymal stem cells under immunohistochemistry and similar conditions.

iv) Specific fucosylated N-acetyllactosamines including type I lactosmine structure Galβ(Fucα3)GlcNAc (Lewis a, Lea) and type II lactosamine H type 2, Fucα2Galβ4GlcNAc. Both of the structures comprise specific α-fucose epitopes on different positions and conformations. It is realized that the epitopes are useful in a panel of different type I and Type 2 lactosamine recognizing antibodies for specific recognition of stem cells under various condition. The Lewis a antigen and corresponding antibodies are especially directed to analysis of differentiated mesenchymal stem cells under FACS and similar conditions.

An additional type I N-acetyllactosamine structure H type 1 (Fucα2Galβ4GlcNAc) and corresponding antibodies (like GF303) are especially preferred for analysis of differentiated mesenchymal stem cells under immunohistochemistry and similar conditions.

The preferred antibodies for recognition of preferred epitopes includes GF275 (CA15-3), GF296 (asialo GM1), GF297 (GL4), GF298 (Gb3), GF300 (asialo GM2), GF302 (H type 2), and GF304 (Lea), GF276 (oncofetal antigen) and GF303 (H Type 1) and antibodies with similar specificities.

Trypsin Sensitive Epitopes and Cryptic Epitopes

Trypsin Sensitive Epitopes

The data revealed that part of the structures are sensitive for trypsin treatment as indicated in Table 23. The FACS results with trypsin release are also indicated as second FACS column for MSC and osteogenic cells. Trypsin is protease and it can be assumed that at least part of the trypsin sensitive epitopes especially including protein epitopes are released by the trypsin trestment

Cryptic Epitopes Revealed More by Trypsin

FACS analysis reveled epitopes, which are stabile or even increase after trypsin treatment. This may be observable from mesenchymal cell samples Globotriose (increase from 16.9% to 28.4%). The invention is further directed to isolation and studies of the trypsin resistant epitopes.

Increased Trypsin Condition Sensitity Correlates with Negative IHF Staining

Immunohistochemistry appeared to be less sensitive in detecting glycan structures. Interestingly the immunohistochemistry results correlate with trypsin sensitivity of the epitopes. When the epitopes are not visible by immunohistochemistry the amount of positive cells after trypsin in FACS is also very low, in most case 0.5-1.0%. The examples of this includes AsialoGM2 osteogenic, AsialoGM1 osteogenic and Lewis a

There are few cases when the epitopes are visible by immunofluorescence in first cell type, but the versene FACS signal is higher in the second cell type, in these cases the trypsin FACS signals correlate with immunofluorescence and the epitope appears to be more trypsin resistant or even cryptic (increasing after trypsin) in first cell type. Examples of this includes H type I, Tn, and sTn.

Expanded MSC Binder Target table for Selecting Effective Positive and/or Negative Binders and Combinations Thereof

Table 27 describes combined results of the inventors' structural assignments of MSC and differentiated cell specific glycosylation (Examples of the present invention describing mass spectrometric profiling, NMR, glycosidase, and glycan fragmentation experiments, as well as structure-revealing comparison of N-glycan profiles including Tables 28-30 and other Tables and Examples of the present invention), biosynthetic information including knowledge of biosynthetic pathways and glycosylation gene expression, as well as binder specificities as described in the present invention (Examples of the present invention describing lectin, antibody, and other binder molecule binding to specific cell types and molecule classes).

Table 27 describes suitable binder targets in specific cell types by q, ±, +, and ++ codes, especially preferably by + and ++ codes; as well as useful absence or low expression by −, q, and ± codes, especially preferably by − and ± codes. The inventors realized that such data can be used to recognize specifically selected cell types. The invention is directed to such use with various different principles as specific embodiments of the present invention: positive selection using binders recognizing specific cell type associated targets, negative selection by utilizing targets with low abundance on specific cells, as well as combined positive and negative selection, or further combined use of more than one positive and/or negative targets to increase specificity and/or efficiency according to the present invention.

Below are described especially preferred targets for binders according to the present invention.

1) MSC Binder Structures:

The invention is directed to recognizing MSC based on terminal glycan epitopes as indicated in Table 27, preferably selected from:

LN type 1 (Lec, Galβ3GlcNAc),

sLex, more specifically sLexβ3Galβ4Glc[NAc]β,

large high-mannose type N-glycans, more specifically containing Manα2Man terminal epitopes,

glucosylated N-glycans, more specifically containing Glcα, preferably terminal Glcα3Manα,

core-fucosylated N-glycans,

terminal GlcNAcβ epitopes, more specifically in N-glycans with preferentially GlcNAcβ2Man terminal structure, preferably also including another GlcNAcβ2Man terminal structure, further preferably also including GlcNAcβ4Man terminal structure;

an especially preferred binder structure is sLex, more specifically sLexβ3Galβ4Glc[NAc]β, optionally together with one or more other epitopes from the list above.

In a further embodiment, the invention is directed to recognizing MSC and osteoblast-differentiated cells as indicated in Table 27, preferably based on LN type 2, more preferably N-glycan terminal epitope LNβ2Man.

In a further embodiment, the invention is directed to recognizing MSC and adipocyte-differentiated cells as indicated in Table 27, preferably based on epitopes including:

Lex, Gb5 (SSEA-3), SAα3Galβ3GalNAcβ, and/or SSEA-4 (SAα3Galβ3GalNAcβ3Galα4Galβ4Glc);

an especially preferred binder structure is SSEA-4, optionally together with one or more other epitopes from the list above, preferably together with Lex.

In a further embodiment, the invention is directed to recognizing MSC, osteoblast-differentiated and adipocyte-differentiated cells as indicated in Table 27, preferably based on GD2.

2) Binder Structures Directed to Cells Differentiated from MSC

The invention is directed to specific recognition of cells differentiated from MSC, preferably adipocyte, osteoblast, and/or chondrocyte-differentiated as described in the invention, based on terminal glycan epitopes as indicated in Table 27, preferably selected from:

Lea,

sLea,

α3′-sialyl Lec,

LNβ4Man, more preferably in branched N-glycan structure

LNβ2(LNβ4)Manα3(LNβ2Manα6)Man

Lex, more preferably Lexβ3Galβ4Glc[NAc]β

H type 2,

Galβ3GalNAcβ,

asialo-GM1,

GalNAcβ, more preferably asialo-GM2,

Gb4,

Gb3,

GalNAcα, more preferably in Tn epitope,

sialyl Tn,

oligosialic acid, more preferably NeuAcα8NeuAcα terminal epitope,

GD3,

Low-mannose, small high-mannose, or hybrid-type N-glycans, preferably containing

terminal Manα3Man, and/or Manα6Man,

Manα3 (Manα6)Manβ4GlcNAc[β4GlcNAc],

Manβ, preferably in Manβ4GlcNAc terminal epitope;

wherein especially preferred binder structures are asialo-GM1, asialo-GM2, Tn, sialyl-Tn, Lea, and sLea;

from which preferably one or more other epitopes are selected for use in a specific embodiment of the present invention, more preferably including either asialo-GM1, asialo-GM2, Tn, or sialyl-Tn;

optionally together with one or more other epitopes from the full list above.

In a further embodiment, the invention is directed to recognizing adipocyte-differentiated cells as indicated in Table 27, preferably based on epitopes including:

Lea, sLea, sialyl Lec, and/or Galβ3GalNAcβ;

especially preferred binder structures are Lea or sLea, optionally together with one or more other epitopes from the list above.

In a further embodiment, the invention is directed to recognizing osteoblast-differentiated cells as indicated in Table 27, preferably based on epitopes including: Gb3, Gb4, and/or LNβ4Man, the latter preferably within in a branched N-glycan structure;

especially preferred binder structures are Gb3 and/or Gb4, optionally together with one or more other epitopes from the list above.

Preferred Lex/sLex Antibody Binders

The inventors found that specific cell types carry Lex/sLex epitopes on different glycan backbones according to the invention. Useful such reagents are described in the present invention, and further useful reagents are listed below. The invention is specifically directed to use of one or more of listed antibodies for structure-specific recognition of Lex/sLex epitopes in different cell types and on different glycan backbones. The list is ordered according to preferred glycan backbone specificities. Suitable binders against Lex and/or sLex on each backbone can be selected according to the present invention for different cell types.

| Code | Producer code | Manufacturer/reference | Clone |
|---|---|---|---|
| | Anti-Lex antibodies: | | |
| GF 305 | CBL144 (anti CD15) Le^{x} | Chemicon | 28 |
| GF 517 | ab34200 (CD15) | Abcam | TG-1 |
| GF 515 | 557895 anti-human CD15 | BD Pharmingen | W6D3 |
| GF 525 | ab17080-1 (CD15) | | MMA |
| | ab20138 | Abcam | 29 |
| | ab1252 | Abcam | BRA4F1 |
| | ab49758 | Abcam | BY87 |
| | ab51369 | Abcam | CLB-gran/2, |
| | | | B4 |
| | ab13453 | Abcam | DU- |
| | | | HL60-3 |
| | ab53997 | Abcam | LeuM1 |
| | ab6414 | Abcam | MC-1 |
| | ab665 | Abcam | MEM- |
| | | | 158 |
| | ab754 | Abcam | MY-1 |
| | ab15614 | Abcam | VIM-C6 |
| | Lewis x | Abcam | |
| | ab3358 | Abcam | P12 |
| | anti CD15 | Beckman Coulter | 80H5 |
| | anti CD15 | BioLegend | HI98 |
| | anti CD15 | Chemicon | ZC-18C |
| | anti CD15 | Chemicon | MCS-1 |
| | anti CD15 | Chemicon | DT07 & |
| | | | BC97 |
| | anti CD15 | Labvision | 15C02 |
| | anti CD15 | Labvision | SPM490 |
| | anti CD15 | Ancell | AHN1.1 |
| | anti CD15 | Quartett Immunodiagnostika, Berlin | Tu9 |
| | anti CD15 | Patricell | B-H8 |
| | anti CD15 | Patricell | HIM . . . |
| | anti CD15 | Santa Cruz | C3D-1 |
| | anti CD15 | Santa Cruz | 3G75 |
| | anti Lewis x | Santa Cruz | 4C9 |
| | anti CD15 | ScyTek Laboratories | FR4A5 |
| | anti CD15 | USBio | 5F17 |
| | anti CD15 | USBio | 8.S.288 |
| | anti CD15 | USBio | 0.N.80 |
| | Anti-Lex antibodies with poly- | | |
| | LacNAc and/or glycolipid- | | |
| | specificity: | | |
| GF 518 | ab16285 (SSEA1) | Abcam | MC480 |
| | Anti-Lex antibodies for N- | | |
| | glycans: | | |
| | Anti-Lex in neutral N-glycan | Lucka et al. Glycobiology 15: 87- | L5 |
| | | 100, 2005 | |
| | Anti-Lex in neutral N-glycan | Lanctot et al. Current Opinion in | 3A8 |
| | | Chemical Biology 11, Issue 4, 2007, | |
| | | 373-380; Lanctot et al. 2006, Poster | |
| | | presentation in Glycobiology Society | |
| | | Meeting, Universal City, CA, poster | |
| | | 238 | |
| | Anti-Lex antibodies for Core 2 O- | | |
| | glycans: | | |
| | Anti-Lex in Core 2 O-glycan | Sekine et al. Eur. J. Biochem. | SA024 |
| | | 268: 1129-1135, 2001 | |
| | Anti-sulfo-Lex antibodies: | | |
| | antiCD15u = sulfoCD15 | USBio | 5F18 |
| | Anti-sLex antibodies: | | |
| GF 516 | 551344 anti-human CD15s | BD Pharmingen | CSLEX1 |
| GF 307 | MAB2096 (anti-sLewis X) | Chemicon | KM93 |
| | anti sLex | Seikagaku | 73-30 |
| | anti sLex | Meridianlifesciences | 258- |
| | | | 12767 |
| | anti sLex | USBio | 2Q539 |
| | Anti-sLex antibodies for Core2 O- | | |
| | glycans: | | |
| GF 526 | MAB996 (anti-hP-selectin- | R&D systems | CHO131 |
| | glycoprotein ligand 1 ab) | | |

Recognition of Glycans of Mesenchymal Cells

General observations. There seems not to be a single specific glycan epitope analyzed absolutely specific only for one total population of MSCs or a cell population differentiated into osteogenic lineage. Instead there seems to be enrichment of certain glycan epitopes in stem cells and in differentiated cells. In some cases the antibodies recognize epitopes, which are highly or several fold enriched in a specific cell type or present above the current FACS detection limit in a part of a cell population but not in the other corresponding cell populations. It is realized that such antibodies are especially useful for specific recognition of the specific cell population. Furthermore, combination of several antibodies recognizing independent populations of specific cell types is useful for recognition of a larger cell population in a positive or negative manner.

The present invention provides reagents common to mesenchymal cell populations in general or for specific differentiation stage of mesenchymal cells such as mesenchymal stem cells, or differentiated mesenchymal stem cells in general or specific for the specifically differentiated cell populations such as adipocytes or osteoblasts. Furthermore the invention reveals specific marker structures for mesenchymal stem cells derived from specific tissue types such as cord blood or bone marrow.

The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificties in order to find high specificity antibodies.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells. It is realized that tissues comprising stem cells usually contain these in primitive stem cell stage and highly expressed markers according can be optimised or selected for the cell isolation. It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

Combination of several antibodies for specific analysis of a mesenchymal cell population would characterize the cell population. In a preferred embodiment at least one “effectively binding antibody”, recognizing major part (over 35%) or most (50%) of the cell population (preferably more than 30%, an in order of increasing preference more than 40%, 50%, 60%, 70%, 80% and most preferably more than 90%), are selected for the analytic method in combination with at least one “non-binding antibody”, recognizing preferably minor part (preferably from detection limit of the method to low level of recognition, in order of preference less than 10%, 7%, 5%, 2% or 1% of cells, e.g 0.2-10% of cells, more preferably 0.2-5% of the cells, and even more preferably 0.5-2% or most preferably 0.5%-1.0%) or no part of the cell population (under or at the detection limit e.g. in order of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably from 7% to 40% and most preferably from 10 to 35%.

The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificties in order to find high specificity antibodies. Furthermore the invention is directed to the search of the structures from phage display libraries.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells.

It is realized that tissues comprising stem cells usually contain these in primitive stem cell stage and highly expressed markers according can be optimised or selected for the cell isolation. In a preferred embodiment the invention is directed to selection of mesenchymal cells by the binders according to the invention such as by or sialyl-Lewis x recognizing proteins including preferably monoclonal antibodies recognizing the glycan epitopes according the invention (Table 27). In a separate embodiments the invention is directed to the use of selectins or selectin homologous proteins optimized for the reconition.

It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

Combinations

Combination of several antibodies for specific analysis of a hematoppietic or associated population for cell population would characterize the cell population. In a preferred embodiment at least one “effectively binding antibody”, recognizing major part (over 35%) or most (50%) of the cell population (preferably more than 30%, an in order of increasing preference more than 40%, 50%, 60%, 70%, 80% and most preferably more than 90%), are selected for the analytic method in combination with at least one “non-binding antibody”, recognizing preferably minor part (preferably from detection limit of the method to low level of recognition, in order of preference less than 10%, 7%, 5%, 2% or 1% of cells, e.g 0.2-10% of cells, more preferably 0.2-5% of the cells, and even more preferably 0.5-2% or most preferably 0.5%-1.0%) or no part of the cell population (under or at the detection limit e.g. in order of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably from 7% to 40% and most preferably from 10 to 35%.

The invention is directed to the use of several reagents recognizing terminal epitopes together, preferably at least two reagents, more preferably at least three epitopes, even more preferably at least four, even more preferably at least five, even more preferably at least six, even more preferably at least seven, and most preferably at least 8 to recognize enough positive and negative targets together. It is realized that with high specificity binders selectively and specifically recognizing elongated epitopes, less binders may be needed e.g. these would be preferably used as combinations of at least two reagents, more preferably at least three epitopes, even more preferably at least four, even more preferably at least five, most preferably at least six antibodies. The high specificity binders selectively and specifically recognizing elongated epitopes binds one of the elongated epitopes at least inorder of increasing preference, 5, 10, 20, 50, or 100 fold affinity, methods for measuring the antibody binding affinities are well known in the art. The invention is also directed to the use of lower specificity antibodies capable of effective recognition of one elongated epitope but also at least one, preferably only one additional elongated epitope with same terminal structure

The reagents are preferably used in arrays comprising in order of increasing preference 5, 10, 20, 40 or 70 or all reagents shown in cell labelling experiments.

The invention is further directed to combinations of fucosylated and/or sialylated structures with structures devoid of these modifications. Combinations of type 1 N-acetyllactosamine with type 2 structures with type 1 (Galβ3GlcNAc) structures and/or with mucin type and/or glyccolipids structures. In apreferred combination at least one binding antibody is combined with non-binding antibody recognizing different structure type

The antibodies recognize certain glycan epitopes revealed as target structures according to the invention. It is realized that specificites and affinities of the antibodies vary between the clones. It was realized that certain clones known to recognize certain glycan structure does not necessarily recognize the same cell population.

Release of Binders or Binder Conjugates from the Cells by Carbohydrate Inhibition

The invention is in a preferred embodiment directed to the release of glycans from binders. This is preferred for several methods including:
a) release of cells from soluble binders after enrichement or isolation of cells by a method invlogin a binder
b) release from solid phase bound binders after enrichment or isolation of cells or during cell cultivation e.g. for passaging of the cells

The inhibitin carbohydrate is selected to correspond to the binding epitope of the lectin or part(s) thereof. The preferred carbohydrates includes oligosaccharides, monosaccharides and conjugates thereof. The preferred concentrations of carbohydrates includes contrations tolerable by the cells from 1 mM to 500 mM, more preferably 10 mM to 250 mM and even more preferably 10-100 mM, higher concentrations are preferred for monosaccharides and method involving solid phase bound binders. Preferred oligosaccharide sequences including oligosaccharides and reducing end conjugates includes Galβ4Glc, Galβ4GlcNAc, Galβ3GlcNAc, Galβ3GalNAc, and sialylated and fucosylated variants of these as described in TABLEs and formulas according to the invention.

The preferred reducing enstructure in conjugates is AR, wherein A is anomeric structure preferably beta for Galβ4Glc, Galβ4GlcNAc, Galβ3GlcNAc, and alfa for Galβ3GalNAc and R is organic residue linked glycosidically to the saccahride, and preferably alkyl such as method, ethyl or propyl or ring structure such as a cyclohexyl or aromatic ring structure optionally modified with further functional group.

Preferred monosaccharides includes terminal or two or three terminal monosaccharides of the binding epitope such as Fuc, Gal, GalNAc, GlcNAc, Man, preferably as anomeric conjugates: as FucαR, GalβR, GalNAcβR, GalNAcαR GlcNAcβR, ManαR. For example PNA lectin is preferably inhibited by Galβ3GalNAc or lactose or Gal, STA is inhibited by Galβ4Glc, Galβ4GlcNAc or oligomers or poly-LacNAc epitopes derived thereof and LTA is inhibited by fucosylalactose Galβ4(Fucα3)Glc, Galβ4(Fucα3)GlcNAc or Fuc or FucαR. Examples of monovalent inhibition condition are shown in Venable A. et al. (2005) BMC Developmental biology, for inhibition when the cells are bound to polyvalently to solid phase larger epitopes and/or concentrations or multi/polyvalent conjugates are preferred.

The invention is further directed to methods of release of binders by protease digestion similarity as known for release of cells from CD34+ magnetic beads.

Immobilized Binders Preferably Binder Proteins Protein

The present invention is directed to the use of the specific binder for or in context of cultivation of the stem cells wherein the binder is immobilized.

The immobilization includes non-covalent immobilization and covalent bond including immobilization method and further site spefic immobilization and unspecific immobilization.

A preferred non-covalent immobilization methods includs passive adsorption methods. In a preferred method a surface such as plastic surface of a cell culture dish or well is passively absorbed with the binder. The preferred method includes absorbtion of the binder protein in a solvent or humid condition to the surface, preferably evenly on the surface. The preferred even distribution is produced using slight shaking during the absorption period preferably form 10 min to 3 days, more preferably from 1 hour to 1 day, and most preferably over night for about 8 to 20 hours. The washing steps of the immobilization are preferably performed gently with slow liquid flow to avoid detachment of the lectin.

Specific Immobilization

The specific immobilization aims for immobilization from protein regions wich does not disturb the the binding of the binding site of the binder to its ligand glycand such as the specific cell surface glycans of stem cells according to the invention.

Preferred specific immobilization methods includes chemical conjugation from specific aminoacid residues from the surface of the binder protein/peptide. In a preferred method specific amino acid residue such as cysteine is cloned to the site of immobilization and the conjugation is performed from the cystein, in another preferred method N-terminal cytsteine is oxidized by periodic acid and conjugated to aldehyde reactive reagents such as amino-oxy-methyl hydroxylamine or hydrazine structures, further preferred chemistries includes “click” chemistry marketed by Invitrogen and aminoacid specifc coupling reagents marketed by Pierce and Molecular probes.

A preferred specific immobilization occurs from protein linked carbohydrate such as O- or N-glycan of the binder, preferably when the glycan is not close to the binding site or longer specar is used.

Glycan Immobilized Binder Protein

Preferred glycan immobilization occurs through a reactive chemoselective ligation group R1 of the glycans, wherein the chemical group can be specifically conjugated to second chemoselective ligation group R2 without major or binding destructutive changes to the protein part of the binder. Chemoselective groups reacting with aldehydes and ketones includes as amino-oxy-methyl hydroxylamine or hydrazine structures. A preferred R1-group is a carbonyl suchas an aldehyde or a ketone chemically synthesized on the surface of the protein. Other preferred chemoselective groups includes maleimide and thiol; and “Click”-reagents including azide and reactive group to it.

Preferred synthesis steps includes
a) chemical oxidation by carbohydrate selectively oxidizing chemical, preferably by periodic acid or
b) enzymatic oxidation by non-reducing end terminal monosaccharide oxidizing enzyme such as galactose oxidase or by transferring a modified monosaccharide residue to the terminal monosaccharide of the glycan.

Use of oxidative enzymes or periodic acid are known in the art has been described in patent application directed conjugating HES-polysaccharide to recombinant protein by Kabi-Frensenius (WO2005EP02637, WO2004EP08821, WO2004EP08820, WO2003EP08829, WO2003EP08858, WO2005092391, WO2005014024 included fully as reference) and a German research institute.

Preferred methods for the transferring the terminal monosaccharide reside includes use of mutant galactosyltransferase as described in patent application by part of the inventors US2005014718 (included fully as reference) or by Qasba and Ramakrishman and colleagues US2007258986 (included fully as reference) or by using method described in glycopegylation patenting of Neose (US2004132640, included fully as reference).

Conjugates Including High Specificity Chemical Tag

In a preferred embodiment the binder is, specifically or non-specifically conjugated to a tag, referred as T, specifically recognizable by a ligand L, examples of tag includes such as biotin biding ligand (strept)avidin or a fluorocarbonyl binding to another fluorocarbonyl or peptide/antigen andspecific antibody for the peptide/antigen

Prefererred Conjugate Structures

The preferred conjugate structures are according to the Formula CONJ

B-(G)ₘR1-R2-(S1-)ₙT-,

wherein B is the binder, G is glycan (when the binder is glycan conjugated), R1 and R2 are chemoselective ligation groups, T is tag, preferably biotin, L is specifically binding ligand for the tag; S1 is an optional spacer group, preferably C₁-C₁₀ alkyls, m and n are integers being either 0 or 1, independently.

Complex of Binder

The invention id further directed to complexes in of the binders involving conjugation to surface including solid phase or a matrix including polymers and like. It is realized that it is epscially useful to conjugate the binder from the glycan because preventing cross binding of of binders or effects of the binders to cells.

A complex comprising structure according to the Formula COMP

B-(G-)ₘR1-R2-(S1-)ₙ(T-)ₚ(L-)ᵣ₋₍S2)ₛ-SOL,
wherein B is the binder, SOL is solid phase or matrix or surface or Label (may be also Ligand conjugated label), G is glycan (when the binder is glycan conjugated), R1 and R2 are chemoselective ligation groups, T is tag, preferably biotin, L is specifically binding ligand for the tag; S1 and S2 are optional spacer groups, preferably C₁-C₁₀ alkyls, m, n, p, r and s are integers being either 0 or 1, independently.
Preferred elongated epitopes

Preferred Elongated Epitopes

It is realized that elongated glycan epitopes are useful for recognition of the mesenchymal cells according to the invention. The invention is directed to use part of the structures for characterizing all the cell types, while certain structural motives are more common on specific differentiatation stage.

It is further realized that part of the terminal structures are especially highly expressed and thus especially useful for the recognition of one or several types of the cells. The terminal epitopes and the glycan types are listed in Table 27, based on the structural analysis of the glycan types following preferred elongated structural epitopes are preferred as novel markers for mesenchymal cells and for the uses according to the invention.

Preferred Terminal GalβB3/4 Structures

Type II N-Acetyllactosamine Based Structures

Terminal Type II N-Acetyllactosamine Structures

The invention revealed preferred type II N-acetyllactosamines including specific O-glycan, N-aglycan and glycolipid epitopes. The invention is in a preferred embodiment especially directed to abundant O-glycan and N-glycan epitopes. The invention is further directed to recognition of characteristic glycolipid type II LacNAc terminal. The invention is especially directed to the use of the Type II LacNAc for recognition of mesenchymal cells and similar cells or for analysis of the differentiation stage. It is however realized that substantial amount of the structures are present in the more differentiated cells.

Elongated type II LacNAc structures are especially expressed on N-glycans. Preferred type II LacNAc structures are β2-linked to biantennary N-glycan core structure, Galβ4GlcNAcβ2Manα3/6Manβ4

The invention further revealed novel O-glycan epitopes with terminal type II N-acetyllactosamine structures expressed effectively the mesenchymal type cells. The analysis of O-glycan structures revealed especially core II N-acetyllactosamines with the terminal structure. The preferred elongated type II N-acetyllactosamines thus includes Galβ4GlcNAcβ6GalNAc, Galβ4GlcNAcβ6GalNAcα, Galβ4GlcNAcβ6(Galβ3)GalNAc, and Galβ4GlcNAcβ6(Galβ3)GalNAcα.

The invention further revealed presence of type II LacNAc on glycolipids. The present invention reveals for the first time terminal type N-acetyllactosamine on glycolipids. The neolacto glycolipid family is an important glycolipid family characteristically expressed on certain tissue but not on others. The preferred glycolipid structures includes epitopes, preferably non-reducing end terminal epitopes of linear neolactoteraosyl ceramide and elongated variants thereof Galβ4GlcNAcβ3Gal, Galβ4GlcNAcβ3Galβ4, Galβ4GlcNAcβ3Galβ4Glc(NAc), Galβ4GlcNAcβ3Galβ4Glc, and Galβ4GlcNAcβ3Galβ4GlcNAc. It is furher realized that specific reagents recognizing the linear polylactosamines can be sued for the recognition of the structures, when these are linked to protein linked glycans. In a preferred embodiment the invention is directed to the poly-N-acetyllactosamines linked to N-glycans, preferably β2-linked structures such as Galβ4GlcNAcβ3Galβ4GlcNAcβ2Man on N-glycans. The invention is further directed to the characterization of the poly-N-acetyllactosmine structures of the preferred cells and their modification by SAα3, SAα6, Fucα2 to non-reducing end Gal and by Fucα3 to GlcNAc residues.

The invention is preferably directed to recognition of tetrasaccharides, hexasaccharides, and octasaccharides. The invention further revealed branched glycolipid polylactosamines including terminal type II lacNAc epitopes, preferably these includes Galβ4GlcNAcβ6Gal, Galβ4GlcNAcβ6Galβ,

Galβ4GlcNAcβ6(Galβ4GlcNAcβ3)Gal, and

Galβ4GlcNAcβ6(Galβ4GlcNAcβ3)Galβ3,

Galβ4GlcNAcβ6(Galβ4GlcNAcβ3)Galβ4Glc(NAc),

Galβ4GlcNAcβ6(Galβ4GlcNAcβ3)Galβ4Glc, and

Galβ4GlcNAcβ6(Galβ4GlcNAcβ3)Galβ4GlcNAc.

It is realized that antibodies specifically binding to the linear branched poly-N-acetyllactosamines are well known in the art. The invention is further directed to reagents recognizing both branched polyLacNAcs and core II O-glycans with similar β6Gal(NAc) epitopes.

Lewis x Structures

Elongated Lewis x structures are especially expressed on N-glycans. Preferred Lewis x structures are β2-linked to biantennary N-glycan core structure, Gal(Fucα3)β4GlcNAcβ2Manα3/6Manβ4

The invention further revealed presence of Lewis x on glycolipids. The preferred glycolipid structures includes Gal(Fucα3)β4GlcNAcβ3Gal, Galβ4(Fucα3)GlcNAcβ3Gal, Galβ4(Fucα3)GlcNAcβ3Galβ4, Galβ4(Fucα3)GlcNAcβ3Galβ4Glc(NAc), Galβ4(Fucα3)GlcNAcβ3Galβ4Glc, and Galβ4(Fucα3)GlcNAcβ3Galβ4GlcNAc.

The invention further revealed presence of Lewis x on O-glycans. The preferred glycolipid structures includes preferably core II structures Galβ4(Fucα3)GlcNAcβ6GAlNAc, Galβ4(Fucα3)GlcNAcβ6GalNAcα, Galβ4(Fucα3)GlcNAcβ6(Galβ3)GalNAc, and Galβ4(Fucα3)GlcNAcβ6(Galβ3)GalNAcα.

H Type II Structures

Specific elongated H type II structure epitopes are especially expressed on N-glycans. Preferred H type II structures are 02-linked to biantennary N-glycan core structure, Fucα2Galβ4GlcNAcβ2Manα3/6Manβ4

The invention further revealed presence of H type II on glycolipids. The preferred glycolipid structures includes Fucα2Galβ4GlcNAcβ3Gal, Fucα2Galβ4GlcNAcβ3Gal, Fucα2Galβ4GlcNAcβ3 Galβ4, Fucα2Galβ4GlcNAcβ3Galβ4Glc(NAc), Fucα2Galβ4GlcNAcβ3Galβ4Glc, and Fucα2Galβ4GlcNAcβ3 Galβ4GlcNAc.

The invention further revealed presence of H type II on O-glycans. The preferred glycolipid structures includes preferably core II structures Fucα2Galβ4GlcNAcβ6GAlNAc, Fucα2Galβ4GlcNAcβ6GalNAcα, Fucα2Galβ4GlcNAcβ6(Galβ3)GalNAc, and Fucα2Galβ4GlcNAcβ6(Galβ3)GalNAcα.

Sialylated Type II N-Acetyllactosamine Structures

The invention revealed preferred sialylated type II N-acetyllactosamines including specific O-glycan, and N-aglycan and glycolipid epitopes. The invention is in a preferred embodiment especially directed to abundant O-glycan and N-glycan epitopes. SA referres here to sialic acid preferably Neu5Ac or Neu5Gc, more preferably Neu5Ac. The sialic acid residues are SAα3 Gal or SAα6Gal, it is realized that these structures when presented as specific elongated epitopes form characteristic terminal structures on glycans.

Sialylated type II LacNAc structure epitopes are especially expressed on N-glycans.

Preferred type II LacNAc structures are β2-linked to biantennary N-glycan core structure, including the preferred terminal epitopes SAα3/6Galβ4GlcNAcβ2Man, SAα3/6Galβ4GlcNAcβ2Manα, and SAα3/6Galβ4GlcNAcβ2Manα3/6Manβ4. The invention is directed to both SAα3-structures (SAα3Galβ4GlcNAcβ2Man, SAα3Galβ4GlcNAcβ2Manα, and SAα3Galβ4GlcNAcβ2Manα3/6Manβ4) and SAα6-epitopes (SAα6Galβ4GlcNAcβ2Man, SAα6Galβ4GlcNAcβ2Manα, and SAα6Galβ4GlcNAcβ2Manα3/6Manβ4) on N-glycans.

The invention further revealed novel O-glycan epitopes with terminal sialylated type II N-acetyllactosamine structures expressed effectively the mesenchymaltype cells. The analysis of O-glycan structures revealed especially core II N-acetyllactosamines with the terminal structure. The preferred elongated type II sialylated N-acetyllactosamines thus includes SAα3/6Galβ4GlcNAcβ6GalNAc, SAα3/6Galβ4GlcNAcβ6GalNAcα, SAα3/6Galβ4GlcNAcβ6(Galβ3)GalNAc, and SAα3/6Galβ4GlcNAcβ6(Galβ3)GalNAcα. The SAα3-structures were revealed as preferred structures in context of the O-glycans including SAα3Galβ4GlcNAcβ6GalNAc, SAα3Galβ4GlcNAcβ6GalNAcα, SAα3Galβ4GlcNAcβ6(Galβ3)GalNAc, and SAα3Galβ4GlcNAcβ6(Galβ3)GalNAcα.

Specific Preferred Tetrasaccharide Type II Lactosamine Epitopes

It is realized that highly effective reagents can in a preferred embodiment recognize epitopes which are larger that trisaccharide. Therefore the invention is further directed to to branched terminal type II lactosamine derivatives Lewis y Fucα2Galβ4(Fucα3)GlcNAc and sialyl-Lewis x SAα3Galβ4(Fucα3)GlcNAc as preferred elongated or large glycan structure epitopes. It realized that the structures are combinations of preferred termina trisaccharide sialyl-lactosamine, H-type II and Lewis x epitopes. The analysis of the epitopes is prefeered as additionally useful method in context of analysis of other terminal type II epitopes. The invention is especially directed to the further defining the core structures carrying the type Lewis y and sialyl-Lewis x epitopes on various types of glycans and optimizing the recognition of the structures by including recognition of preferred glycan core structures.

Structures Analogous to the Type II Lactosamines

The invention is further directed to the recognition of elongated epitopes analogous to the type II N-acetyllactosamines including LacdiNAc especially on N-glycans and lactosylceramide (Galβ4GlcβCer) glycolipid structure. These share similarity with LacNAc with only difference in number of NAc residues on position of the monosaccharide residues.

LacdiNAc Structures

It is realized that LacdiNac is relatively rare and characteristic glycan structure and it is this especially preferred for the characterization of the mesenchymal cells. The invention revealed presence of LacdiNAc on N-glycans with at least β2-linkage. The structures were characterized by specific glycosidase cleavage. The LacdiNAc structures have same mass as structures with two terminal present GlcNAc containing structures in structural Table 13, indicating only single isomeric structure for a specific mass number. The preferred elongated LacdiNAc epitopes thus includes GalNAcβ4GlcNAcβ2Man, GalNAcβ4GlcNAcβ2Manα, and GalNAcβ4GlcNAcβ2Manα3/6Manβ4. The invention further revealed fucosylation LacdiNAc containing glycan structures and the preferred epitopes thus further includes GalNAcβ4(Fucα3)GlcNAcβ2Man, GalNAcβ4(Fucα3)GlcNAcβ2Manα, Gal(Fucα3)β4GlcNAcβ2Manα3/6Manβ4. It is realized that presence of α6-linked sialic acid of LacNac of structure with mass number 2263, table 13 indicates that at least part of the fucose is present on the LacdiNAc arm of the molecule based on the competing nature of α6-sialylation and α3-fucosylation. Type I N-acetyllactosamine based structures

Terminal Type I N-Acetyllactosamine Structures

The invention revealed preferred type I N-acetyllactosamines including specific O-glycan, N-glycan and glycolipid epitopes. The invention is in a preferred embodiment especially directed to abundant glycolipid epitopes. The invention is further directed to recognition of characteristic O-glycan type I LacNAc terminal.

The invention further revealed presence of type I LacNAc on glycolipids. The present invention reveals for the first time terminal type I N-acetyllactosamine on glycolipids. The Lacto glycolipid family is an important glycolipid family characteristically expressed on certain tissue but not on others.

The preferred glycolipid structures includes epitopes, preferably non-reducing end terminal epitopes of linear neolactoteraosyl ceramide and elongated variants thereof Galβ3 GlcNAcβ3Gal, Galβ3GlcNAcβ3Galβ4, Gal3βGlcNAc3βGalβ4Glc(NAc), Gal3βGlcNAcβ3Galβ4Glc, and Galβ3GlcNAc3Galβ4GlcNAc. It is further realized that specific reagents recognizing the linear polylactosamines can be used for the recognition of the structures, when these are linked to protein linked glycans. It is epscially realized that the terminal tri-and terasaccharide epitopes on the preferred O-glycans and glycolipids are essentially the same. The invention is in a preferred embodiment directed to the recognition of the both structures by the same binding reagent such as monoclonal antibody

The invention is further directed to the characterization of the terminal type I poly-N-acetyllactosmine structures of the preferred cells and their modification by SAα3, Fucα2 to non-reducing end Gal and by SAα6 or Fucα3 to GlcNAc residues and other core glycan structures of the derivatized type I N-acetyllactosamines.

A preferred elongated type I LacNAc structure is expressed on N-glycans. Preferred type I LacNAc structures are β2-linked to biantennary N-glycan core structure, with preferred epitopes Galβ3GlcNAcβ2Man, Galβ3GlcNAcβ2Manα and Galβ3GlcNAcβ2Manα3/6Manβ4.

The invention is directed to method of evaluating the status of a mesenchymal cell preferably mesenchymal stem cell preparation comprising the step of detecting the presence of an elongated glycan structure or a group, at least two, of glycan structures in said preparation, wherein said glycan structure or a group of glycan structures is according to Formula T1

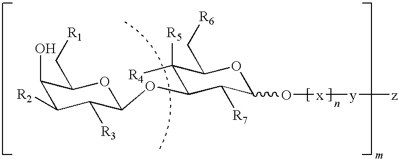

wherein X is linkage position

R₁, R₂, and R₆ are OH or glycosidically linked monosaccharide residue Sialic acid, preferably Neu5Acα2 or Neu5Gc α2, most preferably Neu5Acα2 or

R₃, is OH or glycosidically linked monosaccharide residue Fucα1 (L-fucose) or N-acetyl (N-acetamido, NCOCH₃);

R₄, is H, OH or glycosidically linked monosaccharide residue Fucα1 (L-fucose),

R₅ is OH, when R₄ is H, and R₅ is H, when R₄ is not H;

R7 is N-acetyl or OH

X is natural oligosaccharide backbone structure from the cells, preferably N-glycan, O-glycan or glycolipid structure; or X is nothing, when n is 0,

Y is linker group preferably oxygen for O-glycans and O-linked terminal oligosaccharides and glycolipids and N for N-glycans or nothing when n is 0;

Z is the carrier structure, preferably natural carrier produced by the cells, such as protein or lipid, which is preferably a ceramide or branched glycan core structure on the carrier or H;

The arch indicates that the linkage from the galactopyranosyl is either to position 3 or to position 4 of the residue on the left and that the R4 structure is in the other position 4 or 3;

n is an integer 0 or 1, and m is an integer from 1 to 1000, preferably 1 to 100, and most preferably 1 to 10 (the number of the glycans on the carrier),

With the provisions that one of R2 and R3 is OH or R3 is N-acetyl, R6 is OH, when the first residue on left is linked to position 4 of the residue on right:

X is not Galα4Galβ4Glc, (the core structure of SSEA-3 or 4) or R3 is Fucosyl, for the analysis of the status of stem cells and/or manipulation of the stem cells, and wherein said cell preparation is mesenchymal cell preparation.

and when the glycan structure is an elongated structure, wherein the binder binds to the structure and additionally to at least one reducing end elongation epitope, preferably monosaccharide epitope, (replacing X and/or Y) according to the Formula E1:

AxHex(NAc)ₙ, wherein A is anomeric structure alfa or beta, X is linkage position 2, 3, or 6; and Hex is hexopyranosyl residue Gal, or Man, and n is integer being 0 or 1, with the provisions that

when n is 1 then AxHexNAc is β4GalNAc or β6GalNAc,

when Hex is Man, then AxHex is β2Man, and

when Hex is Gal, then AxHex is β3Gal or β6Gal or α3Gal or α4Gal; or

the binder epitope binds additionally to reducing end elongation epitope

Ser/Thr linked to reducing end GalNAcα-comprising structures or

βCer linked to Galβ4Glc comprising structures, and the glycan structure is the stem cell population determined from associated or contaminating cell population.
The invention is directed to method for the analysis of the status of the stem cells and/or
for manipulation of stem cells comprising a step of detecting an elongated glycan structure or at least two glycan structures from a sample of stem cells, wherein said glycan structure is selected from the group consisting of: a terminal lactosamine structure
   (R1)ₙ₁Gal(NAc)ₙ₃β3/4(Fucα4/3)ₙ₂GlcNAcOR wherein R1 is Fucα2, or SAα3, or SAα6 linked to Galβ4GlcNAc, and
   R is the reducing end core structure of N-glycan, O-glycan and/or glycolipid; a,
   or structure
   (SAα3)ₙ₁Galβ3(SAα6)ₙ₂GalNAc; wherein
      n1, n2 and n3 are 0 or 1 indicating presence or absence of a structure wherein SA is a sialic acid; or branched epitope
   Galβ3(GlcNAcβ6)GalNAc or
   R₁Galβ4(R₃)GlcNAcβ6(R₂Galβ3)GalNAc,
      wherein R₁ and R₂ are independently either nothing or SAα3; and R₃ is independently either nothing or Fucα3; or
   Manβ4GlcNAc structure in the core structure of N-linked glycan; or epitope Galβ4Glc,
   or terminal mannose
   or terminal SAα3/6Gal, wherein SA is a sialic acid, with the provisions that
      i) the stem cells are not cells of a cancer cell line and
      ii) cells are not hematopoietic CD34⁺ cells and when the the structure is comprises N-acetyllactosamine it is specific elongated structure being fucosylated or not SAα3Galβ4GlcNAcβ3Gal structure.

The invention is directed to methods and binding agents recognizing type II

Lactosmine based structures according to the structure according to the Formula T8Ebeta

[Mα]ₘGalβ1-3/4[Nα]ₙGlcNAcβxHex(NAc)ₚ

wherein

wherein x is linkage position 2, 3, or 6

wherein m, n and p are integers 0, or 1, independently

M and N are monosaccharide residues being

i) independently nothing (free hydroxyl groups at the positions) and/or

ii) SA which is Sialic acid linked to 3-position of Gal or/and 6-position of GlcNAc and/or

iii) Fuc (L-fucose) residue linked to 2-position of Gal and/or 3 or 4 position of GlcNAc,

when Gal is linked to the other position (4 or 3) of GlcNAc,

with the provision that m, n and p are 0 or 1, independently.

Hex is hexopyranosyl residue Gal, or Man,

with the provisions that when p is 1 then βxHexNAc is β6GalNAc,

when p is 0

then Hex is Man and βxHex is β2Man, or Hex is Gal and βxHex is β3Gal or β6Gal.

The invention is directed to methods and binding agents recognizing type II Lactosmine based structures according to the Formula T10E

[Mα]ₘGalβ1-4[Nα]ₙGlcNAβxHex(NAc)ₚ

with the provisions that when p is 1 then βxHexNAc is β6GalNAc,

when p is 0, then Hex is Man and OxHex is β2Man, or Hex is Gal and βxHex is β6Gal.

The invention is directed to methods and binding agents recognizing type II Lactosmine based structures according to the Formula T10EMan:

[Mα]ₘGalβ1-4[Nα]ₙGlcNAcβ2Man,

wherein the variables are as described for Formula T8Ebeta in claim 2.

A method of evaluating the status of a human blood related, preferably hematopietic, stem cell preparation and/or contaminating cell population comprising the step of detecting the presence of an elongated glycan structure or a group, at least two, of glycan structures in said preparation, wherein said glycan structure or a group of glycan Tn and sialyl-Tn structures is according to Formula MUC

(R)ₙGalNAcα(Ser/Thr)ₘ

wherein n and m are 0 or 1, independently and R is SAα6 or Galβ3, SA is sialic acid preferably Neu5Ac, and when R is Galβ3 n is 1, preferably Tn antiges:

(SAα6)ₙGalNAcα(Ser/Thr)ₘ,

wherein n and m are 0 or 1, idependently and SA is sialic acid preferably Neu5Ac, or TF antigen

Galβ3GalNAcα(Ser/Thr)ₘ.

### Examples

### Example 1

### MALDI-TOF Mass Spectrometric N-Glycan Profiling, Glycosidase and Lectin Profiling of Cord Blood Derived and Bone Marrow Derived Mesenchymal Stem Cell Lines

Examples of Stem Cell Sample Production

Cord Blood Derived Mesenchymal Stem Cell Lines

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min). The mononuclear cell fragment was collected from the gradient and washed twice with PBS.

Umbilical cord blood cell isolation and culture. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http://www.invitrogen.com) 40% MCDB-201 (Sigma-Aldrich) 2% fetal calf serum (FCS), 1× penicillin-streptomycin (both form Gibco), 1× ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/ cm². In later passages the cells were detached with trypsin/EDTA from defined area at defined time points, counted with hematocytometer and replated at density of 2000-3000 cells/cm².

Bone Marrow Derived Mesenchymal Stem Cell Lines

Isolation and culture of bone marrow derived stem cells. Bone marrow (BM)—derived MSCs were obtained as described by Leskela et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

Experimental Procedures

Flow cytometric analysis of mesenchymal stem cell phenotype. Both UBC and BM derived mesenchymal stem cells were phenotyped by flow cytometry (FACSCalibur, Becton Dickinson). Fluorescein isothicyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abcam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD14, CD34, CD45 and HLA-DR and positive for CD13, CD29, CD44, CD90, CD 105 and HLA-ABC.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Osteogenic differentiation. To induce the osteogenic differentiation of the BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

Cell harvesting for glycome analysis. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times.

The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

Lectin stainings. FITC-labeled Maackia amurensis agglutinin (MAA) was purchased from EY Laboratories (USA) and FITC-labeled Sambucus nigra agglutinin (SNA) was purchased from Vector Laboratories (UK). Bone marrow derived mesenchymal stem cell lines were cultured as described above. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10 minutes. After fixation, cells were washed 3 times with PBS and non-specific binding sites were blocked with 3% HSA-PBS (FRC Blood Service, Finland) or 3% BSA-PBS (>99% pure BSA, Sigma) for 30 minutes at RT. According to manufacturers' instructions cells were washed twice with PBS, TBS (20 mM Tris-HCl, pH 7.5, 150 mM NaCl, 10 mM CaCl₂) or HEPES-buffer (10 mM HEPES, pH 7.5, 150 mM NaCl) before lectin incubation. FITC-labeled lectins were diluted in 1% HSA or 1% BSA in buffer and incubated with the cells for 60 minutes at RT in the dark. Furthermore, cells were washed 3 times 10 minutes with PBS/TBS/HEPES and mounted in Vectashield mounting medium containing DAPI-stain (Vector Laboratories, UK). Lectin stainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Results

Glycan isolation from mesenchymal stem cell populations. The present results are produced from two cord blood derived mesenchymal stem cell lines and cells induced to differentiate into adipogenic direction, and two marrow derived mesenchymal stem cell lines and cells induced to differentiate into osteogenic direction. The caharacterization of the cell lines and differentiated cells derived from them are described above. N-glycans were isolated from the samples, and glycan profiles were generated from MALDI-TOF mass spectrometry data of isolated neutral and sialylated N-glycan fractions as described in the preceding examples.

Cord Blood Derived Mesenchymal Stem Cell (CB MSC) Lines

Neutral N-glycan structuralfeatures. Neutral N-glycan groupings proposed for the two CB MSC lines resemble each other closely, indicating that there are no major differences in their neutral N-glycan structural features. However, CB MSCs differ from the CB mononuclear cell populations, and they have for example relatively high amounts of neutral complex-type N-glycans, as well as hybrid-type or monoantennary neutral N-glycans, compared to other structural groups in the profiles.

Identification of soluble glycan components. Similarly to CB mononuclear cell populations, in the present analysis neutral glycan components were identified in all the cell types that were assigned as soluble glycans based on their proposed monosaccharide compositions including components from the glycan group Hex₂₋₁₂HexNAc₁ (see Figures). The abundancies of these glycan components in relation to each other and in relation to the other glycan signals vary between individual samples and cell types.

Sialylated N-glycan profiles. Sialylated N-glycan profiles obtained from two CB MSC lines resemble closely each other with respect to their overall sialylated N-glycan profiles. However, minor differences between the profiles are observed, and some glycan signals can only be observed in one cell line, indicating that the two cell lines have glycan structures that differ them from each other. The analysis revealed in each cell type the relative proportions of about 50-70 glycan signals that were assigned as acidic N-glycan components. Typically, significant differences in the glycan profiles between cell populations are consistent throughout multiple experiments.

Differentiation-associated changes in glycan profiles. Neutral N-glycan profiles of CB MSCs change upon differentation in adipogenic cell culture medium. The present results indicate that relative abundancies of several individual glycan signals as well as glycan signal groups change due to cell culture in differentiation medium. The major change in glycan structural groups associated with differentation is increase in amounts of neutral complex-type N-glycans, such as signals at m/z 1663 and m/z 1809, corresponding to the Hex₅HexNAc₄ and Hex₅HexNAc₄dHex₁ monosaccharide compositions, respectively. Changes were also observed in sialylated glycan profiles.

Glycosidase analyses of neutral N-glycans. Specific exoglycosidase digestions were performed on isolated neutral N-glycan fractions from CB MSC lines as described in Examples. The results of α-mannosidase analysis show in detail which of the neutral N-glycan signals in the neutral N-glycan profiles of CB MSC lines are susceptible to α-mannosidase digestion, indicating for the presence of non-reducing terminal α-mannose residues in the corresponding glycan structures. As an example, the major neutral N-glycan signals at m/z 1257, 1419, 1581, 1743, and 1905, which were preliminarily assigned as high-mannose type N-glycans according to their proposed monosaccharide compositions Hex₅₋₉HexNAc₂, were shown to contain terminal α-mannose residues thus confirming the preliminary assignment. The results indicate for the presence of non-reducing terminal β1,4-galactose residues in the corresponding glycan structures. As an example, the major neutral complex-type N-glycan signals at m/z 1663 and m/z 1809 were shown to contain terminal β1,4-linked galactose residues.

Bone Marrow Derived Mesenchymal Stem Cell (BM MSC) Lines

Neutral N-glycan profiles and differentiation-associated changes in glycan profiles. Neutral N-glycan profiles obtained from a BM MSC line, grown in proliferation medium and in osteogenic medium resemble CB MSC lines with respect to their overall neutral N-glycan profiles. However, differences between cell lines derived from the two sources are observed, and some glycan signals can only be observed in one cell line, indicating that the cell lines have glycan structures that differ them from each other. The major characteristic structural feature of BM MSCs is even more abundant neutral complex-type N-glycans compared to CB MSC lines. Similarly to CB MSCs, these glycans were also the major increased glycan signal group upon differentiation of BM MSCs. The analysis revealed in each cell type the relative proportions of about 50-70 glycan signals that were assigned as non-sialylated N-glycan components. Typically, significant differences in the glycan profiles between cell populations are consistent throughout multiple experiments.

Sialylated N-glycan profiles. Sialylated N-glycan profiles obtained from a BM MSC line, grown in proliferation medium and in osteogenic medium. The undifferentiated and differentiated cells resemble closely each other with respect to their overall sialylated N-glycan profiles. However, minor differences between the profiles are observed, and some glycan signals can only be observed in one cell line, indicating that the two cell types have glycan structures that differ them from each other. The analysis revealed in each cell type the relative proportions of about 50 glycan signals that were assigned as acidic N-glycan components. Typically, significant differences in the glycan profiles between cell populations are consistent throughout multiple experiments.

Sialidase analysis. The sialylated N-glycan fraction isolated from BM MSCs was digested with broad-range sialidase as described in the preceding Examples. After the reaction, it was observed by MALDI-TOF mass spectrometry that the vast majority of the sialylated N-glycans were desialylated and transformed into corresponding neutral N-glycans, indicating that they had contained sialic acid residues (NeuAc and/or NeuGc) as suggested by the proposed monosaccharide compositions. Glycan profiles of combined neutral and desialylated (originally sialylated) N-glycan fractions of BM MSCs grown in proliferation medium and in osteogenic medium correspond to total N-glycan profiles isolated from the cell samples (in desialylated form). It is calculated that in undifferentiated BM MSCs (grown in osteogenic medium), approximately 53% of the N-glycan signals correspond to high-mannose type N-glycan monosaccharide compositions, 8% to low-mannose type N-glycans, 31% to complex-type N-glycans, and 7% to hybrid-type or monoantennary N-glycan monosaccharide compositions. In differentiated BM MSCs (grown in osteogenic medium), approximately 28% of the N-glycan signals correspond to high-mannose type N-glycan monosaccharide compositions, 9% to low-mannose type N-glycans, 50% to complex-type N-glycans, and 11% to hybrid-type or monoantennary N-glycan monosaccharide compositions.

Lectin binding analysis of mesenchymal stem cells. As described under Experimental procedures, bone marrow derived mesenchymal stem cells were analyzed for the presence of ligands of α2,3-linked sialic acid specific (MAA) and α2,6-linked sialic acid specific (SNA) lectins on their surface. It was revealed that MAA bound strongly to the cells whereas SNA bound weakly, indicating that in the cell culture conditions, the cells had significantly more α2,3-linked than α2,6-linked sialic acids on their surface glycoconjugates. The present results suggest that lectin staining can be used as a further means to distinguish different cell types and complements mass spectrometric profiling results.

Detection of Potential Glycan Contaminations from Cell Culture Reagents

In the sialylated N-glycan profiles of MSC lines, specific N-glycan signals were observed that indicated contamination of mesenchymal stem cell glycoconjugates by abnormal sialic acid residues. First, when the cells were cultured in cell culture media with added animal sera, such as bovine of equine sera, potential contamination by N-glycolylneuraminic acid (Neu5Gc) was detected. The glycan signals at m/z 1946, corresponding to the [M−H]⁻ ion of NeuGc₁Hex₅HexNAc₄, as well as m/z 2237 and m/z 2253, corresponding to the [M−H]⁻ ions of NeuGc₁NeuAc₁Hex₅HexNAc₄ and NeuGc₂Hex₅HexNAc₄, respectively, were indicative of the presence of Neu5Gc, i.e. a sialic acid residue with 16 Da larger mass than N-acetylneuraminic acid (Neu5Ac). Moreover, when the cells were cultured in cell culture media with added horse serum, potential contamination by O-acetylated sialic acids was detected. Diagnostic signals used for detection of O-acetylated sialic acid containing sialylated N-glycans included [M−H]⁻ ions of Ac₁NeuAc₁Hex₅HexNAc₄, Ac₁NeuAc₂Hex₅HexNAc₄, and Ac₂NeuAc₂Hex₅HexNAc₄, at calculated m/z 1972.7, 2263.8, and 2305.8, respectively.

Conclusions

Uses of the glycan profiling method. The results indicate that the present glycan profiling method can be used to differentiate CB MSC lines and BM MSC lines from each other, as well as from other cell types such as cord blood mononuclear cell populations. Differentation-induced changes as well as potential glycan contaminations from e.g. cell culture media can also be detected in the glycan profiles, indicating that changes in cell status can be detected by the present method. The method can also be used to detect MSC-specific glycosylation features including those discussed below.

Differences in glycosylation between cultured cells and native human cells. The present results indicate that BM MSC lines have more high-mannose type N-glycans and less low-mannose type N-glycans compared to the other N-glycan structural groups than mononuclear cells isolated from cord blood. Taken together with the results obtained from cultured human embryonal stem cells in the following Examples, it is indicated that this is a general tendency of cultured stem cells compared to native isolated stem cells. However, differentiation of BM MSCs in osteogenic medium results in significantly increased amounts of complex-type N-glycans and reduction in the amounts of high-mannose type N-glycans. Mesenchymal stem cell line specific glycosylation features. The present results indicate that mesenchymal stem cell lines differ from the other cell types studied in the present study with regard to specific features of their glycosylation, such as:
1) Both CB MSC lines and BM MSC lines have unique neutral and sialylated N-glycan profiles;
2) The major characteristic structural feature of both CB and BM MSC lines is abundant neutral complex-type N-glycans;
3) An additional characteristic feature is low sialylation level of complex-type N-glycans.

### Example 2

### Lectin and Antibody Profiling of Human Mesenchymal Stem Cells

Experimental Procedures

Cell samples. Bone marrow derived human mesenchymal stem cell lines (MSC) were generated and cultured in proliferation medium as described above.

FITC-labeled lectins. Fluorescein isotiocyanate (FITC) labelled lectins were purchased from several manufacturers: FITC-GNA, -HHA, -MAA, -PWA, -STA and -LTA were from EY Laboratories (USA); FITC-PSA and -UEA were from Sigma (USA); and FITC-RCA, -PNA and -SNA were from Vector Laboratories (UK). Lectins were used in dilution of 5 pg/10⁵ cells in 1% human serum albumin (HSA; FRC Blood Service, Finland) in phosphate buffered saline (PBS).

Flow cytometry. Flow cytometric analysis of lectin binding was used to study the cell surface carbohydrate expression of MSC. 90% confluent MSC layers on passages 9-11 were washed with PBS and harvested into single cell suspensions by 0.25% trypsin −1 mM EDTA solution (Gibco). The trypsin treatment was aimed to gentle, but it is realized that part of the structures recognized when compared to experiments by antibodies may be partially lost or reduced. Detached cells were centrifuged at 600 g for five minutes at room temperature. Cell pellet was washed twice with 1% HSA-PBS, centrifuged at 600 g and resuspended in 1% HSA-PBS. Cells were placed in conical tubes in aliquots of 70000-83000 cells each. Cell aliquots were incubated with one of the FITC labelled lectin for 20 minutes at room temperature. After incubation cells were washed with 1% HSA-PBS, centrifuged and resuspended in 1% HSA-PBS. Untreated cells were used as controls. Lectin binding was detected by flow cytometry (FACSCalibur, Becton Dickinson). Data analysis was made with Windows Multi Document Interface for Flow Cytometry (WinMDI 2.8). Two independent experiments were carried out.

Fluorescence Microscopy Labeling Experiments were Conducted as Described in the Preceding Examples.

Results and Discussions

Table 16 shows the tested FITC-labelled lectins, examples of their target saccharide sequences, and the amount of cells showing positive lectin binding (%) in FACS analysis after mild trypsin treatment. Table 17 shows the tested FITC-labelled lectins, examples of their target saccharide sequences, and the graded lectin binding intensities as described in the Table legend, in fluorescence microscopy of fixed cells grown on microscopy slides. Binding specificities of the used lectins are described in the art and in general the binding of a lectin in the present experiments means that the cells express specific ligands for the lectin on their surface. The examples of some of the specificities discussed below and those marked in the Tables are therefore non-exclusive in nature.

α-linked mannose. Abundant labelling of the cells by both Hippeastrum hybrid (HHA) and Pisum sativum (PSA) lectins suggests that they express mannose, more specifically α-linked mannose residues on their surface glycoconjugates such as N-glycans. Possible α-mannose linkages include α1→2, α1→3, and α1→6. The lower binding of Galanthus nivalis (GNA) lectin suggests that some α-mannose linkages on the cell surface are more prevalent than others.

β-linked galactose. Abundant labelling of the cells by Ricinus communis lectin I (RCA-I) and less intense labelling by peanut lectin (PNA) suggests that the cells express β-linked non-reducing terminal galactose residues on their surface glycoconjugates such as N- and/or O-glycans. More specifically, the intense RCA-I binding suggests that the cells contain high amounts of unsubstituted Galβ epitopes on their surface. The binding of RCA-I was increased by sialidase treatment of the cells before lectin binding, indicating that the ligands of RCA-I on MSC were originally partly covered by sialic acid residues. PNA binding suggests for the presence of another type of unsubstituted Galp epitopes such as Core 1 O-glycan epitopes on the cell surface. The binding of PNA was also increased by sialidase treatment of the cells before lectin binding, indicating that the ligands of PNA on MSC were originally mostly covered by sialic acid residues. These results suggest that both RCA-I and PNA can be used to assess the amount of their specific ligands on the cell surface of BM MSC, and with or without conjunction with sialidase treatment to assess the sialylation level of their specific epitopes.

Sialic acids. Abundant labelling of the cells by Maackia amurensis (MAA) and less intense labelling by Sambucus nigra (SNA) lectins suggests that the cells express sialic acid residues on their surface glycoconjugates such as N- and/or O-glycans and/or glycolipids. More specifically, the intense MAA binding suggests that the cells contain high amounts of α2,3-linked sialic acid residues on their surface. SNA binding suggests for the presence of also α2,6-linked sialic acid residues on the cell surface, however in lower amounts than α2,3-linked sialic acids. Both of these lectin binding activities could be reduced by sialidase treatment, indicating that the specificities of the lectins in BM MSC are mostly targeted to sialic acids.

Poly-N-acetyllactosamine sequences. Labelling of the cells by Solanum tuberosum (STA) and less intense labelling by pokeweed (PWA) lectins suggests that the cells express poly-N-acetyllactosamine sequences on their surface glycoconjugates such as N- and/or O-glycans and/or glycolipids. Higher intensity labelling with STA than with PWA suggests that most of the cell surface poly-N-acetyllactosamine sequences are linear and not branched or substituted chains.

Fucosylation. Labelling of the cells by Ulex europaeus (UEA) and less intense labelling by Lotus tetragonolobus (LTA) lectins suggests that the cells express fucose residues on their surface glycoconjugates such as N- and/or O-glycans and/or glycolipids. More specifically, the UEA binding suggests that the cells contain α-linked fucose residues, including α1,2-linked fucose residues, on their surface. LTA binding suggests for the presence of also α-linked fucose residues, including α1,3-linked fucose residues on the cell surface, however in lower amounts than UEA ligand fucose residues.

Mannose-binding lectin labelling. Low labelling intensity was also detected with human serum mannose-binding lectin (MBL) coupled to fluorescein label, suggesting that ligands for this innate immunity system component may be expressed on in vitro cultured BM MSC cell surface.

Binding of a NeuGc polymeric probe (Lectinity Ltd., Russia) to non-fixed hESC indicates the presence of NeuGc-specific lectin on the cell surfaces. In contrast, polymeric NeuAc probe did not bind to the cells with same intensity in the present experiments.

The binding of the specific antibodies to hESC indicates the presence of Lex and sialyl-Lewis x epitopes on their surfaces, and binding of NeuGc-specific antibody to hESC indicates the presence of NeuGc epitopes on their surfaces.

### Example 3

### Lectin and Antibody Profiling of Human Cord Blood Cell Populations

Results and Discussions

FIG. 1 shows the results of FACS analysis of FITC-labelled lectin binding to seven individual cord blood mononuclear cell (CB MNC ) as an example of mainly associated / control cells in context of CB MSC preparations (experiments performed as described above). Strong binding was observed in all samples by GNA, HHA, PSA, MAA, STA, and UEA FITC-labelled lectins, indicating the presence of their specific ligand structures on the CB MNC cell surfaces. Also mediocre binding (PWA), variable binding between CB samples (PNA), and low binding (LTA) was observed, indicating that the ligands for these lectins are either variable or more rare on the CB MNC cell surfaces as the lectins above.

### Example 4

### Analysis of Total N-Glycomes of Human Stem Cells and Cell Populations

Experimental Procedures

Cell and glycan samples were prepared as described in the Examples and PCT FI 2007 050336.

Relative proportions of neutral and acidic N-glycan fractions were studied by desialylating isolated acidic glycan fraction with A. ureafaciens sialidase as described in the Examples/PCT and then combining the desialylated glycans with neutral glycans isolated from the same sample. Then the combined glycan fractions were analyzed by positive ion mode MALDI-TOF mass spectrometry as described in the Examples/PCT. The proportion ofsialylated N-glycans of the combined N-glycans was calculated by calculating the percentual decrease in the relative intensity of neutral N-glycans in the combined N-glycan fraction compared to the original neutral N-glycan fraction, according to the equation:

$$proportion = \frac{{I}^{neutral} - {I}^{combined}}{{I}^{neutral}} \times 100 \% ,$$

wherein I^{neutral} and I^{combined} correspond to the sum of relative intensities of the five high-mannose type N-glycan [M+Na]⁺ ion signals at m/z 1257, 1419, 1581, 1743, and 1905 in the neutral and combined N-glycan fractions, respectively.

Results and Discussion

The relative proportions of acidic N-glycan fractions in studied stem cell types were as follows: in human embryonic stem cells (hESC) approximately 35% (proportion of sialylated and neutral N-glycans is approximately 1:2), in human bone marrow derived mesenchymal stem cells (BM MSC) approximately 19% (proportion of sialylated and neutral N-glycans is approximately 1:4), in osteoblast-differentiated BM MSC approximately 28% (proportion of sialylated and neutral N-glycans is approximately 1:3), and in human cord blood (CB) CD 133+cells approximately 38% (proportion of sialylated and neutral N-glycans is approximately 2:3).

In conclusion, BM MSC differ from hESC and CB CD 133+ cells in that they contain significantly lower amounts of sialylated N-glycans compared to neutral N-glycans. However, after osteoblast differentiation of the BM MSC the proportion of sialylated N-glycans increases.

### Example 5

### Analysis of Human and Murine Fibroblast (Feeder) Cell Lines

Murine (mEF) and human (hEF) fibroblast feeder cells were prepared and their N-glycan fractions analyzed as described in the preceding Examples.

Results and Discussions

The results showed that mEF and hEF cellular N-glycan fractions differ significantly from each other. The differencies include differential proportions of glycan groups, major glycan signals, and the glycan profiles obtained from the cell samples. In addition, the major difference is the presence of Galα3Gal epitopes in the mEF cells.

### Example 6

### Influence of Lectins on Stem Cell Proliferation Rate

Experimental Procedures

Lectins (EY laboratories, USA) were passively adsorbed on 48-well plates (Nunclon surface, catalog No 150687, Nunc, Denmark) by overnight incubation in phosphate buffered saline.

Human bone marrow derived mesenchymal stem cells (BM MSC) were cultured in minimum essential α-medium (α-MEM) supplemented with 20 mM HEPES, 10% FCS, penicillin-streptomycin, and 2 mM L-glutamine (all from Gibco) on 48-well plates coated with different lectins. Cells were cultivated in Cell IQ (ChipMan Technologies, Tampere, Finland) at +37° C. with 5% CO₂. Images were taken every 15 minutes. Data were analyzed with Cell IQ Analyzer software by analyzer protocol built by Dr. Ulla Impola (Finnish Red Cross Blood Service, Helsinki, Finland).

Results and Discussions

The growth rates of BM MSC varied on different lectin-coated surfaces compared to each other and uncoated plastic surface (Table 18), indicating that proteins with different glycan binding specificities binding to stem cell surface glycans specifically influence their proliferation rate.

Lectins that had an enhancing effect on BM MSC growth rate included in order of relative efficacy:

GS II (β-GlcNAc)>ECA (LacNAc/β-Gal)>PWA (I-branched poly-LacNAc)>LTA (α1,3-Fuc)>PSA (α-Man),

wherein the preferred oligosaccharide specificities of the lectins are indicated in parenthesis. However, PSA was nearly equal to plastic in the present experiments.

Lectins that had an inhibitory effect on BM MSC growth rate included in order of relative efficacy:

RCA (β-Gal/LacNAc)>>UEA (α1,2-Fuc)>WFA (β-GalNAc)>STA (linear poly-LacNAc)>NPA (α-Man)>SNA (α2,6-linked sialic acids)=MAA (α2,3-linked sialic acids/α3′-sialyl LacNAc),

wherein the preferred oligosaccharide specificities of the lectins are indicated in parenthesis. However, NPA, SNA, and MAA were nearly equal to plastic in the present experiments.

### Example 7

### Glycosphingolipid Glycans of Human Stem Cells

Experimental Procedures

Samples from MSC, and a cell population for comparison (CB MSC associated cell type) CB MNC were produced as described in the Examples and PCT/FI2007 050336. Neutral and acidic glycosphingolipid fractions were isolated from cells essentially as described (Miller-Podraza et al., 2000). Glycans were detached by Macrobdella decora endoglycoceramidase digestion (Calbiochem, USA) essentially according to manuacturer's instructions, yielding the total glycan oligosaccharide fractions from the samples. The oligosaccharides were purified and analyzed by MALDI-TOF mass spectrometry as described in the preceding Examples for the protein-linked oligosaccharide fractions.

Results and Discussions

Human Mesenchymal Stem Cells (MSC)

Bone marrow derived (BM) MSC neutral lipid glycans. The analyzed mass spectrometric profile of the BM MSC glycosphingolipid neutral glycan fraction is shown in FIG. 8. The six major glycan signals, together comprising more than 94% of the total glycan signal intensity, corresponded to monosaccharide compositions Hex₃HexNAc₁ (730), Hex₂HexNAc₁ (568), Hex₂dHex₁ (511), Hex₂HexNAc₂dHex₂ (1063), Hex₃HexNAc₂dHex₂ (1225), and Hex₃HexNAc₂dHex₁ (1079). The four most abundant signals (730, 568, 511, and 1063) together comprised more than 75% of the total intensity.

Cord blood derived (CB) MSC neutral lipid glycans. The analyzed mass spectrometric profile of the CB MSC glycosphingolipid neutral glycan fraction is shown in FIG. 8. The ten major glycan signals, together comprising more than 92% of the total glycan signal intensity, corresponded to monosaccharide compositions Hex₂HexNAc₁ (568), Hex₃HexNAc₁ (730), Hex₄HexNAc₂ (1095), Hex₅HexNAc₃ (1460), Hex₃HexNAc₂ (933), Hex₂dHex₁ (511), Hex₂HexNAc₂dHex₂ (1063), Hex₄HexNAc₃ (1298), Hex₃HexNAc₂dHex₂ (1225), and Hex₂HexNAc₂ (771). The five most abundant signals (568, 730, 1095, 1460, and 933) together comprised more than 82% of the total intensity.

In β1,4-galactosidase digestion, the relative signal intensities of 1095, 1460, and 730 were reduced by about 90%, 95%, and 20%, respectively. This suggests that CB MSC contained major glycan components with non-reducing terminal β1,4-Gal epitopes, preferably including the structures Galβ4GlcNAcβ[Hex₁HexNAc₁]Lac, Galβ4GlcNAc[Hex₂HexNAc₂]Lac, and Galβ4GlcNAcLac. Further, the glycan signal Hex₅HexNAc₃ (1460) was digested into Hex₄HexNAc₃ (1298) and mostly into Hex₃HexNAc₃ (1136), indicating that the original signal contained glycan structures containing either one or two β1,4-Gal, and that the majority of the original glycans contained two β1,4-Gal, preferentially including the structure Galβ4GlcNAc(Galβ4GlcNAc)[Hex₁HexNAc₁]Lac. Similarly, 1095 was digested into Hex₂HexNAc₂ (771) in addition to 933, indicating that the original signal contained glycan structures containing either one or two β1,4-Gal, and that the minority of the original glycans contained two β1,4-Gal, preferentially including the structure Galβ4GlcNAc(Galβ4GlcNAc)Lac.

The experimental structures of the major CB MSC glycosphingolipid neutral glycan signals were thus determined (‘>’ indicates the order of preference among the lipid glycan structures of MSC; ‘[ ]’ indicates that the oligosaccharide sequence in brackets may be either branched or unbranched; ‘( )’ indicates a branch in the structure):

568 Hex₂HexNAc₁>HecNAcLac

730 Hex₃HexNAc₁>Hex₁HexNAc₁Lac>Galβ4GlcNAcLac

1095 Hex₄HexNAc₂>[Hex₂HecNAc₂]Lac >Galβ4GlcNAc[Hex₁HecNAc₁]Lac>Galβ4GlcNAc(Galβ4GlcNAc)Lac

1460 Hex₅HexNAc₃>[Hex₃HecNAc₃]Lac>Galβ4GlcNAc[Hex₂HecNAc₂]Lac>Galβ4GlcNAc(Galβ4GlcNAc)[Hex₁HecNAc₁]Lac

933 Hex₃HexNAc₂>Hex₁HexNAc₂Lac

Sialylated lipid glycans. The analyzed mass spectrometric profile of the hESC glycosphingolipid sialylated glycan fraction is shown in FIG. 9. The five major glycan signals of BM MSC, together comprising more than 96% of the total glycan signal intensity, corresponded to monosaccharide compositions NeuAc₁Hex₂HexNAc₁ (835), NeuAc₁Hex₁HexNAcldHex₁ (819), NeuAc₁Hex₃HexNAc₁ (997), NeuAc₁Hex₃HexNAc₁dHex₁ (1143), and NeuAc₂Hex₁HexNAc₂dHex, (1313). The six major glycan signals of CB MSC, together comprising more than 92% of the total glycan signal intensity, corresponded to monosaccharide compositions NeuAc₁Hex₂HexNAc₁ (835), NeuAc₁Hex₃HexNAc₁ (997), NeuAc₂Hex₂ (905), NeuAc₁Hex₄HexNAc₂ (1362), NeuAc₁Hex₅HexNAc₃ (1727), and NeuAc₂Hex₂HexNAc₁ (1126).

Human Cord Blood Mononuclear Cells (CB MNC)

CB MNC neutral lipid glycans. The analyzed mass spectrometric profile of the CB MNC glycosphingolipid neutral glycan fraction is shown in FIG. 8. The five major glycan signals, together comprising more than 91% of the total glycan signal intensity, corresponded to monosaccharide compositions Hex₃HexNAc₁ (730), Hex₂HexNAc₁ (568), Hex₃HexNAc₁dHex₁ (876), Hex₄HexNAc₂ (1095), and Hex₄HexNAc₂dHex₁ (1241).

In β1,4-galactosidase digestion, the relative signal intensities of 730 and 1095 were reduced by about 50% and 90%, respectively. This suggests that the signals contained major components with non-reducing terminal β1,4-Gal epitopes, preferably including the structures Galβ4GlcNAcβLac and Galβ4GlcNAcβ[Hex₁HexNAc₁]Lac. Further, the glycan signal Hex₅HexNAc₃ (1460) was digested to Hex₄HexNAc₃ (1298) and Hex₃HexNAc₃ (1136), indicating that the original signal contained glycan structures containing either one or two β1,4-Gal.

The experimental structures of the major CB MNC glycosphingolipid neutral glycan signals were thus determined (‘>’ indicates the order of preference among the lipid glycan structures; ‘[ ]’ indicates that the oligosaccharide sequence in brackets may be either branched or unbranched; ‘( )’ indicates a branch in the structure):

730 Hex₃HexNAc₁>Hex₁HexNAc₁Lac>Galβ4GlcNAcLac

568 Hex₂HexNAc₁>HecNAcLac

876 Hex₃HexNAc₁dHex₁>[Hex₁HecNAc₁dHex₁]Lac>Fuc[Hex₁HecNAc₁]Lac

1095 Hex₄HexNAc₂>[Hex₂HecNAc₂]Lac>Galβ4GlcNAc[Hex₁HecNAc₁]Lac

1241 Hex₄HexNAc₂dHex₁>[Hex₂HecNAc₂dHex₁]Lac>Fuc[Hex₂HecNAc₂]Lac

1460 Hex₅HexNAc₃>[Hex₃HecNAc₃]Lac>Galβ4GlcNAc[Hex₂HecNAc₂]Lac>Galβ4GlcNAc(Galβ4GlcNAc)[Hex₁HecNAc₁]Lac

Sialylated lipid glycans. The analyzed mass spectrometric profile of the CB MNC glycosphingolipid sialylated glycan fraction is shown in FIG. 9. The three major glycan signals of CB MNC, together comprising more than 96% of the total glycan signal intensity, corresponded to monosaccharide compositions NeuAc₁Hex₃HexNAc₁(997), NeuAc₁Hex₄HexNAc₂ (1362), and NeuAc₁Hex₅HexNAc₃ (1727).

Overview of Human Stem Cell Glycosphingolipid Glycan Profiles

The neutral glycan fractions of all the present sample types altogether comprised 45 glycan signals. The proposed monosaccharide compositions of the signals were composed of 2-7 Hex, 0-5 HexNAc, and 0-4 dHex. Glycan signals were detected at monoisotopic m/z values between 511 and 2263 (for [M+Na]⁺ ion).

Major neutral glycan signals common to all the sample types were 730, 568, 1095, and 933, corresponding to the glycan structure groups Hex₀₋₁HexNAc₁Lac (568 or 730) and Hex₁₋₂HexNAc₂Lac (933 or 1095), of which the former glycans were more abundant and the latter less abundant. A general formula of these common glycans is HexₘHexNAcₙLac, wherein m is either n or n-1, and n is either 1 or 2.

Neutral Glycolipid Profiles of Human Stem Cell Types:

Glycan signals typical to both CB and BM MSC preferentially include 771, 1063, 1225; more preferentially including compositions dHex_{0/2}Hex_{0/1}HexNAc₂Lac.

Glycan signals typical to especially BM MSC preferentially include 511 and fucosylated structures, preferentially multifucosylated structures.

Glycan signals typical to especially CB MSC preferentially include 1460 and 1298, as well as large neutral glycolipids, especially Hex₂₋₃HexNAc₃Lac. In addition, low fucosylation and/or high expression of terminal β1,4-Gal was typical to especially CB MSC.

Glycan signals typical to CB MNC preferentially include compositions dHex₀₋₁[HexHexNAc]₁₋₂Lac, more preferentially high relative amounts of 730 compared to other signals; and fucosylated structures; and glycan profiles with less variability and/or complexity than other stem cell types.

The acidic glycan fractions of all the present sample types altogether comprised 38 glycan signals. The proposed monosaccharide compositions of the signals were composed of 0-2 NeuAc, 2-9 Hex, 0-6 HexNAc, 0-3 dHex, and/or 0-1 sulphate or phosphate esters. Glycan signals were detected at monoisotopic m/z values between 786 and 2781 (for [M−H]⁻ ion).

The acidic glycosphingolipid glycans of CB MNC were mainly composed of NeuAc₁Hexₙ₊₂HexNAcₙ, wherein 1≦n≦3, indicating that their structures were NeuAc₁[HexHexNAc]₁₋₃Lac.

Terminal glycan epitopes that were demonstrated in the present experiments in stem cell glycosphingolipid glycans include:

Gal

Galβ4Glc (Lac)

Galβ4GlcNAc (LacNAc type 2)

Galβ3

Non-reducing terminal HexNAc

Fuc

α1,2-Fuc

α1,3-Fuc

Fucα2Gal

Fucα2Galβ4GlcNAc (H type 2)

Fucα2Galβ4Glc (2′-fucosyllactose)

Fucα3GlcNAc

Galβ4(Fucα3)GlcNAc (Lex)

Fucα3Glc

Galβ4(Fucα3)Glc (3-fucosyllactose)

Neu5Ac

Neu5Acα2,3

Neu5Acα2,6

Development-related glycan epitope expression. According to the present invention, the glycosphingolipid glycan composition Hex₄HexNAc₁ preferentially corresponds to (iso)globo structures. The glycan sequence of the SSEA-3 glycolipid antigen has been determined to be Galβ3GalNAcβ3Galα4Galβ4Glc, which also corresponds to the glycan signal Hex₄HexNAc₁ (892) detected in the present experiments. In higher-resolution analysis (Example 12) the glycan signals Hex₄HexNAc₁ and NeuAc₁Hex₄HexNAc₁ were detected in small amounts also in MSC, indicating that globoside-type glycosphingolipids were relatively minor but yet significant structures in MSC (Tables 20 and 21). In contrast to mouse ES cells, hESC do not express the SSEA-1 antigen; consistent with this we found only low expression levels of α1,3/4-fucosylated neutral glycolipid glycans. In contrast, we were able to show that the major fucosylated structures of hESC glycosphingolipid glycans contain α1,2-Fuc, which is a molecular level explanation to the mouse-human difference in SSEA-1 reactivity.

### Example 8

### Immunohistochemical Staining of Mesenchymal Cells

Detection of Carbohydrate Structures on Cell Surface in Stem Cell Samples by Secific Antibodies

Materials and Methods

Cell samples. Mesenchymal stem cells (MSCS) from bone marrow were generated and cultured in proliferation medium as described above. MSCs were cultured in differentiation medium (proliferation medium including 4 ng/ml dexamethasone, 10 mmol/L β-glycerophosphate, and 50 μmol/L ascorbic acid) for 6 weeks to induce osteogenic differentiation. Differentiation medium was refreshed twice a week throughout the differentiation period.

Antibodies. Primari anti-glycan antibodies are listed in Table 25.

Immunostainings. Bone-marrow derived mesenchymal stem cells on passages 9-12 were grown on 0.01% poly-L-lysine (Sigma, USA) coated glass 8-chamber slides (Lab-Tekll, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK). Immunostainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Fluorescence activated cell sorting (FACS) analysis. Proliferating MSCs on passage 12 were detached from culture plates by 0.02% Versene solution (pH 7.4) for 45 minutes at 37° C. Cells were washed twice with 0.3% HSA-PBS solution before antibody labelling. Primary antibodies were incubated (4 μl/100 μl cell suspension/50 000 cells) for 30 minutes at RT and washed once with 0.3% HSA-PBS before secondary antibody detection with Alexa Fluor 488 goat anti-mouse (1:500) for 30 minutes at RT in the dark. As a negative control cells were incubated without primary antibody and otherwise treated similar to labelled cells. Cells were analysed with BD FACSAria (Becton Dickinson) using FITC detector at wavelength 488. Results were analysed with BD FACSDiva software version 5.0.1 (Becton Dickinson).

See Table 15 for results, for antibodies see Table 25.

### Example 9

### Exoglycosidase Analysis of Human Mesenchymal Stem Cells

The changes in the exoglycosidase digestion result Tables are relative changes in the recorded mass spectra and they do not reflect absolute changes in the glycan profiles resulting from glycosidase treatments. The experimental procedures are described in the preceding Example.

Results

Undifferentiated BM MSC

Neutral and acidic N-glycan fractions were isolated from BM MSC as described. The results of parallel exoglycosidase digestions of the neutral (Table 10) and acidic (data not shown) glycan fractions are discussed below. In the following chapters, the glycan signals are referred to by their proposed monosaccharide compositions according to the Tables of the present invention and the corresponding m/z values can be read from the Tables.

α-mannosidase sensitive structures. All the glycan signals that showed decrease upon α-mannosidase digestion of the neutral N-glycan fraction (Table 10) are indicated to correspond to glycans that contain terminal a.-mannose residues. The present results indicate that the majority of the neutral N-glycans of BM MSC contain terminal α-mannose residues. On the other hand, increased signals correspond to their reaction products. Structure groups that form series of α-mannosylated glycans in the neutral N-glycan fraction as well as individual α-mannosylated glycans are discussed below in detail.

The Hex₁ gHexNAc₁ glycan series was digested so that Hex₃-₉HexNAc₁ were digested and transformed into Hex₁HexNAc₁ (data not shown), indicating that they had contained terminal α-mannose residues. Because they were transformed into Hex₁HexNAc₁, their experimental structures were (Manα)₁₋₈Hex₁HexNAc₁.

The Hex-₁₋₁₀HexNAc₂ glycan series was digested so that Hex₄₋₁₀HexNAc₂ were digested and transformed into Hexl-₄HexNAc₂ and especially into Hex₁HexNAc₂ that had not existed before the reaction and was the major reaction product. This indicates that 1) glycans Hex₄₋₁₀HexNAc₂ include glycans containing terminal α-mannose residues, 2) glycans Hex₁₋₄HexNAc₂ could be formed from larger α-mannosylated glycans, and 3) majority of the glycans Hex₄₋₁₀HexNAc₂ were transformed into newly formed Hex₁HexNAc₂ and therefore had the experimental structures (Manα)ₙHex₁HexNAc₂, wherein n≧1. The fact that the α-mannosidase reaction was only partially completed for many of the signals suggests that also other glycan components are included in the the Hex₁₋₁₀HexNAc₂ glycan series. In particular, the Hex₁₀HexNAc₂ component contains one hexose residue more than the largest typical mammalian high-mannose type N-glycan, suggesting that it contains glucosylated structures including (Glcα→)Hex₈HexNAc₂, preferentially α3-linked Glc and even more preferentially present in the glucosylated N-glycan (Glcα3→)Man₉GlcNAc₂.

The Hex₁₋₆HexNAc₁dHex₁ glycan series was digested so that Hex₃₋₉HexNAc₁dHex₁ were digested and transformed into Hex₁HexNAc₁dHex₁, indicating that they had contained terminal α-mannose residues and their experimental structures were (Manα)₂₋₅Hex₁HexNAc₁dHex₁. Hex₁HexNAc₁dHex₁ appeared as a new signal indicating that glycans with structures (Manα)₁Hex₁HexNAc₁dHex₁, wherein n≧1, had existed in the sample.

The Hex₂₋₇HexNAc₃ glycan series was digested so that Hex₆₋₇HexNAc₃ were digested and transformed into other glycans in the series, indicating that they had contained terminal α-mannose residues. Hex₂HexNAc₃ appeared as a new signal indicating that glycans with structures (Manα)ₙHex₂HexNAc₃, wherein n≧1, had existed in the sample.

The Hex₂₋₇HexNAc₃dHex₁ glycan series was digested so that Hex₆₋₇HexNAc₃dHex₁ were digested and transformed into other glycans in the series, indicating that they had contained terminal α-mannose residues. Hex₂HexNAc₃dHex₁ appeared as a new signal indicating that glycans with structures (Manα)ₙHex₂HexNAc₃dHex₁, wherein n≧1, had existed in the sample.

Hex₃HexNAc₃dHex₂ and Hex₃HexNAc₄ appeared as new signals indicating that glycans with structures (Manα)ₙHex₃HexNAc₃dHex₂ and (Manα)ₙHex₃HexNAc₄, respectively, wherein n≧1, had existed in the sample.

β8-glucosaminidase sensitive structures. The Hex₃HexNAc₂₋₅dHex₁ glycan series was digested so that Hex₃₋₉HexNAc₁dHex₁ were digested and transformed into Hex₁HexNAc₁dHex₁, indicating that they had contained terminal α-mannose residues and their experimental structures were (Manα)₂₋₅Hex₁HexNAc₁dHex₁.

Hex₁HexNAc₁dHex₁ appeared as a new signal indicating that glycans with structures (Manα)ₙHex₁HexNAc₁dHex₁, wherein n≧1, had existed in the sample. However, Hex₃HexNAc₆dHex₁ was not digested indicating that it contained other terminal HexNAc residues than β-linked GlcNAc residues.

Hex₂HexNAc₃ and Hex₂HexNAc₃dHex₁ were digested into Hex₂HexNAc₂ and Hex₂HexNAc₂dHex₁ indicating they had the structures (GlcNAcβ→)Hex₂HexNAc₂ and (GlcNAcβ→)Hex₂HexNAc₂dHex₁, respectively.

Hex₄HexNAc₄dHex₁, Hex₄HexNAc₄dHex₂, Hex₄HexNAc₅dHex₂, and Hex₅HexNAc₅dHex₁ were also digested indicating they contained structures including (GlcNAcβ→)Hex₄HexNAc₃dHex₁, (GlcNAcβ→)Hex₄HexNAc₃dHex₂, (GlcNAcβ→)Hex₄HexNAc₄dHex₂, and (GlcNAcβ→)Hex₅HexNAc₄dHex₁, respectively.

β1,4-galactosidase sensitive structures. Glycan signals that were sensitive to β1,4-galactosidase comprised a major proportion of BM MSC glycans, indicating that β1,4-linked galactose is a common terminal epitope in BM MSC neutral N-glycans.

Hex₅HexNAc₄ and Hex₅HexNAc₄dHex₁ were digested into Hex₃HexNAc₄ and Hex₃HexNAc₄dHex₁ indicating they had the structures (Galβ4GlcNAcβ→)₂Hex₃HexNAc₂ and (Galβ4GlcNAcβ→)₂Hex₃HexNAc₂dHex₁, respectively. In contrast, Hex₅HexNAc₄dHex₂ was digested into Hex₄HexNAc₄dHex₂ indicating that it had the structure (Galβ4GlcNAc⊖→)Hex₄HexNAc₃dHex₂, respectively, and Hex₅HexNAc₄dHex₃ was not digested at all. Taken together, in BM MSC, n-1 hexose residues are protected by deoxyhexose residues from the action of β1,4-galactosidase in the N-glycan structures Hex₅HexNAc₄dHexₙ, wherein 0≦n≦3. Such dHex-protected structures containing β1,4-linked galactose include Galβ4(Fucα3)GlcNAc and Fucα2Galβ4GlcNAc.

Similarly, Hex₆HexNAc₅, Hex₅HexNAc₅dHex₁, Hex₆HexNAc₅, and Hex₅HexNAc₅dHex₁ were digested into Hex₃HexNAc₅, Hex₃HexNAc₅dHex₁, and Hex₃HexNAc₆dHex₁ indicating they had the structures (Galβ4GlcNAcβ→)₃Hex₃HexNAc₂, (Galβ4GlcNAcβ→)₂Hex₃HexNAc₃dHex₁, and (Galβ4GlcNAcβ→)₃Hex₃HexNAc₃dHex₁, respectively. In contrast, Hex₄HexNAc₅dHex₂, Hex₅HexNAc₅dHex₃, Hex₆HexNAc₅dHex₂, and Hex₆HexNAc₅dHex₃ were not digested, indicating that hexose residues in these structures were protected by deoxyhexose residues. Such dHex-protected structures containing β1,4-linked galactose include Galβ4(Fucα3)GlcNAc and Fucα2Galβ4GlcNAc. However, Hex₄HexNAc₅dHex₃ was digested indicating that it contained one or more terminal β1,4-linked galactose residues.

Hex₇HexNAc₃, Hex₆HexNAc₃dHex₁, Hex₆HexNAc₃, and Hex₅HexNAc₃dHex₁ were digested into products including Hex₅HexNAc₃ and Hex₄HexNAc₃dHex₁, indicating they had the structures (Galβ4GlcNAcβ→)Hex₅₋₆HexNAc₂ and (Galβ4GlcNAcβ→)Hex₄₋₅HexNAc₃dHex₁, respectively. The relative amounts of Hex₃HexNAc₃, and Hex₃HexNAc₃dHex, were increased indicating that they were products of (Galβ4GlcNAcβ→)Hex₃HexNAc₂ and (Galβ4GlcNAcβ→)Hex₃HexNAc₂dHex₁, respectively.

β1,3-galactosidase sensitive structures. Because only few structures in BM MSC neutral N-glycan fraction are sensitive to the action of β1,3-galactosidase, the majority of terminal galactose residues appear to be β1,4-linked. The glycan signals corresponding to β1,3-galactosidase sensitive glycans include Hex₅HexNAc₅dHex₁ and Hex₄HexNAc₅dHex₃.

Glycosidase resistant structures. In the present experiments, Hex₂HexNAc₃dHex₂, Hex₄HexNAc₃dHex₂, and Hex₁₁HexNAc₂ were resistant to the tested exoglycosidases. The first two proposed monosaccharide compositions contain more than one deoxyhexose residues suggesting that they are protected from glycosidase digestions by the second dHex residues such as α2-, α3-, or α4-linked fucose residues, preferentially present in Fucα2Gal, Fucα3GlcNAc, and/or Fucα4GlcNAc epitopes. The last proposed monosaccharide composition contains two hexose residues more than the largest typical mammalian high-mannose type N-glycan, suggesting that it contains glucosylated structures including (Glc60 →)₂Hex₉HexNAc₂, preferentially α- and/or α3-linked Glc and even more preferentially present in the diglucosylated N-glycan (GlcαGlcα→)Man₉GlcNAc₂.

The compiled neutral N-glycan fraction glycan structures based on the exoglycosidase digestions of BM MSC are presented in Table 11

Osteoblast-Differentiated BM MSC

The analysis of osteoblast differentiated BM MSC are presented in Table 12 allowing comparison of differentiation specific changes in CB MSC. The exoglycosidase profiles produced for BM MSC and osteoblast differentiated BM MSC are characteristic for the two cell types. For example, signals at m/z 1339, 1784, and 2466 are digested differentially in the two experiments. Specifically, the presence of β1,3-galactosidase sensitive neutral N-glycan signals in osteoblast differentiated BM MSC indicate that the differentiated cells contain more β1,3-linked galactose residues than the undifferentiated cells.

The sialidase analysis performed for the acidic N-glycan fraction of BM MSC supported the proposed monosaccharide compositions based on sialylated (NeuAc or NeuGc containing) N-glycans in the acidic N-glycan fraction.

Analysis of CB MSC Neutral Glycan Graction by Exoglycosidases

The results of the analysis by β1,4-galactosidase and β-glucosaminidase are presented in Table 13 The results suggest that also in CB MSC neutral N-glycans containing non-reducing terminal β1,4-linked galactose residues are abundant, and they suggest the presence of characteristic non-reducing terminal epitopes for most of the observed glycan signals. The analysis of adipocyte differentiated CB MSC are presented in Table 14, allowing comparison of differentiation specific changes in CB MSC, similarly as described above for BM MSC.

The sialidase analysis performed for the acidic N-glycan fraction of CB MSC supported the proposed monosaccharide compositions based on sialylated (NeuAc or NeuGc containing) N-glycans in the acidic N-glycan fraction.

### Example 10

### Revealing Protease Sensitive and Insensitive Antibody Target Structures

Bone marrow mesenchymal stem cells as described in examples above were analyzed by FACS analysis. Several antigen structures are essentially not observed or these are observed in reduced amount in FACS analysis of cell surface antigens when cells are treated (released from cultivation) by trypsin but observable after Versene treatment (0.02% EDTA in PBS). This was observed for example by labelling of the mesenchymal stem cells by the antibody GF354, and GF275, with major part trypsin sensitive target structures and by the antibody GF302, which target structure is practically totally trypsin sensitive.

### Example 11

### Isolation and Characterization of Protease Released Glycopeptides Comprising Specific Binder Target Structures

Glycopeptides are released by treatment of stem cells by protease such as trypsin. The glycopeptides are isolated chromatographically, a preferred method uses gel filtration chromatography in Superdex (Amersham Pharmacia (GE)) column (Superdex peptide or superdex 75), the peptides can be observed in chromatogram by tagging the peptides with specific labels or by UV absorbance of the peptide (or glycans). Preferred samples for the method includes mesenchymal stem cells in relatively large amounts (millions of cells) and preferred antibodies, which are used in this example includes e.g. antibodies GF354, GF275 or GF 302 or antibodies or other binders such as lectins with similar specificty.

The isolated glycopeptides are then run through a column of immobilized antibody (e.g. antibody immobilized to cyanogens promide activated column of Amersham Pharmacia (GE healthcare division or antibody immobilized as described by Pierce catalog)). The bound and/or weakly bound and chromatographically retarded fraction(s) is(are) collected as target peptide fraction. In case of high affinity binding the glycan is eluted with 100-1000 mM monosaccharide or monosaccharides cprresponding to the target epitope of the antibody or by mixture of monosaccharides or oligosaccharides and/or with high salt concentration such as 500-1000 mM NaCl. The glycopeptides are analysed by glycoproteomic methods using mass spectrometry to obtain molecular mass and preferably also fragmentation mass spectrometry in order to sequence the peptide and/or the glycan of the glycopeptide.

In alternative method the glycopeptides are isolated by single affinity chromatography step by the binder affinity chromatography and analysed by mass spectrometry essentially similarily as described e.g. in Wang Y et al (2006) Glycobiology 16 (6) 514-23, but lectin affinity chromatography is replaced by affinity chromatography by immobilized antibodies, such as preferred antibodies or binder described above in this example.

### Example 12

### Glycolipid and O-Glycan Analysis of Cellular Glycan Types

The glycosphingolipid glycan and reducing O-glycan samples were isolated from studied cell types, analyzed by mass spectrometry, and further analyzed by expoglycosidase digestions combined with mass spectrometry as described in the present invention and the preceding Examples. Non-reducing terminal epitopes were analyzed by digestion of the glycan samples with S. pneumoniae β1,4-galactosidase (Calbiochem), bovine testes β-galactosidase (Sigma), A. ureafaciens sialidase (Calbiochem), S. pneumoniae α2,3-sialidase (Calbiochem), S. pneumoniae β-N-acetylglucosaminidase (Calbiochem), X. manihotis α1,3/4-fucosidase (Calbiochem), and α1,2-fucosidase (Calbiochem). The results were analyzed by quantitative mass spectrometric profiling data analysis as described in the present invention. The results with glycosphingolipid glycans are summarized in Table 21 including also core structure classification determined based on proposed monosaccharide compositions as described in the footnotes of the Table. Analysis of neutral O-glycan fractions revealed quantitative differences in terminal epitope glycosylation as follows: non-reducing terminal type 1 LacNAc (β1,3-linked Gal) had above 5% proportion only in hESC and non-reducing terminal type 2 LacNAc (β1,4-linked Gal) had above 95% proportion in CB MNC, CB MSC, and BM MSC. Fucosylation degree of type 2 LacNAc containing O-glycan signals at m/z 771 (Hex₂HexNAc₂) and 917 (Hex₂HexNAc₂dHex₁) was 64% in CB MNC, 47% in CB MSC, and 28% in hESC.

In conclusion, these results from O-glycans and glycosphingolipid glycans demonstrated significant cell type specific differences and also were significantly different from N-glycan terminal epitopes within each cell type analyzed in the present invention.

### Example 13

### Endo-β-Galactosidase Analysis of Cellular Glycan Types

Endo-β-Galactosidase Reaction Conditions

The substrate glycans were dried in 0.5 ml reaction tubes. The endo-β-galactosidase (E. freundii, Seikagaku Corporation, cat no 100455, 2.5 mU/reaction) reactions were carried out in 50 mM Na-acetate buffer, pH 5.5 at 37° C. for 20 hours. After the incubation the reactions mixtures were boiled for 3 minutes to stop the reactions. The substrate glycans were purified using chromatographic methods according to the present invention, and analyzed with MALDI-TOF mass spectrometry as described in the preceding Examples.

In similar reaction conditions with with 2 nmol of each defined oligosaccharide control, the reaction produced signal at m/z 568 (Hex₂HexNAc₁) as the major reaction product from lacto-N-neotetraose and para-lacto-N-neohexaose, but not from lacto-N-neohexaose or para-lacto-N-neohexaose monofucosylated at the 3-position of the inner GlcNAc residue; and sialylated signal corresponding to NeuAc₁Hex₂HexNAc₁ from α3′-sialyl-lacto-N-neotetraose. These results confirmed the reported specificities for the enzyme in the employed reaction conditions.

BM and CB MSC O-glycans. The major digestion product in both BM MSC and CB MSC neutral O-glycans was the signal at m/z 568 (Hex₂HexNAc₁), corresponding to a non-reducing non-fucosylated terminal glycan fragment. CB MNC O-glycans also contained a major digestion product at m/z 714 (Hex₂HexNAc₁dHex₁), corresponding to a fucosylated fragment.

BM MSC N-glycans. The major digestion product in BM MSC neutral N-glycans was the signal at m/z 568 (Hex₂HexNAc₁), indicating the presence of poly-LacNAc sequences in the N-glycans. The major sensitive structures were the signals at 1825 (Hex₆HexNAc₄) and 1987 (Hex₇HexNAc₄), indicating that the N-glycan structures included in these signals contained hybrid-type and poly-N-acetyllactosamine sequences.

CB MNC glycosphingolipid glycans. The major digestion product in CB MNC neutral glycosphingolipid glycans was the signal at m/z 568 (Hex₂HexNAc₁), indicating the presence of non-fucosylated poly-LacNAc sequences. Further, signals at 714 (Hex₂HexNAc₁dHex₁) and 1225 (Hex₃HexNAc₂dHex₂) indicated the presence of fucosylated poly-LacNAc sequences.

Major sensitive signals included 1095 (Hex₄HexNAc₂), 1241 (Hex₄HexNAc₂dHex₁), 876 (Hex₃HexNAc₁dHex₁), 1606 (Hex₅HexNAc₃dHex₁), 1460 (Hex₅HexNAc₃), and 933 (Hex₃HexNAc₂), indicating presence of both linear non-fucosylated and multifucosylated poly-LacNAc. Residual signals left in the sensitive signals after digestion indicated presence of lesser amounts of also branched poly-LacNAc sequences.

CB MSC glycosphingolipid glycans. The major digestion product in CB MSC neutral glycosphingolipid glycans was the signal at m/z 568 (Hex₂HexNAc₁), indicating the presence of non-fucosylated poly-LacNAc sequences. Major sensitive signals were signals at m/z 1095 (H4N2), 933 (Hex₃HexNAc₂), and 1460 (Hex₅HexNAc₃). Compared to CB MNC results, CB MSC had less sensitive structures although the glycan profiles contained same original signals than CB MNC, indicating that in CB MSC the poly-N-acetyllactosamine sequences of glycosphingolipid glycans were more branched than in CB MNC.

In conclusion, the profiles of endo-β-galactosidase reaction products efficiently reflected cell type specific glycosylation features as described in the preceding Examples and they represent an alternative and complementary method for analysis of cellular glycan types. Further, the present results demonstrated the presence of linear, branched, and fucosylated poly-LacNAc in all studied cell types and in different glycan types including N- and O-glycans and glycosphingolipid glycans; and further quantitative and cell-type specific proportions of these in each cell type, which are characteristic to each cell type.

### Example 14

### Analysis of O-Glycosylation in Stem Cells and Differentiated Cells

Comparison of bone marrow mesenchymal stem cells (BM MSC) and osteoblast-differentiated BM MSC (OB) with regard to their O-glycosylation was performed.

Experimental Procedures

Cell samples were prepared as described in the preceding Examples. O-glycans were detached from cellular glycoproteins by non-reductive β-elimination with saturated ammonium carbonate in concentrated ammonia at 60° C. essentially as described by Huang et al. (Anal. Chem. 2000, 73 (24) 6063-9) and purified by solid-phase extraction steps with C18 silica, cation exchange resin, and graphitized carbon. O-glycan profiles were analyzed by MALDI-TOF mass spectrometry separately for isolated neutral and acidic O-glycan fractions, and the result was expressed as % of total O-glycan profile for each detected O-glycan component. The purification and analysis steps were performed essentially as described in WO2007012695.

Results

Acidic O-Glycans

Table 22 describes the analysis results of O-glycans in BM MSC and OB and their comparison.

In BM MSC compared to OB, over 2 times overexpressed non-sialylated O-glycan components with sulphate or phosphate ester, preferentially sulphate ester, included: H7N2P2, H5N4P2, H6N2F1P1, H6N4P2, H3N3P1, H5N4F1P1, H6N2P2, H4N3P1, H5N4F1P1, and H4N3F1P1.

Further, over 2 times overexpressed O-glycan components with non-fucosylated chain and H3N3 or larger core composition, included in BM MSC: S1H3N3, H3N3P1, S2H3N3, S1H4N4; while OB expressed only a fucosylated variant S1H3N3F1 that was not expressed in BM MSC.

Further, major overexpressed O-glycan components in BM MSC, with sialylation, fucosylation, and core composition wherein n(Hex)=n(HexNAc)+1, included: S2H2N1F1 and S2H3N2F1.

OB expressed preferentially sialylated O-glycan components with H1N1 or H2N2 core composition: S2H2N2, S1H2N2, S2H1N1, and S1H2N2P1, whose expression was not as prominent in BM MSC.

Non-sialylated O-glycan component with H2N2 core composition, H2N2P 1, was expressed as a major O-glycan in both BM MSC and OB.

Neutral O-Glycans

Four most common neutral O-glycan components were detected as follows: in BM MSC, they were H3N1, H2N2, H2N1, and H1N2; and in OB, they were H2N2, H3N1, H2N1, and H1N2. Therefore, no significant difference was detected between the cell types.

Conclusions

BM MSC and OB differentiated from them were characterized by following O-glycosylation features:

Expression in both BM MSC and OB:

1) Prominent sulphation and/or phosphorylation, preferentially sulphation, more preferentially when sulphation replaces sialylation as the acidic determinant in the O-glycan chain. A major sulphated O-glycan component in both cell types is preferentially H2N2P1, wherein sulphate or phosphate replaces sialic acid. Preferentially, the structure includes sulphate ester of H2N2 O-glycan, more preferentially of a sulphated mucin-type O-glycan with N-acetyllactosamine at the non-reducing end and Galβ3GalNAc at the reducing end, most preferentially a Core 2 type O-glycan.

Overexpression in BM MSC compared to OB:

1) Sulphated or phosphorylated O-glycans without sialylation, preferentially sulphated O-glycans.

2) O-glycan components with non-fucosylated chain and H3N3 or larger core composition, preferentially including poly-N-acetyllactosamine modified O-glycans.

3) O-glycan components with sialylation, fucosylation, and core composition wherein n(Hex)=n(HexNAc)+1, including preferentially S2H2N1F1 and S2H3N2F1.

Overexpression in OB compared to BM MSC:

1) Sialylated O-glycan components with H1N1 or H2N2 core composition. Preferentially, the structures include sialylated mucin-type O-glycans with or without N-acetyllactosamine at the non-reducing end and Galβ3GalNAc at the reducing end, most preferentially Core 1 and/or Core 2 type O-glycans.

### Example 15

### Immunohistochemical Stainings of Mesenchymal Stem Cells and Osteogenic Cells Differentiated from Them

Experimental Procedures

Cell samples. Mesenchymal stem cells (MSCs) from bone marrow were generated and cultured in proliferation medium as described above. MSCs were cultured in differentiation medium (proliferation medium including 0.1 μmol/L dexamethasone, 10 mmol/L β-glycerophosphate, and 50 μmol/L ascorbic acid) for 6 weeks to induce osteogenic differentiation. Differentiation medium was refreshed twice a week throughout the differentiation period.

Antibodies. Antibodies, their antigens/epitopes and codes used in the immunostainings are listed in Table 25.

Immunohistochemistry (IHC). Bone-marrow derived mesenchymal stem cells on passages 9-12 were grown on CC2 treated glass 8-chamber slides (Lab-TekII, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) were diluted in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK). Immunostainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400×magnification.

Fluorescence activated cell sorting (FACS) analysis. Proliferating MSCs on passage 12 were detached from culture plates by 0.02% Versene solution (pH 7.4) for 45 minutes at 37° C. Cells were washed twice with 0.3% HSA-PBS solution before antibody labelling. Primary antibodies were incubated (4 μl/100 μl cell suspension/50 000 cells) for 30 minutes at RT and washed once with 0.3% HSA-PBS before secondary antibody detection with Alexa Fluor 488 goat anti-mouse (1:500) for 30 minutes at RT in the dark. As a negative control cells were incubated without primary antibody and otherwise treated similar to labelled cells. Cells were analysed with BD FACSAria (Becton Dickinson) using FITC detector at wavelength 488. Results were analysed with BD FACSDiva software version 5.0.1 (Becton Dickinson).

Results and Discussion

Based on both FACS and IHC results, antibodies GF307 (sLex), GF353 (SSEA-3) and GF354 (SSEA-4) are markers for mesenchymal stem cells, since their expression on the cell surface clearly decreases during osteogenic differentiation (Table 23, FIG. 19). Additionally, in FACS analysis antibodies GF277 (sTn), GF278 (Tn), GF295 (pLN) and GF306 (sLea) show more reactivity with MSCs than with osteogenic cells, indicating that these markers would also be associated with mesenchymal stem cells.

When BM-MSCs were differentiated for osteogenic direction for 6 weeks, their cell surface expressed more of the following glycans: GF275 (CA15-3), GF296 (asialo GM1), GF297 (GL4), GF298 (Gb3), GF300 (asialo GM2), GF302 (H type 2), and GF304 (Lea) based on FACS analysis (Table 23, FIG. 19). On the other hand, IHC results showed that staining of GF276 (oncofetal antigen), GF277 (sTn), GF278 (Tn), and GF303 (H Type 1) clearly increased during osteogenic differentiation (Table 23). Interestingly, antibodies GF276 (oncofetal antigen) and GF303 (H Type 1) showed no reactivity when used in FACS, but instead showed clear staining in IHC only in osteogenic cells, being therefore markers for osteogenic differentiation. Additionally, antibodies GF296 (asialo GM1), GF300 (asialo GM2) and GF304 (Lea) were totally negative in IHC, but showed reactivity in FACS analysis, being markers for osteogenic lineage.

The discrepancy between FACS and IHC with some antibodies may result from several reasons. First, cells undergo different treatments before incubation with antibodies, e.g. cells are fixed for IHC, but not for FACS, and cells are adherent in IHC and in suspension for FACS analysis. Furthermore, glycan epitopes that are usually attached to lipids, e.g. GF296 (asialo GM1) and GF300 (asialo GM2), may behave differently in IHC and FACS due to the biochemical differences in experimental procedures. Additionally, the affinity and avidity of the antibodies may be different affecting to the results in stable IHC compared to fluidic system in FACS analysis. However, both methods are widely used in biological studies and the results should be considered valid with both methodologies.

### Example 16

### Revealing Protease Sensitive and Insensitive Antibody Target Structures

Bone marrow mesenchymal stem cells and osteogenic cells derived thereof as described in examples above were analyzed by FACS analysis. Several antigen structures are essentially not observed or these are observed in reduced amount in FACS analysis of cell surface antigens when cells are treated (released from cultivation) by trypsin (0.25%), but observable after Versene treatment (0.02% EDTA in PBS). Several glycan epitopes, e.g. GF277 (sTn), GF278 (Tn), GF295 (pLN), GF296 (asialo GM1), GF299 (Forssman antigen), GF300 (asialo GM2), GF302 (H Type 2), GF304 (Lea), and GF306 (sLea), were practically totally destroyed by trypsin treatment in both BM-MSCs and osteogenic cells derived thereof (Table 24). Some glycan epitopes, such as GF275 (CA15-3), GF307 (sLex), and GF354 (SSEA-4) were partially sensitive for trypsin treatment.

### Example 17

### Comparison of Differentiated and Non-Differentiated MSCs and Identification of a Fucosyl Glycan Marker

Mesenchymal Stem Cells

Mesenchymal stem cells (MSC:s) are fibroblast-like adult multipotent progenitor cells that can be isolated from various sources such as bone marrow or cord blood. MSC:s are capable of differentiating into mesenchymal cell types like osteoblasts, chondroblasts and adipocytes.

Objectives

This study was carried out to characterize the N-glycome of human mesenchymal stem cells. Stem cells hold an enormous therapeutic potential in regenerative medicine. However, before stem cells can be used in the clinical practice, there is a need for methods to thoroughly characterize them, to distinguish them from other cells, and to control variation within and between different cell lines. A glycomic approach to study stem cells provides an ideal platform to solve these issues. Modern mass spectrometric methods provide the means to characterize the glycome even when the amount of sample available is very limited.

Materials and Methods

Human mesenchymal stem cells were isolated from bone marrow and cultured. Osteogenic differentiation was induced by placing the cells in osteogenic induction medium. The N-linked glycans were enzymatically released with protein N-glycosidase F from about 100 000-1 000 000 cells. The total glycan pools (picomole quantities) were purified with microscale solid-phase extraction and divided into neutral and sialylated glycan fractions. The glycan fractions were analyzed by MALDI-TOF mass spectrometry with a Bruker Ultraflex TOF/TOF instrument. Exoglycosidase digestions were carried out to further characterize terminal epitopes. In addition, carbohydrate epitopes were studied by immunofluorescent staining to support the mass spectrometric data.

Results and Conclusions

More than one hundred glycan signals were detected for both cell types. Of these some signals were characteristic of stem cells and decreased upon differentiation, whereas other signals became more prominent upon differentiation. Specific structural features associated with either stem cells or differentiated cells could be seen by exoglycosidase digestions and immunofluorescent stainings. In conclusion, mesenchymal stem cells have a characteristic N-glycan profile that changes upon differentiation. The information on the stem cell glycome can be used to evaluate the differentiation stage of stem cells and to develop new stem cell markers (e.g. for antibody development) as well as to study the interactions of stem cells with their niches and thus develop improved in vitro culture systems.

The FIG. 1 shows difference in N-glycan profiles of MSC cells and their differentiated variant. The differences of signals in FIG. 1b for neutral glycans and FIG. 1d for acidic glycans were used to identify key structures altering during differentiation. FIG. 2 shows cleavage of fucosylresidue by specific fucosidase from di- and trifucosylated biantennary neutral N-glycans. Combination of the result with cleavage by β4-galactosidase indicates presence of Lewis x structure on N-glycans. FIG. 3 shows staining by an anti-sialyl-Lewis x antibody binding to the sialylated terminal epitope analogous to Lewis x, see Example 19 for details.

### Example 18

### Mesenchymal Stem Cell Glycosylation

Stem cell and differentiated cell samples were obtained and analyzed essentially as described in WO/2007/006870, more specific procedures are listed below.

Isolation and culture of bone marrow derived stem cells. Bone marrow (BM)—derived MSCs were obtained as described by Leskelä et al. (2003). Briefly, bone marrow obtained during orthopedic surgery was cultured in Minimum Essential Alpha-Medium (α-MEM), supplemented with 20 mM HEPES, 10% FCS, 1×penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

Five BM MSC lines and osteoblast differentiated cells derived therefrom were analyzed in the present analyses to obtain statistically significant results about MSC and differentiated cell glycosylation.

Glycan isolation. Asparagine-linked glycans were detached from cellular glycoproteins by F. meningosepticum N-glycosidase F digestion (Calbiochem, USA) essentially as described (Nyman et al., 1998). The detached glycans were divided into sialylated and non-sialylated fractions based on the negative charge of sialic acid residues. Cellular contaminations were removed by precipitating the glycans with 80-90% (v/v) aqueous acetone at −20° C. and extracting them with 60% (v/v) ice-cold methanol essentially as described previously (Verostek et al., 2000). The glycans were then passed in water through C₁₈ silica resin (BondElut, Varian, USA) and adsorbed to porous graphitized carbon (Carbograph, Alltech, USA) based on previous method (Davies et al., 1993). The carbon column was washed with water, then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C₁₈ silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used. The glycan analysis method was validated by subjecting human cell samples to analysis by five different persons. The results were highly comparable, especially by the terms of detection of individual glycan signals and their relative signal intensities, showing that the reliability of the present methods is suitable for comparing analysis results from different cell types.

Mass spectrometry and data analysis. MALDI-TOF mass spectrometry was performed with a Bruker Ultraflex TOF/TOF instrument (Bruker, Germany) essentially as described (Saarinen et al., 1999). Relative molar abundancies of both neutral and sialylated glycan components can be accurately assigned based on their relative signal intensities in the mass spectra (Naven and Harvey, 1996; Papac et al., 1996; Saarinen et al., 1999; Harvey, 1993). Each step of the mass spectrometric analysis methods were controlled for their reproducibility by mixtures of synthetic glycans or glycan mixtures extracted from human cells. The mass spectrometric raw data was transformed into the present glycan profiles by carefully removing the effect of isotopic pattern overlapping, multiple alkali metal adduct signals, products of elimination of water from the reducing oligosaccharides, and other interfering mass spectrometric signals not arising from the original glycans in the sample. The resulting glycan signals in the presented glycan profiles were normalized to 100% to allow comparison between samples.

Glycosidase analysis. Glycan fractions were subjected to specific exoglycosidase digestions, preferably with the following enzymes: Jack bean α-mannosidase (Canavalia ensiformis; Sigma, USA); β1,4-galactosidase from S. pneumoniae (recombinant in E. coli; Calbiochem, USA); recombinant β1,3-galactosidase (Calbiochem, USA); β-glucosaminidase from S. pneumoniae (Calbiochem, USA); α2,3 -sialidase from S. pneumoniae (Calbiochem, USA), α2,3/6/8/9-sialidase from A. ureafaciens (Calbiochem, USA); α1,2-fucosidase and α1,3/4-fucosidase from X. manihotis (Calbiochem, USA). Reactions were performed and analyzed with mass spectrometry by comparison to the undigested samples essentially as described (Saarinen et al., 1999). The specificity of the enzymes was controlled with glycans isolated from human tissues as well as purified oligosaccharides, analyzed similarly by mass spectrometry as the analytical reactions.

Results

Relative comparison of MALDI-TOF mass spectrometric profiling results about N-glycan fractions isolated from BM MSC and osteoblast differentiated cell samples are presented in Tables 1 and 3, revealing specific MSC-associated and differentiated cell associated glycan signals, glycan structural features, and glycan signal groups expressing such structural features, as analyzed in the detailed description of the present invention. Variation analysis between the analyzed five cell lines are presented in Tables 2 and 4, showing which glycan signals and glycan signal groups, and subsequently which glycan structural features are subject to either little or much variation between the analyzed samples.

Structural assignments for the proposed monosaccharide compositions within the detected N-glycan signals in BM MSC are presented in Tables 5 and 6.

1H NMR analysis results from the BM MSC samples are presented in Tables 7 and 8, showing major N-glycan components and glycan structural features in the MSC samples.

Table 9 exemplifies exoglycosidase digestion results from BM MSC neutral and sialylated N-glycan fractions, and shows major non-reducing glycan epitopes within glycan structures under each detected glycan signal; the table also revealed combinations of epitopes within same structures, revealing structural data of the detected glycan components according to the present invention.

Major structures detected to carry β1,4-linked galactose were:

H4N3 1298 0-1 β1,4-Gal residues

H5N3 1460 0-2 β1,4-Gal residues

H6N2F1 1565 1 β1,4-Gal residue

H5N3F1 1606 0-1 β1,4-Gal residues

H6N3 1622 0-3 β1,4-Gal residues

H5N4 1663 2 β1,4-Gal residues

H6N3F2 1768 1-2 β1,4-Gal residues

H7N3 1784 1-4 β1,4-Gal residues

H5N4F1 1809 1-2 β1,4-Gal residues

H5N4F2 1955 1 β1,4-Gal residue

H6N4F1 1971 2-3 β1,4-Gal residues

H5N5F1 2012 2 β1,4-Gal residues

H6N5 2028 3 β1,4-Gal residues

H4N5F3 2142 0-1 β1,4-Gal residues

H6N5F1 2174 3 β1,4-Gal residues

H11N2 2229 1 β1,4-Gal residue

H7N6 2393 1-4 β1,4-Gal residues

H7N6F1 2539 4 β1,4-Gal residues

The detected structures included hybrid-type (e.g. H7N3), biantennary complex-type (e.g. H5N4, H5N4F1, H5N4F2), triantennary (e.g. H6N5) and tetrantennary complex-type (e.g. H7N6F1) N-glycans, and sialylated counterparts of the detected neutral N-glycans (e.g. sialylated H5N4F1, H5N4F2); and Table 9 shows more detailed data. The results indicate non-reducing type II N-acetyllactosamine (LacNAc, Galβ4GlcNAc) epitopes in the structures.

Major structures detected to carry α1,3/4-linked fucose were:

H2N2F1 917 0-1 α1,3- or α1,4-linked fucose residues

H3N2F1 1079 0-1 α1,3- or α1,4-linked fucose residues

H4M2F1 1241 0-1 α1,3- or α1,4-linked fucose residues

H3N3F1 1282 0-1 α1,3- or α1,4-linked fucose residues

H5N2F1 1403 0-1 α1,3- or α1,4-linked fucose residues

H4N3F1 1444 0-1 α1,3- or α1,4-linked fucose residues

H3N4F1 1485 0-1 α1,3- or α1,4-linked fucose residues

H4N3F2 1590 0-2 α1,3- or α1,4-linked fucose residues

H5N3F1 1606 0-1 α1,3- or α1,4-linked fucose residues

H3N5F1 1688 0-1 α1,3- or α1,4-linked fucose residues

H5N3F2 1752 0-2 α1,3- or α1,4-linked fucose residues

H6N3F1 1768 0-1 α1,3- or α1,4-linked fucose residues

H4N4F2 1793 1 α1,3- or α1,4-linked fucose residue

H5N4F1 1809 0-1 α1,3- or α1,4-linked fucose residues

H6N4F1 1971 0-1 α1,3- or α1,4-linked fucose residues

H6N5F1 2174 0-1 α1,3- or α1,4-linked fucose residues

H5N5F3 2304 0-3 α1,3- or α1,4-linked fucose residues

H6N5F2 2320 0-2 α1,3- or α1,4-linked fucose residues

H6N5F4 2612 0-4 α1,3- or α1,4-linked fucose residues

The detected structures included hybrid-type (e.g. H5N3F2), biantennary complex-type (e.g. H5N4F2, H5N4F3), triantennary (e.g. H6N5F2) complex-type N-glycans, and sialylated counterparts of the detected neutral N-glycans (e.g. sialylated H5N4F1, H5N4F2); and Table 9 shows more detailed data. The results indicate Lewis x epitopes (Lex, Galβ4(Fucα3)GlcNAc) in the structures wherein type II LacNAc forms the N-glycan antennae backbones; and in BM MSC the type II LacNAc was shown to be the major antenna backbone.

The presence of corresponding sialylated glycan compositions as shown in Table 9, indicates that the major similar sialylated epitopes were sialyl-LacNAc, predominantly α2,3-sialylated type II LacNAc, and sialyl-fucosylated LacNAc, predominantly sialyl-Lex (sLex, Neu5Acα3Galβ4(Fucα3)GlcNAc). Corresponding structural assignments are shown in the Tables of the present invention and described in the detailed description of the invention.

The digestion results also indicated α1,2-linked fucose epitopes indicating H type 2 epitopes (H-2, Fucα2Galβ4GlcNAc) in the structures wherein type II LacNAc forms the N-glycan antennae backbones; and monoclonal antibody results with anti-H-2 antibodies further showed that such epitopes were more common in osteoblast differeantiated cells than in BM MSC.

Similarly, the present results as exemplified in Table 9 indicated the presence of non-reducing terminal α-mannose, β1,3-linked galactose, β-linked N-acetylglucosamine, and linear poly-N-acetyllactosamine; more specifically in the N-glycan compositions and exemplary amounts as specified in Table 9. These are described in more detail under the detailed description of the invention.

According to the present invention and as described in the detailed description of the invention, the combination of the present exoglycosidase digestion results as exemplified in Table 9 with the other structural characterization and classification data presented by the inventors, revealed major non-reducing terminal N-glycan structures of BM MSC and cells derived therefrom.

### Example 19

### Immunostaining

Immunohistochemistry (IHC). Bone-marrow derived mesenchymal stem cells on passages 9-12 were grown on CC2 treated glass 8-chamber slides (Lab-TekII, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) were diluted in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK). Immunostainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

The results with staining mesenchymal cells by specific clone of antibody to sialyl Lewis x (GF307) are shown in FIG. 3. The specific antibody type show specificity for non-differentiated hMSCs. The specification of antibody is in Table 25.

### Example 20

Antibody profiling of bone marrow derived and cord blood derived mesenchymal stem cell lines

Experimental Procedures

Bone marrow derived mesenchymal stem cell lines (BM-MSC). Isolation and culture of BM-MSCs, as well as osteogenic differentiation of BM-MSCs, were performed as described in Example 1.

Umbilical cord blood mesenchymal stem cell (CB-MSC) isolation and culture. The isolation and culture of CB-MSCs was performed as described in Example 1 with some modifications. Osteogenic differentiation of CB-MSCs was induced as described for BM-MSCs for 16 days.

Adipogenic differentiation of CB-MSCs. Cells were grown in proliferation medium to almost confluence after which the adipogenic induction medium including α-MEM Glutamax supplemented with 10% FCS, 20 mM Hepes, 1×penicillin-streptomycin, 0.1 mM Indomethasin (all from Sigma), 0.5 mM IBMX-22, 0.4 μg/ml dexamethasone and 0.5 μg/ml Insulin (all three from Promocell) was added. After 3 days, terminal adipogenic differentiation medium including α-MEM Glutamax supplemented with 10% FCS, 20 mM Hepes, 1×penicillin-streptomycin, 0.1 mM Indomethasin (all from Sigma), 0.5 μg/ml Insulin and 3.0 μg/ml Ciglitazone (both two from Promocell) was added and cells were grown for 14 days (altogether 17 days) in 5% CO₂ at 37° C. Differentiation medium was refreshed twice a week throughout the differentiation period.

Flow cytometric analysis of mesenchymal stem cell phenotype. Both BM and CB derived MSCs were phenotyped by flow cytometry (BD FACSAria, Becton Dickinson). FITC, APC or PE conjugated antibodies against CD13, CD14, CD29, CD34, CD44, CD45, CD49e, CD73, CD90, HLA-DR and HLA-ABC (all from BD Biosciences) and CD105 (Abcam Ltd.) were used for direct labelling. For staining, cells in a small volume, i.e. 5×10⁴ cells/100 μl 0.3% ultra pure BSA, 2 mM EDTA-PBS buffer, were aliquoted to FACS-tubes. One microliter of each antibody was added to cells and incubated for 30 min at +4° C. Cells were washed with 2 ml of buffer and centrifuged at 300×g for 4 min. Cells were suspended in 200 μl of buffer for flow cytometric analysis.

Cell harvesting for antibody staining. Both BM and CB-MSCs were detached from cell culture plates with 2 mM EDTA-PBS solution (Versene), pH 7.4, for approximately 30 minutes at 37° C. Both osteogenic and adipogenic cells were detached with 10 mM EDTA-PBS solution, pH 7.4, for 30 minutes and 5 minutes at 37° C., respectively. Since the differentiated cells detached from culture plates as clusters, they were suspended by pipetting with Pasteur-pipette or by vortexing and by suspending through an 18 gauge needle to get a single cell suspension. Finally, the cell suspension was filtered through a 50 μm filter to get rid of unsuspended cell aggregates. Harvested cells were centrifuged at 300×g for 4 minutes and suspended for small volume of 0.3% ultra pure BSA (Sigma), 2 mM EDTA-PBS buffer.

Primary antibody staining. BM and CB derived cells were aliquoted to FACS-tubes in a small volume, i.e. 5-7×10⁴ cells/100 μl 0.3% ultra pure BSA, 2 mM EDTA-PBS buffer. Four microliters of anti-glycan primary antibody was added to cell suspension, vortexed and incubated for 30 min at room temperature. Cells were washed with 2 ml of buffer and centrifuged for 4 min at 300×g, after which the supernatant was removed. Primary antibodies used for staining are listed in Table 25.

Secondary antibody staining. AlexaFluor 488-conjugated anti-mouse (1:500, Invitrogen) and anti-rabbit (1:500, Molecular Probes), as well as FITC-conjugated anti-rat (1:320, Sigma) and anti-human λ (1:1000, Southern Biotech) secondary antibodies were used for appropriate primary antibodies. Secondary antibodies were diluted in 0.3% ultra pure BSA, 2 mM EDTA-PBS buffer and 100 μl of dilution was added to the cell suspension. Samples were incubated for 30 min at room temperature in the dark. Cells were washed with 2 ml of buffer and centrifuged for 4 min at 300×g. Supernatant was removed and cells were suspended in 200 μl of buffer for flow cytometric analysis. As a negative control cells were incubated without primary antibody and otherwise treated similarly to labelled cells.

Flow cytometric analysis. Cells with fluorescently labelled antibodies were analysed with BD FACSAria (Becton Dickinson) using FITC detector at wavelength 488. Results were analysed with BD FACSDiva software version 5.0.1 (Becton Dickinson).

Results and Discussion

Flow cytometric analysis of mesenchymal stem cell phenotype. Both BM and CB-MSCs were negative for hematopoietic markers CD34, CD45 and CD14. The cells stained positively for the CD13 (aminopeptidase N), CD29 (β1-integrin), CD44 (hyaluronan receptor), CD73 (SH3), CD90 (Thy-1), CD105 (SH2/endoglin) and CD49e. The cells stained also positively for HLA-ABC, but negatively for HLA-DR.

Anti-glycan antibody profiling of BM-MSCs. BM-MSCs and osteogenic cells (BM-OG) differentiated thereof were analyzed with up to 60 anti-glycan antibodies by flow cytometry and also with 29 antibodies by immunohistochemistry (IHC). The results of BM-MSC staining are presented in Table 26 and in Figures.

General observations. There seems not to be a single specific glycan epitope analyzed absolutely specific only for one total population of specific MSCs or a cell population differentiated into osteogenic lineage, but not for other cell population. Instead there seems to be enrichment of certain glycan epitopes in stem cells and in differentiated cells. In some cases the antibodies recognize epitopes, which are highly or several fold enriched in a specific cell type or present above the current FACS detection limit in a part of a cell population but not in the other corresponding cell populations. It is realized that such antibodies are especially useful for specific recognition of the specific cell population.

Furthermore combination of several antibodies recognizing independent subpopulations of specific cell type cells is useful for recognition positive or negative recognition of larger cell population.

The present invention provides reagents common to mesenchymal cell populations in general or for specific differentiation stage of mesenchymal cells such as mesenchymal stem cells, or differentiated mesenchymal stem cells in general or specific for the specific differentiated cell populations such as adipocytes or osteoblasts. Furthermore the invention reveals specific marker structures for mesenchymal stem cell derived from specific tissue types such as cord blood or bone marrow. The invention is further directed to the use of the target structures and specific marker

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells. It is realized that tissues comprising stem cells usually contain these in privitive stem cell stage and highly expressed markers according can be optimised or selected for the cell isolation. It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

Combination of several antibodies for specific analysis of a mesenchymal cell population would characterize the cell population. In a preferred embodiment at least gone “effectively binding antibody”, recognizing major part (over 35%) or most (50%) of the cell population (preferably more than 30%, an in order of increasing preference more than 40%, 50%, 60%, 70%, 80% and most preferably more than 9%), are selected for the analytic method in combination with at least one “non-binding antibody”, recognizing preferably minor part (preferably from detection limit of the method to low level of recognition, in order of preference less than 10%, 7%, 5%, 2% or 1% of cell, e.g 0.2-10% of cells, more preferably 0.2-5% of the cells, and even more preferably 0.5-2% or most preferably 0.5%-1.0%) or no part of the cell population (under or at the the detection limit e.g. inorder of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably 7% 40% and most preferably 10 to 35%. The antibodies are preferanly

The antibodies recognize certain glycan epitopes revealed as target structures according to the invention. It is realized that specificites and affinites of the antibodies vary between the clones. It was realized that certain clones known to recognize certain glycan structure does not necessarily recognize the same call population, actually any of the FACS results with different antibody clones does produce exactly the same recognition pattern of recognition.

The most prominent enrichment in stem cells is SSEA-4 and in osteogenic cells some glycolipid epitopes ganglioseries such as asialo GM1, asialo GM2 and globoseries structures: globotriasyl ceramide Gb3 and globotetraose also known as globoside (GL4 or Gb4) as well as Lewis a and sialylated Ca15-3.

Lewis x structures seems not to be present in quantity over detection level under FACS analysis conditions in a larger part of the MSC cells in the preparations of MSCs or in differentiated cells based on staining with 5 different anti-Lex antibodies. There is however specific Lewis x expression recognizable by specific anti-Lewis x clones.

On the other hand, sialyl Lewis x structures are present on both stem cells and in osteogenic cells and the proportions differ between different anti-sLex antibodies, which is most probably due to the different carriers for sLex epitopes. For example GF526 anti-sLex antibody recognizes only sLex epitope carried by specific O-glycan core II structure. The binding of GF 526 has been determined to be related to P-selectin ligand glycoprotein PSGL-1, which represent the O-glycan effectively in large quantities on certain non-stem cell materials. It is however realised that core II O-glycans have reported on several mucin type O-glycans and the present invention is not limited to analysis of the Core II sLex on PSGL-1 on the mesenchymal stem cells. The carrier and the exact binding epitope of sLex recognized by two other anti-sLex antibodies (GF516 and GF307) appears to include structures other than core II with optimal fine specificty different from the GF. The antibodies with different fine and core/carrier glycan specifity cell populations with different sizes.

Anti-glycan antibody profiling of CB-MSCs. CB-MSCs and both osteogenic and adipocytic cells differentiated thereof were analysed with up to 61 different anti-glycan antibodies by flow cytometry. The results of CB-MSC staining are presented in Table 26 and in Figures. Likewise in BM derived antibody profiling, there seems not to be a single specific glycan epitope determining either CB-MSCs or cells differentiated into osteogenic or adipocytic lineages. Some glycans, e.g. H disaccharide (GF394), TF (GF281), Glycodelin (GF375), Lewis x (GF517) and Galα3Gal (GF413), are highly enriched in CB derived MSCs, but their proportion in the whole stem cell population is rather low (10% or below). Interestingly, there seems to be also glycans, e.g. SSEA-4 (GF354), Lewis c (GF295), SSEA-3 (VPU009), GD2 (GF406), sialyl Lewis x (GF307) and Tra-1-60 (GF415), enriched in stem cells and in adipocytic cells, but not in osteogenic cells. BM-derived cells have not been differentiated into adipocytic direction, so we can not compare the data between different adipocytes from different sources. Osteogenic differentiation induces similar enrichment of glycans both in BM and CB derived cells. Only Gb3, increasing in BM derived osteogenic cells is not increased in CB derived osteogenic cells. Furthermore, gangliosides GT1b, GD2, GD3 and A2B5, not tested in BM-derived cells, are highly enriched in CB derived osteogenic cells. Most of the glycan epitopes revealed by specifc antibodies of the example enriched in CB-derived osteoblasts are also enriched (even with higher percentage) in CB-derived adipocytes, but the invention reveals even for these targets there is differences in expression levels between the cell types allowing characterization of both differentiation lineages. An interesting group of glycan epitopes after differentiation is glycan epitopes recognizable by known antibodies against gangliosides, in general increasing from stem cells (<10%) into osteoblasts and adipocytes (50-100%). Unlike in BM-derived MSCs, there seems to be some positivity with anti-Lewis x antibodies GF517 and GF525 in CB derived cells. The results with anti-sialyl-Lewis x antibodies are parallel with both cell types.

### Example 21

### Structures from CB MSC and Osteoblast-Differentiated Cells

Cord blood MSC and cells osteoblast-differentiated from were gathered, their cellular glycosphingolipid glycans isolated and permethylated essentially as described in the preceding Examples, and analyzed by MS/MS analysis (fragmentation mass spectrometry). In the following result listings, the fragments are mainly Na+ adduct ions unless otherwise specified and [ ] indicates undefined monosaccharide sequence. The following glycans produced structure-indicating signals (nomenclature is as described by Domon and Costello, 1988, Glycoconjugate J.).

Acidic Glycolipid Glycans from Osteoblast-Differentiated Cells

m/z 838.39 corresponding monosaccharide composition NeuAcHex2 corresponding to a structure with identical isobaric monosaccharide sequence as the structure GM3; NeuAcα2-3Galβ1-4Glc. This structure is confirmed with fragments B, (m/z 375.94 (M+H⁺)) and Y₂ (m/z 463.01).

m/z 1083.56 corresponding monosaccharide composition corresponding to a structure with identical isobaric monosaccharide sequence as the structure GM2; NeuAcα2-3(GalNAcβ1-4)Galβ1-4Glc. This structure is confirmed with fragments B₁ (m/z 376.03 (M+H+)), Y_{2α} m/z (m/z 708.21), Y_{2β} (m/z 824.30), Y_{2α}/Y_{2β} (m/z 449.03), Y₁ (m/z 258.95).

m/z 1199.63 corresponding monosaccharide composition NeuAc2Hex2 corresponding to a structure with identical isobaric monosaccharide sequence as the structure GD3; NeuAcα2-8NeuAcα2-3Galβ1-4Glc. This structure is confirmed with fragments fragments B₁ (m/z 375.94 (M+H+)), B₂ (m/z 759.13), Y₂ (m/z 463.0) and Y₃ (m/z 824.22).

m/z 1532.83 corresponding monosaccharide composition (NeuAcHex3HexNAc2) corresponding to a structure with identical isobaric monosaccharide sequence as the structure NeuAcα2-3(GlcNAcβ1-4)Galβ1-3/4GlcNAcβ1-3/4Galβ1-3/4Glc which could be confirmed with obtained fragments B₁ (m/z 375.88 (M+H+)), B₂/Y_{4α} (m/z 471.87), Y₃ (m/z 708.04), B₂ (m/z 847.12), Y_{4α} (m/z 1157.50) and Y_{4β} (m/z 1273.66).

m/z 1736.90 corresponding monosaccharide composition (NeuAcHex4HexNAc2) corresponding to a structure with identical isobaric monosaccharide sequence as the structure NeuAcα2-3Galβ1-3/4GlcNAcβ1-3/4Galβ1-3/4GlcNAcβ1-3/4Galβ1-3/4Glc which could be confirmed with obtained fragments B₁ (m/z 375.73 (M+H+)), Y₂ (m/z 462.76), Y₆/B₄ or Y₄/B₆ (m/z 707.73), B₃(m/z 846.93), Y₄ (m/z 911.98), Y₅ (m/z 1156.36), B₅ (m/z 1296.24) and Y₆ (m/z 1359.95).

Neutral Glycolipid Glycans from Osteoblast-Differentiated Cells

m/z 1375.70 corresponding monosaccharide composition (Hex4HexNAc2) corresponding to a structure with identical isobaric monosaccharide sequence as the structure Galβ1-3/4GlcNAcβ1-3/4Galβ1-3/4GlcNAcβ1-3/4Galβ1-3/4Glc which could be confirmed with obtained fragments Y₂ (m/z 462.83), B₂ (m/z 485.78), Y₅/B₃ or Y₃/B₅ (m/z 471.83), Y₃ (m/z 707.88), Y₄ (m/z 912.10) and Y₅ (m/z 1157.42). This sample contained also a minor component representing a branched structure, namely disubstituted Hexβ1-3/4-unit. This observation is based on fragment Y_{3α}/Y_{3β} (m/z 897.46) as well as fragment Y_{2α}/Y_{2β} (m/z 448.80).

Taken together, the present results yielded especially direct evidence for the following specific structures in osteoblast-differentiated MSC glycolipid glycans: GM3, GD3, and GM2 ganglioside-type structures, specifically with disialic acid residues, as well as linear and branched poly-N-acetyllactosamine chains with and without sialylated non-reducing termini further verifying structural assignments according to the invention.

Specific Structures from MSC Neutral Lipid Glycans

m/z 1375.77 corresponding monosaccharide composition (Hex4HexNAc2) corresponding to a structure with identical isobaric monosaccharide sequence as the structure Galβ1-3/4GlcNAcβ1-3/4Galβ1-3/4GlcNAβ1-3/4Galβ1-3/4Glc which could be confirmed with obtained fragments Y₂ (m/z 463.00), B₂ (m/z 485.79), Y₅/B₃ or Y₃/B₅ (m/z 471.86), Y₃ (m/z 707.90) and Y₄ (m/z 912.35). Fragment signals showing branched structures were not observed (m/z 897 or 448).

Taken together, the present results yielded especially direct evidence for the following specific structures in MSC glycolipid glycans: linear poly-N-acetyllactosamine chain (see m/z 1375) with less branched poly-N-acetyllactosamine chain than in the differentiated cells, further verifying structural assignments according to the invention.

### Example 22

### Cord Blood MSC O-Glycosylation Analyses

Exoglycosidase Analysis of O-Glycans

Cord blood derived MSC (UCB-MSC; see previous examples) cell lineages, which were already treated with N-glycosidase F to get rid of N-glycans, were subjected to non-reductive O-elimination to harvest O-glycans. Major peaks [M−H]⁻ emerging from acidic O-glycan pool using MALDI-TOF analysis were m/z 673.23 (NeuAcHexHexNAc), m/z 964.33 (NeuAc2HexHexNAc), m/z 1038.36 (NeuAcHex2HexNAc2), and m/z 1329.46 (NeuAc2Hex2HexNAc2). These peaks were not present in acidic N-glycan spectrum. Possible minor acidic O-glycan peaks [M−H]⁻ detected were m/z 835.28 (NeuAcHex2HexNAc), m/z 876.31 (NeuAcHexHexNAc2), m/z 973.28 (Hex2HexNAc2dHexSP), m/z 981.34 (NeuAcHex2HexNAcdHex), m/z 997.34 (NeuAcHex3HexNAc), m/z 1030.30 (Hex2HexNAc3SP), m/z 1110.38 (NeuAc2HexHexNAcdHex), m/z 1126.38 (NeuAc2Hex2HexNAc), m/z 1200.42 (NeuAcHex3HexNAc2), m/z 1272.44 (NeuAc2Hex2HexNAcdHex), m/z 1354.41 (Hex4HexNAc3SP), m/z 1370.48 (NeuAc2HexHexNAc3), m/z 1395.44 (Hex3HexNAc4SP), m/z 1403.49 (NeuAcHex3HexNAc3), m/z 1428.53 (NeuAcHexHexNAc4dHex) and m/z 1475.44 (NeuAc2Hex2HexNAc2dHex).

Acidic O-glycans were treated with α2,3-sialidase. Major acidic O-glycans were digested with this treatment. Peaks m/z 1038.36 [M−H]⁻ (NeuAcHex2HexNAc2) and m/z 1329.46 [M−H]⁻ (NeuAc2Hex2HexNAc2) minus sialic acid(s) were detectable in the mass spectrum of neutral O-glycan pool (m/z 771.26 [M+Na]⁺=Hex2HexNAc2). Therefore, disappearance of peaks m/z 1038.36 [M−H]⁻ (NeuAcHex2HexNAc2) and m/z 1329.46 [M−H]⁻ (NeuAc2Hex2HexNAc2) and simultaneous appearance of peak m/z 771.26 [M+Na]⁺ indicates that both sialic acids were preferentially α2,3-linked. Peak m/z 673 minus sialic acid (m/z 406.13 [M+Na]⁺) was hided by matrix peaks. Peak m/z 964.33 [M−H]⁻ (NeuAc2HexHexNAc) was not seen after α2,3-sialidase treatment indicating that at least one of the sialic acids was digested with α2,3-sialidase. All these structures were further confirmed with permetylation of original O-glycans and their fragmentation analysis.

The substrate specificity of α2,3-sialidase was tested using two synthetic oligosaccharides, namely NeuAcα2,3Galβ1,4GlcNAcβ1,3Galβ1,4Glc and NeuAcα2,6[Galβ1,4GlcNAcβ1-3(Galβ1,4GlcNAcβ1,6)Galβ1,4Glc. The enzyme was capable of using α2,3-linked sialic acid as substrate leaving α2,6-linked sialic acid intact.

After α2,3-sialidase treatment, these neutral O-glycans were subjected to β1,4-galactosidase treatment. Major neutral O-glycan peak (m/z 771.26) [M+Na]⁺ was lost as a result of this exo-glycosidase treatment giving rise to a new major neutral O-glycan peak m/z 609.21 [M+Na]⁺ (HexHexNAc2). This peak represented m/z 771.26 peak minus hexose monosaccharide, in this case galactose. Combining this data with the common knowledge of O-glycan core structures, the lost galactose was preferably β1,4-linked to GlcNAcβ1,6 branch of core 2 O-glycan structure.

The substrate specificity of β1,4-galactosidase was tested using a mixture of synthetic oligosaccharides. These control saccharides carried either terminal β1,3-linked or β1,4-linked galactose residues. The enzyme was capable of using β1,4-linked galactose as substrate leaving β1,3-linked galactose intact.

One minor acidic O-glycan peak (m/z 1475.44 [M−H]⁻=NeuAc2Hex2HexNAc2dHex) was characterized in the acidic O-glycan pool of adipocyte-differentiated UCB-MSC. This glycan was subjected in succession to the following exo-glycosidase treatments. First it was digested with α2,3-sialidase, then with α1,2-fucosidase and finally, with α1,3/4-fucosidase. After α2,3-sialidase treatment two sialic acid units were lost indicating that they were α2,3-linked. The remaining neutral O-glycan (m/z=917.32) [M+Na]⁺ was not digested with α1,2-fucosidase, but then again α1,3/4-fucosidase removed the fucose residue. Again, combining this exoglycosidase data with the common knowledge of O-glycan core structures, the structure would be NeuAcα2,3Galβ1,3[NeuAcα2,3Galβ1,4(Fucα1,3)GlcNAcβ1,6]GalNAc.

The substrate specificities of α1,2- and α1,3/4-fucosidases were tested using a mixture of synthetic oligosaccharides. These control saccharides carried either α1,2-linked or α1,3/4-linked fucose residues. α1,2-fucosidase cleaved α1,2-linked fucose leaving α1,3/4-linked fucose residue intact. α1,3/4-fucosidase acted just differently using α1,3/4-linked fucose as substrate leaving α1,2-linked fucose intact.

Fragmentation Analysis of Permetylated O-Glycan Structures

m/z 879.50 (NeuAcHexHexNAc) yielded fragments: B₁ (m/z 375.92 with H⁺ adduct ion), C₂ (m/z 620.18 with Na⁺ adduct ion) and Y₂ (m/z 504.09 with Na⁺ adduct ion) corresponding to a structure with identical isobaric monosaccharide sequence as core 1 O-glycan structure NeuAcα2,3/6Galβ1,3GalNAc.

m/z 1240.63 (NeuAc2HexHexNAc) yielded fragments: B_{1α} or B_{1β} (m/z 375,88 with H⁺ adduct ion), Y_{2α}/Y_{2β} (m/z 489.92 with Na⁺ adduct ion), C_{2α} (m/z 620.01 with Na⁺ adduct ion), Z_{1α} (m/z 643.03 with Na⁺ adduct ion), Y_{1α} (m/z 660.96 with Na⁺ adduct ion) and Y_{2α} or Y_{1β} (m/z 865.17 with Na⁺ adduct ion) corresponding to a structure with identical isobaric monosaccharide sequence as core 1 O-glycan structure NeuAcα2,3/6Galβ1,3(NeuAcα2,6)GalNAc.

m/z 1328.71 (NeuAcHex2HexNAc2) yielded fragments: B_{1α} or B_{1β} (m/z 375.87 with H⁺ adduct ion), C_{2α} or C_{2β} (m/z 619.95 with Na⁺ adduct ion), Z_{2α} or Z_{1β} (m/z 731.08 with Na⁺ adduct ion), Y_{2α} or Y_{1β} (m/z 749.06 with Na⁺ adduct ion), Y_{1α} or C_{3α} (m/z 865.01 with Na⁺ adduct ion) and Y_{3α} or Y_{2β} (m/z 953.24 with Na⁺ adduct ion) corresponding to a structure with identical isobaric monosaccharide sequence as core 2 O-glycan structure NeuAcα2,3/6Galβ1,3(Galβ1,3/4GlcNAcβ1,6)GalNAc or Galβ1,3(NeuAcα2,3/6Galβ1,3/4GlcNAcβ1,6)GalNAc.

m/z 1689.86 (NeuAc2Hex2HexNAc2) yielded fragments: B_{1α} or B_{1β} (m/z 375.75 with H⁺ adduct ion), Z_{3α}/Z_{1α} or Z_{2β}/Z_{1α} (m/z 471.68 with Na⁺ adduct ion), Y_{2α}/Y_{1β} (m/z 530.64 with Na⁺ adduct ion), C_{2α}/C_{2β} (m/z 619.86 with Na⁺ adduct ion), Z_{3α}/Z_{1β} or Z_{2α}/Z_{2β} (m/z 716.77 with Na⁺ adduct ion), C_{3α}/Y_{1α} (m/z 864.95 with Na⁺ adduct ion), Y_{3α}/Y_{2β} (m/z 939.48 with Na⁺ adduct ion), Z_{1β}/Z_{2α} (m/z 1092.16 with Na⁺ adduct ion) and Y_{3α}/Y_{2β} (m/z 1314.71 with Na⁺ adduct ion) corresponding to a structure with identical isobaric monosaccharide sequence as core 2 O-glycan structure NeuAcα2,3/6Galβ1,3(NeuAcα2,3/6Galβ1,3/4GlcNAcβ1,6)GalNAc.

Determined O-Glycan Structures

Combining the exoglycosidase data and the fragmentation data with the common knowledge of O-glycan core structures, the major acidic O-glycan structures in UCB-MSC cell lineages studied are the following: m/z 673.23 [M−H]⁻=NeuAcα2,3Galβ1,3GalNAc, m/z 964.33 [M−H]⁻=NeuAcα2,3Galβ1,3(NeuAcα2,6)GlcNAc, m/z 1038.36 [M−H]⁻=NeuAcα2,3 Galβ1,3(Galβ1,4GlcNAcβ1,6)GalNAc or Galβ1,3(NeuAcα2,3Galβ1,4GlcNAcβ1,6)GalNAc, and m/z 1329.46 [M−H]⁻=NeuAcα2,3Galβ1,3(NeuAcα2,3Galβ1,4GlcNAcβ1,6)GalNAc.

According to the exoglycosidase data, one minor acidic O-glycan structure is the following: m/z 1475.44 [M−H]⁻=NeuAcα2,3Galβ1,3[NeuAcα2,3Galβ1,4(Fucα1,3)GlcNAcβ1,6]GalNAc.

In conclusion, Core 1 and Core 2 were major detected O-glycan cores, with fucosylation occurring preferentially as Core 2 sialyl Lewis x epitope and Core 2 Lewis x epitope in acidic and neutral fractions, respectively. Sulphated/fosforylated glycans were also detected and by similarity to N-glycans they were assigned as sulphate esters. All detected sialic acids in Core 2 and larger O-glycans were α2,3-linked, and all analyzed Core 2 branch galactose residues were β1,4-linked.

### Example 23

### Fragmentation Analysis of Permethylated N-Glycan Structures of Cord Blood MSC

N-glycans were MS/MS-analyzed as permethylated glycans from cord blood derived MSC and cells differentiated from them into adipocyte direction, as well as bone marrow derived MSC and cells differentiated from them into osteoblast direction, and the results are presented as described in the preceding Examples.

### Adipocyte-Differentiated MSC Desialylated Total N-Glycans

m/z 1865.78 (Hex4HexNAc4) yielded fragments: Y₁ (m/z 299.66 with Na⁺ adduct ion), Y₂ (m/z 544.66 with Na⁺ adduct ion), B_{2α} (m/z 485.68 with Na⁺ adduct ion), Y_{3α}/Y_{3β} (m/z 734.78 with Na⁺ adduct ion), B_{5α}/Y_{4α}/Y_{4β} (m/z 865.67 with Na⁺ adduct ion), B_{3β}/Y_{4α} (m/z 879.24 with Na⁺ adduct ion), B_{3β}/Y_{5α} (m/z 1124.8 with Na⁺ adduct ion), Y_{4α}/Y_{4β} (m/z 1142.6 with Na⁻ adduct ion), B_{4α} (m/z 1343.9 with Na⁺ adduct ion), Y_{3β} (m/z 1402.33 with Na⁺ adduct ion), corresponding to structure Hex-HexNAc-Hex-(HexNAc-Hex-)Hex-HexNAc-HexNAc, further corresponding to a structure with identical isobaric monosaccharide sequence as Galβ1-3/4GlcNAcβ1-2Manα1-3-(GlcNAcβ1-2Manα1-6)Manβ1-4GlcNAcβ1-4GlcNAc.

m/z 1824.76 (Hex5HexNAc3) yielded fragments: Y₁ (m/z 299.72 with Na⁺ adduct ion), B_{2α} (m/z 485.74 with Na⁺ adduct ion), B_{5α}/Y_{4α}/Y_{3β} (m/z 661.61 with Na⁻ adduct ion), Y_{3α}/Y_{3β} (m/z 734.8 with Na⁺ adduct ion), B_{4α}/Y_{3β} (m/z 1083.38 with Na⁺ adduct ion), B_{4α}/Y_{4β} (m/z 1360.95 with Na⁺ adduct ion), corresponding to structure Hex-HexNAc-Hex-(Hex-Hex-)Hex-HexNAc-HexNAc, further corresponding to a structure with identical isobaric monosaccharide sequence as Galβ1-3/4GlcNAcβ1-2Manα1-3(Manα1-3/6Manα1-6)Manβ1-4GlcNAcβ1-4GlcNAc.

m/z 1794.75 (Hex4HexNAc3dHex1) yielded fragments: I; Y₁ (m/z 473.959 with Na⁻ adduct ion), B₄/Y₄ (m/z 635.13 with Na⁻ adduct ion), B_{3β}/Y_{3α} (m/z 675.89 with Na⁺ adduct ion), B_{4α}/Y_{3β} (m/z 880.11 with Na⁺ adduct ion), B_{5α} (m/z 1343.56 with Na⁺ adduct ion), corresponding to structure Hex-HexNAc-Hex-(Hex-)Hex-HexNAc-(dHex-)HexNAc, further corresponding to a structure with identical isobaric monosaccharide sequence as Galβ1-3/4GlcNAcβ1-2Manα1-3-(Manα1-6-)Manβ1-4GlcNAcβ1-4(Fucα1-6)GlcNAc. II; Y1 (m/z 299.86 with Na⁺ adduct ion), Y2 (m/z 544.87 with Na⁺ adduct ion), B4/Y4 (m/z 635.13 with Na⁺ adduct ion), B2α (m/z 661.97 with Na⁺ adduct ion), B3β/Y3α 675.89 with Na⁺ adduct ion), Y3α/Y3β (m/z 734.98 with Na⁺ adduct ion), B3α (m/z 865.99 with Na⁺ adduct ion), Y3α (m/z 953.16 with Na⁺ adduct ion), corresponding to structure Hex-(dHex-)HexNAc-Hex-(Hex-)Hex-HexNAc-HexNAc, further corresponding to a structure with identical isobaric monosaccharide sequence as Galβ1-3/4(Fucα1-2/3/4-)GlcNAcβ1-2Manα1-3-(Manα1-6-)Manβ1-4GlcNAcβ1-4GlcNAc.

m/z 2418.03 (Hex5HexNAc4dHex2) yielded fragments: Y₁ (m/z 473.57 with Na⁺ adduct ion), B_{2β} (m/z 485.6 with Na⁺ adduct ion), B_{3β} (m/z 689.6 with Na⁺ adduct ion), B_{2α} (m/z 659.68 with Na⁺ adduct ion), B_{4α}/B_{4β}/Y_{4α}/Y_{4β} (m/z 620.38 with Na⁺ adduct ion), B_{5α}/Y_{4α}/Y_{4β} or B_{3α} (m/z 865.74 with Na⁺ adduct ion), Y_{4α}/Y_{4β} (m/z 1316.38 with Na⁻ adduct ion), Y_{3α} (m/z 1779.32 with Na⁺ adduct ion), corresponding to structure Hex-(dHex-)HexNAc-Hex-(Hex-HexNAc-Hex-)Hex-HexNAc-(dHex-)HexNAc, further corresponding to a structure with identical isobaric monosaccharide sequence as Galβ1-3/4(Fucα1-3/4)GlcNAcβ1-2(Galβ1-3/4GlcNAcβ1-2Manα1-6-)Manβ1-4GlcNAcβ1-4(Fucα1-6-)GlcNAc.

m/z 3142.43 (Hex7HexNAc6dHex1) yielded fragments: Y₁ (m/z 473), B_{2α}/B_{2β} (m/z 485.56), B_{4α}/B_{4β} (m/z 934.72), Y_{4α}/Y_{3β} (m/z 1112.41), Y_{3α}/Y₆β (m/z 1561.27), Y_{3α} (m/z 2025.3), Y_{4α} (m/z 2228.93), Y_{6α} (m/z 2679.27), corresponding to structure Hex-HexNAc-Hex-HexNAc-Hex-(Hex-HexNAc-Hex-HexNAc-Hex-)Hex-HexNAc-(dHex-)HexNAc, further corresponding to a structure with identical isobaric monosaccharide sequence as Galβ1-3/4GlcNAcβ1-3/4Galβ1-3/4GlcNAcβ1-2Manα1-3(Galβ1-3/4GlcNAcβ1-3/4Galβ1-3/4GlcNAcβ1-2Manα1-6)Manβ1-4GlcNAcβ1-3/4(Fucα1-6)GlcNAc.

m/z 1345.58 (Hex3HexNAc2dHex1) yielded fragments: Y₁ (m/z 473.9), B₂ (m/z 648.95), C₂ (666.9), B₃ (894.08), Y_{3α} (m/z 1127.3), corresponding to structure Hex-(Hex-)Hex-HexNAc-(dHex-)HexNAc, possibly corresponding to structure Manα1-3(Manα1-6-)Manβ1-4GlcNAc1-4(Fucα1-6-)GlcNAc.

m/z 1620.69 (Hex4HexNAc3) yielded fragments: Y₁ (m/z 299.83), B_{2α} (m/z 485.8), Y₂ (m/z 544.68), B_{5α}/Y_{4α}/Y_{3β} (m/z 661.74), B_{3α} (m/z 689.92), Y_{3α}/Y_{3β} (m/z 734.4), B_{4α}/Y_{3β} (m/z 879.75), Y_{3α} (m/z 952.24), Y_{4α} (m/z 1157.25), Y_{5α} (m/z 1402.29), B_{3β}/Y_{3α} (m/z 675.49), corresponding to structure Hex-HexNAc-Hex-(Hex-)Hex-HexNAc-HexNAc, possibly corresponding to structure Galβ1-3/4GlcNAcβ1-2Manα1-3(Manα1-6-)Manβ1-4GlcNAcβ1-4GlcNAc.

m/z 967.45 (Hex2HexNAc2) yielded fragments: Y₁ (m/z 299.88), B₂ (m/z 444.48), B₃/Y₃ (m/z 471.78), B₃ (m/z 690.12), Y₃ (m/z 749.05), C₂ (m/z 462.95), corresponding to structure Hex-Hex-HexNAc-HexNAc, possibly corresponding to linear structure Manα1-3Manβ1-4GlcNAcβ1-4GlcNAc.

m/z 1171.61 (Hex3HexNAc2) yielded fragments: Y, (m/z 299.87), B₃/Y_{3α}/Y_{3β} (m/z 457.77), Y₂ (m/z 544.99), B₃ (m/z 894.29), B₃/Y₃ (m/z 676) Y_{3α}/Y_{3β} (735), Y_{3β} (m/z 953.3) corresponding to structure Hex-(Hex-)Hex-HexNAc-HexNAc, possibly corresbonding to structure Manα1-3(Manα1-6-)Manβ1-4GlcNAcβ1-4GlcNAc.

Cord Blood Derived MSC Desialylated Total N-Glycans

m/z 2693.2 (Hex6HexNAc5dHex1) yielded fragments: I; Y₁ (m/z 474), B_{2α} (m/z 485.53), Y_{6α}/Y_{4β} (m/z 1766.68), Y_{4α} (m/z 1781.41) corresponding to structure Hex-HexNAc-Hex-HexNAc-Hex-(Hex-HexNAc-Hex-)Hex-HexNAc-(dHex-)HexNAc, possibly corresponding to structure Galβ1-3/4GlcNAcβ1-3/4Galβ1-3/4GlcNAcβ1-3/4Manα1-3(Galβ1-3/4GlcNAcβ1-3/4Manα1-6)Manβ1-4GlcNAcβ1-4(Fucα1-6-)GlcNAc. II; B_{2β} (m/z 485.53), B_{2α} (m/z 661.66), Y_{4α} (m/z 2230.23), corresponding to structure Hex-(dHex-)HexNAc-Hex-HexNAc-Hex-(Hex-HexNAc-Hex-)Hex-HexNAc-HexNAc, further corresponding to a structure with identical isobaric monosaccharide sequence as Galβ1-3/4(Fucα1-2/3/4-)GlcNAcβ1-3/4Galβ1-3/4GlcNAcβ1-2Manα1-3(Galβ1-3/4GlcNAcβ1-2Manα1-6-)Manβ1-4GlcNAc-β1-4GlcNAc.

m/z 2243.97 (Hex5HexNAc4dHex1) yielded fragments: I; Y₁ (m/z 473.58), B_{2α}/B_{2β} (m/z 485.71), B₅/Y_{5α}/Y_{5β} (m/z 865.8) Y_{4α}/Y_{3β} (m/z 1112.84) Y_{4α}/Y_{4β} (m/z 1316.99), corresponding to structure Hex-HexNAc-Hex-(Hex-HexNAc-Hex-)Hex-HexNAc-(dHex-)HexNAc, further corresponding to a structure with identical isobaric monosaccharide sequence as Galβ1-3/4GlcNAcβ1-2Manα1-3(Galβ1-3/4GlcNAcβ1-2Manα1-6-)Manβ1-4GlcNAcβ1-4(Fucα1-6-)GlcNAc. II; B_{2β} (m/z 485.71), Y₂ (m/z 544.8), B_{2α} (m/z 661.39), Y_{3α}/Y_{3β} (m/z 734.77), B_{3α} (865.8), Y_{3β} (m/z 1576.22), Y_{4β} (m/z 1780.7), corresponding to structure Hex-(dHex-)HexNAc-Hex-(Hex-HexNAc-Hex-)Hex-HexNAc-HexNAc, further corresponding to a structure with identical isobaric monosaccharide sequence as Galβ1-3/4(Fucα1-2/3/4-)GlcNAcβ1-2Manα1-3(Galβ1-3/4GlcNAcβ1-2Manα1-6-)Manβ1-4GlcNAcβ1-4GlcNAc.

m/z 3142.56 (Hex7HexNAc6dHex1) yielded fragments: I; B_{2α}/B_{2β} (m/z 487.36), Y_{5α} (m/z 2297.15), Y_{6β} (m/z 2683.25), corresponding to structure Hex-(dHex-)HexNAc-Hex-HexNAc-Hex-(Hex-HexNAc-Hex-HexNAc-Hex-)Hex-HexNAc-HexNAc, further corresponding to a structure with identical isobaric monosaccharide sequence as Galβ1-3/4(Fucα1-2/3/4-)GlcNAcβ1-3/4Galβ1-3/4GlcNAcβ1-2Manα1-3(Galβ1-3/4GlcNAcβ1.3/4Galβ1-3/4GlcNAcβ1-2Manα1-6-)Manβ1-4GlcNAcβ1-4GlcNAc. II; B_{2α}/B_{2β} (m/z 487.36), Y_{6β} (m/z 2683.25), corresponding to structure Hex-HexNAc-Hex-HexNAc-Hex-(Hex-HexNAc-Hex-HexNAc-Hex-)Hex-HexNAc-(dHex-)HexNAc, further corresponding to a structure with identical isobaric monosaccharide sequence as Galβ1-3/4GlcNAcβ1-3/4Galβ1-3/4GlcNAcβ1-2Manα1-3(Galβ1-3/4GlcNAcβ1-3/4Galβ1-3/4GlcNAcβ1-2Manα1-6-)Manβ1-4GlcNAcβ1-4(Fucα1-2/3/4-)GlcNAc.

Contrary to the UCB mesenchymal stem cells which have differentiated to adipocyte direction, the MSC have two isomeric (m/z 2539 Hex7HexNAc6dHex1) structures.

m/z 1171.61 (Hex3HexNAc2) yielded fragments: Y₁ (m/z 300.12), B₃/Y_{3α}/Y_{3βl (m/z} 457.91), Y₂ (m/z 544.21), B₃ (m/z 894.41), corresponding to structure Hex-(Hex-)Hex-HexNAc-HexNAc, further corresponding to a structure with identical isobaric monosaccharide sequence as Manα1-3(Manα1-6-)Manβ1-4GlcNAcβ1-4GlcNAc.

Osteoblast-Differentiated MSC Desialylated Total N-Glycans

m/z 2156.15 (NeuAcHex4HexNAc3dHex1) yielded fragments: B_{1α} (m/z 375.9 with H⁺ adduct ion), B_{3α}/Y_{6α} (m/z 471.97), B_{3α} (m/z 847.27), B_{5α}/Y_{6α}/Y_{3β} (m/z 866.08), Y_{4α}/Y_{4β} (m/z 1331.31), Y_{6α} (m/z 1780.25), corresponding to structure NeuAc-Hex-HexNAc-Hex-(Hex-)-Hex-HexNAc-(dHex-)HexNac, further corresponding to a structure with identical isobaric monosaccharide sequence as NeuAcα1-2/3/6Galβ1-3/4GlcNAcβ1-2Manα1-3(Manα1-6-)Manβ1-4GlcNAcβ1-4(Fucα1-6)GlcNAc.

BM MSC differentiated to osteoblasts, neutral N-glycans, masses are with Na⁻ adduct ion unless otherwise spesified

m/z 2070.03 (Hex5HexNAc4) yielded fragments: Y₂ (m/z 544.8), B_{2α}/B_{2β} (m/z 485.95), B_{5α}/Y3α/Y4β (m/z 662.05), Y_{4α} (m/z 938.99), B₅/Y_{4α}/Y_{4β} (m/z 866.16), Y_{4α}/Y_{4β} (m/z 1143.51), Y_{3β} (m/z 1402.65), Y_{4α}/Y_{4β} (m/z 1607.44), corresponding to structure Hex-HexNAc-Hex-(Hex- HexNAc-Hex-)Hex-HexNAc-HexNAc, further corresponding to a structure with identical isobaric monosaccharide sequence as Galβ1-3/4GlcNAcβ1-2Manα1-3-(Galβ1-3/4GlcNAcβ1-2Manα1-6-)Manβ1-4GlcNAcβ1-4GlcNAc.

m/z 1620.69 (Hex4HexNAc3) yielded fragments: Y₁ (m/z 299.89), B_{2α} (m/z 485.89), Y₂ (m/z 544.83), B₅/Y_{4α}/Y_{3β} (m/z 661.71), Y_{3α}/Y_{3β} (m/z 734.95), B_{4α}/Y3_{β} (m/z 879.99), Y_{3α} (m/z 952.54), Y_{4α} (m/z 1157.18), B_{3β}/Y_{3α} (m/z 675.96), B_{4α}/Y_{4α} (m/z 634.93), B_{5α}/Y_{3α}/Y_{3β} (m/z 457.87), corresponding to structure Hex-HexNAc-Hex-(Hex-)Hex-HexNAc-HexNAc, further corresponding to a structure with identical isobaric monosaccharide sequence as Galβ1-3/4GlcNAcβ1-2Manα1-3(Manα1-6-)Manβ1-4GlcNAcβ1-4GlcNAc.

m/z 2245.14 (Hex5HexNAc4dHex1) yielded fragments: I; Y₁ (m/z 474.17), B_{5α}/Y_{3α}/Y_{4β} (m/z 662.39), Y₂ (m/z 719.28), B_{5α}/Y_{4α}/Y_{4β} (m/z 866.54), Y_{4α}/Y_{4β} (m/z 1318), B_{2α}/B_{2β} (m/z 486.28), Y_{4α} (m/z 1782.03), corresponding to structure Hex-HexNAc-Hex-(Hex-HexNAc-Hex-)Hex-HexNAc-(dHex-)HexNAc, further corresponding to a structure with identical isobaric monosaccharide sequence as Galβ1-3/4GlcNAcβ1-2Manα1-3(Galβ1-3/4GlcNAcβ1-2Manα1-6-)Manβ1-4GlcNAcβ1-4(Fucα1-6-)GlcNAc. II; Y₂ (m/z 545.13), B_{2α}/B_{2β} (m/z 486.28), B_{5α}/Y_{3α}/Y_{4β} (m/z 662.39), B_{2α} (m/z 660), B_{5α}/Y_{4α}/Y_{4β} (m/z 866.54), Y_{4α}/Y_{4β} (m/z 1143), corresponding to structure Hex-(dHex-)HexNAc-Hex-(Hex-HexNAc-Hex-)Hex-HexNAc-HexNAc, further corresponding to a structure with identical isobaric monosaccharide sequence as Galβ1-3/4(Fucα1-2/3/4-)GlcNAcβ1-2Manα1-3(Galβ1-3/4GlcNAcβ1-2Manα1-6-)Manβ1-4GlcNAcβ1-4GlcNAc.

BM MSC differentiated to osteoblasts, acidic N-glycans, all m/z are presented as (M+Na⁺) unless otherwise stated

m/z 1981.99 (NeuAc1Hex4HexNAc3) yielded fragments: Y₂ (m/z 544.92), B_{4α}/Y_{6α} (m/z 675.93), B_{5α}/Y_{4α}/Y_{3β} (m/z 416.99), B₅/Y₅/Y_{3β} (m/z 661.95), B_{3α} (m/z 846.74), Y₄, (m/z 1157.41), Y₆ (m/z 1606.98), B_{1α} (m/z 375.95 with H⁺ adduct ion, m/z 397.82 with Na⁺ adduct ion), B_{3α}/Y_{6α} (m/z 471.91), corresponding to structure NeuAc-Hex-HexNAc-Hex-(Hex-)Hex-HexNAc-HexNAc, further corresponding to a structure with identical isobaric monosaccharide sequence as NeuAcα1-2/3/6Galβ1-3/4GlcNAcβ1-2Manα1-3(Manα1-6-)Manβ1-4GlcNAcβ1-4GlcNAc.

m/z 3054.59 (NeuAcHex6HexNAc5dHex1) yielded fragments: B_{1β} (m/z 376.96 with H⁺ adduct ion), B_{3β}/Y_{6β} (m/z 472.98), B_{3β} (m/z 848.39), Y_{4α} (m/z 2141.69), Y_{4β} (m/z 2232.73), Y_{6α} (m/z 2594.6), Y_{5β} (m/z 2682.92), corresponding to structure Hex-HexNAc-Hex-HexNAc-Hex-(NeuAc-Hex-HexNAc-Hex-)Hex-HexNAc-(dHex-)HexNAc, further corresponding to a structure with identical isobaric monosaccharide sequence as Galβ1-3/4GlcNAcβ1-3/4Galβ1-3/4GlcNAcβ1-2Manα1-3(NeuAcα2/3/6Galβ1-3/4GlcNAcβ1-2Manα1-6-)Manβ1-4GlcNAcβ1-4(Fucα6-)GlcNAc.

m/z 1777.79 (NeuAc1Hex3HexNAc3) yielded fragments: B₁ (m/z 375.52 with H⁺ adduct ion), B₃/Y₆ or B₄/Y₅ or B₆/Y₃ (m/z 471.8), B₄/Y₆ (m/z 675.67), Y₄ (m/z 952.43), B₃ (m/z 847.46), C₃ (m/z 865.73), corresponding to structure NeuAc-Hex-HexNAc-Hex-Hex-HexNAc-HexNAc, further corresponding to a structure with identical isobaric monosaccharide sequence as NeuAcα1-2/3/6Galβ1-3/4GlcNAcβ1-2Manα1-3Manβ1-4GlcNAcβ1-4GlcNAc.

m/z 2605.24 (NeuAcHex5HexNAc4dHex1) yielded fragments: B, (m/z 375.84 with H⁺ adduct ion), B_{3α}/Y_{6α} (m/z 472), B_{5α}/Y_{5α}/Y3β (m/z 661.83), B_{3α} (m/z 846.81), B_{5α}/Y_{6α}/Y_{3β} (m/z 865.68), Y_{4α}/Y_{3β} (m/z 1112.78), Y_{4α}/Y_{4β} (m/z 1317.15), Y_{5α}/Y_{3β} (m/z 1575.67), Y_{5α}/Y_{4β} (m/z 1780.56), B_{6α} (m/z 2141.62), Y_{6α} (m/z 2230.4), corresponding to structure NeuAc-Hex-HexNAc-Hex-(Hex-HexNAc-Hex-)Hex-HexNAc-(dHex-)HexNAc, further corresponding to a structure with identical isobaric monosaccharide sequence as NeuAcα1-2/3/6Galβ1-3/4GlcNAcβ1-2Manα1-3(Galβ1-3/4GlcNAcβ1-2Manα1-6-)Manβ1-4GlcNAcβ1-4(Fucα1-6)GlcNAc.

m/z 2185.97 (NeuAcHex5HexNAc3) yielded fragments: B_{1α} (m/z 375.9 with H⁺ adduct ion), B_{3α}/Y_{6α} (m/z 471.89), B_{5α}/Y_{5α}/Y_{3β} (m/z 661.9), B_{4α}/Y_{4α}/Y_{3β} or B_{5α}/Y_{6α}/Y_{3β} (m/z 866), Y_{4α}/Y_{4β} (m/z 1143.11), Y_{4α} (1361.61), Y_{6α} (m/z 1810.67), corresponding to structure NeuAc-Hex-HexNAc-Hex-(Hex-Hex-)Hex-HexNAc-HexNAc, further corresponding to a structure with identical isobaric monosaccharide sequence as NeuAcα1-2/3/6Galβ1-3/4GlcNAcβ1-2Manα1-3(Man-α1-3/6Manα1-6-)Manβ1-4GlcNAcβ1-4GlcNAc.

m/z 3603.09 (NeuAc3Hex6HexNAc5) yielded fragments: Y_{8α} or Y_{6β} (m/z 378.23), B_{3α}/Y_{8α} or B_{3β}/Y_{6β} (m/z 474.4), B_{3α} or _{B3β} (m/z 850.54), Y_{4β} or Y_{6α} (m/z 2786.01), corresbonding to structure which is at least biantennary and has at least one N-acetylneuraminic acid residue in both branches.

Taken together, the present results yielded direct evidence for especially the following specific structures in MSC N-glycans as well as in cells differentiated from them: N-glycan monoantennary core structure, N-glycan biantennary core structure, hybrid-type N-glycan core structure, poly-N-acetyllactosamine antennae, tri-antennary core structure, non-reducing GlcNAc antennae, non-reducing terminal Lex on sialylated biantennary N-glycan non-sialylated antenna, non-reducing terminal Lex on poly-N-acetyllactosamine antenna, and low-mannose type N-glycans with Man-3 branched structure, further verifying structural assignments according to the invention; in cell type specific manner as presented and/or discussed above.

### Example 24

### Differential Analysis of Cord Blood MSC Differentiation Related Changes in N-Glycan Profiles

Cord blood MSC and cells differentiated from them into 1) adipocyte, 2) osteoblast, and 3) chondrocyte direction, were analyzed by their N-glycan profiles as described in the preceding Examples. The results of analysis are described in Tables 28, 29, and 30 which were constructed as in the preceding Examples.

Results and conclusions: The larger diff. variables in each of the Tables 28, 29, and 30 indicate differentiation association in each differentiation direction, and The larger diff. variables in each of the Tables 28, 29, and 30 indicate differentiation association in each differentiation direction. When the results in the Tables are correlated and analyzed relative to the other analyses of the present invention, it can be concluded that they show clear differentiation line specific structure variabilities, most pronouncedly in non-sialylated terminal LacNAc expression in N-glycans, low-mannose type N-glycan expression, and core-fucosylation of N-glycans. These and additional cell type specific results are further analyzed and included in Table 27 as evidence of cell-type specific terminal epitope and glycan core structure expression in different differentiation lineages.

### Example 25

### Antibody Profiling of Bone Marrow Derived and Cord Blood Derived Mesenchymal Stem Cell Lines

Experimental Procedures

Bone marrow derived mesenchymal stem cell lines (BM-MSC). Isolation and culture of BM-MSCs, as well as osteogenic differentiation of BM-MSCs, were performed as described in Example 1.

Umbilical cord blood mesenchymal stem cell (CB-MSC) isolation and culture. The isolation and culture of CB-MSCs was performed as described in Example 1 with some modifications. Osteogenic differentiation of CB-MSCs was induced as described for BM-MSCs for 16 days.

Adipogenic differentiation of CB-MSCs. Cells were grown in proliferation medium to almost confluence after which the adipogenic induction medium including α-MEM

Glutamax supplemented with 10% FCS, 20 mM Hepes, 1×penicillin-streptomycin, 0.1 mM Indomethasin (all from Sigma), 0.5 mM IBMX-22, 0.4 μg/ml dexamethasone and 0.5 μg/ml Insulin (all three from Promocell) was added. After 3 days, terminal adipogenic differentiation medium including α-MEM Glutamax supplemented with 10% FCS, 20 mM Hepes, 1×penicillin-streptomycin, 0.1 mM Indomethasin (all from Sigma), 0.5 μg/ml Insulin and 3.0 μg/ml Ciglitazone (both two from Promocell) was added and cells were grown for 14 days (altogether 17 days) in 5% CO₂ at 37° C. Differentiation medium was refreshed twice a week throughout the differentiation period.

Flow cytometric analysis of mesenchymal stem cell phenotype. Both BM and CB derived MSCs were phenotyped by flow cytometry (BD FACSAria, Becton Dickinson). FITC, APC or PE conjugated antibodies against CD13, CD14, CD29, CD34, CD44, CD45, CD49e, CD73, CD90, HLA-DR and HLA-ABC (all from BD Biosciences) and CD105 (Abcam Ltd.) were used for direct labelling. For staining, cells in a small volume, i.e. 5×10⁴ cells/100 μl 0.3% ultra pure BSA, 2 mM EDTA-PBS buffer, were aliquoted to FACS-tubes. One microliter of each antibody was added to cells and incubated for 30 min at +4° C. Cells were washed with 2 ml of buffer and centrifuged at 300×g for 4 min. Cells were suspended in 200 μl of buffer for flow cytometric analysis.

Cell harvesting for antibody staining. Both BM and CB-MSCs were detached from cell culture plates with 2 mM EDTA-PBS solution (Versene), pH 7.4, for approximately 30 minutes at 37° C. Both osteogenic and adipogenic cells were detached with 10 mM EDTA-PBS solution, pH 7.4, for 30 minutes and 5 minutes at 37° C., respectively. Since the differentiated cells detached from culture plates as clusters, they were suspended by pipetting with Pasteur-pipette or by vortexing and by suspending through an 18 gauge needle to get a single cell suspension. Finally, the cell suspension was filtered through a 50 μm filter to get rid of unsuspended cell aggregates. Harvested cells were centrifuged at 300×g for 4 minutes and suspended for small volume of 0.3% ultra pure BSA (Sigma), 2 mM EDTA-PBS buffer. Primary antibody staining. BM and CB derived cells were aliquoted to FACS-tubes in a small volume, i.e. 5-7×10⁴ cells/100 μl 0.3% ultra pure BSA, 2 mM EDTA-PBS buffer. Four microliters of anti-glycan primary antibody was added to cell suspension, vortexed and incubated for 30 min at room temperature. Cells were washed with 2 ml of buffer and centrifuged for 4 min at 300×g, after which the supernatant was removed. Primary antibodies used for staining are listed in Table 26.

Secondary antibody staining. AlexaFluor 488-conjugated anti-mouse (1:500, Invitrogen) and anti-rabbit (1:500, Molecular Probes), as well as FITC-conjugated anti-rat (1:320, Sigma) and anti-human λ (1:1000, Southern Biotech) secondary antibodies were used for appropriate primary antibodies. Secondary antibodies were diluted in 0.3% ultra pure BSA, 2 mM EDTA-PBS buffer and 100 μl of dilution was added to the cell suspension. Samples were incubated for 30 min at room temperature in the dark. Cells were washed with 2 ml of buffer and centrifuged for 4 min at 300×g. Supernatant was removed and cells were suspended in 200 μl of buffer for flow cytometric analysis. As a negative control cells were incubated without primary antibody and otherwise treated similarly to labelled cells.

Flow cytometric analysis. Cells with fluorescently labelled antibodies were analysed with BD FACSAria (Becton Dickinson) using FITC detector at wavelength 488. Results were analysed with BD FACSDiva software version 5.0.1 (Becton Dickinson).

Results and Discussion

Flow cytometric analysis of mesenchymal stem cell phenotype. Both BM and CB-MSCs were negative for hematopoietic markers CD34, CD45 and CD14. The cells stained positively for the CD13 (aminopeptidase N), CD29 (β1-integrin), CD44 (hyaluronan receptor), CD73 (SH3), CD90 (Thy-1), CD105 (SH2/endoglin) and CD49e. The cells stained also positively for HLA-ABC, but negatively for HLA-DR.

Anti-glycan antibody profiling of BM-MSCs. BM-MSCs and osteogenic cells (BM-OG) differentiated thereof were analyzed with up to 60 anti-glycan antibodies by flow cytometry and also with 29 antibodies by immunohistochemistry (IHC). The results of BM-MSC staining are presented in Table 26 and in FIG. 20.

The most prominent enrichment in stem cells is SSEA-4 and in osteogenic cells some glycolipid epitopes such as ganglioseries asialo GM1 and asialo GM2; globoseries structures globotriasyl ceramide Gb3 and globotetraose also known as globoside (GL4 or Gb4); as well as Lewis a and sialylated Ca15-3.

Lewis x structures seems not to be present in quantity over detection level under FACS analysis conditions in a major part of the MSCs in the preparations of MSCs or in differentiated cells based on staining with 5 different anti-Lex antibodies. There is however specific Lewis x expression recognizable by specific anti-Lewis x clones. On the other hand, sialyl Lewis x structures are present on both stem cells and in osteogenic cells and the proportions differ between different anti-sLex antibodies, which is most probably due to the different carriers for sLex epitopes. For example GF526 anti-sLex antibody recognizes only sLex epitope carried by a specific O-glycan core II structure. The binding of GF526 has been determined to be related to P-selectin ligand glycoprotein PSGL-1, which represents the O-glycan effectively in large quantities on certain non-stem cell materials. It is however realised that core II O-glycans have been reported on several mucin type O-glycans and the present invention is not limited to analysis of the Core II sLex on PSGL-1 on the mesenchymal stem cells. The carrier and the exact binding epitope of sLex recognized by two other anti-sLex antibodies (GF516 and GF307) appears to include structures other than core II with optimal fine specificity different from the core two including polylactosamines with β3Gal elongation The antibodies with different fine and core/carrier glycan specifiy cell populations of different sizes.

Anti-glycan antibody profiling of CB-MSCs. CB-MSCs and both osteogenic and adipocytic cells differentiated thereof were analysed with up to 61 different anti-glycan antibodies by flow cytometry. The results of CB-MSC staining are presented in Table 27 and in FIG. 21. Likewise in BM derived antibody profiling, there seems not to be a single specific glycan epitope determining either CB-MSCs or cells differentiated into osteogenic or adipocytic lineages. Some glycans, e.g. H disaccharide (GF394), TF (GF281), Glycodelin (GF375), Lewis x (GF517) and Galα3Gal (GF413), are highly enriched in CB derived MSCs, but their proportion in the whole stem cell population is rather low (10% or below). Interestingly, there seems to be also glycans, e.g. SSEA-4 (GF354), Lewis c (GF295), SSEA-3 (VPU009), GD2 (GF406), sialyl Lewis x (GF307) and Tra-1-60 (GF415), enriched in stem cells and in adipocytic cells, but not in osteogenic cells. BM-derived cells have not been differentiated into adipocytic direction, so we can not compare the data between different adipocytes from different sources. Osteogenic differentiation induces similar enrichment of glycans both in BM and CB derived cells. Only Gb3, increasing in BM derived osteogenic cells is not increased in CB derived osteogenic cells. Furthermore, gangliosides GT1b, GD2, GD3 and A2B5, not tested in BM-derived cells, are highly enriched in CB derived osteogenic cells. Most of the glycan epitopes revealed by specifc antibodies of the example enriched in CB-derived osteoblasts are also enriched (even with higher percentage) in CB-derived adipocytes, but the invention reveals even for these targets that there are differences in expression levels between the cell types allowing characterization of both differentiation lineages. An interesting group of glycan epitopes after differentiation is glycan epitopes recognizable by known antibodies against gangliosides, in general increasing from stem cells (<10%) into osteoblasts and adipocytes (50-100%). Unlike in BM-derived MSCs, there seems to be some positivity with anti-Lewis x antibodies GF517 and GF525 in CB derived cells. The results with anti-sialyl-Lewis x antibodies are parallel with both cell types.

Tables

TABLE 1
Differential expression of acidic N-glycan signals in bone marrow
mesenchymal stem cells (MSC) versus osteoblast-differentiated cells
(OB) as analyzed by MALDI-TOF mass spectrometric profiling.

| | | | | | | |
|---|---|---|---|---|---|---|
| Composition | Structure | m/z | MSC | OB | relat. | diff. |

Not detected in OB:

| | | | | | | | | | | | | | |
|---|---|---|---|---|---|---|---|---|---|---|---|---|---|
| S2H5N3F2P1 | A | S2 | H | | E | P | | | 2390 | 0.62 | 0.00 | ∞ | 0.62 |
| S1H6N5F4 | A | S1 | C | R | E | | | | 2880 | 0.27 | 0.00 | ∞ | 0.27 |
| S1H5N5 | A | S1 | C | | | | Q | | 2133 | 0.20 | 0.00 | ∞ | 0.20 |
| S2H3N3F1 | A | S2 | H | N | F | | | | 1840 | 0.13 | 0.00 | ∞ | 0.13 |
| H5N3F2P1 | A | | H | | E | P | | | 1808 | 0.11 | 0.00 | ∞ | 0.11 |
| S1H4N5 | A | S1 | C | | | | T | | 1971 | 0.11 | 0.00 | ∞ | 0.11 |
| S1H8N7 | A | S1 | C | R | | | | | 3026 | 0.11 | 0.00 | ∞ | 0.11 |
| S2H5N3F1 | A | S2 | H | | F | | | | 2164 | 0.09 | 0.00 | ∞ | 0.09 |
| S3H7N6F3 | A | S3 | C | R | E | | | | 3681 | 0.08 | 0.00 | ∞ | 0.08 |
| S2H4N2F1 | A | S2 | O | | F | | | | 1799 | 0.07 | 0.00 | ∞ | 0.07 |
| S1H11N10 | A | S1 | C | R | | | | | 4121 | 0.06 | 0.00 | ∞ | 0.06 |
| S2H4N3 | A | S2 | H | N | | | | | 1856 | 0.06 | 0.00 | ∞ | 0.06 |
| S3H7N6F4 | A | S3 | C | R | E | | | | 3827 | 0.06 | 0.00 | ∞ | 0.06 |
| S2H2N2 | A | S2 | O | | | | | | 1329 | 0.06 | 0.00 | ∞ | 0.06 |
| S1H4N3F3 | A | S1 | H | N | E | | | | 2003 | 0.06 | 0.00 | ∞ | 0.06 |
| S2H5N5 | A | S2 | C | | | | Q | | 2424 | 0.05 | 0.00 | ∞ | 0.05 |
| S2H3N5F2 | A | S2 | C | | E | | T | | 2392 | 0.05 | 0.00 | ∞ | 0.05 |
| G1H3N2 | A | S1 | O | | | | | Y | 1216 | 0.05 | 0.00 | ∞ | 0.05 |
| S3H6N4F1P1 | A | S3 | C | | F | P | X | | 2900 | 0.04 | 0.00 | ∞ | 0.04 |
| G1H4N3 | A | S1 | H | N | | | | Y | 1581 | 0.04 | 0.00 | ∞ | 0.04 |
| S1H7N6F5 | A | S1 | C | R | E | | | | 3391 | 0.04 | 0.00 | ∞ | 0.04 |
| S1H3N4 | A | S1 | C | | | | T | | 1606 | 0.04 | 0.00 | ∞ | 0.04 |
| S2H4N4 | A | S2 | C | | | | Q | | 2059 | 0.04 | 0.00 | ∞ | 0.04 |
| S1H5N4F4 | A | S1 | C | B | E | | | | 2514 | 0.03 | 0.00 | ∞ | 0.03 |
| S2H3N4F2 | A | S2 | C | | E | | T | | 2189 | 0.03 | 0.00 | ∞ | 0.03 |
| S1H7N6F4 | A | S1 | C | R | E | | | | 3245 | 0.02 | 0.00 | ∞ | 0.02 |
| S2H3N3 | A | S2 | O | | | | | | 1694 | 0.02 | 0.00 | ∞ | 0.02 |
| S1H4N4F2 | A | S1 | C | | E | | Q | | 2060 | 0.02 | 0.00 | ∞ | 0.02 |
| G1H5N3 | A | S1 | H | | | | | Y | 1743 | 0.02 | 0.00 | ∞ | 0.02 |
| S1H8N7F3 | A | S1 | C | R | E | | | | 3464 | 0.02 | 0.00 | ∞ | 0.02 |

Over 2 times overexpressed in MSC:

| | | | | | | | | | | | | |
|---|---|---|---|---|---|---|---|---|---|---|---|---|
| S1H8N7F1 | A | S1 | C | R | F | | | 3172 | 0.49 | 0.03 | 14.52 | 0.46 |
| S1H7N6F3 | A | S1 | C | R | E | | | 3099 | 0.80 | 0.10 | 8.16 | 0.71 |
| S1H4N2 | A | S1 | O | | | | | 1362 | 2.39 | 0.43 | 5.50 | 1.95 |
| S1H2N2 | A | S1 | O | | | | | 1038 | 0.11 | 0.02 | 4.92 | 0.09 |
| S3H8N7F1 | A | S3 | C | R | F | | | 3754 | 0.06 | 0.01 | 4.48 | 0.05 |
| H4N2P1 | A | | L | | | P | | 1151 | 0.07 | 0.02 | 3.94 | 0.05 |
| S1H7N5F1A1 | A | S1 | C | | F | | X | 2645 | 0.11 | 0.03 | 3.70 | 0.08 |
| S1H4N3F1P1 | A | S1 | H | N | F | P | | 1791 | 0.11 | 0.04 | 2.88 | 0.07 |
| S2H3N2F1 | A | S2 | O | | F | | | 1637 | 0.08 | 0.03 | 2.84 | 0.05 |
| S1H6N5F3 | A | S1 | C | R | E | | | 2733 | 0.96 | 0.37 | 2.60 | 0.59 |
| S2H6N3F1P1 | A | S2 | H | | F | P | | 2406 | 0.67 | 0.26 | 2.60 | 0.41 |
| S1H4N4 | A | S1 | C | | | | Q | 1768 | 1.29 | 0.50 | 2.60 | 0.80 |
| S2H7N6F3 | A | S2 | C | R | E | | | 3390 | 0.47 | 0.19 | 2.46 | 0.28 |
| S2H2N3F1 | A | S2 | O | | F | | | 1678 | 1.60 | 0.66 | 2.44 | 0.95 |
| S1H7N6F1 | A | S1 | C | R | F | | | 2807 | 2.67 | 1.22 | 2.20 | 1.46 |
| S1H7N6F2 | A | S1 | C | R | E | | | 2953 | 0.06 | 0.03 | 2.15 | 0.03 |
| S2H4N3F1 | A | S2 | H | N | F | | | 2002 | 0.31 | 0.15 | 2.07 | 0.16 |
| S1H7N3 | A | S1 | H | | | | | 2051 | 0.09 | 0.04 | 2.03 | 0.05 |
| S1H4N3 | A | S1 | H | N | | | | 1565 | 2.70 | 1.33 | 2.03 | 1.36 |

Over 1.5 times overexpression in MSC:

| | | | | | | | | | | | | |
|---|---|---|---|---|---|---|---|---|---|---|---|---|
| H3N6F3P1 | A | | C | | E | P | T | 2239 | 0.27 | 0.15 | 1.77 | 0.12 |
| S1H5N2 | A | S1 | O | | | | | 1524 | 0.04 | 0.02 | 1.75 | 0.02 |
| S3H6N5F1 | A | S3 | C | R | F | | | 3024 | 0.32 | 0.19 | 1.70 | 0.13 |
| S2H5N4F1 | A | S2 | C | B | F | | | 2367 | 5.31 | 3.13 | 1.70 | 2.19 |
| S2H7N6F4 | A | S2 | C | R | E | | | 3536 | 0.06 | 0.04 | 1.61 | 0.02 |
| S2H6N5F1 | A | S2 | C | R | F | | | 2732 | 1.84 | 1.16 | 1.58 | 0.68 |
| S1H5N5F1 | A | S1 | C | | F | | Q | 2279 | 0.61 | 0.40 | 1.55 | 0.22 |
| S1H6N5F1 | A | S1 | C | R | F | | | 2441 | 7.44 | 4.89 | 1.52 | 2.55 |
| S2H7N6 | A | S2 | C | R | | | | 2952 | 0.18 | 0.12 | 1.50 | 0.06 |

Less than 1.5 times overexpression in MSC:

| | | | | | | | | | | | | | |
|---|---|---|---|---|---|---|---|---|---|---|---|---|---|
| S2H5N4F2 | A | S2 | C | B | E | | | | 2513 | 0.07 | 0.05 | 1.47 | 0.02 |
| S1H7N5 | A | S1 | C | | | | X | | 2457 | 0.07 | 0.05 | 1.43 | 0.02 |
| G1S2H5N4F1 | A | S3 | C | B | F | | | Y | 2674 | 0.13 | 0.09 | 1.41 | 0.04 |
| S1H5N3 | A | S1 | H | | | | | | 1727 | 2.36 | 1.68 | 1.40 | 0.68 |
| S1H5N3F1 | A | S1 | H | | F | | | | 1873 | 1.92 | 1.43 | 1.34 | 0.48 |
| H3N2P1 | A | | L | | | P | | | 989 | 0.04 | 0.03 | 1.31 | 0.01 |
| S1H3N2 | A | S1 | O | | | | | | 1200 | 0.75 | 0.57 | 1.31 | 0.18 |
| S1H6N5 | A | S1 | C | R | | | | | 2295 | 2.15 | 1.65 | 1.31 | 0.50 |
| S1H7N4 | A | S1 | C | | | | X | | 2254 | 0.13 | 0.10 | 1.29 | 0.03 |
| S1H4N4F1 | A | S1 | C | | F | | Q | | 1914 | 1.19 | 0.95 | 1.24 | 0.23 |
| S2H5N4 | A | S2 | C | B | | | | | 2221 | 4.54 | 3.66 | 1.24 | 0.88 |
| S1H6N3 | A | S1 | H | | | | | | 1889 | 2.70 | 2.28 | 1.18 | 0.41 |
| S2H5N3 | A | S2 | H | | | | | | 2018 | 0.21 | 0.19 | 1.13 | 0.02 |
| S2H7N6F1 | A | S2 | C | R | F | | | | 3098 | 0.46 | 0.40 | 1.13 | 0.05 |
| S1H6N5F2 | A | S1 | C | R | E | | | | 2587 | 0.44 | 0.40 | 1.09 | 0.04 |
| S1H5N4F2 | A | S1 | C | B | E | | | | 2222 | 1.70 | 1.58 | 1.08 | 0.12 |
| S1H4N3F1 | A | S1 | H | N | F | | | | 1711 | 2.28 | 2.15 | 1.06 | 0.13 |
| S1H6N6F1 | A | S1 | C | R | F | | Q | | 2644 | 0.10 | 0.09 | 1.05 | 0.00 |
| S2H1N3F1 | A | S2 | O | | F | | | | 1516 | 0.06 | 0.06 | 1.04 | 0.00 |
| S1H3N3 | A | S1 | H | N | | | | | 1403 | 0.30 | 0.29 | 1.03 | 0.01 |
| S2H3N5F1 | A | S2 | C | | F | | T | | 2246 | 0.10 | 0.09 | 1.02 | 0.00 |
| S1H5N4 | A | S1 | C | B | | | | | 1930 | 10.39 | 10.24 | 1.01 | 0.14 |

Less than 1.5 times overexpression in OB:

| | | | | | | | | | | | | |
|---|---|---|---|---|---|---|---|---|---|---|---|---|
| S1H6N4F1 | A | S1 | C | | F | X | | 2238 | 0.82 | 0.86 | −1.04 | −0.04 |
| S1H6N3F1 | A | S1 | H | | F | | | 2035 | 0.32 | 0.34 | −1.04 | −0.01 |
| S1H6N4F1A1 | A | S1 | C | | F | X | | 2280 | 0.15 | 0.16 | −1.05 | −0.01 |
| S1H7N6 | A | S1 | C | R | | | | 2660 | 0.36 | 0.40 | −1.09 | −0.03 |
| S2H8N7F1 | A | S2 | C | R | F | | | 3463 | 0.14 | 0.17 | −1.19 | −0.03 |
| S1H2N1 | A | S1 | O | | | | | 835 | 0.04 | 0.05 | −1.23 | −0.01 |
| S1H5N4F1 | A | S1 | C | B | F | | | 2076 | 27.61 | 33.96 | −1.23 | −6.35 |
| S2H6N5 | A | S2 | C | R | | | | 2586 | 0.61 | 0.76 | −1.24 | −0.14 |
| S1H5N4F3 | A | S1 | C | B | E | | | 2368 | 1.00 | 1.33 | −1.32 | −0.32 |
| S1H9N8F1 | A | S1 | C | R | F | | | 3537 | 0.12 | 0.17 | −1.34 | −0.04 |
| G1H3N5 | A | S1 | C | | | T | Y | 1825 | 0.06 | 0.07 | −1.34 | −0.02 |
| S1H3N3F1 | A | S1 | H | N | F | | | 1549 | 0.20 | 0.29 | −1.44 | −0.09 |

Over 1.5 times overexpressed in OB:

| | | | | | | | | | | | |
|---|---|---|---|---|---|---|---|---|---|---|---|
| G1H5N4F1 | A | S1 | C | B | F | Y | 2092 | 0.68 | 1.23 | −1.80 | −0.55 |
| G1H5N4 | A | S1 | C | B | | Y | 1946 | 0.11 | 0.21 | −1.84 | −0.09 |
| S2H2N2F1 | A | S2 | O | | F | | 1475 | 0.13 | 0.26 | −1.96 | −0.13 |

Over 2 times overexpressed in OB:

| | | | | | | | | | | | | |
|---|---|---|---|---|---|---|---|---|---|---|---|---|
| S2H6N5F2 | A | S2 | C | R | E | | | 2879 | 0.23 | 0.53 | −2.26 | −0.30 |
| S3H6N5 | A | S3 | C | R | | | | 2878 | 0.56 | 1.59 | −2.85 | −1.03 |
| H10N2F1P2 | A | | M | | F | P | | 2349 | 0.07 | 0.20 | −2.86 | −0.13 |
| S2H4N3F1P1 | A | S2 | H | N | F | P | | 2082 | 0.05 | 0.18 | −3.64 | −0.13 |
| S1H7N5F1 | A | S1 | C | | F | | X | 2603 | 0.02 | 0.08 | −4.81 | −0.06 |
| S3H7N6F1 | A | S3 | C | R | F | | | 3389 | 0.01 | 0.05 | −4.96 | −0.04 |
| H4N3F1P1 | A | | H | | F | P | | 1500 | 0.12 | 0.60 | −5.03 | −0.48 |
| S2H6N4 | A | S2 | C | | | | X | 2383 | 0.03 | 0.19 | −5.88 | −0.16 |
| S2H6N5F4 | A | S2 | C | R | E | | | 3171 | 0.07 | 0.39 | −6.00 | −0.33 |
| H5N4P1 | A | | C | B | | P | | 1719 | 0.50 | 3.85 | −7.75 | −3.35 |
| S2H6N5F3 | A | S2 | C | R | E | | | 3025 | 0.02 | 0.17 | −8.97 | −0.16 |
| H4N3P1 | A | | H | | | P | | 1354 | 0.06 | 0.55 | −9.55 | −0.50 |
| H5N4F1P1 | A | | C | B | F | P | | 1865 | 0.09 | 2.38 | −25.3 | −2.28 |

Not detected in MSC:

| | | | | | | | | | | | | | |
|---|---|---|---|---|---|---|---|---|---|---|---|---|---|
| S1H9N8F3 | A | S1 | C | R | E | | | | 3829 | 0.00 | 0.01 | —∞ | −0.01 |
| S1H5N5F3 | A | S1 | C | | E | | Q | | 2571 | 0.00 | 0.02 | —∞ | −0.02 |
| H5N3F1P1 | A | | H | | F | P | | | 1662 | 0.00 | 0.02 | —∞ | −0.02 |
| S1H6N6F3 | A | S1 | C | R | E | | Q | | 2937 | 0.00 | 0.03 | —∞ | −0.03 |
| S3H4N4 | A | S3 | C | | | | Q | | 2350 | 0.00 | 0.03 | —∞ | −0.03 |
| H4N3F2P1 | A | | H | | E | P | | | 1646 | 0.00 | 0.03 | —∞ | −0.03 |
| S2H5N4F1P1 | A | S2 | C | B | F | P | | | 2447 | 0.00 | 0.03 | —∞ | −0.03 |
| S2H6N5F1P1 | A | S2 | C | R | F | P | | | 2812 | 0.00 | 0.03 | —∞ | −0.03 |
| S3H7N6 | A | S3 | C | R | | | | | 3243 | 0.00 | 0.03 | —∞ | −0.03 |
| H3N6F1P1 | A | | C | | F | P | T | | 1947 | 0.00 | 0.03 | —∞ | −0.03 |
| H4N5F2P1 | A | | C | | E | P | T | | 2052 | 0.00 | 0.03 | —∞ | −0.03 |
| H3N5F1P1 | A | | C | | F | P | T | | 1744 | 0.00 | 0.03 | —∞ | −0.03 |
| H3N4F1P1 | A | | C | | F | P | T | | 1541 | 0.00 | 0.03 | —∞ | −0.03 |
| S1H3N3F1P2 | A | S1 | H | N | F | P | | | 1709 | 0.00 | 0.03 | —∞ | −0.03 |
| S1H4N5F3 | A | S1 | C | | E | | T | | 2409 | 0.00 | 0.04 | —∞ | −0.04 |
| S2H4N5 | A | S2 | C | | | | T | | 2262 | 0.00 | 0.04 | —∞ | −0.04 |
| S3H8N7F3 | A | S3 | C | R | E | | | | 4046 | 0.00 | 0.04 | —∞ | −0.04 |
| S2H5N4F3 | A | S2 | C | B | E | | | | 2659 | 0.00 | 0.04 | —∞ | −0.04 |
| S2H8N7F2 | A | S2 | C | R | E | | | | 3609 | 0.00 | 0.04 | —∞ | −0.04 |
| S4H7N6F1 | A | S4 | C | R | F | | | | 3680 | 0.00 | 0.05 | —∞ | −0.05 |
| H7N4P1 | A | | C | | | P | X | | 2043 | 0.00 | 0.05 | —∞ | −0.05 |
| H7N6F1P1 | A | | C | R | F | P | | | 2595 | 0.00 | 0.05 | —∞ | −0.05 |
| S2H8N7 | A | S2 | C | R | | | | | 3317 | 0.00 | 0.06 | —∞ | −0.06 |
| S2H9N8F1 | A | S2 | C | R | F | | | | 3828 | 0.00 | 0.06 | —∞ | −0.06 |
| H6N5F3P1 | A | | C | R | E | P | | | 2522 | 0.00 | 0.06 | —∞ | −0.06 |
| S1H6N5F1P1 | A | S1 | C | R | F | P | | | 2521 | 0.00 | 0.06 | —∞ | −0.06 |
| H3N3F1P1 | A | | H | N | F | P | | | 1338 | 0.00 | 0.06 | —∞ | −0.06 |
| H6N4F3P1 | A | | C | | E | P | X | | 2319 | 0.00 | 0.06 | —∞ | −0.06 |
| S1H9N8F2 | A | S1 | C | R | E | | | | 3683 | 0.00 | 0.07 | —∞ | −0.07 |
| H3N3P1 | A | | H | N | | P | | | 1192 | 0.00 | 0.07 | —∞ | −0.07 |
| G1S1H5N3 | A | S2 | H | | | | | Y | 2034 | 0.00 | 0.07 | —∞ | −0.07 |
| S1H5N5F2 | A | S1 | C | | E | | Q | | 2425 | 0.00 | 0.08 | —∞ | −0.08 |
| S1H3N5 | A | S1 | C | | | | T | | 1809 | 0.00 | 0.08 | —∞ | −0.08 |
| S2H8N7F4 | A | S2 | C | R | E | | | | 3901 | 0.00 | 0.08 | —∞ | −0.08 |
| S2H4N5F2P2 | A | S2 | C | | E | P | T | | 2714 | 0.00 | 0.08 | —∞ | −0.08 |
| S2H4N4F1 | A | S2 | C | | F | | Q | | 2205 | 0.00 | 0.08 | —∞ | −0.08 |
| S1H10N9 | A | S1 | C | R | | | | | 3756 | 0.00 | 0.09 | —∞ | −0.09 |
| H3N4P1 | A | | C | | | P | T | | 1395 | 0.00 | 0.09 | —∞ | −0.09 |
| H5N4F2P1 | A | | C | B | E | P | | | 2011 | 0.00 | 0.10 | —∞ | −0.10 |
| S2H5N3P2 | A | S2 | H | | | P | | | 2178 | 0.00 | 0.11 | —∞ | −0.11 |
| S2H5N5F1 | A | S2 | C | | F | | Q | | 2570 | 0.00 | 0.11 | —∞ | −0.11 |
| H5N4F3P1 | A | | C | B | E | P | | | 2157 | 0.00 | 0.12 | —∞ | −0.12 |
| S1H4N6 | A | S1 | C | | | | T | | 2174 | 0.00 | 0.12 | —∞ | −0.12 |
| G1S2H6N5 | A | S3 | C | R | | | | Y | 2893 | 0.00 | 0.12 | —∞ | −0.12 |
| S1H5N4P1 | A | S1 | C | B | | P | | | 2010 | 0.00 | 0.17 | —∞ | −0.17 |
| G1H6N4P1 | A | S1 | C | | | P | X | Y | 2188 | 0.00 | 0.19 | —∞ | −0.19 |
| H4N4F1P1 | A | | C | | F | P | Q | | 1703 | 0.00 | 0.25 | —∞ | −0.25 |
| S1H5N4F1P1 | A | S1 | C | B | F | P | | | 2156 | 0.00 | 0.58 | —∞ | −0.58 |
| H4N4P1 | A | | C | | | P | Q | | 1557 | 0.00 | 0.75 | —∞ | −0.75 |
| H6N5F1P1 | A | | C | R | F | P | | | 2230 | 0.00 | 0.88 | —∞ | −0.88 |

Data are average of 5 analyzed cell lines.
The relative change (relat.) and absolute change (diff.) in signal intensity (% of total profile) are indicated.
Composition codes:
S, N-acetylneuraminic acid;
G, N-glycolylneuraminic acid;
H, hexose;
N, N-acetylhexosamine;
F, deoxyhexose;
P, sulfate or phosphate ester;
A, acetyl ester.
Structure codes:
A, acidic glycan;
Sx, x sialic acid groups;
M, high-mannose type;
L, low-mannose type;
S, soluble glycan;
H, hybrid-type;
C, complex-type;
N, monoantennary;
B, biantennary-size;
R, large complex-type;
F, one fucose;
E, multifucosylated;
P, sulfated or phosphorylated;
T/Q, terminal N-acetylhexosamine;
X, terminal hexose;
Y, Neu5Gc;
A, acetylated.
The signals are arranged according to relative expression in MSC compared to OB (relat.) as indicated in the subtitles.

TABLE 2
Variation in acidic N-glycans
expressed as relation to the glycan signal.

| | | | |
|---|---|---|---|
| Composition | m/z | MSC | OB |

Large variation in MSC:

| | | | |
|---|---|---|---|
| S3H7N6F3 | 3681 | 4.08 | 0.00 |
| S1H11N10 | 4121 | 4.08 | 0.00 |
| S3H7N6F4 | 3827 | 4.08 | 0.00 |
| S1H4N3F3 | 2003 | 4.08 | 0.00 |
| S3H8N7F1 | 3754 | 4.08 | 4.08 |
| S2H3N5F1 | 2246 | 4.08 | 2.04 |
| S2H4N3F1P1 | 2082 | 4.08 | 1.53 |
| S1H6N5F4 | 2880 | 4.03 | 0.00 |
| S1H9N8F1 | 3537 | 3.35 | 1.83 |
| S2H6N5F4 | 3171 | 2.88 | 1.71 |
| S1H2N1 | 835 | 2.04 | 2.04 |
| H5N3F2P1 | 1808 | 2.04 | 0.00 |
| S2H2N2 | 1329 | 2.04 | 0.00 |
| S2H3N5F2 | 2392 | 2.04 | 0.00 |
| S3H6N4F1P1 | 2900 | 2.04 | 0.00 |
| S1H7N6F5 | 3391 | 2.04 | 0.00 |
| S1H3N4 | 1606 | 2.04 | 0.00 |
| S2H4N4 | 2059 | 2.04 | 0.00 |
| S1H7N6F4 | 3245 | 2.04 | 0.00 |
| S2H3N3 | 1694 | 2.04 | 0.00 |
| G1H5N3 | 1743 | 2.04 | 0.00 |
| S1H8N7F3 | 3464 | 2.04 | 0.00 |
| S1H7N5F1A1 | 2645 | 2.04 | 2.04 |
| S1H5N2 | 1524 | 2.04 | 2.04 |
| H3N2P1 | 989 | 2.04 | 2.04 |
| S1H7N5F1 | 2603 | 2.04 | 2.04 |
| S3H7N6F1 | 3389 | 2.04 | 1.17 |
| S2H6N4 | 2383 | 2.04 | 1.61 |
| S1H4N5 | 1971 | 2.04 | 0.00 |
| S2H4N3 | 1856 | 2.04 | 0.00 |
| S2H5N5 | 2424 | 2.04 | 0.00 |
| G1H3N2 | 1216 | 2.04 | 0.00 |
| G1H4N3 | 1581 | 2.04 | 0.00 |
| S2H3N4F2 | 2189 | 2.04 | 0.00 |
| S1H4N4F2 | 2060 | 2.04 | 0.00 |
| S1H4N3F1P1 | 1791 | 2.04 | 2.04 |
| G1S2H5N4F1 | 2674 | 2.04 | 2.04 |
| H10N2F1P2 | 2349 | 2.04 | 2.04 |
| S2H7N6F3 | 3390 | 1.98 | 1.71 |
| S1H6N5F2 | 2587 | 1.82 | 0.98 |
| S3H6N5F1 | 3024 | 1.79 | 1.27 |
| S2H7N6F4 | 3536 | 1.67 | 4.08 |
| S2H5N3 | 2018 | 1.61 | 1.67 |
| S2H6N5F2 | 2879 | 1.60 | 1.33 |
| S1H8N7 | 3026 | 1.58 | 0.00 |
| S1H7N6F2 | 2953 | 1.56 | 2.04 |
| S2H8N7F1 | 3463 | 1.51 | 1.48 |
| S2H5N3F2P1 | 2390 | 1.50 | 0.00 |
| S1H5N5F1 | 2279 | 1.50 | 0.61 |

Medium variation:

| | | | |
|---|---|---|---|
| S1H7N6F3 | 3099 | 1.49 | 1.42 |
| H3N6F3P1 | 2239 | 1.48 | 0.92 |
| S2H6N3F1P1 | 2406 | 1.44 | 2.04 |
| S2H1N3F1 | 1516 | 1.43 | 2.04 |
| S2H6N5F3 | 3025 | 1.42 | 1.68 |
| S1H5N4F4 | 2514 | 1.39 | 0.00 |
| S1H7N5 | 2457 | 1.38 | 4.08 |
| S2H3N2F1 | 1637 | 1.37 | 2.04 |
| H5N4F1P1 | 1865 | 1.37 | 0.94 |
| S1H6N6F1 | 2644 | 1.35 | 1.65 |
| H4N3F1P1 | 1500 | 1.35 | 0.98 |
| H4N2P1 | 1151 | 1.35 | 2.04 |
| H4N3P1 | 1354 | 1.33 | 0.94 |
| S2H4N2F1 | 1799 | 1.33 | 0.00 |
| S2H3N3F1 | 1840 | 1.32 | 0.00 |
| S1H5N5 | 2133 | 1.32 | 0.00 |
| S1H6N3F1 | 2035 | 1.32 | 0.78 |
| S2H7N6 | 2952 | 1.31 | 2.04 |
| S2H4N3F1 | 2002 | 1.31 | 0.97 |
| G1H5N4 | 1946 | 1.31 | 1.08 |
| S2H5N3F1 | 2164 | 1.30 | 0.00 |
| S1H7N4 | 2254 | 1.30 | 1.34 |
| G1H3N5 | 1825 | 1.30 | 2.04 |
| S2H5N4F2 | 2513 | 1.30 | 2.04 |
| S1H2N2 | 1038 | 1.29 | 2.04 |
| S1H3N3F1 | 1549 | 1.29 | 0.76 |
| S1H7N3 | 2051 | 1.29 | 2.04 |
| S2H2N2F1 | 1475 | 1.29 | 0.66 |
| S2H7N6F1 | 3098 | 1.26 | 1.03 |
| S1H3N3 | 1403 | 1.23 | 0.82 |
| H5N4P1 | 1719 | 1.18 | 0.97 |
| S3H6N5 | 2878 | 1.18 | 1.08 |
| S1H6N5F3 | 2733 | 1.11 | 0.76 |
| S1H8N7F1 | 3172 | 1.08 | 2.04 |
| S1H3N2 | 1200 | 1.02 | 0.40 |
| S2H6N5 | 2586 | 1.01 | 0.87 |

Slight variation in MSC:

| | | | |
|---|---|---|---|
| S1H7N6 | 2660 | 0.98 | 0.74 |
| S2H6N5F1 | 2732 | 0.98 | 0.65 |
| S1H5N4F3 | 2368 | 0.96 | 0.42 |
| S1H6N4F1A1 | 2280 | 0.95 | 0.91 |
| G1H5N4F1 | 2092 | 0.76 | 0.27 |
| S2H2N3F1 | 1678 | 0.72 | 0.58 |
| S1H6N4F1 | 2238 | 0.69 | 0.43 |
| S2H5N4F1 | 2367 | 0.57 | 0.71 |
| S1H5N3F1 | 1873 | 0.56 | 0.33 |
| S1H4N2 | 1362 | 0.54 | 0.76 |
| S1H6N3 | 1889 | 0.49 | 0.32 |
| S1H4N4F1 | 1914 | 0.47 | 0.15 |
| S1H7N6F1 | 2807 | 0.44 | 0.49 |
| S2H5N4 | 2221 | 0.43 | 0.64 |
| S1H4N3 | 1565 | 0.40 | 0.29 |
| S1H4N4 | 1768 | 0.39 | 0.16 |
| S1H5N4F2 | 2222 | 0.37 | 0.63 |
| S1H5N4 | 1930 | 0.28 | 0.19 |
| S1H6N5F1 | 2441 | 0.25 | 0.15 |
| S1H6N5 | 2295 | 0.24 | 0.15 |
| S1H5N3 | 1727 | 0.22 | 0.25 |
| S1H4N3F1 | 1711 | 0.16 | 0.21 |
| S1H5N4F1 | 2076 | 0.16 | 0.20 |

Detected only in OB:

| | | | |
|---|---|---|---|
| S1H9N8F3 | 3829 | 0.00 | 4.08 |
| S1H5N5F3 | 2571 | 0.00 | 2.04 |
| H5N3F1P1 | 1662 | 0.00 | 2.04 |
| S1H6N6F3 | 2937 | 0.00 | 2.04 |
| S3H4N4 | 2350 | 0.00 | 2.04 |
| H4N3F2P1 | 1646 | 0.00 | 2.04 |
| S2H5N4F1P1 | 2447 | 0.00 | 4.08 |
| S2H6N5F1P1 | 2812 | 0.00 | 2.04 |
| S3H7N6 | 3243 | 0.00 | 2.04 |
| H3N6F1P1 | 1947 | 0.00 | 2.04 |
| H4N5F2P1 | 2052 | 0.00 | 2.04 |
| H3N5F1P1 | 1744 | 0.00 | 2.04 |
| H3N4F1P1 | 1541 | 0.00 | 2.04 |
| S1H3N3F1P2 | 1709 | 0.00 | 2.04 |
| S1H4N5F3 | 2409 | 0.00 | 2.04 |
| S2H4N5 | 2262 | 0.00 | 2.04 |
| S3H8N7F3 | 4046 | 0.00 | 2.04 |
| S2H5N4F3 | 2659 | 0.00 | 4.08 |
| S2H8N7F2 | 3609 | 0.00 | 2.04 |
| S4H7N6F1 | 3680 | 0.00 | 2.04 |
| H7N4P1 | 2043 | 0.00 | 2.04 |
| H7N6F1P1 | 2595 | 0.00 | 2.04 |
| S2H8N7 | 3317 | 0.00 | 2.04 |
| S2H9N8F1 | 3828 | 0.00 | 3.30 |
| H6N5F3P1 | 2522 | 0.00 | 1.40 |
| S1H6N5F1P1 | 2521 | 0.00 | 1.34 |
| H3N3F1P1 | 1338 | 0.00 | 2.04 |
| H6N4F3P1 | 2319 | 0.00 | 2.04 |
| S1H9N8F2 | 3683 | 0.00 | 2.04 |
| H3N3P1 | 1192 | 0.00 | 2.04 |
| G1S1H5N3 | 2034 | 0.00 | 2.04 |
| S1H5N5F2 | 2425 | 0.00 | 2.04 |
| S1H3N5 | 1809 | 0.00 | 4.08 |
| S2H8N7F4 | 3901 | 0.00 | 2.00 |
| S2H4N5F2P2 | 2714 | 0.00 | 2.04 |
| S2H4N4F1 | 2205 | 0.00 | 2.04 |
| S1H10N9 | 3756 | 0.00 | 2.04 |
| H3N4P1 | 1395 | 0.00 | 2.04 |
| H5N4F2P1 | 2011 | 0.00 | 2.65 |
| S2H5N3P2 | 2178 | 0.00 | 4.08 |
| S2H5N5F1 | 2570 | 0.00 | 1.33 |
| H5N4F3P1 | 2157 | 0.00 | 1.78 |
| S1H4N6 | 2174 | 0.00 | 1.91 |
| G1S2H6N5 | 2893 | 0.00 | 2.04 |
| S1H5N4P1 | 2010 | 0.00 | 1.75 |
| G1H6N4P1 | 2188 | 0.00 | 1.70 |
| H4N4F1P1 | 1703 | 0.00 | 1.37 |
| S1H5N4F1P1 | 2156 | 0.00 | 0.61 |
| H4N4P1 | 1557 | 0.00 | 1.58 |
| H6N5F1P1 | 2230 | 0.00 | 0.65 |

Data are from 5 cell lines and differentiated cells.
MSC: bone marrow mesenchymal cell lines;
OB: osteblast differentiated.

TABLE 3
Differential expression of neutral N-glycan signals in bone marrow
mesenchymal stem cells (MSC) versus osteoblast-differentiated
cells (OB) as analyzed by MALDI-TOF mass spectrometric
profiling.

| | | | | | | |
|---|---|---|---|---|---|---|
| Composition | Structure | m/z | MSC | OB | relat. | diff. |

Not detected in OB:

| | | | | | | | | |
|---|---|---|---|---|---|---|---|---|
| H3N2F4 | O | E | | 1517 | 0.02 | 0.00 | ∞ | 0.02 |
| H4N5F3 | C | E | T | 1850 | 0.02 | 0.00 | ∞ | 0.02 |
| H9N1 | S | | | 1702 | 0.21 | 0.00 | ∞ | 0.21 |

Over 2 times overexpressed in MSC:

| | | | | | | | | |
|---|---|---|---|---|---|---|---|---|
| H7N1 | S | | | 1378 | 0.70 | 0.12 | 5.84 | 0.58 |
| H6N1 | S | | | 1216 | 1.92 | 0.48 | 4.01 | 1.44 |
| H3N1 | S | | | 730 | 1.93 | 0.50 | 3.90 | 1.44 |
| H5N1 | S | | | 1054 | 3.65 | 0.97 | 3.76 | 2.68 |
| H4N1 | S | | | 892 | 2.74 | 0.75 | 3.64 | 1.99 |
| H4N5F3 | C | E | T | 2142 | 0.03 | 0.01 | 3.57 | 0.02 |
| H2N1 | S | | | 568 | 0.77 | 0.23 | 3.41 | 0.55 |
| H2N2F3 | O | E | | 1209 | 0.06 | 0.02 | 3.02 | 0.04 |

Over 1.5 times overexpression in MSC:

| | | | | | | | |
|---|---|---|---|---|---|---|---|
| H8N1 | S | | 1540 | 0.57 | 0.34 | 1.69 | 0.24 |
| H9N2 | M | | 1905 | 12.31 | 7.70 | 1.60 | 4.61 |
| H6N2F1 | M | F | 1565 | 0.20 | 0.13 | 1.57 | 0.07 |
| H8N2 | M | | 1743 | 13.88 | 8.96 | 1.55 | 4.92 |

Less than 1.5 times overexpression in MSC:

| | | | | | | | | | |
|---|---|---|---|---|---|---|---|---|---|
| H3N5F1 | C | | F | T | 1688 | 0.41 | 0.28 | 1.47 | 0.13 |
| H6N2 | M | | | | 1419 | 13.73 | 10.06 | 1.37 | 3.68 |
| H7N2 | M | | | | 1581 | 10.76 | 8.31 | 1.29 | 2.45 |
| H11N2 | M | G | | | 2229 | 0.06 | 0.05 | 1.23 | 0.01 |
| H3N4F1 | C | | F | T | 1485 | 0.73 | 0.60 | 1.22 | 0.13 |
| H10N2 | M | G | | | 2067 | 0.88 | 0.75 | 1.17 | 0.13 |
| H2N2 | L | | | | 771 | 1.09 | 0.94 | 1.16 | 0.15 |
| H12N2 | M | G | | | 2391 | 0.03 | 0.02 | 1.07 | 0.00 |

Less than 1.5 times overexpression in OB:

| | | | | | | | | | |
|---|---|---|---|---|---|---|---|---|---|
| H3N2F1 | L | | F | | 1079 | 2.93 | 3.03 | −1.03 | −0.09 |
| H4N5F2 | C | | E | T | 1996 | 0.12 | 0.12 | −1.06 | −0.01 |
| H3N2 | L | | | | 933 | 1.92 | 2.04 | −1.06 | −0.11 |
| H4N2 | L | | | | 1095 | 2.07 | 2.22 | −1.07 | −0.15 |
| H4N4F2 | C | | E | Q | 1793 | 0.19 | 0.23 | −1.19 | −0.04 |
| H3N4 | C | | | T | 1339 | 0.04 | 0.05 | −1.21 | −0.01 |
| H5N2 | M | | | | 1257 | 7.18 | 8.76 | −1.22 | −1.58 |
| H3N3 | H | N | | | 1136 | 0.55 | 0.67 | −1.23 | −0.12 |
| H7N3 | H | | | | 1784 | 0.19 | 0.27 | −1.44 | −0.08 |
| H5N4F3 | C | B | E | | 2101 | 0.23 | 0.33 | −1.46 | −0.11 |
| H3N3F1 | H | N | F | | 1282 | 0.53 | 0.78 | −1.47 | −0.25 |
| H4N2F1 | L | | F | | 1241 | 0.37 | 0.55 | −1.49 | −0.18 |

Over 1.5 times overexpressed in OB:

| | | | | | | | | | |
|---|---|---|---|---|---|---|---|---|---|
| H5N2F1 | M | | F | | 1403 | 0.32 | 0.51 | −1.59 | −0.19 |
| H4N3 | H | | | | 1298 | 1.06 | 1.81 | −1.71 | −0.75 |
| H6N5F4 | C | R | E | | 2612 | 0.02 | 0.03 | −1.72 | −0.01 |
| H5N5F3 | C | | E | Q | 2304 | 0.02 | 0.03 | −1.76 | −0.01 |
| H5N5 | C | | | Q | 1866 | 0.03 | 0.05 | −1.77 | −0.02 |
| H2N2F1 | L | | F | | 917 | 1.08 | 2.00 | −1.85 | −0.92 |
| H5N3F1 | H | | F | | 1606 | 0.92 | 1.76 | −1.91 | −0.84 |
| H2N3F1 | H | N | F | T | 1120 | 0.01 | 0.02 | −1.93 | −0.01 |

Over 2 times overexpressed in OB:

| | | | | | | | | | |
|---|---|---|---|---|---|---|---|---|---|
| H5N4 | C | B | | | 1663 | 3.72 | 7.72 | −2.07 | −4.00 |
| H4N4F1 | C | | F | Q | 1647 | 0.28 | 0.60 | −2.13 | −0.32 |
| H4N3F1 | H | | F | | 1444 | 0.65 | 1.42 | −2.18 | −0.77 |
| H5N5F1 | C | | F | Q | 2012 | 0.06 | 0.13 | −2.19 | −0.07 |
| H7N6F1 | C | R | F | | 2539 | 0.04 | 0.10 | −2.40 | −0.06 |
| H6N3F1 | H | | F | | 1768 | 0.31 | 0.75 | −2.41 | −0.44 |
| H6N3 | H | | | | 1622 | 1.73 | 4.35 | −2.51 | −2.62 |
| H5N3 | H | | | | 1460 | 1.07 | 2.69 | −2.52 | −1.62 |
| H6N5 | C | R | | | 2028 | 0.61 | 1.66 | −2.72 | −1.05 |
| H7N4 | C | | | X | 1987 | 0.04 | 0.11 | −2.81 | −0.07 |
| H7N6 | C | R | | | 2393 | 0.08 | 0.24 | −2.94 | −0.16 |
| H8N7 | C | R | | | 2758 | 0.01 | 0.03 | −2.99 | −0.02 |
| H5N4F1 | C | B | F | | 1809 | 2.31 | 7.12 | −3.08 | −4.81 |
| H5N4F2 | C | B | E | | 1955 | 0.33 | 1.02 | −3.14 | −0.70 |
| H6N5F1 | C | R | F | | 2174 | 0.65 | 2.09 | −3.21 | −1.44 |
| H6N4F2 | C | | E | X | 2117 | 0.01 | 0.03 | −3.32 | −0.02 |
| H4N4 | C | | | Q | 1501 | 0.20 | 0.85 | −4.32 | −0.66 |
| H6N5F3 | C | R | E | | 2466 | 0.01 | 0.02 | −4.33 | −0.02 |
| H6N4F1 | C | | F | X | 1971 | 0.06 | 0.26 | −4.64 | −0.21 |
| H4N3F2 | H | | E | | 1590 | 0.05 | 0.25 | −4.84 | −0.20 |
| H6N4 | C | | | X | 1825 | 0.05 | 0.25 | −5.30 | −0.20 |
| H6N5F2 | C | R | E | | 2320 | 0.01 | 0.08 | −8.70 | −0.07 |
| H5N3F2 | H | | E | | 1752 | 0.02 | 0.17 | −11.19 | −0.16 |

Not detected in MSC:

| | | | | | | | | | |
|---|---|---|---|---|---|---|---|---|---|
| H8N4 | C | | | X | 2149 | 0.00 | 0.01 | —∞ | −0.01 |
| H6N6 | C | R | | Q | 2231 | 0.00 | 0.01 | —∞ | −0.01 |
| H2N3 | H | N | | T | 974 | 0.00 | 0.01 | —∞ | −0.01 |
| H5N5F2 | C | | E | Q | 2158 | 0.00 | 0.01 | —∞ | −0.01 |
| H4N5 | C | | | T | 1704 | 0.00 | 0.02 | —∞ | −0.02 |
| H3N3F2 | H | N | E | | 1428 | 0.00 | 0.02 | —∞ | −0.02 |
| H8N2F1 | M | | F | | 1889 | 0.00 | 0.03 | —∞ | −0.03 |
| H7N4F1 | C | | F | X | 2133 | 0.00 | 0.03 | —∞ | −0.03 |
| H3N6F1 | C | | F | T | 1891 | 0.00 | 0.03 | —∞ | −0.03 |
| H1N2 | L | | | | 609 | 0.00 | 0.05 | —∞ | −0.05 |
| H1N6 | O | | | | 1421 | 0.00 | 0.10 | —∞ | −0.10 |

Data are average of 5 analyzed cell lines. The signals are arranged according to relative expression in MSC compared to OB (relat.) as indicated in the subtitles. Codes are as in preceding Table.

TABLE 4
Variation in neutral N-glycans expressed as relation to the glycan signal.
Data are from 5 cell lines and differentiated cells. MSC: bone marrow
mesenchymal cell lines; OB: osteblast differentiated.

| | | | |
|---|---|---|---|
| Composition | m/z | MSC | OB |

Large variation in MSC:

| | | | |
|---|---|---|---|
| H3N4 | 1339 | 2.45 | 1.23 |
| H2N3F1 | 1120 | 2.45 | 1.49 |
| H4N5F3 | 1850 | 2.45 | 0.00 |
| H4N5F3 | 2142 | 2.45 | 2.04 |
| H5N3F2 | 1752 | 2.45 | 0.25 |
| H6N4F2 | 2117 | 2.40 | 0.67 |
| H8N7 | 2758 | 1.75 | 0.80 |
| H6N4 | 1825 | 1.59 | 0.65 |
| H6N5F2 | 2320 | 1.59 | 0.40 |
| H3N2F4 | 1517 | 1.57 | 0.00 |
| H5N5 | 1866 | 1.55 | 1.30 |
| H6N5F3 | 2466 | 1.55 | 0.58 |
| H4N3F2 | 1590 | 1.55 | 0.57 |
| H5N5F3 | 2304 | 1.55 | 0.89 |

Medium variation in MSC:

| | | | |
|---|---|---|---|
| H2N2F3 | 1209 | 1.25 | 1.56 |
| H2N1 | 568 | 1.25 | 1.20 |
| H6N5F4 | 2612 | 1.19 | 0.56 |
| H7N4 | 1987 | 1.18 | 0.41 |
| H12N2 | 2391 | 1.13 | 1.10 |
| H7N6F1 | 2539 | 0.79 | 0.48 |
| H5N5F1 | 2012 | 0.65 | 0.38 |
| H4N1 | 892 | 0.65 | 0.94 |
| H6N4F1 | 1971 | 0.61 | 0.28 |
| H4N4 | 1501 | 0.58 | 0.45 |
| H5N1 | 1054 | 0.55 | 0.83 |
| H7N1 | 1378 | 0.53 | 1.42 |
| H9N1 | 1702 | 0.52 | 0.00 |
| H3N1 | 730 | 0.51 | 0.96 |

Slight variation in MSC:

| | | | |
|---|---|---|---|
| H6N1 | 1216 | 0.47 | 0.74 |
| H6N3F1 | 1768 | 0.42 | 0.27 |
| H6N5 | 2028 | 0.41 | 0.52 |
| H6N5F1 | 2174 | 0.40 | 0.49 |
| H5N4F1 | 1809 | 0.40 | 0.14 |
| H2N2 | 771 | 0.37 | 0.13 |
| H4N4F1 | 1647 | 0.37 | 0.33 |
| H10N2 | 2067 | 0.36 | 0.19 |
| H11N2 | 2229 | 0.35 | 0.69 |
| H6N3 | 1622 | 0.35 | 0.21 |
| H7N6 | 2393 | 0.33 | 0.62 |
| H7N3 | 1784 | 0.30 | 0.30 |
| H5N3F1 | 1606 | 0.28 | 0.17 |
| H5N2F1 | 1403 | 0.27 | 0.28 |
| H5N4 | 1663 | 0.27 | 0.11 |
| H4N2F1 | 1241 | 0.26 | 0.30 |
| H8N1 | 1540 | 0.26 | 0.16 |
| H4N5F2 | 1996 | 0.26 | 0.65 |
| H3N5F1 | 1688 | 0.25 | 0.35 |
| H6N2F1 | 1565 | 0.24 | 0.54 |
| H4N4F2 | 1793 | 0.23 | 0.35 |
| H5N4F3 | 2101 | 0.23 | 0.23 |
| H3N2F1 | 1079 | 0.21 | 0.21 |
| H5N3 | 1460 | 0.20 | 0.20 |
| H5N4F2 | 1955 | 0.19 | 0.23 |
| H3N4F1 | 1485 | 0.18 | 0.25 |
| H3N3F1 | 1282 | 0.18 | 0.28 |
| H4N3F1 | 1444 | 0.18 | 0.26 |
| H9N2 | 1905 | 0.17 | 0.13 |
| H8N2 | 1743 | 0.16 | 0.10 |
| H5N2 | 1257 | 0.15 | 0.15 |
| H3N2 | 933 | 0.15 | 0.22 |
| H6N2 | 1419 | 0.14 | 0.13 |
| H2N2F1 | 917 | 0.14 | 0.16 |
| H3N3 | 1136 | 0.14 | 0.23 |
| H4N3 | 1298 | 0.13 | 0.24 |
| H7N2 | 1581 | 0.12 | 0.10 |
| H4N2 | 1095 | 0.10 | 0.17 |

Not detected in MSC:

| | | | |
|---|---|---|---|
| H8N4 | 2149 | 0.00 | 2.04 |
| H6N6 | 2231 | 0.00 | 2.04 |
| H2N3 | 974 | 0.00 | 2.04 |
| H5N5F2 | 2158 | 0.00 | 1.78 |
| H4N5 | 1704 | 0.00 | 2.04 |
| H3N3F2 | 1428 | 0.00 | 2.04 |
| H8N2F1 | 1889 | 0.00 | 2.04 |
| H7N4F1 | 2133 | 0.00 | 0.85 |
| H3N6F1 | 1891 | 0.00 | 1.30 |
| H1N2 | 609 | 0.00 | 1.08 |
| H1N6 | 1421 | 0.00 | 2.04 |

TABLE 5
Structure assignments of BM MSC acidic N-glycans

| m/z | Structure |
|---|---|
| 989 | 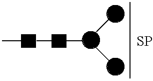 |
| 1151 | 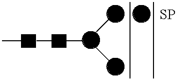 |
| 1297 | 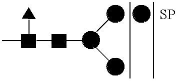 |
| 1338 | 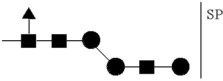 |
| 1354 | 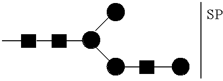 |
| 1395 | 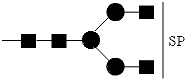 |
| 1403 | 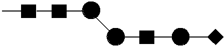 |
| 1500 | 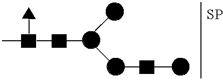 |
| 1549 | 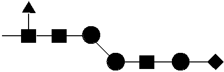 |
| 1555 | 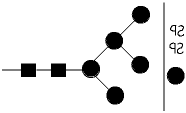 |
| 1557 | 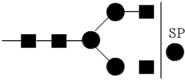 |
| 1565 | 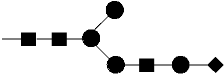 |
| 1581 | 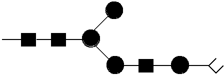 |
| 1646 | 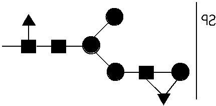 |
| 1703 | 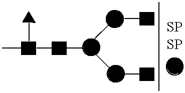 |
| 1709 | 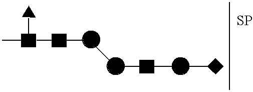 |
| 1711 | 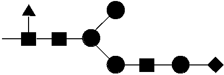 |
| 1719 | 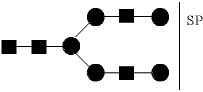 |
| 1727 | 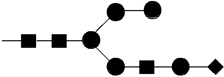 |
| 1744 | 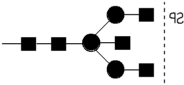 |
| 1758 | 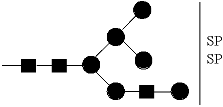 |
| 1768 | 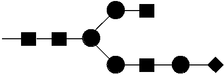 |
| 1791 | 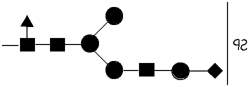 |
| 1808 | 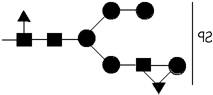 |
| 1840 | 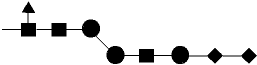 |
| 1856 | 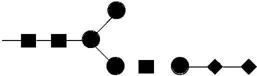 |
| 1865 | 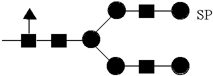 |
| 1873 | 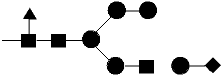 |
| 1889 | 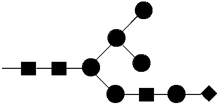 |
| 1914 | 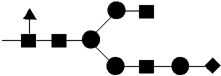 |
| 1930 | 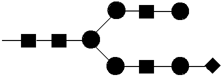 |
| 1946 | 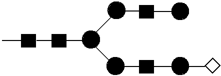 |
| 2002 | 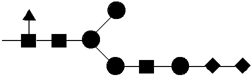 |
| 2003 | 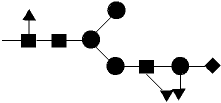 |
| 2010 | 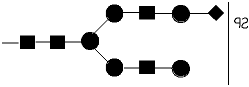 |
| 2011 | 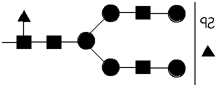 |
| 2018 | 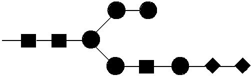 |
| 2019 | 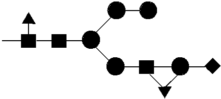 |
| 2035 | 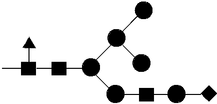 |
| 2059 | 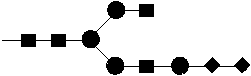 |
| 2060 | 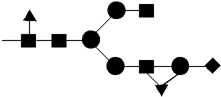 |
| 2076 | 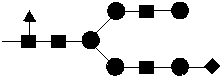 |
| 2082 | 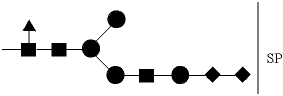 |
| 2092 | 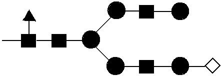 |
| 2133 | 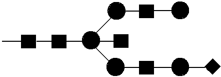 |
| 2156 | 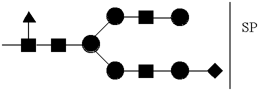 |
| 2157 | 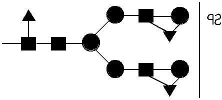 |
| 2164 | 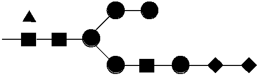 |
| 2178 | 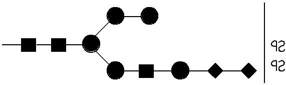 |
| 2221 | 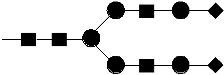 |
| 2222 | 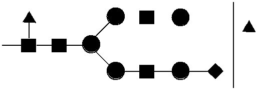 |
| 2230 | 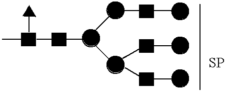 |
| 2237 | 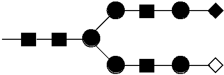 |
| 2238 | 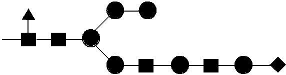 |
| 2254 | 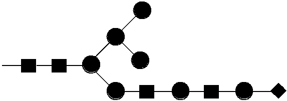 |
| 2262 | 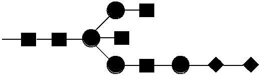 |
| 2279 | 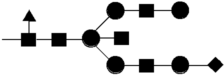 |
| 2280 | 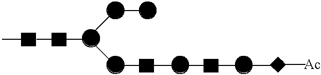 |
| 2295 | 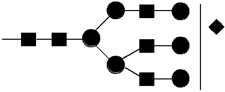 |
| 2349 | 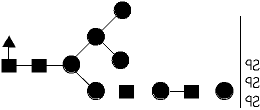 |
| 2367 | 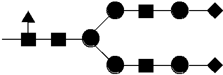 |
| 2368 | 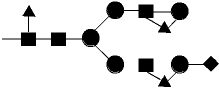 |
| 2382 | 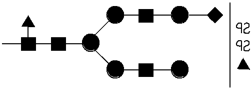 |
| 2383 | 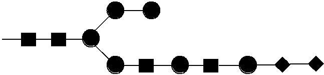 |
| 2389 | 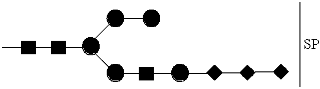 |
| 2390 | 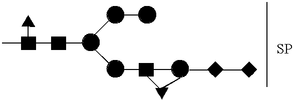 |
| 2406 | 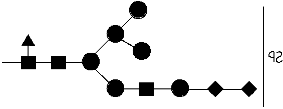 |
| 2424 | 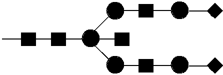 |
| 2425 | 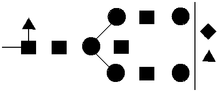 |
| 2441 | 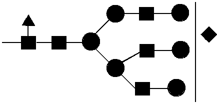 |
| 2447 | 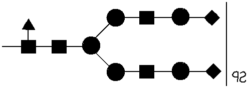 |
| 2457 | 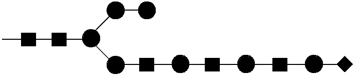 |
| 2513 | 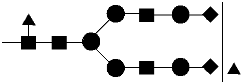 |
| 2514 | 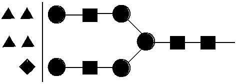 |
| 2521 | 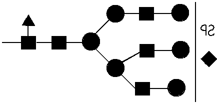 |
| 2570 | 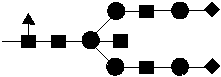 |
| 2571 | 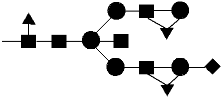 |
| 2586 | 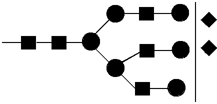 |
| 2587 | 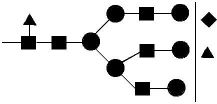 |
| 2595 | 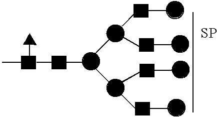 |
| 2603 | 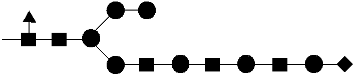 |
| 2644 | 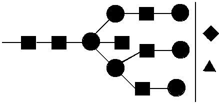 |
| 2645 | 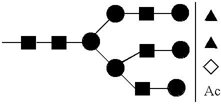 |
| 2659 | 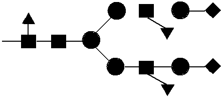 |
| 2660 | 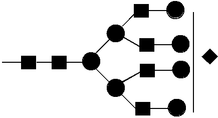 |
| 2674 | 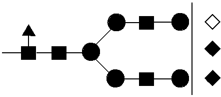 |
| 2714 | 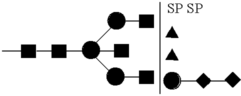 |
| 2732 | 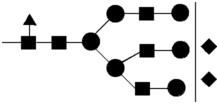 |
| 2733 | 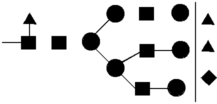 |
| 2807 | 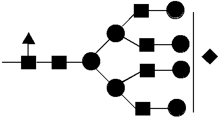 |
| 2878 | 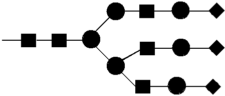 |
| 2879 | 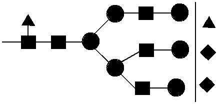 |
| 2880 | 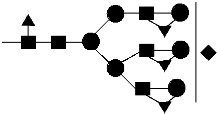 |
| 2900 | 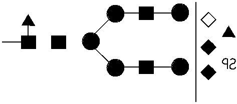 |
| 2952 | 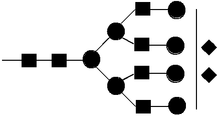 |
| 2953 | 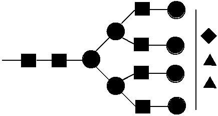 |
| 3024 | 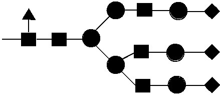 |
| 3025 | 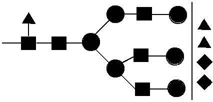 |
| 3026 | 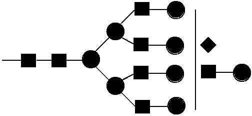 |
| 3098 | 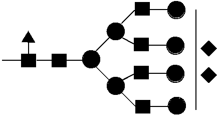 |
| 3099 | 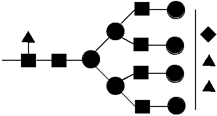 |
| 3170 | 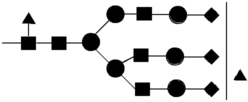 |
| 3171 | 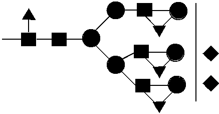 |
| 3172 | 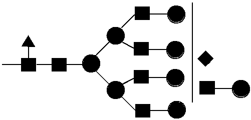 |
| 3243 | 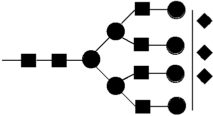 |
| 3245 | 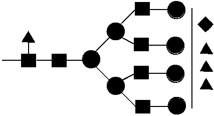 |
| 3389 | 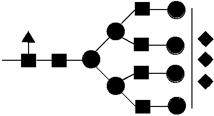 |
| 3390 | 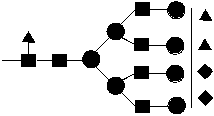 |
| 3391 | 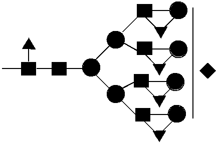 |
| 3463 | 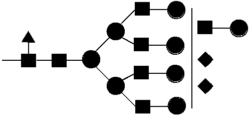 |
| 3536 | 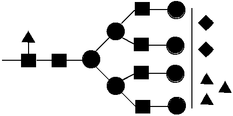 |
| 3537 | 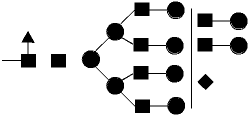 |
| 3609 | 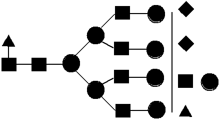 |
| 3680 | 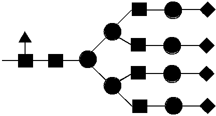 |
| 3681 | 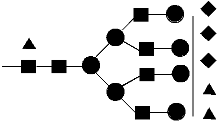 |
| 3683 | 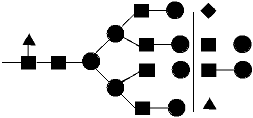 |
| 3754 | 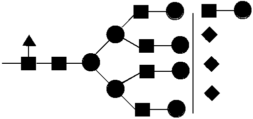 |
| 3756 | 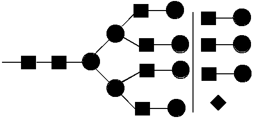 |
| 3827 | 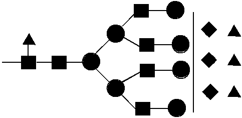 |
| 3828 | 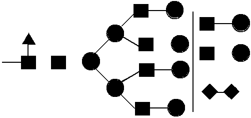 |
| 3901 | 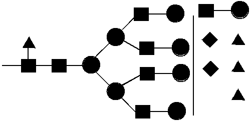 |
| 3974 | 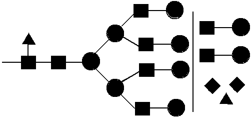 |
| 4046 | 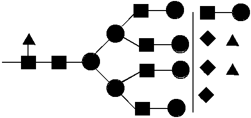 |
| 4121 | 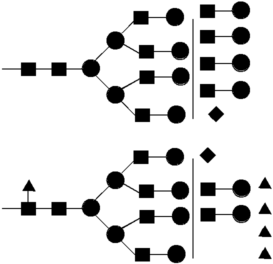 |

TABLE 6
Structure assignments of BM MSC neutral N-glycans.

| m/z | Structure |
|---|---|
| 568 | 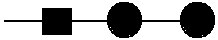 |
| 609 | 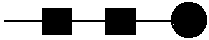 |
| 730 | 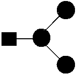 |
| 755 | 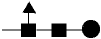 |
| 771 | 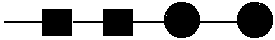 |
| 892 | 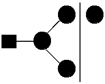 |
| 917 | 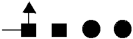 |
| 933 | 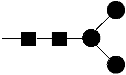 |
| 974 | 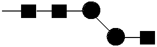 |
| 1054 | 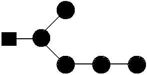 |
| 1079 | 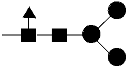 |
| 1095 | 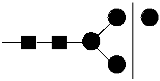 |
| 1120 | 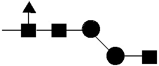 |
| 1136 | 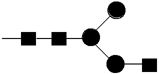 |
| 1216 | 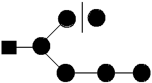 |
| 1241 | 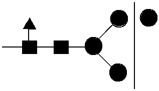 |
| 1257 | 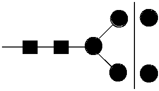 |
| 1282 | 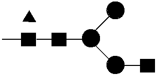 |
| 1298 | 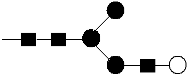 |
| 1323 | 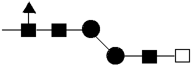 |
| 1339 | 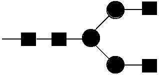 |
| 1378 | 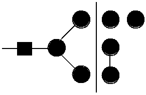 |
| 1403 | 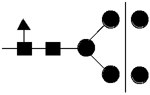 |
| 1419 | 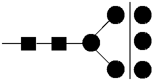 |
| 1444 | 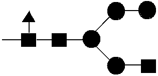 |
| 1460 | 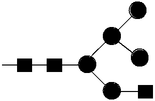 |
| 1485 | 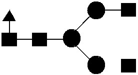 |
| 1501 | 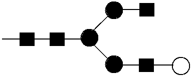 |
| 1540 | 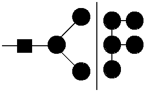 |
| 1542 | 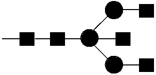 |
| 1565 | 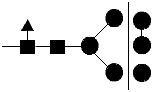 |
| 1581 | 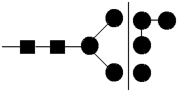 |
| 1590 | 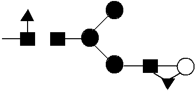 |
| 1606 | 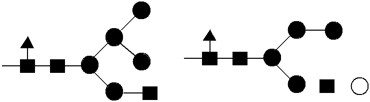 |
| 1622 | 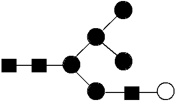 |
| 1647 | 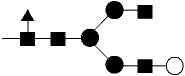 |
| 1663 | 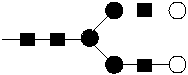 |
| 1688 | 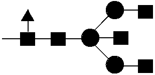 |
| 1702 | 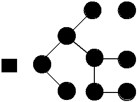 |
| 1704 | 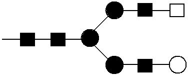 |
| 1743 | 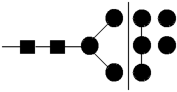 |
| 1752 | 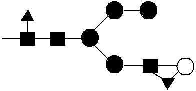 |
| 1768 | 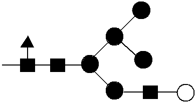 |
| 1793 | 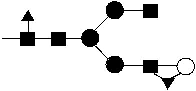 |
| 1809 | 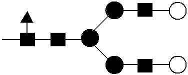 |
| 1825 | 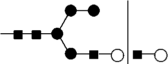 |
| 1850 | 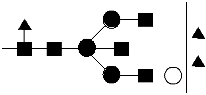 |
| 1866 | 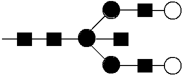 |
| 1905 | 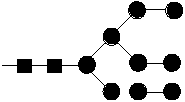 |
| 1955 | 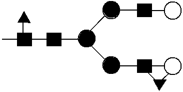 |
| 1971 | 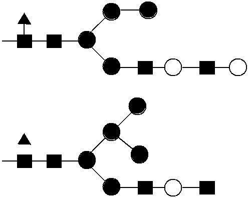 |
| 1987 | 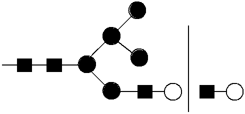 |
| 1996 | 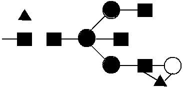 |
| 2012 | 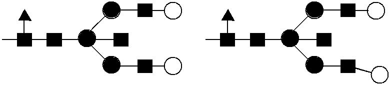 |
| 2028 | 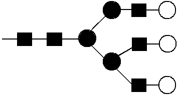 |
| 2067 | 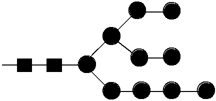 |
| 2101 | 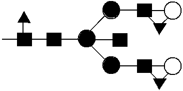 |
| 2117 | 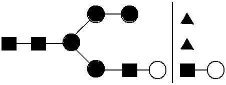 |
| 2133 | 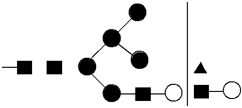 |
| 2158 | 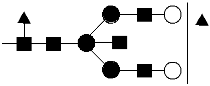 |
| 2174 | 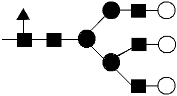 |
| 2190 | 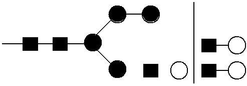 |
| 2215 | 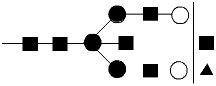 |
| 2229 | 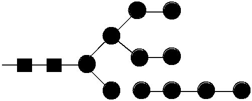 |
| 2231 | 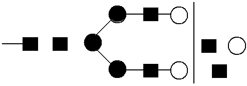 |
| 2304 | 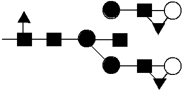 |
| 2320 | 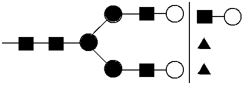 |
| 2336 | 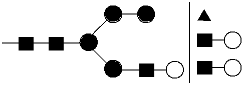 |
| 2352 | 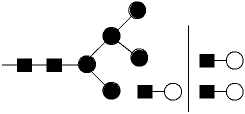 |
| 2377 | 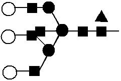 |
| 2391 | 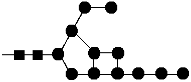 |
| 2393 | 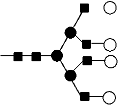 |
| 2466 | 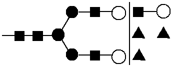 |
| 2539 | 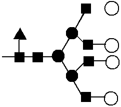 |
| 2612 | 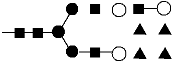 |
| 2685 | 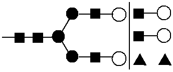 |
| 2742 | 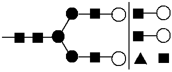 |
| 2758 | 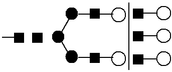 |
| 2905 | 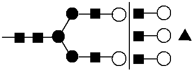 |
| 3124 | 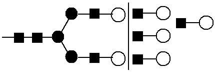 |
| 3270 | 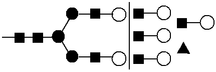 |
| 3635 | 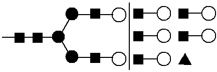 |

TABLE 7
NMR analysis of the major sialylated N-glycan core structures of BM MSC.

| | | | |
|---|---|---|---|
| 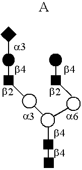 | 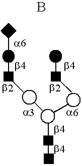 | 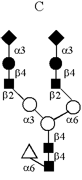 | 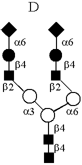 |

| Glycan residue | | | ¹H-NMR chemical shift (ppm) | | | | |
|---|---|---|---|---|---|---|---|
| Residue | Linkage | Proton | A | B | C | D | MSC ¹⁾ |
| D-GlcNAc | | H-1α | 5.188 | 5.189 | 5.181 | 5.189 | 5.185 |
| | | NAc | 2.038 | 2.038 | 2.039 | 2.038 | 2.039 |
| α-L-Fuc | 6 | H-1α | — | — | 4.892 | — | 4.9 |
| | | H-1β | — | — | 4.900 | — | 4.9 |
| | | CH₃α | — | — | 1.211 | — | 1.206 |
| | | CH₃β | — | — | 1.223 | — | 1.216 |
| β-D-GlcNAc | 4 | H-1β | 4.604 | 4.606 | n.a. | 4.604 | — |
| | | NAc | 2.081 | 2.081 | 2.096 | 2.084 | 2.077/2.097 |
| β-D-Man | 4, 4 | H-1 | n.a. | n.a. | n.a. | n.a. | n.a. |
| | | H-2 | 4.246 | 4.253 | 4.248 | 4.258 | 4.255 |
| α-D-Man | 6, 4, 4 | H-1 | 4.928 | 4.930 | 4.922 | 4.948 | 4.929 |
| | | H-2 | 4.11 | 4.112 | 4.11 | 4.117 | n.a. |
| β-D-GlcNAc | 2, 6, 4, 4 | H-1 | 4.581 | 4.582 | 4.573 | 4.604 | n.a. |
| | | NAc | 2.047 | 2.047 | 2.043 | 2.066 | 2.039/n.a. |
| β-D-Gal | 4, 2, 6, 4, 4 | H-1 | 4.473 | 4.473 | 4.550 | 4.447 | 4.477/4.554 |
| | | H-4 | n.a. | n.a. | n.a. | n.a. | — |
| α-D-Man | 3, 4, 4 | H-1 | 5.118 | 5.135 | 5.116 | 5.133 | 5.120/n.a. |
| | | H-2 | 4.190 | 4.196 | 4.189 | 4.197 | 4.2/4.218 |
| β-D-GlcNAc | 2, 3, 4, 4 | H-1 | 4.573 | 4.606 | 4.573 | 4.604 | — |
| | | NAc | 2.047 | 2.069 | 2.048 | 2.070 | n.a./2.077 |
| β-D-Gal | 4 ,2, 3, 4, 4 | H-1 | 4.545 | 4.445 | 4.544 | 4.443 | 4.554 |
| | | H-3 | 4.113 | n.a. | 4.113 | n.a. | 4.110 |

¹⁾ Chemical shifts determined from the center of the signal.
n.a.: Not assigned.
The identified signals were consistent with sialylated biantennary complex-type N-glycan structures such as the structures A-D that have monosaccharide compositions S₁₋₂H₅N₄F₀₋₁. Reference data is after Hård et al. (Hård, K., et al., 1992, Eur. J. Biochem. 209, 895-915) and Helin et al. (Helin, J., et al., 1995, Carbohydr. Res. 266, 191-209). The major signals in the obtained NMR spectrum can be explained by structural components of these referencestructures, which can also occur in other N-glycan backbones and branching structures. The spectrum also revealed that α2,3-linked sialic acid is more common than α2,6-linked sialic acid in the N-glycans according to the characteristic sialic acid signals (data not shown). Monosaccharide symbols are: open circle, D-mannose; black square, N-acetyl-D-glucosamine; black circle, D-galactose; black diamond, N-acetylneuraminic acid; open triangle, L-fucose.

TABLE 8
NMR analysis of the major neutral N-glycans of BM MSC.

| | | | |
|---|---|---|---|
| 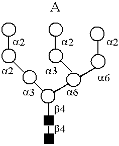 | 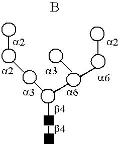 | 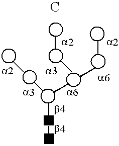 | 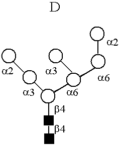 |

| Glycan residue | | | ¹H-NMR chemical shift (ppm) | | | | |
|---|---|---|---|---|---|---|---|
| Residue | Linkage | Proton | A | B | C | D | MSC ¹⁾ |
| D-GlcNAc | | H-1α | 5.191 | 5.187 | 5.187 | 5.188 | 5.190 |
| | | H-1β | 4.690 | 4.693 | 4.693 | 4.695 | — |
| | | NAc | 2.042 | 2.037 | 2.037 | 2.038 | 2.039 |
| β-D-GlcNAc | 4 | H-1 | 4.596 | 4.586 | 4.586 | 4.600 | 4.591 |
| | | NAc | 2.072 | 2.063 | 2.063 | 2.064 | 2.065 |
| β-D-Man | 4, 4 | H-1 | 4.775 | 4.771 | 4.771 | 4.780 | 2) |
| | | H-2 | 4.238 | 4.234 | 4.234 | 4.240 | 4.236 |
| α-D-Man | 6, 4, 4 | H-1 | 4.869 | 4.870 | 4.870 | 4.870 | 4.869 |
| | | H-2 | 4.149 | 4.149 | 4.149 | 4.150 | 4.152 |
| α-D-Man | 6, 6, 4, 4 | H-1 | 5.153 | 5.151 | 5.151 | 5.143 | 5.148 |
| | | H-2 | 4.025 | 4.021 | 4.021 | 4.020 | n.d. |
| α-D-Man | 2, 6, 6, 4, 4 | H-1 | 5.047 | 5.042 | 5.042 | 5.041 | 5.042 |
| | | H-2 | 4.074 | 4.069 | 4.069 | 4.070 | 4.071 |
| α-D-Man | 3, 6, 4, 4 | H-1 | 5.414 | 5.085 | 5.415 | 5.092 | 5.408/5.090 |
| | | H-2 | 4.108 | 4.069 | 4.099 | 4.070 | 4.109/4.071 |
| α-D-Man | 2, 3, 6, 4, 4 | H-1 | 5.047 | — | 5.042 | — | 5.042 |
| | | H-2 | 4.074 | — | 4.069 | — | 4.071 |
| α-D-Man | 3, 4, 4 | H-1 | 5.343 | 5.341 | 5.341 | 5.345 | 5.342 |
| | | H-2 | 4.108 | 4.099 | 4.099 | 4.120 | 4.109 |
| α-D-Man | 2, 3, 4, 4 | H-1 | 5.317 | 5.309 | 5.050 | 5.055 | 5.310/5.06 |
| | | H-2 | 4.108 | 4.099 | 4.069 | 4.070 | 4.109/4.071 |
| α-D-Man | 2, 2, 3, 4, 4 | H-1 | 5.047 | 5.042 | — | — | 5.042 |
| | | H-2 | 4.074 | 4.069 | — | — | 4.071 |

¹⁾ Chemical shifts determined from the center of the signal.
2) Signal under HDO.
n.d. Not determined.
The identified signals were consistent with high-mannose type N-glycan structures such as the structures A-D that have monosaccharide compositions H₇₋₉N₂. The major signals in the NMR spectrum can be explained by structural components of these reference structures, which can also occur in other N-glycan backbones and branching structures. Reference data is after Fu et al. (Fu, D., et al., 1994, Carbohydr. Res. 261, 173-186) and Hård et al. (Hård, K., et al., 1991,Glycoconj. J. 8, 17-28). Monosaccharide symbols: open circle, D-mannose; black square, N-acetyl-D-glucosamine.

TABLE 9
Exoglycosidase analysis results of BM MSC showing proposed non-reducing
terminal structures present in neutral and sialylated N-glycan components
studied in the present invention. The numbers in the table refer to detected
amounts of each terminal structure or the detected ranges of their amounts.
In case of mixtures of isomeric structures within a glycan signal, the ranges
inducate variation in detected multiple structures. For explanation of symbols
see bottom of table.

| | | | | | β1,4- | α1,2- | α1,3/4- | poly- | Sialyl- |
|---|---|---|---|---|---|---|---|---|---|
| | | α-Man | β-Gn | β1,3-Gal | Gal | Fuc | Fuc | LN | form |
| H2N1 | 568 | 0-1 | 1 | | | | | | |
| H1N2 | 609 | | | | | | | | |
| H2N1F1 | 714 | | | | | | | | |
| H3N1 | 730 | 0-2 | 1 | | | | | | |
| H1N2F1 | 755 | | | | | | | | |
| H2N2 | 771 | 0-1 | | | | | | | |
| H2N1F2 | 860 | | | | | | | | |
| H3N1F1 | 876 | | | | | | | | |
| H4N1 | 892 | 1-3 | | | | | | | |
| H1N2F2 | 901 | | | | | | | | |
| H2N2F1 | 917 | 0-1 | | | | 0-1 | 0-1 | | |
| H3N2 | 933 | 0-2 | | | | | | | |
| H1N3F1 | 958 | | | | | | | | |
| H2N3 | 974 | | | | | | | | |
| H3N1F2 | 1022 | | | | | | | | |
| H5N1 | 1054 | 2-4 | | | | | | | |
| H3N2F1 | 1079 | 0-2 | | | | 0-1 | 0-1 | | |
| H4N2 | 1095 | 0-3 | | | | | | | |
| H2N3F1 | 1120 | | 1 | | | | | | |
| H3N3 | 1136 | | 0-1 | | | | | | + |
| H2N4 | 1177 | | | | | | | | |
| H2N2F3 | 1209 | 1 | 1 | | | | | | |
| H6N1 | 1216 | 2-5 | 0-1 | | | | | | |
| H3N2F2 | 1225 | | | | | | | | |
| H4N2F1 | 1241 | 1-3 | | | | 0-1 | 0-1 | | |
| H5N2 | 1257 | 0-4 | | | | | | | |
| H2N3F2 | 1266 | | | | | | | | |
| H3N3F1 | 1282 | | 0-1 | | | 0-1 | 0-1 | | + |
| H4N3 | 1298 | | 0-1 | | 0-1 | | | | + |
| H2N4F1 | 1323 | | | | | | | | |
| H3N4 | 1339 | | | | | | | 1 | |
| H2N2F4 | 1355 | | | | | | | | |
| H3N2F3 | 1371 | | | | | | | | |
| H7N1 | 1378 | 2-6 | | | | | | | |
| H5N2F1 | 1403 | 2-4 | 0-2 | | | 0-1 | 0-1 | | |
| H6N2 | 1419 | 0-5 | | | | | | | |
| H1N6 | 1421 | | | | | | | | |
| H3N3F2 | 1428 | | | | | | | | |
| H4N3F1 | 1444 | | 0-1 | | | 0-1 | 0-1 | | + |
| H5N3 | 1460 | 0-1 | 0-1 | | 0-2 | | | | + |
| H3N4F1 | 1485 | 0-1 | 0-2 | | | 0-1 | 0-1 | 0-1 | + |
| H4N4 | 1501 | | | | | | | 0-1 | + |
| H3N2F4 | 1517 | | | | | | | | |
| H4N2F3 | 1533 | | | | | | | | |
| H8N1 | 1540 | 2-7 | 0-1 | | | | | | |
| H3N5 | 1542 | | | | | | | | |
| H5N2F2 | 1549 | | | | | | | | |
| H6N2F1 | 1565 | 3-5 | 0-1 | 1 | 1 | 0-1 | | | |
| H7N2 | 1581 | 0-6 | 0-1 | | | | | | |
| H2N6 | 1583 | | | | | | | | |
| H4N3F2 | 1590 | 1 | | | | 0-2 | 0-2 | | |
| H5N3F1 | 1606 | 0-1 | 0-1 | | 0-1 | 0-1 | 0-1 | | + |
| H6N3 | 1622 | 0-2 | 0-1 | | 0-3 | | | | + |
| H3N4F2 | 1631 | | | | | | | | |
| H4N4F1 | 1647 | | 1-2 | | | 0-1 | | | + |
| H5N4 | 1663 | | 0-2 | | 2 | | | 0-1 | + |
| H3N5F1 | 1688 | | 1 | | | 0-1 | 0-1 | | + |
| H9N1 | 1702 | 3-8 | 1 | | | | | | |
| H4N5 | 1704 | | | | | | | | + |
| H3N3F4 | 1720 | | | | | | | | |
| H8N2 | 1743 | 1-7 | | | | | | | |
| H3N6 | 1745 | | | | | | | | |
| H5N3F2 | 1752 | | | | | 0-2 | 0-2 | | |
| H6N3F1 | 1768 | 0-2 | | | 1-2 | 0-1 | 0-1 | | + |
| H7N3 | 1784 | 1-3 | 1-2 | | 1-4 | | | | + |
| H4N4F2 | 1793 | | 1 | | | 0-2 | 1 | | + |
| H5N4F1 | 1809 | | 0-2 | | 1-2 | 0-1 | 0-1 | | + |
| H6N4 | 1825 | | | | | | | 1 | + |
| H4N5F3 | 1850 | | | | | | | | |
| H10N1 | 1864 | | | | | | | | |
| H5N5 | 1866 | | | | | | | | + |
| H4N3F4 | 1882 | | | | | | | | |
| H8N2F1 | 1889 | | | | | | | | |
| H3N6F1 | 1891 | | | | | | | | |
| H9N2 | 1905 | 2-8 | 0-2 | | | | | | |
| H6N3F2 | 1914 | | | | | | | | |
| H7N3F1 | 1930 | | | | | | | | |
| H8N3 | 1946 | | | | | | | | |
| H5N4F2 | 1955 | | 0-1 | | 1 | 0-2 | 0-2 | | + |
| H6N4F1 | 1971 | 0-1 | 1 | | 2-3 | 0-1 | 0-1 | | + |
| H3N5F3 | 1980 | | | | | | | | |
| H7N4 | 1987 | | | | | | | 1 | |
| H4N5F2 | 1996 | | 2 | | | 0-2 | | | |
| H5N5F1 | 2012 | | 1-2 | 1 | 2 | | | | + |
| H7N2F3 | 2019 | | | | | | | | |
| H2N6F3 | 2021 | | | | | | | | |
| H11N1 | 2026 | | | | | | | | |
| H6N5 | 2028 | 0-1 | 0-1 | | 3 | | | 0-1 | + |
| H3N6F2 | 2037 | | | | | | | | |
| H5N3F4 | 2044 | | | | | | | | |
| H4N6F1 | 2053 | | | | | | | | |
| H10N2 | 2067 | 3-8 | 0-1 | | | | | | |
| H5N4F3 | 2101 | 0-1 | 1 | | | 0-3 | | | + |
| H6N4F2 | 2117 | | | | | | | | |
| H3N5F4 | 2126 | | | | | | | | |
| H7N4F1 | 2133 | | | | | | | | |
| H4N5F3 | 2142 | | | 1 | 0-1 | | | | |
| H8N4 | 2149 | | | | | | | | |
| H5N5F2 | 2158 | | | | | | | | |
| H6N5F1 | 2174 | 0-1 | 1-2 | | 3 | 0-1 | 0-1 | | + |
| H3N6F3 | 2183 | | | | | | | | |
| H7N5 | 2190 | | | | | | | | |
| H4N6F2 | 2199 | | | | | | | | |
| H5N6F1 | 2215 | | | | | | | | |
| H11N2 | 2229 | 4-8 | 1 | | 1 | | | | |
| H6N6 | 2231 | | | | | | | | |
| H5N4F4 | 2247 | | | | | | | | |
| H4N7F1 | 2256 | | | | | | | | |
| H6N4F3 | 2263 | | | | | | | | |
| H5N7 | 2272 | | | | | | | | |
| H5N5F3 | 2304 | 1 | 2 | | | 0-3 | 0-3 | | |
| H9N4 | 2311 | | | | | | | | |
| H6N5F2 | 2320 | 1 | 1 | | | 0-2 | 0-2 | | + |
| H7N5F1 | 2336 | | | | | | | | |
| H8N5 | 2352 | | | | | | | | |
| H5N6F2 | 2361 | | | | | | | | |
| H6N6F1 | 2377 | | | | | | | | + |
| H12N2 | 2391 | | | | | | | | |
| H7N6 | 2393 | 0-1 | 0-1 | | 1-4 | | | 0-1 | + |
| H6N4F4 | 2409 | | | | | | | | |
| H6N5F3 | 2466 | 1 | 1 | | | | | | |
| H8N5F1 | 2498 | | | | | | | | |
| H9N5 | 2514 | | | | | | | | |
| H6N6F2 | 2523 | | | | | | | | |
| H7N6F1 | 2539 | 1 | 1 | | 4 | | | | + |
| H8N6 | 2555 | | | | | | | | |
| H6N5F4 | 2612 | 1 | 1 | | | 0-4 | 0-4 | | |
| H7N6F2 | 2685 | | | | | | | | |
| H7N7F1 | 2742 | | | | | | | | |
| H8N7 | 2758 | | | | | | | | + |
| H7N6F3 | 2832 | | | | | | | | |
| H8N7F1 | 2905 | | | | | | | | + |
| H7N6F4 | 2978 | | | | | | | | |
| H9N8 | 3124 | | | | | | | | |
| H8N6F4 | 3140 | | | | | | | | |
| H9N8F1 | 3270 | | | | | | | | + |
| H10N9F1 | 3635 | | | | | | | | |

α-Man, β-Gn, β1,3-Gal, β1,4-Gal, α1,2-Fuc, α1,3/4-Fuc, and poly-LN: number of non-reducing α-Man, β-GlcNAc, β1,3-linked Gal, β1,4-linked Gal, α1,2-linke Fuc, α1,3/4-linked Fuc, and poly-LacNAc residues detected by the specific glycosidase enzymes as described in the Examples.

Sialyl-form: sialylated hybrid-type and complex-type N-glycans that were analyzed as neutral N-glycans after digestion with sialidase enzyme are marked by “+”. The structures present in BM MSC are sialylated derivatives of the shown structures, as described in the Examples

TABLE 10

| Proposed composition | m/z | α-Man | β-GlcNAc | β4-Gal | β3-Gal |
|---|---|---|---|---|---|
| Hex2HexNAc | 568 | | −−− | | |
| HexHexNAc2 | 609 | +++ | | | |
| Hex2HexNAcdHex | 714 | +++ | | | |
| Hex3HexNAc | 730 | −− | −−− | | |
| HexHexNAc2dHex | 755 | +++ | | | |
| Hex2HexNAc2 | 771 | ++ | ++ | | |
| Hex4HexNAc | 892 | −−− | + | | |
| Hex2HexNAc2dHex | 917 | | + | | |
| Hex3HexNAc2 | 933 | ++ | ++ | | |
| Hex2HexNAc3 | 974 | +++ | | | |
| Hex5HexNAc | 1054 | −− | | | |
| Hex3HexNAc2dHex | 1079 | −− | + | | |
| Hex4HexNAc2 | 1095 | − | + | | |
| Hex2HexNAc3dHex | 1120 | +++ | −−− | | |
| Hex3HexNAc3 | 1136 | ++ | −− | + | |
| Hex2HexNAc2dHex3 | 1209 | −−− | −−− | | |
| Hex6HexNAc | 1216 | −− | | | |
| Hex4HexNAc2dHex | 1241 | −−− | | | |
| Hex5HexNAc2 | 1257 | −− | | | |
| Hex2HexNAc3dHex2 | 1266 | | | | |
| Hex3HexNAc3dHex | 1282 | ++ | −− | + | |
| Hex4HexNAc3 | 1298 | ++ | − | | |
| Hex3HexNAc4 | 1339 | +++ | | +++ | |
| Hex7HexNAc | 1378 | −− | | | |
| Hex5HexNAc2dHex | 1403 | −−− | | | |
| Hex6HexNAc2 | 1419 | −− | + | | |
| Hex3HexNAc3dHex2 | 1428 | +++ | | | |
| Hex4HexNAc3dHex | 1444 | + | − | + | |
| Hex5HexNAc3 | 1460 | + | − | ++ | |
| Hex3HexNAc4dHex | 1485 | | −− | ++ | |
| Hex4HexNAc4 | 1501 | ++ | | | |
| Hex8HexNAc | 1540 | − | | | |
| Hex3HexNAc5 | 1542 | | | +++ | |
| Hex6HexNAc2dHex | 1565 | −−− | | −−− | −−− |
| Hex7HexNAc2 | 1581 | −− | | | |
| Hex4HexNAc3dHex2 | 1590 | | | | |
| Hex5HexNAc3dHex | 1606 | | −− | −− | |
| Hex6HexNAc3 | 1622 | −− | − | −− | |
| Hex4HexNAc4dHex | 1647 | | −−− | | |
| Hex5HexNAc4 | 1663 | | −− | −−− | |
| Hex3HexNAc5dHex | 1668 | | −−− | ++ | |
| Hex9HexNAc | 1702 | −−− | −−− | | |
| Hex8HexNAc2 | 1743 | −− | + | | |
| Hex6HexNAc3dHex | 1768 | | | −−− | |
| Hex7HexNAc3 | 1784 | −−− | −−− | −−− | |
| Hex4HexNAc4dHex2 | 1793 | | −−− | ++ | |
| Hex5HexNAc4dHex | 1809 | | −− | −−− | |
| Hex3HexNAc6dHex | 1891 | | | +++ | |
| Hex9HexNAc2 | 1905 | −−− | − | | |
| Hex5HexNAc4dHex | 1955 | | − | −−− | |
| Hex6HexNAc4dHex | 1971 | | −−− | −−− | |
| Hex4HexNAc5dHex2 | 1996 | | −−− | | |
| Hex5HexNAc5dHex | 2012 | | −−− | −−− | −−− |
| Hex6HexNAc5 | 2028 | | − | −−− | |
| Hex10HexNAc2 | 2067 | −−− | − | | |
| Hex5HexNAc4dHex3 | 2101 | | −−− | | |
| Hex4HexNAc5dHex3 | 2142 | | | −− | −−− |
| Hex6HexNAc5dHex | 2174 | | −− | −−− | |
| Hex11HexNAc2 | 2229 | | | | |
| Hex5HexNAc5dHex3 | 2304 | | −−− | | |
| Hex6HexNAc5dHex2 | 2320 | | −−− | | |
| Hex7HexNAc6 | 2393 | | | −−− | |
| Hex6HexNAc5dHex3 | 2466 | | −−− | | |
| Hex7HexNAc6dHex | 2539 | | −−− | −−− | |

TABLE 11

| | Preferred | | | |
|---|---|---|---|---|
| | monosaccharide | Terminal | Experimental structures included in the glycan | |
| m/z* | compositions | epitopes | signal according to the invention^{§} | Group^{#} |
| 568 | Hex2HexNAc | Manα | Manα→Hex₁HexNAc₁ | S |
| 730 | Hex3HexNAc | Manα | (Manα→)₂Hex₁HexNAc₁ | S |
| | | GlcNAc | GlcNAc→Hex₃ | |
| 771 | Hex2HexNAc2 | Manα | Manα→Hex₁HexNAc₂ | LO |
| 892 | Hex4HexNAc | Manα | (Manα→)₃Hex₁HexNAc₁ | S |
| 917 | Hex2HexNAc2dHex | Manα | Manα→Hex₁HexNAc₂dHex₁ | LO, F |
| 933 | Hex3HexNAc2 | Manα | (Manα→)₂Hex₁HexNAc₂ | LO |
| 1054 | Hex5HexNAc | Manα | (Manα→)₄Hex₁HexNAc₁ | S |
| 1079 | Hex3HexNAc2dHex | Manα | (Manα→)₂Hex₁HexNAc₂dHex₁ | LO, F |
| 1095 | Hex4HexNAc2 | Manα | (Manα→)₃Hex₁HexNAc₂ | LO |
| 1120 | Hex2HexNAc3dHex | GlcNAcβ | GlcNAcβ→Hex₂HexNAc₂dHex₁ | HY, F, |
| | | | | N > H |
| 1136 | Hex3HexNAc3 | GlcNAcβ | GlcNAcβ→Hex₃HexNAc₂ | HY, |
| | | | | N = H |
| 1209 | Hex2HexNAc2dHex3 | Manα | Manα→Hex₁HexNAc₂dHex₃ | FC, |
| | | GlcNAc | GlcNAc→Hex₂HexNAc₁dHex₃ | N = H |
| 1216 | Hex6HexNAc | Manα | (Manα→)₅Hex₁HexNAc₁ | S |
| 1241 | Hex4HexNAc2dHex | Manα | (Manα)₃Hex₁HexNAc₂dHex₁ | LO, F |
| 1257 | Hex5HexNAc2 | Manα | (Manα→)₄Hex₁HexNAc₂ | HI |
| 1266 | Hex2HexNAc3dHex2 | Fuc | Fuc→Hex₂HexNAc₃dHex₁ | HY, FC |
| 1282 | Hex3HexNAc3dHex | GlcNAcβ | GlcNAcβ→Hex₃HexNAc₂dHex₁ | HY, F, |
| | | | | N = H |
| 1298 | Hex4HexNAc3 | | | HY |
| 1378 | Hex7HexNAc | Manα | (Manα→)₆Hex₁HexNAc₁ | S |
| 1403 | Hex5HexNAc2dHex | Manα | (Manα)₄Hex₁HexNAc₂dHex₁ | HF |
| 1419 | Hex6HexNAc2 | Manα | (Manα→)₅Hex₁HexNAc₂ | HI |
| 1444 | Hex4HexNAc3dHex | GlcNAcβ | GlcNAcβ→Hex₄HexNAc₂dHex₁ | HY, F |
| 1460 | Hex5HexNAc3 | GlcNAcβ | GlcNAcβ→Hex₅HexNAc₂ | HY |
| 1485 | Hex3HexNAc4dHex | 2 × GlcNAcβ | (GlcNAcβ→)₂Hex₃HexNAc₂dHex₁ | CO, F, |
| | | | | N > H |
| 1501 | Hex4HexNAc4 | | | CO, |
| | | | | N = H |
| 1540 | Hex8HexNAc | Manα | (Manα→)₇Hex₁HexNAc₁ | S |
| 1565 | Hex6HexNAc2dHex | Manα | (Manα)₅Hex₁HexNAc₂dHex₁ | HF |
| 1581 | Hex7HexNAc2 | Manα | (Manα→)₆Hex₁HexNAc₂ | HI |
| 1590 | Hex4HexNAc3dHex2 | Fucα | Fuca→Hex₄HexNAc₃dHex₁ | HY, FC |
| 1606 | Hex5HexNAc3dHex | GlcNAcβ | GlcNAcβ→Hex₅HexNAc₂dHex₁ | HY, F |
| | | Galβ4 | Galβ4GlcNAc→Hex₄HexNAc₂dHex₁ | |
| 1622 | Hex6HexNAc3 | Manα | Manα→Hex₅HexNAc₃ | HY |
| | | GlcNAcβ | GlcNAcβ→Hex₆HexNAc₂ | |
| | | Galβ4 | Galβ4GlcNAc→Hex₅HexNAc₂ | |
| | | | Manα→[GlcNAcβ→]Hex₅HexNAc₂ | |
| | | | Manα→[Galβ4GlcNAc→]Hex₄HexNAc₂ | |
| 1647 | Hex4HexNAc4dHex | GlcNAcβ | GlcNAcβ→Hex₄HexNAc₃dHex₁ | CO, F, |
| | | | | N = H |
| 1663 | Hex5HexNAc4 | 2 × Galβ4 | (Galβ4GlcNAc→)₂Hex₃HexNAc₂ | CO |
| | | GlcNAcβ | GlcNAcβ→Hex₅HexNAc₃ | |
| 1688 | Hex3HexNAc5dHex | 3 × GlcNAcβ | (GlcNAcβ→)₃Hex₃HexNAc₂dHex₁ | CO, F, |
| | | | | N > H |
| 1702 | Hex9HexNAc | Manα | (Manα→)₈Hex₁HexNAc₁ | S |
| 1743 | Hex8HexNAc2 | Manα | (Manα→)₇Hex₁HexNAc₂ | HI |
| 1768 | Hex6HexNAc3dHex | Galβ4 | Galβ4GlcNAc→Hex₅HexNAc₂dHex₁ | HY, F |
| 1784 | Hex7HexNAc3 | Manα | Manα→Hex₆HexNAc₃ | HY |
| | | GlcNAcβ | GlcNAcβ→Hex₇HexNAc₂ | |
| | | Galβ4 | Galβ4GlcNAc→Hex₆HexNAc₂ | |
| | | | Manα→[GlcNAcβ→]Hex₆HexNAc₂ | |
| | | | Manα→[Galβ4GlcNAc→]Hex₅HexNAc₂ | |
| 1793 | Hex4HexNAc4dHex2 | GlcNAcβ | GlcNAcβ→Hex₄HexNAc₃dHex₂ | CO, FC, |
| | | Fuc | Fuc→Hex₄HexNAc₄dHex₁ | N = H |
| | | | GlcNAcβ→[Fuc→]Hex₄HexNAc₃dHex₁ | |
| 1809 | Hex5HexNAc4dHex | 2 × Galβ4 | (Galβ4GlcNAc→)₂Hex₃HexNAc₂dHex₁ | CO, F |
| | | GlcNAcβ | GlcNAcβ→Hex₅HexNAc₃dHex₁ | |
| 1891 | Hex3HexNAc6dHex | | | CO, F, |
| | | | | N > H |
| 1905 | Hex9HexNAc2 | Manα | (Manα→)₈Hex₁HexNAc₂ | HI |
| 1955 | Hex5HexNAc4dHex2 | Galβ4 | Galβ4GlcNAc→Hex₄HexNAc₃dHex₂ | CO, FC |
| | | Fuc | Fuc→Hex₅HexNAc₄dHex₁ | |
| | | | Galβ4GlcNAc→[Fuc→]Hex₄HexNAc₃dHex₁ | |
| 1971 | Hex6HexNAc4dHex | GlcNAcβ | GlcNAcβ→Hex₆HexNAc₃dHex₁ | CO, F |
| | | Galβ4 | Galβ4GlcNAc→Hex₅HexNAc₃dHex₁ | |
| 1996 | Hex4HexNAc5dHex2 | 2 × GlcNAcβ | (GlcNAcβ→)₂Hex₄HexNAc₃dHex₂ | CO, FC, |
| | | | | N > H |
| 2012 | Hex5HexNAcSdHex | GlcNAcβ | GlcNAcβ→Hex₅HexNAc₄dHex₁ | CO, F, |
| | | 2 × Galβ4 | (Galp4GlcNAc→)₂Hex₃HexNAc₃dHex₁ | N = H |
| | | Galβ3 | Galp3GlcNAc→Hex₄HexNAc₄dHex₁ | |
| | | | (Galβ4GlcNAc→)₂[GlcNAcβ→]Hex₃HexNAc₂dHex₁ | |
| 2028 | Hex6HexNAc5 | 3 × Galβ4 | (Galβ4GlcNAc→)₃Hex₃HexNAc₂ | CO |
| 2067 | Hex10HexNAc2 | Manα | Glc→(Manα→)₈Hex₁HexNAc₂ | G |
| | | Glc | | |
| 2101 | Hex5HexNAc4dHex3 | GlcNAcβ | GlcNAcβ→Hex₅HexNAc₃dHex₃ | CO, FC |
| 2142 | Hex4HexNAc5dHex3 | Galβ4 | Galβ4GlcNAc→Hex₃HexNAc₄dHex₃ | CO, FC, |
| | | | | N > H |
| 2174 | Hex6HexNAc5dHex | GlcNAcβ | GlcNAcβ→Hex₆HexNAc₄dHex₁ | CO, F |
| | | 3 × Galβ4 | (Galβ4GlcNAc→)₃Hex₃HexNAc₂dHex₁ | |
| 2229 | Hex11HexNAc2 | Glc | Glc₂→(Manα→)₈Hex₁HexNAc₂ | G |
| | | Manα | | |
| 2304 | Hex5HexNAc5dHex3 | GlcNAcβ | GlcNAcβ→Hex₅HexNAc₄dHex₃ | CO, FC, |
| | | | | N = H |
| 2320 | Hex6HexNAc5dHex2 | GlcNAcβ | GlcNAcβ→Hex₆HexNAc₄dHex₂ | CO, FC |
| 2393 | Hex7HexNAc6 | Galβ4 | Galβ4GlcNAc→Hex₆HexNAc₅ | CO |
| 2466 | Hex6HexNAc5dHex3 | GlcNAcβ | GlcNAcβ→Hex₆HexNAc₄dHex₃ | CO, FC |
| 2539 | Hex7HexNAc6dHex | GlcNAcβ | GlcNAcβ→Hex1HexNAc₅dHex₁ | CO, F |
| | | 4 × Galβ4 | (Galβ4GlcNAc→)₄Hex₃HexNAc₂dHex₁ | |

*[M + Na]⁺ ion, first isotope.
^{§}“→” indicates linkage to a monosaccharide in the rest of the structure; “[ ]” indicates branch in the structure.
^{#}Preferred structure group based on monosaccharide compositions according to the present invention. HI, high-mannose; LO, low-mannose; S, soluble mannosylated; HF, fucosylated high-mannose; G, glucosylated high-mannose; HY, hybrid-type or monoantennary; CO, complex-type; F, fucosylation; FC, complex fucosylation; N = H, terminal HexNAc (HexNAc = Hex); N > H, terminal HexNAc (HexNAc > Hex).

TABLE 12

| Proposed composition | m/z | α-Man | β-GlcNAc | β4-Gal | β3-Gal |
|---|---|---|---|---|---|
| Hex2HexNAc | 568 | −−− | −−− | | |
| HexHexNAc2 | 609 | +++ | | | −−− |
| Hex2HexNAcdHex | 714 | +++ | | | |
| Hex3HexNAc | 730 | − | | | |
| HexHexNAc2dHex | 755 | +++ | | | |
| Hex2HexNAc2 | 771 | ++ | ++ | − | − |
| Hex4HexNAc | 892 | −−− | −−− | | |
| Hex2HexNAc2dHex | 917 | − | ++ | − | − |
| Hex3HexNAc2 | 933 | ++ | ++ | − | − |
| HexHexNAc3dHex | 958 | | | | |
| Hex2HexNAc3 | 974 | +++ | | ++ | −−− |
| Hex5HexNAc | 1054 | −−− | | | |
| Hex3HexNAc2dHex | 1079 | −− | ++ | − | − |
| Hex4HexNAc2 | 1095 | −− | + | − | − |
| Hex2HexNAc3dHex | 1120 | +++ | | + | −−− |
| Hex3HexNAc3 | 1136 | ++ | −−− | ++ | −− |
| Hex2HexNAc2dHex3 | 1209 | −−− | −−− | | |
| Hex6HexNAc | 1216 | −−− | | +++ | +++ |
| Hex4HexNAc2dHex | 1241 | −−− | | | − |
| Hex5HexNAc2 | 1257 | −− | | | |
| Hex3HexNAc3dHex | 1282 | ++ | −−− | + | − |
| Hex4HexNAc3 | 1298 | +++ | + | − | − |
| Hex3HexNAc4 | 1339 | | | +++ | −−− |
| Hex7HexNAc | 1378 | | +++ | +++ | |
| Hex5HexNAc2dHex | 1403 | −−− | | | − |
| Hex6HexNAc2 | 1419 | −− | + | | |
| Hex3HexNAc3dHex2 | 1428 | +++ | | | |
| Hex4HexNAc3dHex | 1444 | ++ | | − | − |
| Hex5HexNAc3 | 1460 | + | −− | + | − |
| Hex3HexNAc4dHex | 1485 | | −−− | ++ | − |
| Hex4HexNAc4 | 1501 | + | −−− | −− | − |
| Hex8HexNAc | 1540 | | | −−− | − |
| Hex3HexNAc5 | 1542 | | | +++ | |
| Hex6HexNAc2dHex | 1565 | −−− | | | −−− |
| Hex7HexNAc2 | 1581 | −− | | | |
| Hex4HexNAc3dHex2 | 1590 | | | | |
| Hex5HexNAc3dHex | 1606 | | −− | −− | − |
| Hex6HexNAc3 | 1622 | −− | −− | −− | − |
| Hex4HexNAc4dHex | 1647 | | −−− | | − |
| Hex5HexNAc4 | 1663 | | − | −− | |
| Hex3HexNAc5dHex | 1688 | | −−− | ++ | −−− |
| Hex4HexNAc5 | 1704 | | | +++ | |
| Hex8HexNAc2 | 1743 | −− | | | |
| Hex5HexNAc3dHex2 | 1752 | | −−− | −−− | |
| Hex6HexNAc3dHex | 1768 | −− | −− | −− | |
| Hex7HexNAc3 | 1784 | − | | −−− | |
| Hex4HexNAc4dHex2 | 1793 | | −−− | ++ | −−− |
| Hex5HexNAc4dHex | 1809 | | −− | −−− | |
| Hex6HexNAc4 | 1825 | +++ | +++ | −− | |
| Hex4HexNAc5dHex | 1850 | | | +++ | |
| Hex5HexNAc5 | 1866 | | | −−− | −−− |
| Hex3HexNAc6dHex | 1891 | | | ++ | −−− |
| Hex9HexNAc2 | 1905 | −−− | | | |
| Hex5HexNAc4dHex2 | 1955 | | −−− | −− | − |
| Hex6HexNAc4dHex | 1971 | | −−− | −−− | |
| Hex7HexNAc4 | 1987 | | −−− | | −−− |
| Hex4HexNAc5dHex2 | 1996 | | −−− | +++ | |
| Hex5HexNAc5dHex | 2012 | | −−− | −− | |
| Hex6HexNAc5 | 2028 | | − | −−− | − |
| Hex10HexNAc2 | 2067 | −−− | | | − |
| Hex5HexNAc4dHex3 | 2101 | | − | | |
| Hex6HexNAc4dHex2 | 2117 | | | −−− | −−− |
| Hex7HexNAc4dHex | 2133 | | −−− | | −−− |
| Hex4HexNAc5dHex3 | 2142 | | | −−− | −−− |
| Hex6HexNAc5dHex | 2174 | | −− | −−− | − |
| Hex5HexNAc7 | 2272 | | | +++ | |
| Hex5HexNAc5dHex3 | 2304 | | −−− | +++ | |
| Hex6HexNAc5dHex2 | 2320 | | −−− | −−− | |
| Hex7HexNAc6 | 2393 | | −− | −−− | |
| Hex6HexNAc5dHex3 | 2466 | | | −−− | −−− |
| Hex7HexNAc6dHex | 2539 | | −−− | −−− | |
| Hex8HexNAc7 | 2758 | | | −−− | −−− |

TABLE 13

| Proposed composition | m/z | β4-Gal | β-GlcNAc |
|---|---|---|---|
| Hex2HexNAc | 568 | − | −−− |
| HexHexNAc2 | 609 | +++ | |
| Hex3HexNAc | 730 | | |
| Hex2HcxNAc2 | 771 | | −− |
| Hex4HexNAc | 892 | | −−− |
| Hex2HexNAc2dHex | 917 | | − |
| Hex3HexNAc2 | 933 | | − |
| Hex2HexNAc3 | 974 | +++ | |
| Hex5HexNAc | 1054 | | |
| Hex3HexNAc2dHex | 1079 | | |
| Hex4HexNAc2 | 1095 | | |
| Hex2HexNAc3dHex | 1120 | +++ | |
| Hex3HexNAc3 | 1136 | ++ | −−− |
| Hex2HexNAc2dHex3 | 1209 | −−− | −−− |
| Hex6HexNAc | 1216 | | |
| Hex4HexNAc2dHex | 1241 | | |
| Hex5HexNAc2 | 1257 | | |
| Hex3HexNAc3dHex | 1282 | + | −− |
| Hex4HexNAc3 | 1298 | | |
| Hex3HexNAc4 | 1339 | +++ | |
| Hex2HexNac2dHex4 | 1355 | +++ | |
| Hex7HexNAc | 1378 | | |
| Hex5HexNAc2dHex | 1403 | | |
| Hex6HexNAc2 | 1419 | | |
| Hex4HexNAc3dHex | 1444 | + | |
| Hcx5HexNAc3 | 1460 | ++ | − |
| Hex3HcxNAc4dHex | 1485 | ++ | −−− |
| Hex4HexNAc4 | 1501 | −− | −−− |
| Hex8HexNAc | 1540 | | |
| Hex3HexNAc5 | 1542 | +++ | |
| Hex6HexNAc2dHex | 1565 | | |
| Hex7HexNAc2 | 1581 | | |
| Hex4HexNAc3dHex2 | 1590 | +++ | +++ |
| Hex5HexNAc3dHex | 1606 | − | |
| Hex6HexNAc3 | 1622 | −− | − |
| Hex4HexNAc4dHex | 1647 | | −−− |
| Hex5HexNAc4 | 1663 | −−− | ++ |
| Hex3HexNAc5dHex | 1688 | ++ | −−− |
| Hex9HexNAc | 1702 | −−− | −−− |
| Hex4HexNAc5 | 1704 | +++ | −−− |
| Hex8HexNAc2 | 1743 | | |
| Hex5HexNAc3dHex2 | 1752 | +++ | |
| Hex6HexNAc3dHex | 1768 | − | |
| Hex7HexNAc3 | 1784 | −−− | −−− |
| Hex4HexNAc4dHex2 | 1793 | +++ | |
| Hex5HexNAc4dHex | 1809 | −−− | + |
| Hex4HexNAc5dHex | 1850 | | −−− |
| Hex3HexNAc6dHex | 1891 | ++ | −−− |
| Hex9HexNAc2 | 1905 | | |
| Hex5HexNAc4dHex2 | 1955 | −−− | |
| Hex4HexNAc5dHex2 | 1996 | | −−− |
| Hex5HexNAc5dHex | 2012 | −−− | −−− |
| Hex6HexNAc5 | 2028 | −−− | |
| Hex10HexNAc2 | 2067 | | |
| Hex5HexNAc4dHex3 | 2101 | | + |
| Hex6HexNAc5dHex | 2174 | −−− | |
| Hex7HexNAc6 | 2393 | −−− | −−− |
| Hex7HexNAc6dHex | 2539 | −−− | −−− |

TABLE 14

| Proposed composition | m/z | α-Man | β4-Gal | β-GlcNAc |
|---|---|---|---|---|
| Hex2HexNAc | 568 | −−− | − | −−− |
| HexHexNAc2 | 609 | +++ | − | −−− |
| Hex3HexNAc | 730 | −− | − | |
| HexHexNAc2dHex | 755 | +++ | | −−− |
| Hex2HexNAc2 | 771 | ++ | − | −− |
| Hex4HexNAc | 892 | −−− | − | −−− |
| Hex2HexNAc2dHex | 917 | −− | − | −− |
| Hex3HexNAc2 | 933 | − | − | −− |
| Hex2HexNAc3 | 974 | ++ | + | −−− |
| Hex5HexNAc | 1054 | −−− | | |
| Hex3HexNAc2dHex | 1079 | −−− | − | −− |
| Hex4HexNAc2 | 1095 | −− | − | − |
| Hex2HcxNAc3dHex | 1120 | ++ | + | −−− |
| Hex3HexNAc3 | 1136 | + | ++ | −− |
| Hex6HexNAc | 1216 | −− | | |
| Hex4HexNAc2dHex | 1241 | −−− | | |
| Hex5HexNAc2 | 1257 | −−− | | |
| Hex3HexNAc3dHex | 1282 | | + | −− |
| Hex4HexNAc3 | 1298 | + | | |
| Hex3HexNAc4 | 1339 | | ++ | −−− |
| Hex7HexNAc | 1378 | −−− | | |
| Hex5HexNAc2dHex | 1403 | −−− | | |
| Hex6HexNAc2 | 1419 | −− | | |
| Hex3HexNAc3dHex2 | 1428 | +++ | | |
| Hex4HexNAc3dHex | 1444 | | | |
| Hex5HexNAc3 | 1460 | | + | |
| Hex3HexNAc4dHex | 1485 | | ++ | −−− |
| Hex4HexNAc4 | 1501 | | −− | −−− |
| Hex8HexNAc | 1540 | −−− | | −−− |
| Hex3HexNAc5 | 1542 | + | ++ | −−− |
| Hex6HexNAc2dHex | 1565 | −−− | | − |
| Hex7HcxNAc2 | 1581 | −− | | |
| Hex4HexNAc3dHex2 | 1590 | −−− | | ++ |
| Hex5HexNAc3dHex | 1606 | − | −− | + |
| Hex6HexNAc3 | 1622 | −− | −− | ++ |
| Hex4HexNAc4dHex | 1647 | | −− | −−− |
| Hex5HexNAc4 | 1663 | | −−− | + |
| Hex3HcxNAc5dHex | 1688 | | ++ | −−− |
| Hex4HexNAc5 | 1704 | | +++ | |
| Hex8HexNAc2 | 1743 | −− | | |
| Hex5HexNAc3dHex2 | 1752 | | | +++ |
| Hex6HexNAc3dHex | 1768 | − | −− | + |
| Hex7HexNAc3 | 1784 | −−− | −− | |
| Hex4HexNAc4dHex2 | 1793 | | + | −−− |
| Hex5HexNAc4dHex | 1809 | | −−− | |
| Hex6HexNAc4 | 1825 | −−− | − | + |
| Hex4HexNAc5dHex | 1850 | −−− | | −−− |
| Hex5HexNAc5 | 1866 | | −−− | −−− |
| Hex3HexNAc6dHex | 1891 | −−− | ++ | −−− |
| Hex9HexNAc2 | 1905 | −−− | | |
| Hex5HexNAc4dHex2 | 1955 | | | ++ |
| Hex6HexNAc4dHex | 1971 | | −−− | + |
| Hex7HexNAc4 | 1987 | | +++ | |
| Hex4HexNAc5dHex2 | 1996 | | | −−− |
| Hex5HexNAc5dHex | 2012 | | −−− | −−− |
| Hex6HexNAc5 | 2028 | | −−− | |
| Hex10HexNAc2 | 2067 | −−− | | |
| Hex5HexNAc4dHex3 | 2101 | | | + |
| Hex6HexNAc5dHex | 2174 | | −−− | |
| Hex6HexNAc6 | 2231 | | −−− | −−− |
| Hex5HexNAc5dHex3 | 2304 | | | −−− |
| Hex6HexNAc5dHex2 | 2320 | | −−− | −−− |
| Hex6HexNAc6dHex | 2377 | | −−− | −−− |
| Hex7HexNAc6 | 2393 | | −−− | −− |
| Hex6HexNAc5dHex3 | 2466 | | | |
| Hex7HexNAc6dHex | 2539 | | −−− | −−− |
| Hex5HexNAc6dHex4 | 3140 | | −−− | −−− |

TABLE 15
See also Example 8.
Summary of antibody stainings and FACS analysis of bone marrow derived mesenchymal stem
cells and osteogenic cells derived from them.

| | | BM- | | | posit | |
|---|---|---|---|---|---|---|
| Code | Antigen | MSC | posit (%) | Osteog | (%) | Change |
| GF274 | PNAd (peripheral lymph node addressin; CD62L ligand) closely | − | 0% | − | 0% | |
| | associated with L-selectin (CD34, GlyCAM-1, MAdCAM-1), sulfomucin | | | | | |
| GF275 | CA15-3 (Cancer antigen 15-3; sialylated carbohydrate epitope of the | +* | ~50% | + | 100% | |
| | MUC-1 glycoprotein) | | | | | |
| GF276 | oncofetal antigen, tumor associated glycoprotein (TAG-72) or CA 72-4 | −* | 0% | + | ~90% | ↑↑ |
| GF277 | human sialosyl-Tn antigen (STn, sCD175) | (+)* | >50% | + | ~90% | ↑ |
| GF278 | human Tn antigen (Tn, CD175 B1.1) | (+)* | >50% | + | ~80% | ↑ |
| GF295 | Blood group antigen precursor (BG1), Lewis c Gb3GN (pLN) | − | 0% | − | 0% | |
| GF280 | TF-antigen isoform (Nemod TF2) | −* | 0% | − | 0% | |
| GF281 | TF-antigen isoform (A68-E/E3) | −* | 0% | − | 0% | |
| GF296 | asialoganglioside GM1 | − | 0% | − | 0%** | |
| GF297 | Globoside GL4 | + | 100% | + | ~75% | |
| GF298 | Human CD77 (=blood group substance pk), GB3 | + | 80-90% | + | ~50% | |
| GF299 | Forssman antigen, glycosphingolipid (FO GSL) differentiation ag | − | 0% | − | 0% | |
| GF300 | Asialo GM2 | − | 0% | − | 0%** | |
| GF301 | Lewis b blood group antigen | −* | 0% | − | 0% | |
| GF302 | H type 2 blood group antigen | +* | ~50% | + | <50% | |
| GF303 | Blood group H1(O) antigen (BG4) | −* | 0% | + | >50% | ↑↑ |
| GF288 | Globo-H | −* | 0% | NT | | |
| GF304 | Lewis a | − | 0% | − | ** | |
| GF305 | Lewis x, CD15, 3-FAL, SSEA-1,3-fucosyl-N-acetyllactosamine | (+/−) | <5% | − | 0% | ↓ |
| GF306 | Sialyl Lewis a | − | 0% | − | 0% | |
| GF307 | Sialyl Lewis x | + | ~20% | (+/−) | <10%** | ↓ |
| GF353 | SSEA-3 (stage-specific embryonic antigen-3) | + | ~50% | (+/−) | ~10% | ↓↓ |
| GF354 | SSEA-4 (stage-specific embryonic antigen-4) | +* | ~75% | − | <5% | ↓↓ |
| GF365 | Nemod TFI, DC176, GalB1-3GalNAc | − | 0% | − | 0% | |
| GF374 | Glycodelin A, GdA, PP14 (A87-D/F4) | (+/−) | <5% | − | 0% | |
| GF375 | Glycodelin A, GdA, PP14 (A87-D/C5) | − | 0% | − | 0% | |
| GF376 | Glycodelin A, GdA, PP14 (A87-B/D2) | − | 0% | − | 0% | |

+ = positive,
(+) = weak positive,
(+/−) = single positive cells,
− = negative;
NT = not tested;
*= result has been confirmed by FACS analysis,
**= in certain cell batches higher binding or binding cells were observed and in the invention is directed to these markers.

TABLE 16

| Lectins | Target | % of positive cells |
|---|---|---|
| FITC-GNA | α-Man | 27.8 |
| FITC-HHA | α-Man | 95.3 |
| FITC-PSA | α-Man | 95.5 |
| FITC-RCA | β-Gal (Galβ4GlcNAc) | 94.8 |
| FITC-PNA | β-Gal (Galβ3GalNAc) | 31.1 |
| FITC-MAA | α2,3-sialylation | 89.9 |
| FITC-SNA | α2,6-sialylation | 14.3 |
| FITC-PWA | I-antigen | 1.9 |
| FITC-STA | i-antigen | 11.9 |
| FITC-LTA | α-Fuc | 2.8 |
| FITC-UEA | α-Fuc | 8.0 |

TABLE 17

| | | BM MSC | | | | | | | |
|---|---|---|---|---|---|---|---|---|---|
| | | lectin concentration, μg/ml | | | | | | | |
| Lectin | Target | 0.25 | 0.5 | 1 | 2.5 | 5 | 10 | 20 | 40 |
| FITC-GNA | α-Man | −¹⁾ | − | ++ | ++ | ++ | ++ | ++ | ++ |
| FITC-HHA | α-Man | ++ | ++ | +++ | +++ | +++ | +++ | +++ | +++ |
| FITC-PSA | α-Man | ++ | ++ | ++ | +++ | +++ | +++ | +++ | +++ |
| FITC-RCA | β-Gal (Galβ4GlcNAc) | − | − | +/− | +/− | + | + | ++ | ++ |
| FITC-PNA | β-Gal (Galβ3GalNAc) | − | − | − | − | +/− | +/− | +/− | + |
| FITC-MAA | α2,3-sialylation | − | − | − | +/− | + | ++ | ++ | ++ |
| FITC-SNA | α2,6-sialylation | − | − | − | − | +/− | +/− | + | + |
| FITC-PWA | I-antigen | − | − | − | − | − | − | +/− | +/− |
| FITC-STA | i-antigen | − | − | − | − | − | +/− | +/− | +/− |
| FITC-LTA | α-Fuc | − | − | − | − | − | − | − | − |
| FITC-UEA | α-Fuc | − | − | − | +/− | +/− | + | ++ | ++ |
| FITC-MBL | α-Man/β-GlcNAc | − | − | − | − | − | − | +/− | + |

¹⁾Grading of staining/labelling:
+++ very intense,
++ intense,
+ low,
+/− barely detectable,
− not labelled.

TABLE 18
Summary of the results of BM MSC grown on
different immobilized lectin surfaces.

| | Proliferation | Effect vs. |
|---|---|---|
| Coating | factor | plastic |
| plastic | 3.8 | |
| RCA | 1.0 | n.g. |
| PSA | 3.9 | (+) |
| LTA | 4.0 | + |
| SNA | 3.7 | (−) |
| GS II | 4.9 | + |
| UEA | 2.1 | − |
| EGA | 4.4 | + |
| MAA | 3.7 | (−) |
| STA | 3.1 | − |
| PWA | 4.2 | + |
| WFA | 2.9 | − |
| NPA | 3.6 | (−) |

Proliferation factor = the number of cells on day 3/the number of cells on day 1. Triplicates were used in calculations. Effect vs. plastic: ‘n.g.’ = no growth; ‘−’ = slower growth rate; ‘+’ = faster growth rate than on plastic; ‘( )’ nearly equal to plastic.

TABLE 19

| | | | | | CB CD34+ | | BM & CB | |
|---|---|---|---|---|---|---|---|---|
| Trivial name | Terminal epitope | hESC 1) | EB | st. 3 | & CD133+ | CB MNC | MSC | adipo/osteo |
| LN type 1, Le^{c} | GalβGlcNAc | N+ | 2) | +/− | q | N+/− | q | |
| | | O+ | +/− | | | O+/− | | |
| | | L++ | | | | L+ | | |
| Lea | Galβ3(Fucα4)GlcNAc | L+ | +/− | +/− | +/− | +/− | +/− | +/− |
| H type 1 | Fucα2Galβ3GlcNAc | L++ | +/− | +/− | +/− | +/− | +/− | +/− |
| Leb | Fucα2Galβ3(Fucα4)GlcNAc | + | +/− | +/− | +/− | +/− | +/− | +/− |
| sialyl Le^{a} | SAα3Galβ3(Fucα4)GlcNAc | | +/− | +/− | | | | |
| α3′-sialyl Le^{c} | SAα3Galβ3GlcNAc | | | | +/− | +/− | +/− | +/− |
| LN type 2 | Galβ4GlcNAc | N++ | + | + | N+ | N+ | N++ | N++ |
| | | O++ | | | O+ | O+ | O+ | |
| | | L+/− | | | | L+ | L++ | |
| Le^{x} | Galβ4(Fucα3)GlcNAc | N++ | +/− | +/− | N+ | N+/− | +/− | +/− |
| | | O+/− | | | O+ | O+ | | |
| | | L+/− | | | | L+/− | | |
| H type 2 | Fucα2Galβ4GlcNAc | N+ | +/− | +/− | N+ | +/− | +/− | +/− |
| | | O+/− | | | | | | |
| | | L+/− | | | | | | |
| Le^{y} | Fucα2Galβ4(Fucα3)GlcNAc | + | +/− | +/− | | | | |
| sialyl Le^{x} | SAα3Galβ4(Fucα3)GlcNAc | + | +/− | +/− | +/− | +/− | +/− | +/− |
| α3′-sialyl LN | SAα3Galβ4GlcNAc | N++ | N+ | N+ | N++ | N+ | N++ | N++ |
| | | O+ | | | O+ | O+ | O+ | |
| α6′-sialyl LN | SAα6Galβ4GlcNAc | N+ | N++ | N++ | N+ | N++ | +/− | |
| Core 1 | Galβ3GalNAcα | O+ | +/− | +/− | O+ | O+ | O+ | |
| H type 3 | Fucα2Galβ3GalNAcα | O+ | +/− | +/− | +/− | +/− | +/− | |
| sialyl Core 1 | SAα3Galβ3GalNAcα | O+ | | | O+ | O+ | O+ | |
| disialyl Core 1 | SAα3Galβ3Saα6GalNAcα | O+ | | | O+ | O+ | O+ | |
| type 4 chain | Galβ3GalNAcβ | L+ | +/− | +/− | +/− | L + | L+ | |
| H type 4 | Fucα2Galβ3GalNAcβ | L+ | +/− | +/− | +/− | +/− | +/− | |
| α3′-sialyl type 4 | SAα3Galβ3GalNAcβ | L++ | +/− | +/− | +/− | +/− | +/− | |
| LacdiNAc | GalNAcβ4GlcNAc | N+ | +/− | +/− | +/− | +/− | +/− | +/− |
| Lac | Galβ4Glc | L+ | q | q | q | L+ | L+ | |
| GlcNAcβ | GlcNAcβ | N+/− | q | q | N+ | +/− | +/− | q |
| | | L+ | | | | | | |
| Tn | GalNAcα | q | | | q | q | O+ | |
| sialyl Tn | SAα6GalNAcα | | | | | | O+ | |
| GalNAcβ | GalNAcβ | L+ | q | q | +/− | +/− | N+/− | |
| | | N+ | | | | | L+ | |
| poly-LN, i | repeats of Galβ4GlcNAcβ3 | + | q | q | + | + | ++ | q |
| poly-LN, I | Galβ4GlcNAcβ3(Galβ4GlcNAcβ6)Gal | L+ | +/− | +/− | +/− | L+ | L+ | q |

1) Stem cell and differentiated cell types are abbreviated as in other parts of the present document;
st. 3 indicates stage 3 differentiated, preferentially neuronal-type differentiated cells;
adipo/osteo indicates cells differentiated into adipocyte or osteoblast direction from MSC.
2) Occurrence of terminal epitopes in glycoconjugates and/or specifically in N-glycans (N), O-glycans (O), and/or glycosphingolipids (L).
Code:
q, qualitative data;
+/−, low expression;
+, common;
++, abundant.

TABLE 20

| | | Neutral | | | Sialylated | | |
|---|---|---|---|---|---|---|---|
| | | glycans | | | glycans | | |
| Class | Definition | hESC | MSC | CB MNC | hESC | MSC | CB MNC |
| Examples of | | | | | | | |
| glycosphingolipid glycan classification | | | | | | | |
| Lac | n_{Hex} = 2 | 1 | 1 | 2 | 1 | a) | |
| Ltri | n_{Hex} = 2 and n_{HexNAc} = 1 | 18 | 33 | 12 | 25 | | |
| L1 | n_{Hex} = and n_{HexNAc} = 1 | 46 | 32 | 46 | 56 | | |
| L2 | 3 ≦ n_{Hex} ≦ 4 and n_{HexNAc} = 2 | 11 | 15 | 4 | <1 | | |
| L3+ | i + 1 ≦ n_{Hex} ≦ i + 2 and n_{HexNAc} = i ≧ 3 | 1 | 7 | 3 | 1 | | |
| Gb | n_{Hex} = 4 and n_{HexNAc} = 1 | 20 | 1 | 1 | 16 | | |
| O | other types | 23 | 11 | 34 | 1 | | |
| F | fucosylated, n_{dHex} ≧ 1 | 43 | 12 | 7 | 1 | | |
| T | non-reducing terminal HexNAc, | 27 | 47 | 12 | 26 | | |
| | n_{Hex} ≦ n_{HexNAc} + 1 | | | | | | |
| SA1 | monosialylated, n_{Neu5Ac} = 1 | | | | 86 | | |
| SA2 | disialylated, n_{Neu5Ac} = 2 | | | | 14 | | |
| SP | sulphated or phosphorylated, +80 Da | | | | <1 | | |
| Examples of O-linked glycan classification | | | | | | | |
| O1 | n_{Hex} = 1 and n_{HexNAc} = 1 | a) | a) | | 43 | a) | |
| O2 | n_{Hex} = 2 and n_{HexNAc} = 2 | 53 | | | 35 | | |
| O3+ | n_{Hex} = i and n_{HexNAc} = i ≧ 3 | 13 | | | 13 | | |
| O | other types | 34 | | | 9 | | |
| F | fucosylated, n_{dHex} ≧ 1 | 1 | 47 | 64 | 5 | 15 | 15 |

| | | | | | |
|---|---|---|---|---|---|
| T | non-reducing terminal HexNAc, | 12 | a) | <1 | a) |
| | n_{Hex} ≦ n_{HexNAc} + 1 | | | | |
| SA1 | monosialylated, n_{Neu5Ac} = 1 | | | 39 | |
| SA2 | disialylated, n_{Neu5Ac} = 2 | | | 52 | |

| | | | |
|---|---|---|---|
| SP | sulphated or phosphorylated, +80 Da | 8 | 21 |

a) not included in present quantitative analysis.

TABLE 21

| | CB | CB | |
|---|---|---|---|
| | MNC | MSC | hESC |
| Neutral | | | |
| glycosphingolipid glycans^{#} | | | |
| L1 | 1^{§} | 2 | 1 |
| L2 | 49 | 74 | 64 |
| L3 | 7 | 10 | 12 |
| L4 | 4 | 6 | 1 |
| L5+ | 2 | 0.5 | 0.5 |
| Gb | 0.5 | 0.5 | 20 |
| O | 37 | 8 | 2 |
| fucosylated | 11 | 8 | 43 |
| α1,2-Fuc | 11 | 6 | 39 |
| α1,3/4-Fuc | 6 | 2 | 3 |
| β1,4-Gal | 89 | 72 | 4 |
| β1,3-Gal | 48 | 68 | 50 |
| term. HexNAc | 10 | 27 | 27 |
| Acidic | | | |
| glycosphingolipid glycans^{#} | | | |
| L1 | 1^{§} | 10 | n.d. |
| L2 | 62 | 77 | 81 |
| L3 | 26 | 6 | 0.5 |
| L4 | 11 | 4 | 0.5 |
| L5+ | <0.5 | 0.5 | 0.5 |
| Gb | — | 0.5 | 16 |
| O | — | 2 | <0.5 |
| α-NeuAc | 100 | 100 | 100 |
| α2,3-NeuAc | 97 | 86 | 81 |
| fucosylated | 4 | 2 | 1 |
| β1,4-Gal | 97 | 32 | n.d. |

^{#}Abbreviations: L1-6, glycosphingolipid glycan type Li, wherein n_{HexNAc} + 1 ≦ n_{Hex} ≦ n_{HexNAc} + 2, and i = n_{HexNAc} + 1; Gb, (iso)globopentaose, wherein n_{Hex} = 4 and n_{HexNAc} = 1; term. HexNAc, terminal HexNAc in L1-6, wherein n_{HexNAc} + 1 = n_{Hex}; O, other types; n.d., not determined.
^{§}Figures indicate percentage of total detected glycan signals.

TABLE 22
Relative expression levels of acidic
O-glycan components in BM MSC and OB.

| Proposed | | BM MSC | Comparison | OB |
|---|---|---|---|---|
| composition | m/z | % | MSC:OB | % |
| S2H2N3F1 | 1678 | 3.20 | ∞ | 0.00 |
| S1H3N3 | 1403 | 1.96 | ∞ | 0.00 |
| H7N2P2 | 1717 | 1.72 | ∞ | 0.00 |
| H5N4P2 | 1799 | 1.04 | ∞ | 0.00 |
| H6N2F1P1 | 1621 | 1.02 | ∞ | 0.00 |
| H6N4P2 | 1961 | 0.99 | ∞ | 0.00 |
| H3N3P1 | 1192 | 0.95 | ∞ | 0.00 |
| S1H2N2F1 | 1184 | 0.90 | ∞ | 0.00 |
| S1H3N2 | 1200 | 0.89 | ∞ | 0.00 |
| H5N4F1P1 | 1865 | 0.86 | ∞ | 0.00 |
| S2H3N3 | 1694 | 0.80 | ∞ | 0.00 |
| H6N2P2 | 1555 | 0.78 | ∞ | 0.00 |
| S1H6N3 | 1889 | 0.75 | ∞ | 0.00 |
| H4N3P1 | 1354 | 0.73 | ∞ | 0.00 |
| S1H4N2 | 1362 | 0.66 | ∞ | 0.00 |
| S1H5N3 | 1727 | 0.64 | ∞ | 0.00 |
| H5N4F1P1 | 1719 | 0.63 | ∞ | 0.00 |
| S1H4N4 | 1768 | 0.58 | ∞ | 0.00 |
| H4N3F1P1 | 1500 | 0.50 | ∞ | 0.00 |
| S1H5N3F1 | 1873 | 0.13 | ∞ | 0.00 |
| S1H4N3 | 1565 | 0.05 | ∞ | 0.00 |
| S2H2N1F1 | 1475 | 6.62 | 23.4 | 0.28 |
| S2H3N2F1 | 1637 | 4.81 | 4.15 | 1.16 |
| H2N2P1 | 827 | 32.36 | 1.31 | 24.78 |
| H2N2F1P1 | 973 | 1.59 | 0.80 | 1.99 |
| S2H2N2 | 1329 | 9.40 | 0.56 | 16.73 |
| S1H2N2 | 1038 | 19.28 | 0.49 | 39.67 |
| S2H1N1 | 964 | 4.01 | 0.42 | 9.46 |
| S1H2N2P1 | 1118 | 2.17 | 0.39 | 5.62 |
| S1H3N3F1 | 1549 | 0.00 | 0 | 0.32 |

Composition: S = NeuAc, H = Hex, N = HexNAc, F = dHex (Fuc), P = sulphate or phosphate ester
m/z: mass-to-charge ratio of [M − H]- signal.
Comparison: relation of % in BM MSC to % in OB; values over 1 indicate overexpression in BM MSC and values less than 1 indicate overexpression in OB; ∞ indicates that expression was below detection limit in OB; 0 indicates that expression was below detection limit in BM MSC.

TABLE 23
Summary of immunohistochemical stainings (IHC) and FACS analysis of bone
marrow derived mesenchymal stem cells (BM-MSC) and osteogenic cells derived thereof
(osteogenic). FACS results are shown as an average percentage of positive cells in a cell
population (n = 1-3 individual experiment(s)). Trypsin FACS results are from single
Experiment.

| | | BM- | | Tryps. | | Osteog. | Tryps. |
|---|---|---|---|---|---|---|---|
| | | MSC | BM-MSC | FACS | Osteog. | FACS | FACS |
| Code | Antigen | IHC | FACS (%) | (%) | IHC | (%) | (%) |
| GF274 | PNAd (peripheral lymph node addressin; CD62L ligand) closely | − | 0.9 | 0.4 | − | 1.8 | 0.5 |
| | associated with L-selectin (CD34, GlyCAM-1, MAdCAM-1), | | | | | | |
| | sulfo-mucin | | | | | | |
| GF275 | CA15-3 (Cancer antigen 15-3; sialylated carbohydrate epitope of | + | 46.5 | 57.9 | + | 79.1 | 14.1 |
| | the MUC-1 glycoprotein) | | | | | | |
| GF276 | oncofetal antigen, tumor assoctated glycoprotein (TAG-72) or CA | − | 0.8 | 0.5 | + | 0.8 | |
| | 72-4 | | | | | | |
| GF277 | human sialosyl-Tn antigen (STn, sCD175) | (+) | 7.3 | 0.4 | + | 1.0 | 0.7 |
| GF278 | human Tn antigen (Tn, CD175 B1.1) | (+) | 5.9 | 0.5 | + | 3.0 | 0.9 |
| GF295 | Blood group antigen precursor (BG1), Lewis c Gb3GN (pLN) | − | 9.6 | 0.7 | − | 2.7 | 1.0 |
| GF280 | TF-antigen isoform (Nemod TF2) | − | NT | | − | NT | |
| GF281 | TF-antigen isoform (A68-E/E3) | − | NT | | − | NT | |
| GF296 | asialoganglioside GM1 | − | 22 | 1.1 | − | 48.2 | 1.1 |
| GF297 | Globoside GL4 | + | 16.9 | 14.2 | + | 28.4 | 4.9 |
| GF298 | Human CD77 (=blood group substance pk), GB3 | + | 21.8 | 27.2 | + | 52.7 | 4.9 |
| GF299 | Forssman antigen, glycosphingolipid (FO GSL) differentiation ag | − | 4.1 | 0.4 | − | 5.5 | 0.4 |
| GF300 | Asialo GM2 | − | 17.1 | 0.9 | − | 53.8 | 1.7 |
| GF301 | Lewis b blood group antigen | − | 1.2 | | − | 1.3 | 0.7 |
| GF302 | H type 2 blood group antigen | + | 14.7 | 0.7 | + | 26.2 | 2.4 |
| GF303 | Blood group H1 (O) antigen (BG4) | − | 1.4 | 0.3 | + | 0.7 | 0.6 |
| GF288 | Globo-H | − | NT | | NT | NT | |
| GF304 | Lewis a | − | 13 | 1.7 | − | 23.4 | 1.4 |
| GF305 | Lewis x, CD15, 3-FAL, SSEA-1, 3-fucosyl-N-acetyllactosamine | (+/−) | 1 | 0.5 | − | 1.1 | 0.7 |
| GF306 | Sialyl Lewis a | − | 4.9 | 0.8 | − | 2.7 | 0.7 |
| GF307 | Sialyl Lewis x | + | 82.1 | 70.4 | (+/−) | 55.7 | 33 |
| GF353 | SSEA-3 (stage-specific embryonic antigen-3) | + | 33.8 | 6.8 | (+/−) | 6.2 | 0.8 |
| GF354 | SSEA-4 (stage-specific embryonic antigen-4) | + | 77.2 | 53.7 | − | 34.0 | 2.4 |
| GF365 | Nemod TF1, DC176, GalB1-3GalNAc | − | 3.8 | | − | 1.1 | 0.8 |
| GF374 | Glycodelin A, GdA, PP14 (A87-D/F4) | (+/−) | 0.9 | | − | 0.3 | 0.6 |
| GF375 | Glycodelin A, GdA, PP14 (A87-D/C5) | − | 2.4 | | − | 0.6 | 0.8 |
| GF376 | Glycodelin A, GdA, PP14 (A87-B/D2) | − | 3.4 | | − | 0.6 | 0.6 |
| GF393 | Lewis y | − | NT | | − | 0.6 | 0.5 |
| GF394 | H disaccharide | − | NT | | − | 0.5 | 1.2 |

+ = positive,
(+) = weak positive,
(+/−) = single positive cells,
− = negative;
NT = not tested

TABLE 24
Protease sensitive glycan epitopes on the cell surface of BM-MSC and osteogenic
cells derived thereof. Results are shown as a percentage of positive cells in FACS analysis.
Codes for antibodies are as described in Table 25.

| | | BM-MSC | BM-MSC | Osteog | Osteog |
|---|---|---|---|---|---|
| Code | Antigen | Versene (%) | Trypsin (%) | Versene (%) | Trypsin (%) |
| GF275 | CA15-3 (Cancer antigen 15-3; sialylated | | | 96.9 | 14.1 |
| | carbohydrate epitope of the MUC-1 | | | | |
| | glycoprotein) | | | | |
| GF277 | human sialosyl-Tn antigen (STn, sCD175) | 4.0 | 0.4 | | |
| GF278 | human Tn antigen (Tn, CD175 B1.1) | 4.7 | 0.5 | | |
| GF295 | Blood group antigen precursor (BG1), | 4.4 | 0.7 | | |
| | Lewis c Gβ3GN (pLN) | | | | |
| GF296 | asialoganglioside GM1 | 34.3 | 1.1 | 35.5 | 1.1 |
| GF299 | Forssman antigen, glycosphingolipid (FO | 4.1 | 0.4 | 6.7 | 0.4 |
| | GSL) differentiation ag | | | | |
| GF300 | asialoganglioside GM2 | 19.4 | 0.9 | 55.3 | 1.7 |
| GF302 | H type 2 blood group antigen | 6.0 | 0.7 | 23.3 | 2.4 |
| GF304 | Lewis a | 14.3 | 1.7 | 10.4 | 1.4 |
| GF306 | Sialyl Lewis a | 5.9 | 0.8 | 1.3 | 0.7 |
| GF307 | Sialyl Lewis x | 82.1 | 70.4 | 62.3 | 33.0 |
| GF354 | SSEA-4 (stage-specific embryonic antigen- | 77.2 | 53.7 | 21.4 | 2.4 |
| | 4) | | | | |

TABLE 25
Detailed information of the primary anti-glycan antibodies used in these examples.
Alternative antibody clones in italics.

| Code | Epitope | Terminal structure | Company | Cat number | Clone | Host/Class |
|---|---|---|---|---|---|---|
| GF 274 | Sulfo-mucin, PNAD, | Sulfo-mucin | BD | 553863 | MECA-79 | rat/IgM |
| | MECA-79, CD62L, | | Pharmingen | | | |
| | extended core 1 | | | | | |
| GF 275 | Ca15-3 sialyted epitope | SAα-mucin | Acris | BM3359 | 695 | mouse/IgG1 |
| GF 553 | | | | | | |
| GF 276 | TAG-72, CA 72-4, cancer | | Acris | DM288 | B72.3 | mouse/IgG1 |
| | glycoprotein | | | | | |
| GF 277 | Sialosyl-Tn, sCD175 | SA(α6)GalNAcαS/T | Acris | DM3197 | B35.1 | mouse/IgG1 |
| GF 372 | | | | | | |
| GF 278 | Tn, CD175 | GalNAcαS/T | Acris | DM3218 | B1.1 | mouse/IgM |
| VPU008 | | | | | | |
| GF 280 | TF-antigen isoform, CD176 | Gal(β3)GalNAc(α/β) (α 40x > β) | Glycotope | MAB-S301 | Nemod | mouse/IgM |
| | | | | | TF2 | |
| GF 281 | TF-antigen isoform, CD176 | Gal(β3)GalNAcβ | Glycotope | MAB-S305 | A68-E/E3 | mouse/IgG1 |
| GF 285 | H Type 2, Lewis b, Lewis y | Fuc(α2)Gal, Fuc(a2)Gal(β4)GlcNAc, | Acris | DM3014 | B389 | mouse/IgG1 |
| | | Fuc(α2)Gal(β4)[Fuc(α3)]GlcNAc | | | | |
| GF 286 | H Type 2, CD173 | Fuc(α2)Gal(β4)GlcNAc | Acris | BM258P | BRIC 231 | mouse/IgG1 |
| GF 288 | Globo-H | Fuc(α2)Gal(β3)GalNAc(β3)Gal(α4)Gal(β4)GlcβCer | Glycotope | MAB-S206 | A69-A/E8 | mouse/IgM |
| GF 403 | | | | | | |
| GF 295, | Lewis c, pLN, | Gal(β3)GlcNAβ(3Lac) | Abcam | ab3352 | K21 | mouse/IgM |
| GF 279 | Gal(β3)GlcNAc | | | | | |
| GF 555 | | | | | | |
| GF 296, | asialo GM1 | Gal(β3)GalNAc(β4)Gal(β4)GlcβCer | Acris | BP282 | polyclonal | rabbit |
| GF 282 | | | | | | |
| GF 427 | | | | | | |
| GF 297, | Globoside Gb4, GL4, | GalNAc(β3)Gal(α4)Gal(β4)GlcβCer | Abcam | ab23949 | polyclonal | rabbit/IgG |
| GF 366 | globotetraose | | | | | |
| VPU001 | | | | | | |
| GF 298 | Globoside Gb3, | Gal(α4)Gal(β4)GlcβCer | Acris | SM1160P | 38-13 | rat/IgM |
| GF 367 | globotriose, CD77, | | | | | |
| | blood group pk | | | | | |
| GF 299, | Forssman ag, | GalNAc(α3)GalNAc(β4)Gal(α4)Gal(β4)GlcβCer, | Acris | BM4091 | FOM-1 | rat/IgM |
| GF 401 | glycosphingolipid | GalNAc(α3)GalNAcβ-R | | | | |
| GF 554 | | | | | | |
| GF 300 | asialo GM2 | GalNAc(β4)Gal(β4)GlcβCer | Acris | BP283 | polyclonal | rabbit |
| GF 428 | | | | | | |
| GF 301, | Lewis b | Fuc(α2)Gal(β3)[Fuc(α4)]GlcNAc | Acris | SM3092P | 2-25LE | mouse/IgG1 |
| GF 283 | | | | DM3122 | | |
| VPU004 | | | | | | |
| GF 302 | H Type 2 | Fuc(α2)Gal(β4)GlcNAc | Acris | DM3015 | B393 | mouse/IgM |
| GF 284 | | | | | | |
| GF 303 | H Type 1, blood group | Fuc(α2)Gal(β3)GlcNAc | Abcam | ab3355 | 17-206 | mouse/IgG3 |
| GF 287 | antigen H1 | | | | | |
| GF 304 | Lewis a | Gal(β3)[Fuc(α4)]GlcNAc | Chemicon | CBL205 | PR5C5 | mouse/IgG1 |
| GF 429 | | | Abcam | Ab3967 | 7LE | |
| | | | | Ab3356 | T174 | |
| | | | Genetex | GTX28602 | B369 | |
| GF 305 | Lewis x, CD15, SSEA-1 | Gal(β4)[Fuc(α3)]GlcNAc | Chemicon | CBL144 | 28 | mouse/IgM |
| GF 306, | sialyl Lewis a | SA(α3)Gal(β3)[Fuc(α4)]GlcNAc | Chemicon | MAB2095 | KM231 | mouse/IgG1 |
| GF 430 | | | Invitrogen | 18-7240 | 116-NS- | |
| VPU002 | | | | | 19-9 | |
| | | | BioGenex | MU424-UC | C241:5:1:4 | |
| | sialyl Lewis a, c | | Seikagaku | 270443 | 2D3 | mouse/IgM |
| GF 307 | sialyl Lewis x | SA(α3)Gal(β4)[Fuc(α3)]GlcNAc | Chemicon | MAB2096 | KM93 | mouse/IgM |
| GF 353 | SSEA-3, | Gal(β3)GalNAc(α3)Gal | Chemicon | MAB4303 | MC-631 | rat/IgM |
| GF 431 | galactosylgloboside | | | | | |
| GF 354, | SSEA-4, | SA(α3)Gal(β3)GalNAc(β3)Gal | Chemicon | MAB4304 | MC-813- | mouse/IgG3 |
| GF 432 | sialylgalactosylgloboside | | | | 70 | |
| VPU003 | | | | | | |
| GF 355 | Gal(α3)Gal | Gal(α3)Gal | Chemicon | AB2052 | | baboon |
| GF 365 | TF-antigen isoform, CD176 | Gal(β3)GalNAc(α/β) (α 10x > β) | Glycotope | MAB-S302 | Nemod | mouse/IgM |
| | | | | | TF1 | |
| GF 368 | LacdiNAc | GalNAc(β4)GlcNAc | LUMC | anti-LDN | 259-2A1 | IgG3 |
| | | | (Leiden Univ | mAb | | |
| | | | Medical | | | |
| | | | Center) | | | |
| GF 369 | LacdiNAc | GalNAc(β4)GlcNAc | LUMC | anti-LDN | 273-3F2 | IgM |
| | | | (Leiden Univ | mAb | | |
| | | | Medical | | | |
| | | | Center) | | | |
| GF 370 | α3-Fuc-LacdiNAc | GalNAc(β4)[Fuc(α3)]GlcNAc | LUMC | anti LDN-F | 290-2E6 | IgM |
| | | | (Leiden Univ | mAb | | |
| | | | Medical | | | |
| | | | Center) | | | |
| GF 371 | α3-Fuc-LacdiNAc | GalNAc(β4)[Fuc(α3)]GlcNAc | LUMC | anti LDN-F | 291-3E9 | IgM |
| | | | (Leiden Univ | mAb | | |
| | | | Medical | | | |
| | | | Center) | | | |
| GF 374 | Glycodelin A, isoform | LacdiNAc | Glycotope | MAB-S901 | A87-D/C5 | mouse/IgG1, |
| | | | | | | IgG2b, IgM |
| GF 375 | Glycodelin A, isoform | LacdiNAc | Glycotope | MAB-S902 | A87-D/F4 | mouse/IgG1 |
| GF 376 | Glycodelin A, isoform | LacdiNAc | Glycotope | MAB-S903 | A87-B/D2 | mouse/IgG1 |
| GF 377 | PN-15 renal gp200, | | Acris | DM3184P | PN-15 | mouse/IgG1 |
| GF 373 | cancer glycoprotein | | | | | |
| GF 393 | Lewis y, CD174 | Fuc(α2)Gal(β4)[Fuc(α3)]GlcNAcβ | Glycotope | MAB-S201 | A70-C/C8 | mouse/IgM |
| GF 289 | | | | | | |
| GF 394 | H disaccharide | Fuc(α2)Galβ | Glycotope | MAB-S204 | A51-B/A6 | mouse/IgA |
| GF 290 | | | | | | |
| GF 406 | GD2 | GalNAc(β4)(SA(α8)SA)(α3)Gal(β4)Glc | Chemicon | MAB4309 | VIN-2PB- | mouse/IgM |
| GF 558 | | | | | 22 | |
| GF 407 | GD3 | SA(α8)SA(α3)Gal(β4)Glc | Chemicon | MAB4308 | VIN-IS-56 | mouse/IgM |
| GF 408 | blood group | GalNAc(α3)Fuc(α2)Galβ | Acris | DM3108 | B480 | mouse/IgG1 |
| | Ag A-b45.1 (A1, A2) | | | | | |
| GF 409 | blood group A | | Acris | BM255 | HE-195 | mouse/IgM |
| | (A3, Ax, A3B, AxB) | | | | | |
| GF 410 | blood group ABH | | Acris | SM3004 | HE-10 | mouse/IgM |
| GF 411 | blood group B (secretor) | | Acris | BM256 | HEB-29 | mouse/IgM |
| GF 412 | blood group Ag B (general) | | Acris | DM3012 | B460 | mouse/IgM |
| GF 413 | Gal(α3)Gal | Gal(α3)Gal(β4)GlcNAc-R | Alexis | ALX-801- | M86 | mouse/IgM |
| | | | Bio- | 090 | | |
| | | | chemicals | | | |
| GF 414 | TRA-1-81 Ag | | Chemicon | MAB4381 | TRA-1-81 | mouse/IgM |
| GF 556 | | | | | | |
| GF 415 | TRA-1-60 Ag | | Chemicon | MAB4360 | TRA-1-60 | mouse/IgM |
| GF 557 | | | | | | |
| GF 416 | Mannose | Man | | | | mouse/IgM |
| GF 418 | Globo-H | Fuc(α2)Gal(β3)GalNAc(β3)Gal(α4)Gal(β4)GlcβCer | Alexis | ALX-804- | MBr1 | mouse/IgM |
| | | | biochemicals | 550-C050 | | |
| GF 515 | CD15, Lewis x | Gal(β4)[Fuc(α3)]GlcNAc | BD | 557895 | W6D3 | mouse/IgG1, |
| | | | Pharmingen | | | k |
| GF 516 | sCD15, sialyl Lewis x | SA(α3)Gal(β4)[Fuc(α3)]GlcNAc | BD | 551344 | CSLEX1 | mouse/IgM, |
| | | | Pharmingen | | | k |
| GF 517 | CD15, Lewis x | Gal(β4)[Fuc(α3)]GlcNAc | Abcam | ab34200 | TG-1 | mouse/IgM |
| GF 518 | SSEA-1 | Gal(β4)[Fuc(α3)]GlcNAc | Abcam | ab16285 | MC480 | mouse/IgM |
| GF 525 | CD15, reacts with 220 kD | Gal(β4)[Fuc(α3)]GlcNAc | Abcam | ab17080 | MMA | mouse/IgM |
| | protein | | | | | |
| GF 526 | PSGL-1, sLex on core 2 | SA(α3)Gal(β4)[Fuc(α3)]GlcNAc | R&D | MAB996 | CHO131 | mouse/IgM |
| | O-glycans | | Systems | | | |
| GF 621 | GD3 | SA(α8)SA(α3)Gal(β4)Glc | BD | 554274 | MB3.6 | mouse/IgG3 |
| | | | Pharmingen | | | |
| GF 622 | GD2 | GalNAc(β4)(SA(α8)SA)(α3)Gal(β4)Glc | BD | 554272 | 14.G2a | mouse/IgG2 |
| | | | Pharmingen | | | |
| GF 623 | GT1b | | US | G2006-90A | 3C96 | mouse/IgM |
| | | | Biological | | | |
| GF 624 | GD1b | | US | G2004-90B | 2S1 | mouse/IgG3 |
| | | | Biological | | | |
| GF 625 | GD2 | GalNAc(β4)(SA(α8)SA)(α3)Gal(β4)Glc | US | G2205-02 | 2Q549 | mouse/IgG2 |
| | | | Biological | | | |
| GF 626 | GD3 | SA(α8)SA(α3)Gal(β4)Glc | Covalab | mab0014 | 4F6 | mouse/IgG3 |
| GF 627 | OAcGD3 | | US | G2005-67 | 4i283 | mouse/IgG3 |
| | | | Biological | | | |
| GF 628 | A2B5 | | Chemicon | MAB312R | A2B5-105 | mouse/IgM |
| VPU005 | GD3 | SA(α8)SA(α3)Gal | Seikagaku | 270554 | S2-566 | mouse/IgM |
| VPU006 | Tn antigen, CD175 | GalNAcαS/T | Abcam | ab31775 | 0.BG.12 | mouse/IgG |
| VPU007 | sialyl Tn, sCD175 | SA(α6)GalNAcαS/T | Abcam | ab24005 | BRIC111 | mouse/IgG |
| VPU009 | SSEA-3, | Gal(β3)GalNAc(β3)Gal | R&D | MAB1434 | MC-631 | rat/IgM |
| | galactosylgloboside | | Systems | | | |
| | GlcNAcβ1-6R | | Jeffersson | FE-J1 | | mouse/IgM |
| | | | medical | | | |
| | | | college | | | |
| | Galβ1-4GlcNAcβ1-3R | | Jeffersson | FE-A5 | | mouse/IgM |
| | | | medical | | | |
| | | | college | | | |
| | Galβ1-4GlcNAcβ1-6R | | Jeffersson | FE-A6 | | mouse/IgM |
| | | | medical | | | |
| | | | college | | | |

TABLE 26
Flow cytometric (FACS) and immunohistochemical (IHC) analysis of mesenchymal stem cells (MSC) and cells differentiated into osteogenic (OG) and adipogenic (adipo) lineages.

| | | | | BM-MSC¹⁾ | BM-OG | | | |
|---|---|---|---|---|---|---|---|---|
| | | | | FACS (% ± SD) | FACS (% ± SD) | CB-MSC | CB-OG | CB-Adipo |
| Code | Trivial name | Structure | Terminal epitope | IHC²⁾ | IHC | FACS (% ± SD) | FACS (%) | FACS (%) |
| GF416 | Mannose | | Man | 0.8 ± 0.42 | 13.2 | 2.90 ± 2.8 | 8.60 | 34.9 |
| GF278 | Tn | □-S/T | GalNAcαS/T | 5.9 ± 1.7 | 2.95 ± 2.6 | 2.43 ± 2.75 | 0.70 | 1.8 |
| VPU008 | | | | + | ++ | | | |
| VPU006 | Tn antigen, CD175 | □-S/T | GalNAcαS/T | 0.9 ± 0.35 | ND | 0.6 ± 0.17 | 0.5 | 0.6 |
| VPU007 | sialyl Tn, sCD175 | 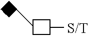 | SAα6GalNAcαS/T | 1.3 ± 0.28 | ND | 0.5 ± 0.17 | 0.8 | 1 |
| GF277 | Sialosyl-Tn | 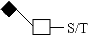 | SAα6GalNAcαS/T | 7.3 ± 4.67 + | 0.95 ± 0.21 ++ | 2.63 ± 1.6 | 0.8 | 5.7 |
| GF276 | TAG-72, CA 72-4 | 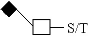 | TAG-72 carried sialyl-Tn, cancer glycoprotein | 0.75 ± 0.36 − | 0.75 ± 0.64 ++ | 0.90 ± 0.28 | 0.6 | 0.6 |
| GF280 | TF-antigen | 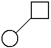 | Galβ3GalNAcα/β (α 40x > β) | 5 − | ND − | 1.97 ± 1.65 | 0.7 | 0.8 |
| GF281 | TF-antigen | 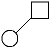 | Galβ3GalNAcβ | 1.3 − | ND − | 6.2 ± 7.3 | 0.9 | 2.5 |
| GF365 | TF-antigen | 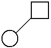 | Galβ3GalNAcα/β (α 10x > β) | 2.95 ± 1.2 − | 1.1 − | 4.25 ± 4.2 | 1.4 | 11.6 |
| GF274 | MECA-79, Sulfo-mucin, PNAD | 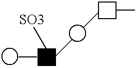 | Sulfo-mucin | 0.9 − | 1.8 ± 0.14 − | 2.4 ± 2.3 | 1.1 | 1.7 |
| GF275 | Cal 5-3 sialyted epitope | | SAα-mucin | 46.5 ± 38.0 | 79.1 ± 25.2 | 2.0 ± 0.0 | 6.9 | 30.8 |
| GF553 | | | | ++ | +++ | | | |
| GF374 | Glycodelin A | 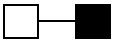 | N-glycan/LacdiNAc | 0.9 ± 0.0 +/− | 0.3 − | 1.80 ± 1.3 | 0.9 | 0.9 |
| GF375 | Glycodelin A | 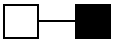 | N-glycan/LacdiNAc | 1.9 ± 0.71 − | 0.6 − | 5.85 ± 6.9 | 0.8 | 1.0 |
| GF376 | Glycodelin A | 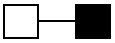 | N-glycan/LacdiNAc | 3.4 − | 0.6 − | 2.2 ± 0.85 | 1.8 | 1.4 |
| GF413 | Galα3Gal | 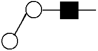 | Galα3Galβ4GlcNAc | 0.9 ± 0.42 | 0.8 | 7.45 ± 3.9 | 0.7 | 1.7 |
| GF295 GF555 | Lewis c | 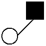 | pLN, Galβ3GlcNAc | 9.6 ± 7.4 − | 2.7 ± 2.5 − | 7.15 ± 2.8 | 1.9 | 17.2 |
| GF300 GF428 | asialo GM2 | 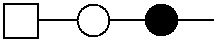 | GalNAcβ4Galβ4GlcβCer | 17.1 ± 3.3 − | 53.8 ± 2.1 − | 7.40 ± 3.4 | 47.9 | 63.4 |
| GF296 GF427 | asialo GM1 | 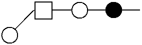 | Galβ3GalNAcβ4Galβ4GlcβCer | 22 ± 17.4 − | 48.2 ± 18.0 − | 10.30 ± 6.8 | 44.5 | 66.1 |
| GF624 | GD1b | 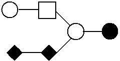 | | 3.5 ± 0.35 | ND | 7.4 ± 8.3 | 10.7 | 22.2 |
| GF623 | GT1b | 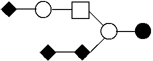 | | 30.7 ± 10.5 | ND | 20.85 ± 15.9 | 72.7 | 74.3 |
| GF406 GF558 | GD2 | 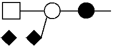 | GalNAcβ4(SAα8SAα3)Galβ4Glc | 0.9 ± 0.71 | 1.2 | 7.45 ± 7.6 | 1.4 | 20.6 |
| GF622 | GD2 | 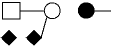 | GalNAcβ4(SAα8SAα3)Galβ4Glc | 50.8 ± 4.45 | ND | 5.25 ± 0.64 | 91.5 | 97.3 |
| GF625 | GD2 | 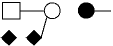 | GalNAcβ4(SAα8SAα3)Galβ4Glc | 44.2 ± 0.42 | ND | 7.2 ± 0.57 | 92.1 | 95.7 |
| GF407 GF559 | GD3 | 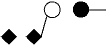 | SAα8SAα3Galβ4Glc | 0.8 | ND | 4.75 ± 0.92 | 1.4 | 58.3 |
| GF621 | GD3 | 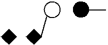 | SAα8SAα3Galβ4Glc | 18.4 ± 7.2 | ND | 2.8 ± 2.1 | 89.4 | 99 |
| GF626 | GD3 | 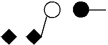 | SAα8SAα3Galβ4Glc | 2.9 ± 0.64 | ND | 1.95 ± 0.6 | 4.1 | 41.5 |
| VPU005 | GD3 | 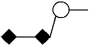 | SAα8SAα3Gal | 27.5 ± 4.45 | 29.9 | 10.1 ± 1.84 | 98.0 | 99.8 |
| GF627 | OAcGD3 | 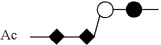 | Acetyl-SAα8SAα3Galβ4Glc | 0.6 ± 0.14 | ND | 1.35 ± 0.78 | 0.8 | 0.7 |
| GF628 | A2B5 | | | 27.6 ± 11.0 | ND | 37.2 ± 15.0 | 58 | 81 |
| GF298 | Gb3 | 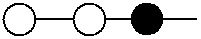 | Galα4Galβ4GlcβCer | 21.8 +++ | 52.7 ± 2.3 ++ | 6.15 ± 0.92 | 5.8 | 6.1 |
| GF297 VPU001 | Globoside GL4 | 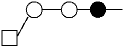 | GalNAcβ3Galα4Galβ4GlcβCer | 16.9 +++ | 28.4 ++ | 9.75 ± 4.2 | 30.1 | 61.2 |
| GF353 GF431 | SSEA-3 | 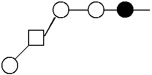 | Galβ3GalNAcβ3Gal | 3.4 ± 2.26 ++ | 6.2 ± 3.3 + | 1.95 ± 1.5 | 0.9 | 1.2 |
| VPU009 | SSEA-3 | 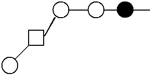 | Galβ3GalNAcβ3Gal | 11.9 ± 8.5 | ND | 75.75 ± 2.8 | 38.3 | 71.7 |
| GF354, GF432 VPU003 | SSEA-4 | 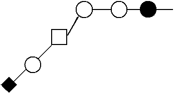 | SAα3Galβ3GalNAcβ3Galα4Galβ4Glc | 58.3 ± 23.6 +++ | 26.5 ± 18.0 +/− | 59.8 ± 0.57 | 32.6 | 80.5 |
| GF299 GF554 | Forssman ag | 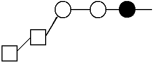 | GalNAcβ3GalNAcβ3Galα4Galβ4Glc | 4.1 − | 5.5 ± 1.7 − | 2.85 ± 2.1 | 0.4 | 2.4 |
| GF630 | Forssman ag | 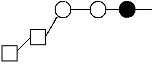 | GalNAcβ3GalNAcβ3Galα4Galβ4Glc | 0.3 | ND | 1.4 | 0.3 | 0.7 |
| GF288 | Globo-H | 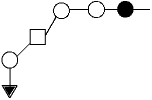 | Fucα2Galβ3GalNAcβ3Galα4Galβ4GlcβCer | 0.4 ± 0.07 − | 0.6 − | 1.35 ± 0.49 | 0.5 | 0.7 |
| GF394 | H disaccharide | 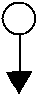 | Fucα2Galβ | 1.5 ± 0.42 − | 0.6 ± 0.14 − | 12.90 ± 8.9 | 0.6 | 0.5 |
| GF303 | H Type 1 | 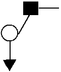 | Fucα2Galβ3GlcNAc | 1.4 ± 0.07 − | 0.7 ± 0.0 ++ | 1.2 ± 0.28 | 0.8 | 1.3 |
| GF304 GF429 | Lewis a | 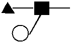 | Galβ3(Fucα4)GlcNAc | 13 ± 1.8 − | 23.4 ± 18.4 − | 11.3 ± 0.79 | 31.1 | 59.3 |
| GF306, GF430 VPU002 | sialyl Lewis a | 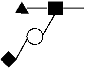 | SAα3Galβ3(Fucα4)GlcNAc | 3.0 ± 2.3 − | 5.1 ± 4.4 − | 7.6 ± 5.1 | 4.9 | 14.6 |
| GF629 | sialyl Lewis a | 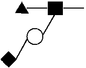 | SAα3Galβ3(Fucα4)GlcNAc | 0.5 | ND | 1.4 | 1.3 | 2.4 |
| GF301 VPU004 | Lewis b | 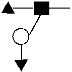 | Fucα2Galβ3(Fucα4)GlcNAc | 1.2 ± 0.0 − | 1.3 ± 0.49 − | 1.2 ± 0.85 | 0.7 | 1.4 |
| GF302 | H Type 2 | 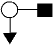 | Fucα2Galβ4GlcNAc | 14.7 ± 12.3 ++ | 26.2 ± 4.0 ++ | 9.4 ± 0.57 | 46.0 | 61.5 |
| GF410 | blood group ABH | 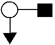 | Fucα2Galβ4GlcNAc | 0.4 ± 0.07 | 0.7 | 0.85 ± 0.21 | 0.7 | 0.7 |
| GF305 | Lewis x | 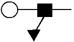 | Galβ4(Fucα3)GlcNAc | 1.0 +/− | 1.1 ± 0.49 − | 3.2 ± 2.5 | 0.8 | 3.0 |
| GF515 | Lewis x, CD15 | 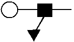 | Galβ4(Fucα3)GlcNAc | 0.3 ± 0.14 | 0.7 | 1.57 ± 0.49 | 0.7 | 2.9 |
| GF517 | Lewis x, CD15 | 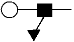 | Galβ4(Fucα3)GlcNAc | 0.3 ± 0.0 | 0.7 | 6.5 ± 8.7 | 0.5 | 2.4 |
| GF518 | SSEA-1 (CD15, Lex) | 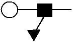 | Galβ4(Fucα3)GlcNAc | 0.3 ± 0.0 | 0.6 | 0.9 ± 0.14 | 1.0 | 1.8 |
| GF525 | CD15 (Lex), reacts with 220 kD protein | 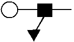 | Galβ4(Fucα3)GlcNAc | 1.1 ± 0.64 | 2.7 | 6.97 ± 2.4 | 2.5 | 48.3 |
| GF516 | sialyl Lewis x, sCD15 | 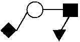 | SAα3Galβ4(Fucα3)GlcNAc | 8.5 ± 13.5 | 10.4 | 7.8 ± 5.9 | 19.0 | 13.5 |
| GF307 | sialyl Lewis x | 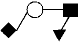 | SAα3Galβ4(Fucα3)GlcNAc | 82.1 ++ | 55.7 ± 9.4 + | 67.5 ± 4.6 | 12.6 | 49.1 |
| GF526 | PSGL-1, sLex on core 2 O-glycans | 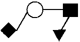 | SAα3Galβ4(Fucα3)GlcNAc | 90.8 ± 11.5 | 97.5 | 99.7 ± 0.12 | 98.6 | 99.9 |
| GF393 | Lewis y | 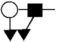 | Fucα2Galβ4(Fucα3)GlcNAcβ | 0.3 ± 0.0 − | 0.6 ± 0.0 − | 1.15 ± 0.92 | 1.0 | 0.8 |
| GF408 | blood group Ag A: (A1, A2) | 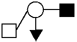 | GalNAcα3(Fucα2)GalβGlcNAc | 0.4 ± 0.21 | 0.6 | 1.40 ± 0.85 | 0.7 | 3.0 |
| GF409 | blood group A: (A3, Ax, A3B, AxB) | 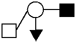 | GalNAcα3(Fucα2)GalβGlcNAc | 0.3 ± 0.0 | 0.5 | 0.95 ± 0.07 | 0.6 | 1.4 |
| GF411 | blood group B (secretor) | 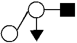 | Galα3(Fucα2)GalβGlcNAc | 0.8 ± 0.57 | 0.8 | 5.0 ± 2.7 | 2.1 | 13.5 |
| GF412 | blood group B (general) | 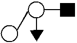 | Galα3(Fucα2)GalβGlcNAc | 3.3 ± 2.6 | 3.0 | 7.95 ± 0.07 | 18.2 | 58.9 |
| GF414 GF556 | TRA-1-81 Ag | | keratan sulphate in podocalyxin | 11.6 ± 13.8 | ND | 12.0 ± 0.71 | 10.4 | 69.7 |
| GF415 GF557 | TRA-1-60 Ag | | sialylated keratan sulphate in podocalyxin | 8.2 ± 10.6 | 2.6 | 10.9 ± 5.8 | 2.0 | 25.2 |
| GF377 | PN-15 renal gp200 | | | ND | ND | 5.35 ± 3.0 | 2.8 | 40.4 |

¹⁾Bone marrow/cord blood derived mesenchymal stem cells (BM/CB-MSC), ostegenic or adipocytic cells differentiated from MSC
(OG/adipo);
²⁾Code for IHC: −, negative; +/−, occasional low expression; +, low expression; ++, common; +++, abundant.

TABLE 27
MSC binder target table based on structural analyses and binder specificities. See
explanation of terms in footnotes 1) and 2).

| Trivial name | Terminal epitope | CB MSC | BM MSC | adipo diff. | osteo diff. | chondro diff. |
|---|---|---|---|---|---|---|
| LN type 1, Lec | Galβ3GlcNAcβ | + | + | + | +/− | q |
| | | L+ | L+ | Lq | L+ | Lq |
| | Lecβ3Galβ4Glc[NAc]β | +/− | +/− | q | +/− | q |
| Lea | Galβ3(Fucα4)GlcNAcβ | + | + | ++ | + | |
| | | L+/− | L+/− | | L+/− | |
| | Leaβ3Galβ4Glc[NAc]β | +/− | +/− | | +/− | |
| H type 1, H1 | Fucα2Galβ3GlcNAcβ | +/− | +/− | +/− | +/− | |
| | | L+ | L+ | | L+ | |
| | H1β3Galβ4Glc[NAc]β | +/− | +/− | | +/− | |
| Leb | Fucα2Galβ3(Fucα4)GlcNAcβ | +/− | +/− | +/− | +/− | |
| sialyl Lea, sLea | SAα3Galβ3(Fucα4)GlcNAcβ | +/− | +/− | ++ | + | |
| | | L+ | L+ | | L+ | |
| | sLeaβ3Galβ4Glc[NAc]β | +/− | +/− | | +/− | |
| α3′-sialyl Lec | SAα3Galβ3GlcNAcβ | +/− | +/− | ++ | + | q |
| | | Lq | Lq | Lq | Lq | |
| LN type 2, LN | Galβ4GlcNAcβ | + | ++ | + | ++ | + |
| | | N+ | N++ | N+ | N++ | N+ |
| | | O+ | O+ | O+ | O+ | O+ |
| | | Lq | Lq | Lq | Lq | Lq |
| | LNβ2Manα3/6 | + | ++ | + | ++ | + |
| | LNβ4Manα3 | +/− | +/− | + | ++ | + |
| | LNβ2Manα3(LNβ2Manα6)Man | + | + | + | + | + |
| | LNβ2(LNβ4)Manα3(LNβ2Manα6)Man | q | q | q | ++ | q |
| | LNβ6(R-Galβ3)GalNAc | + | + | + | + | + |
| | LNβ3Galβ4Glc[NAc]β | q | q | q | q | q |
| | LNβ6(R-GlcNAcβ3)Galβ4Glc[NAc]β | q | q | | q | |
| | LNβ3(R-GlcNAcβ6)Galβ4Glc[NAC]β | q | q | | q | |
| | LNβ3(LNβ6)Galβ4Glc[NAc]β | q | q | | q | |
| Lex | Galβ4(Fucα3)GlcNAcβ | +/− | + | + | +/− | q |
| | | L− | L− | | L− | |
| | Lexβ2Manα3/6 | q | q | q | q | q |
| | Lexβ6(R-Galβ3)GalNAc | q | q | q | q | |
| | Lexβ3Galβ4Glc[NAc]β | q | q | ++ | q | |
| | Lexβ2Manα3(Lexβ2Manα6)Man | q | q | q | q | |
| H type 2, H2 | Fucα2Galβ4GlcNAcβ | + | +/− | ++ | + | q |
| | | L+ | L+ | Nq | L+ | |
| | | Nq | Nq | | Nq | |
| | H2β2Manα3/6 | q | q | q | q | |
| | H2β3Galβ4Glc[NAc]β | + | + | | + | |
| Ley | Fucα2Galβ4(Fucα3)GlcNAcβ | +/− | +/− | +/− | +/− | |
| | | L+ | L+ | | L+ | |
| | Leyβ3Galβ4Glc[NAc]β | q | q | | q | |
| sialyl Lex, sLex | SAα3Galβ4(Fucα3)GlcNAcβ | ++ | ++ | ++ | ++ | q |
| | | O++ | O++ | O++ | O++ | |
| | | L− | L− | | L− | |
| | sLexβ2Manα3/6 | q | q | q | q | |
| | sLexβ6(R-Galβ3)GalNAc | ++ | ++ | ++ | ++ | |
| | sLexβ3Galβ4Glc[NAc]β | + | + | + | +/− | |
| α3′-sialyl LN, | SAα3Galβ4GlcNAcβ | + | + | + | + | + |
| s3LN | | N+ | N+ | N+ | N+ | N+ |
| | | O+ | O+ | O+ | O+ | O+ |
| | | Lq | Lq | Lq | Lq | Lq |
| | s3LNβ2Manα3/6 | + | + | + | + | + |
| | s3LNβ4Manα3 | +/− | +/− | + | ++ | + |
| | s3LNβ2Manα3(s3LNβ2Manα6)Man | + | + | + | + | + |
| | s3LNβ6(R-Galβ3)GalNAc | + | + | + | + | + |
| | s3LNβ3Galβ4Glc[NAc]β | + | + | + | + | + |
| | s3LNβ6(R-GlcNAcβ3)Galβ4Glc[NAc]β | q | q | | q | |
| | s3LNβ3(R-GlcNAcβ6)Galβ4Glc[NAc]β | q | q | | q | |
| α6′-sialyl LN, | SAα3Galβ4GlcNAcβ | q | q | q | q | q |
| s6LN | | Nq | Nq | Nq | Nq | Nq |
| | s6LNβ2Manα3/6 | q | q | q | q | q |
| | s6LNβ4Manα3 | q | q | q | q | q |
| | s6LNβ2Manα3(s6LNβ2Manα6)Man | q | q | q | q | q |
| | s6LNβ3Galβ4Glc[NAc]β | − | − | − | − | − |
| Core 1 | Galβ3GalNAcα | +/− | +/− | +/− | +/− | q |
| H type 3 | Fucα2Galβ3GalNAcα | − | − | − | − | − |
| sialyl Core 1 | SAα3Galβ3GalNAcα | + | + | + | q | q |
| disialyl Core 1 | SAα3Galβ3Saα6GalNAcα | + | + | + | q | q |
| type 4 chain | Galβ3GalNAcβ | +/− | +/− | ++ | +/− | q |
| | | L+ | L+ | | L+ | |
| asialo-GMI | Galβ3GalNAcβ4Galβ4Glc | +/− | + | ++ | ++ | |
| Gb5, “SSEA-3” | Galβ3GalNAcβ3Galα4Galβ4Glc | + | +/− | + | +/− | |
| H type4,“Globo H” | Fucα2Galβ3GalNAcβ | q | q | +/− | q | |
| | | L+/− | L+/− | | L+/− | |
| α3′-sialyl type 4 | SAα3Galβ3GalNAcβ | ++ | ++ | q | + | q |
| | | L+ | L+ | | L+ | |
| “SSEA-4” | SAα3Galβ3GalNAcβ3Galα4Galβ4Glc | ++ | ++ | ++ | + | q |
| GalNAcβ | GalNAcβ | +/− | + | ++ | ++ | q |
| asialo-GM2 | GalNAcβ4Galβ4Glc | +/− | + | ++ | ++ | |
| Gb4 | GalNAcβ3Galα4Galβ4Glc | + | + | | ++ | |
| LacdiNAc | GalNAcβ4GlcNAcβ | | | | | |
| Galα | Galβ4Glc | +/− | +/− | +/− | +/− | q |
| Gb3 | Galα4Galβ4Glc | + | + | + | ++ | q |
| Lac | Galβ4Glc | q | q | q | q | q |
| GalNAcα, “Tn” | GalNAcα | +/− | +/− | + | q | q |
| Forssman | GalNAcα3GalNAcβ | +/− | q | q | q | |
| sialyl Tn | SAα6GalNAcα | +/− | q | + | q | q |
| oligosialic acid | NeuAcα8NeuAcα | + | + | ++ | ++ | q |
| | | L+ | L+ | L++ | L++ | |
| GD3 | NeuAcα8NeuAcα2Galβ4Glc | + | + | ++ | ++ | |
| GD2 | NeuAcα8NeuAcα2(GalNAcβ4)Galβ4Glc | ++ | + | ++ | ++ | |
| GD1b | NeuAcα8NeuAcα2(Galβ3GalNAcβ4)Galβ4Glc | +/− | q | ++ | +/− | |
| GT1b | SAα8SAα2(Saα3Galβ3GalNAcβ4)Galβ4Glc | + | + | ++ | ++ | |
| Manα | Manα | ++ | ++ | ++ | ++ | ++ |
| | Manα2Manα | ++ | ++ | + | + | + |
| | Manα3Manα6/β4 | + | + | ++ | + | ++ |
| | Manα6Manα6/β4 | + | + | ++ | + | ++ |
| | Manα3(Manα6)Manα6/β4 | + | + | ++ | + | ++ |
| | Manα3(Manα6)Manβ4GlcNAc[β4GlcNAc] | N+/− | N+/− | N++ | N+ | Nq |
| Manβ | Manβ | +/− | +/− | + | +/− | + |
| | Manβ4GlcNAc | +/− | +/− | + | +/− | + |
| Glcα | Glcα | + | + | +/− | +/ | +/ |
| | Glcα3Manα | + | + | +/ | +/ | +/ |
| | Glcα2Glcα3[Glcα3Manα] | +/− | +/− | +/ | +/ | +/ |
| core-Fuc | Fucα6GlcNAc | N+ | N+ | N+ | N+/− | N+ |
| | Fucα6(R-GlcNAcβ4)GlcNAc | + | + | + | +/− | + |
| GlcNAcβ, Gn | GlcNAcβ | + | + | + | +/− | +/− |
| | | N+ | N+ | N+ | Nq | Nq |
| | Gnβ2Manα3/6 | + | + | + | q | q |
| | Gnβ4Manα3 | + | + | q | | |
| | Gnβ2Manα3(Gnβ2Manα6)Man | + | + | q | q | q |
| | Gnβ4Gn | q | q | q | q | q |
| | Gnβ4(Fucα6)Gn | q | q | q | q | q |
| | Gnβ6(R-Galβ3)GalNAc | − | − | − | − | − |
| | Gnβ3Galβ4Glc[NAc]β | q | q | q | q | q |
| | Gnβ6(R-GlcNAcβ3)Galβ4Glc[NAc]β | q | q | | q | |
| | Gnβ3(R-GlcNAcβ6)Galβ4Glc[NAc]β | q | q | | q | |

1) Stem cell and differentiated cell types are abbreviated as in other parts of the present document; CB/BM indicates MSC derived from cord blood or bone marrow; adipo/osteo/chondro diff. indicates cells differentiated into adipocyte, osteoblast, or chondrocyte direction from MSC.
2) Occurrence of terminal epitopes in glycoconjugates and/or specifically in N-glycans (N), O-glycans (O), and/or glycosphingolipids (L).
Code:
q, qualitative data;
+/−, low expression;
+, common;
++, abundant.

TABLE 28
Comparison of neutral N-glycan profiles of adipocyte-differentiated
cells and cord blood MSC; relat. = relation of adipocyte-
differentiated cell glycan signals to MSC glycan signals, wherein larger
number indicates differentiation-association and vice versa; structure
indicates N-glycan structure classification according to the
present invention.

| AD relat. | comp. | structure | | | | m/z |
|---|---|---|---|---|---|---|
| new | H3N1 | S | | | | 730 |
| new | H2N1 | S | | | | 568 |
| new | H4N4F1 | C | | F | Q | 1647 |
| new | H6N3F1 | H | | F | | 1768 |
| new | H4N4F2 | C | | E | Q | 1793 |
| new | H3N5 | C | | | T | 1542 |
| new | H3N4 | C | | | T | 1339 |
| new | H8N2F1 | M | | F | | 1889 |
| new | H1N2F2 | O | | E | | 901 |
| new | H7N3 | H | | | | 1784 |
| new | H2N2F4 | O | | E | | 1355 |
| new | H4N5F2 | C | | E | T | 1996 |
| new | H7N4 | C | | | X | 1987 |
| 3.15 | H5N2F1 | M | | F | | 1403 |
| 2.47 | H3N3 | H | N | | | 1136 |
| 1.94 | H5N4 | C | B | | | 1663 |
| 1.68 | H6N4 | C | | | X | 1825 |
| 1.54 | H4N2F1 | L | | F | | 1241 |
| 1.45 | H5N2 | M | | | | 1257 |
| 1.13 | H2N2F1 | L | | F | | 917 |
| 1.10 | | | | | | 1555 |
| 1.03 | H5N3 | H | | | | 1460 |
| 0.94 | H3N3F1 | H | N | F | | 1282 |
| 0.90 | H3N2 | L | | | | 933 |
| 0.84 | H6N3 | H | | | | 1622 |
| 0.79 | H4N2 | L | | | | 1095 |
| 0.69 | H5N4F1 | C | B | F | | 1809 |
| 0.67 | H3N2F1 | L | | F | | 1079 |
| 0.55 | H4N4 | C | | | Q | 1501 |
| 0.46 | H4N3F1 | H | | F | | 1444 |
| 0.41 | H4N3 | H | | | | 1298 |
| 0.15 | H6N2F1 | M | | F | | 1565 |
| 0.08 | H2N2 | L | | | | 771 |
| 0.06 | H6N2 | M | | | | 1419 |
| 0.01 | H7N2 | M | | | | 1581 |
| −0.16 | H8N2 | M | | | | 1743 |
| −0.16 | H5N3F1 | H | | F | | 1606 |
| −0.24 | H4N1 | S | | | | 892 |
| −0.27 | | | | | | 1717 |
| −0.28 | H3N4F1 | C | | F | T | 1485 |
| −0.30 | H5N4F3 | C | B | E | | 2101 |
| −0.37 | H6N5 | C | R | | | 2028 |
| −0.43 | H9N2 | M | | | | 1905 |
| −0.49 | | | | | | 2041 |
| −0.54 | H1N2F1 | L | | F | | 755 |
| −0.65 | H5N4F2 | C | B | E | | 1955 |
| −0.66 | H8N1 | S | | | | 1540 |
| −0.70 | H6N5F1 | C | R | F | | 2174 |
| −0.71 | H6N4F1 | C | | F | X | 1971 |
| −0.73 | H5N1 | S | | | | 1054 |
| −0.74 | | | | | | 1031 |
| −0.74 | H10N2 | M | G | | | 2067 |
| −0.80 | H6N1 | S | | | | 1216 |
| −0.84 | H3N5F1 | C | | F | T | 1688 |
| −0.87 | H9N1 | S | | | | 1702 |
| lost | H2N4F1 | O | | F | T | 1323 |
| lost | H1N3F1 | O | | F | T | 958 |
| lost | H7N1 | S | | | | 1378 |

TABLE 29
Comparison of neutral N-glycan profiles of osteoblast-
differentiated cells and cord blood MSC; relat. = relation of
adipocyte-differentiated cell glycan signals to MSC glycan
signals, wherein larger number indicates differentiation-association
and vice versa; structure indicates N-glycan structure classification
according to the present invention.

| OG relat. | comp. | structure | | | | m/z |
|---|---|---|---|---|---|---|
| new | H3N1 | S | | | | 730 |
| new | H7N3 | H | | | | 1784 |
| new | H6N3F1 | H | | F | | 1768 |
| 3.59 | | | | | | 1555 |
| 2.40 | H5N3 | H | | | | 1460 |
| 2.22 | H6N3 | H | | | | 1622 |
| 1.91 | H5N2 | M | | | | 1257 |
| 1.75 | H3N3 | H | N | | | 1136 |
| 1.28 | H3N2 | L | | | | 933 |
| 1.15 | H4N1 | S | | | | 892 |
| 1.12 | H4N2 | L | | | | 1095 |
| 0.80 | H2N2 | L | | | | 771 |
| 0.79 | H4N4 | C | | | Q | 1501 |
| 0.34 | | | | | | 1717 |
| 0.12 | H6N2 | M | | | | 1419 |
| 0.11 | H4N3 | H | | | | 1298 |
| 0.10 | H7N2 | M | | | | 1581 |
| 0.09 | H4N3F1 | H | | F | | 1444 |
| 0.03 | | | | | | 1031 |
| −0.08 | | | | | | 2041 |
| −0.25 | H9N2 | M | | | | 1905 |
| −0.28 | H5N1 | S | | | | 1054 |
| −0.28 | H8N2 | M | | | | 1743 |
| −0.28 | H5N4 | C | B | | | 1663 |
| −0.39 | H10N2 | M | G | | | 2067 |
| −0.39 | H5N4F1 | C | B | F | | 1809 |
| −0.41 | H6N2F1 | M | | F | | 1565 |
| −0.47 | H6N1 | S | | | | 1216 |
| −0.48 | H5N3F1 | H | | F | | 1606 |
| −0.51 | H6N5 | C | R | | | 2028 |
| −0.57 | H8N1 | S | | | | 1540 |
| −0.81 | H7N1 | S | | | | 1378 |
| −0.81 | H3N2F1 | L | | F | | 1079 |
| lost | H5N2F1 | M | | F | | 1403 |
| lost | H6N4 | C | | | X | 1825 |
| lost | H2N4F1 | O | | F | T | 1323 |
| lost | H4N2F1 | L | | F | | 1241 |
| lost | H6N4F1 | C | | F | X | 1971 |
| lost | H5N4F3 | C | B | E | | 2101 |
| lost | H1N3F1 | O | | F | T | 958 |
| lost | H3N3F1 | H | N | F | | 1282 |
| lost | H6N5F1 | C | R | F | | 2174 |
| lost | H1N2F1 | L | | F | | 755 |
| lost | H3N5F1 | C | | F | T | 1688 |
| lost | H5N4F2 | C | B | E | | 1955 |
| lost | H3N4F1 | C | | F | T | 1485 |
| lost | H9N1 | S | | | | 1702 |
| lost | H2N2F1 | L | | F | | 917 |

TABLE 30
Comparison of neutral N-glycan profiles of chondrocyte-
differentiated cells and cord blood MSC; relat. = relation of
adipocyte-differentiated cell glycan signals to MSC glycan signals,
wherein larger number indicates differentiation-association and vice
versa; structure indicates N-glycan structure classification according
to the present invention.

| CH relat. | comp. | structure | | | | m/z |
|---|---|---|---|---|---|---|
| new | H3N1 | S | | | | 730 |
| new | H4N4F1 | C | | F | Q | 1647 |
| new | H1N2F2 | O | | E | | 901 |
| new | H7N3 | H | | | | 1784 |
| new | H6N3F1 | H | | F | | 1768 |
| new | | | | | | 1393 |
| new | H4N4F2 | C | | E | Q | 1793 |
| new | H11N2 | M | G | | | 2229 |
| new | H9N8 | C | R | | | 3124 |
| new | H6N6 | C | R | | Q | 2231 |
| 4.01 | H5N2F1 | M | | F | | 1403 |
| 2.97 | H5N4 | C | B | | | 1663 |
| 2.53 | H5N4F1 | C | B | F | | 1809 |
| 2.51 | H3N3 | H | N | | | 1136 |
| 2.39 | | | | | | 1555 |
| 2.23 | H3N2F1 | L | | F | | 1079 |
| 2.09 | H4N2F1 | L | | F | | 1241 |
| 1.80 | H5N2 | M | | | | 1257 |
| 1.50 | H5N3 | H | | | | 1460 |
| 1.31 | H4N1 | S | | | | 892 |
| 1.21 | H4N3F1 | H | | F | | 1444 |
| 0.96 | H3N2 | L | | | | 933 |
| 0.86 | H4N3 | H | | | | 1298 |
| 0.80 | H2N2F1 | L | | F | | 917 |
| 0.78 | H3N3F1 | H | N | F | | 1282 |
| 0.77 | H6N3 | H | | | | 1622 |
| 0.62 | H4N2 | L | | | | 1095 |
| 0.28 | H5N4F3 | C | B | E | | 2101 |
| 0.17 | H6N4 | C | | | X | 1825 |
| 0.10 | H5N1 | S | | | | 1054 |
| 0.08 | H6N2 | M | | | | 1419 |
| −0.08 | H7N2 | M | | | | 1581 |
| −0.11 | H5N4F2 | C | B | E | | 1955 |
| −0.22 | H6N2F1 | M | | F | | 1565 |
| −0.24 | H6N1 | S | | | | 1216 |
| −0.25 | H3N4F1 | C | | F | T | 1485 |
| −0.30 | H6N5F1 | C | R | F | | 2174 |
| −0.31 | H6N5 | C | R | | | 2028 |
| −0.32 | H1N2F1 | L | | F | | 755 |
| −0.35 | H8N2 | M | | | | 1743 |
| −0.36 | | | | | | 1031 |
| −0.44 | H4N4 | C | | | Q | 1501 |
| −0.47 | H10N2 | M | G | | | 2067 |
| −0.48 | H8N1 | S | | | | 1540 |
| −0.49 | | | | | | 1717 |
| −0.52 | H9N2 | M | | | | 1905 |
| −0.55 | H2N2 | L | | | | 771 |
| −0.58 | H9N1 | S | | | | 1702 |
| −0.63 | H7N1 | S | | | | 1378 |
| −0.64 | H5N3F1 | H | | F | | 1606 |
| −0.77 | | | | | | 2041 |
| lost | H2N4F1 | O | | F | T | 1323 |
| lost | H6N4F1 | C | | F | X | 1971 |
| lost | H1N3F1 | O | | F | T | 958 |
| lost | H3N5F1 | C | | F | T | 1688 |

### REFERENCES

Altmann, F., et al. (1999) Glycoconj. J. 16:109-23

Harvey, D. J., et al. (1993) Rapid Commun. Mass Spectrom. 7(7):614-9

Hirabayashi, J., et al. (2002) Biochim. Biophys. Acta. 1572:232-54.

Jaatinen, T., et al. (2006) Stem cells. 24:631-41.

Karlsson, H., et al. (2000) Glycobiology 10(12):1291-309

Kretzchmar, E., et al. (1994) Biol. Chem. Hoppe Seyler 375(5):23-7

Kubelka, V., et al. (1994) Arch. Biochem. Biophys. 308(1):148-57

Leskelä, H., et al. (2003) Biochem. Biophys. Res. Commun. 311:1008-13

Miller-Podraza, H., et al. (2000) Glycobiologvy. 10:975-982

Moore (1999) Trends Cell Biol. 9:441-6

Naven, T. J. & Harvey, D. J. (1996) Rapid Commun. Mass Spectrom. 10(1 1):1361-6

Nyman, T. A., et al. (1998) Eur. J. Biochem. 253(2):485-93

Papac, D., et al. (1996) Anal. Chem. 68(18):32i5-23

Saarinen, J., et al. (1999) Eur. J. Biochem. 259(3):829-40

Skottman, H. et al. (2005) Stem cells

Staudacher, E., et al. (1992) Eur. J. Biochem. 207(3):987-93

Thoomson, J. A., et al. (1998) Science 282:1145-7

Venable et al. (2005) BMC Developmental biology.

1-35. (canceled)

36. A method of evaluating the status of a mesenchymal cell preparation comprising the step of detecting the presence of an elongated glycan structure or a group, at least two, of glycan structures in said preparation, wherein said glycan structure or a group of glycan structures is according to Formula T1 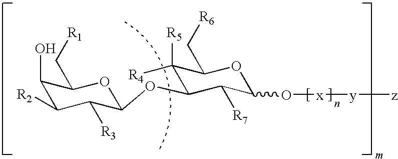
wherein
R₁, R₂, and R₆ are OH or glycosidically linked monosaccharide residue sialic acid, preferably Neu5Acα2 or Neu5Gcα2, most preferably Neu5Acα2;
R₃, is OH or glycosidically linked monosaccharide residue Fucα1 (L-fucose) or N-acetyl (N-acetamido, NCOCH₃);
R₄, is H, OH or glycosidically linked monosaccharide residue Fucα1 (L-fucose),
R₅ is OH, when R₄ is H, and R₅ is H, when R₄ is not H;
R7 is N-acetyl or OH; and
X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0; and
Z is a carrier structure, preferably natural carrier produced by the cells, such as protein or lipid, which is preferably a ceramide or branched glycan core structure on the carrier or H;
the arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
with the provisions that one of R2 and R3 is OH or R3 is N-acetyl,
R6 is OH, when the first residue on left is linked to position 4 of the residue on right: and
the glycan structure is an elongated structure, wherein the binder binds to the structure and additionally to at least one reducing end elongation epitope, which is a monosaccharide epitope replacing X or being a part of X, said monosaccharide epitope being according to Formula E1:
AxHex(NAc)ₙ, wherein A is anomeric structure alfa or beta, x is linkage position 2, 3, or 6; and
Hex is hexopyranosyl residue Gal, or Man, and n is integer being 0 or 1,
with the provisions that
when n is 1 then AxHexNAc is β4GalNAc or β6GalNAc,
when Hex is Man, then AxHex is β2Man, and
when Hex is Gal, then AxHex is β3Gal or β6Gal or α3Gal or α4Gal; or
the binder epitope binds additionally to reducing end elongation epitope
Ser/Thr linked to reducing end GalNAcα-comprising structures or
βCer linked to Galβ4Glc comprising structures, and the glycan structure is the stem cell population determined structure or from associated or contaminating cell population,
and optionally wherein the structure is used together with at least one terminal ManαMan-structure.

37. The method according to claim 36, wherein terminal epitope selected from the group Galβ4Glc, Galβ3GlcNAc, Galβ3GalNAc, Galβ4GlcNAc, Galβ3GlcNAcβ, Galβ3GalNAcβ/α, Galβ4GlcNAcβ, GalNAcβ4GlcNAc, SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, SAα3Galβ4GlcNAcβ, SAα6Galβ4Glc, SAα6Galβ4Glcβ, SAα6Galβ4GlcNAc, SAα6Galβ4GlcNAcβ, Galβ3(Fucα4)GlcNAc (Lewis a), SAα3Galβ3(Fucα4)GlcNAc (sialyl-Lewis a), Fucα2Galβ3GlcNAc (H-type 1), Fucα2Galβ3(Fucα4)GlcNAc (Lewis b), Galβ4GlcNAc (type 2 lactosamine based), Galβ4(Fucα3)GlcNAc (Lewis x), SAα3Galβ3(Fucα4)GlcNAc (sialyl-Lewis x), Fucα2Galβ4GlcNAc (H-type 2) and Fucα2Galβ4(Fucα3)GlcNAc (Lewis y),
linked to an elongation structure according to Formula E1:
AxHex(NAc)ₙ, wherein A is anomeric structure alfa or beta, x is linkage position 2, 3, or 6; and Hex is hexopyranosyl residue Gal, or Man, and n is integer being 0 or 1,
with the provisions that
when n is 1 then AxHexNAc is β6GalNAc,
when Hex is Man, then AxHex is β2Man, and
when Hex is Gal, then AxHex is β3Gal or β6Gal,

38. The method according to claim 36, wherein said binding agent recognizes structure according to the Formula T8Ebeta
[Mα]ₘGalβ1-3/4[Nα]ₙGlcNAcβxHex(NAc)ₚ
wherein x is linkage position 2, 3, or 6;
m, n and p are integers 0, or 1, independently; and
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc,
with the provision that m, n and p are 0 or 1, independently.
Hex is hexopyranosyl residue Gal, or Man,
with the provisions that when p is 1 then βxHexNAc is β6GalNAc,
when p is 0
then Hex is Man and βxHex is β2Man, or Hex is Gal and βxHex is β3Gal or β6Gal.

39. The method according to claim 36, wherein said binding agent recognizes type II
Lactosmine based structures according to the
[Mα]ₘGalβ1-4[Nα]ₙGlNAcβxHex(NAc)ₚ Formula T10E
with the provisions that when p is 1 then βxHexNAc is β6GalNAc,
when p is 0, then Hex is Man and βxHex is β2Man, or Hex is Gal and βxHex is β6Gal.

40. The method according to claim 39, wherein said binding agent recognizes type II
Lactosmine based structures according to the
[Mα]ₘGalβ1-4[Nα]ₙGlcNAcβ2Man, Formula T10EMan
wherein m and n are integers 0 or 1, independently; and
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc, when Gal is linked to the other position (4 or 3) of GlcNAc.

41. The method according to claim 39, wherein said binding agent recognizes type II
Lactosmines according to the
[Mα]ₘGalβ1-4[Nα]ₙGlcNAcβ6Gal(NAc)ₚ Formula T10EGal(NAc)
wherein m, n and p are integers 0 or 1, independently; and
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc.

42. The method according to claim 41, wherein the structure is O-glycan core II sialyl-Lewis x structure SAα3Galβ4(Fucα3)GlcNAcβ6(RGalβ3)GalNAc and it is recognized by antibody CHO131, and optionally wherein the antibody recognized over 50% of the mesenchymal cells.

43. The method according to claim 36, wherein said binding agent recognizes type I
Lactosamine based structures according to the
[Mα]ₘGalβ1-3[Nα]ₙGlcNAcβ3Gal Formula T9E

44. The method according to claim 36, wherein said binding agent recognizes type II
Lactosmine based structures according to the
Formula
[Mα]ₘGalβ1-4[Nα]ₙGlcNAcβ3Gal

45. The method of claim 44, wherein the structure is SAα3Galβ4(Fucα3)GlcNAcβ3Gal to analyze the status of mesenchymal cells using antibody antibody KM93 or CSLEX.

46. The method according to claim 36, wherein the detection is performed by a binder being a recombinant protein selected from the group consisting of monoclonal antibody, glycosidase, glycosyl transferring enzyme, plant lectin, animal lectin and a peptide mimetic thereof.

47. The method according to the claim 36, wherein the binder is used for sorting or selecting human stem cells from biological materials or samples including cell materials comprising other cell types.

48. A cell population obtained by the method according to claim 47.

49. The method according to any of claims 36, wherein the glycan structure is present in a O-glycan subglycome comprising O-Glycans with O-glycan core structure, or the glycan structure is present in a glycolipid subglycome comprising glycolipids with glycolipid core structure and the glycans are releasable by glycosylceramidase or in a N-glycan subglycome comprising N-Glycans with N-glycan core structure and said N-Glycans being releasable from cells by N-glycosidase.

50. The method according to claim 36 wherein the presence or absence of cell surface glycomes of said cell preparation is detected.

51. The method according to claim 36, wherein said cell preparation is evaluated/detected with regard to a contaminating structure in a cell population of said cell preparation, time dependent changes or a change in the status of the cell population by glycosylation analysis using mass spectrometric analysis of glycans in said cell preparation.

52. The method evaluate mesenchymal cells with regard to two terminal epitopes as defined by Formula I in the claim 36, wherein the one of the following combinations of binder reagents are used, said reagents recognizing type I and type II acetyllactosamines and fucosylated variants or non-sialylated facosylated variants thereof; or fucosylated type I and type II N-acetyllactosamine structures preferably comprising Fucα2-terminal and/or Fucα3/4-branch structure; or fucosylated type I and type II N-acetyllactosamine structures preferably comprising Fucα2-terminal.

53. A composition comprising glycan structure as defined in claim 36 derived from a stem cell and a binder that binds to said glycan structure.

54. The composition according to the claim 53, wherein the composition is used in method for identifying a selective stem cell binder to said glycan structure, which comprises:
selecting a glycan structure exhibiting specific expression in/on stem cells and absence of expression in/on feeder cells and/or differentiated somatic cells; and
confirming the binding of the binder to the glycan structure in/on stem cells.

55. The composition according to the claim 53, wherein the composition is part of a kit for enrichment and detection of stem cells within a specimen, comprising: at least one reagent comprising a binder to detect said glycan structure; and instructions for performing stem cell enrichment using the reagent, optionally including means for performing stem cell enrichment or wherein the composition is for isolation of cellular components from stem cells comprising the novel target/marker structures.
